SEQUENCE LISTING (194 sequences)

SEQ ID NO: 1 - DNA, 1168 bp, Homo sapiens
  misc_feature: Incyte ID No 184081.24
aaggaagattcggccactcgttggttctgcagcctcatcagtttctcagcatgttccctctcctcatgagattggtgaagaaagtatttggcaaagttcttcaaagccacatcatcgcggtcaaagtagtaagacatggacaggtaaacgtaggaggcgtagagctccaggttgatctggcggttgatggcggcctctgagtcctggtggtagttctggcgcacctgcgaggtggctggaaagctttccagcaaatgcactgtaggtagaaggcagaggaagcccttatttagcaatgcagaacttggcagaggccccacatctgtcattcttcacagcagtcccttcccacatgctagagggaaggggaagcatgatagggaggtccacttttgtggactcaaaccttgatggggatgttgagcagtcacaacgcttctcagaaaaggcacaagcaccccagacattcaggcccggagaacaggctggctcagcaggtcttcacgatcgggtgtctcgagcccttcttcgggaacgagggccacagctggagctgggcatggaacccaggccagggggtgctcctgggggttggtaaccacactcattggggtccagggggtcccggctgagcagcacccacactgcaggcacatggcggcgcactgtgagggggtctagccctgtgtcaggtgaggctgggacccagctgtcctcagtctgaaggaaacgtagcttggtgagctggtgcaggcctgggacccgccgcttgacaatctcagcgcagctgacagcctttcctgcagccctgccagaacctgagaacactacatgccgagcactgccgccctccaaccgacccagagccagctttccggtagtgctccatcctgcttgctgtcttgtctgtgactctcgctgcccacaaggtcaatctatatcaacccaggtcactcagaaactcgggggcagaggctcgactcggagggctgctcaggacattccaagacagccttggccgtttccatagccacggtctcttccagggtcttgccggccgctgcagccaagccacaggcgtcccccaaccctacgagcgcccctctgcccgcccggttccggctctcaccgaagccgcctcacgcacgatccggagccaagatggcgc

SEQ ID NO: 2 - DNA, 404 bp, Homo sapiens
  misc_feature: Incyte ID No 995839.2
cctccaagtcccagcgaacccgcgtgcaacctgtcccgactctagccgcctcttcagctcgccatggatcccaactggctcctgcgccgccggtgactcctgcacctgcgccggctcctgcaaatgcaaagagtggcaaatgcacctcctgcaagaaaagctgctgctcctgctgccctgtgggctgtgccaagtgtgcccagggctgcatctgcaaaggggcgtcggacaagtgcagctgctgcgcctgatgctgggacagccccgctcccagatgtaaagaacgcgacttccacaaacctggattttttatgtacaaccctgaaccgtggaccgtttgctatattccctttttctatgganataatgtggaatggataatanaaacagcttttgacttgaaa

SEQ ID NO: 3 - DNA, 1015 bp, Homo sapiens
  misc_feature: Incyte ID No 3200830CB1
ctcgcgccttcgtttattcctccgcgcgctgggacaggctgcttcttcgccagaaccaaccggttgcttgctgtcccagcggcgccccctcatcaccgtcgccatgcccggaggtctgcttctcggggacgtggctcccaactttgaggccaataccaccgtcggccgcatccgtttccacgactttctgggagactcatggggcattctcttctcccaccctcgggactttaccccagtgtgcaccacagagcttggcagagctgcaaagctggcaccagaatttgccaagaggaatgttaagttgattgccctttcaatagacagtgttgaggaccatcttgcctggagcaaggatatcaatgcttacaattgtgaagagcccacagaaaagttaccttttcccatcatcgatgataggaatcgggagcttgccatcctgttgggcatgctggatccagcagagaaggatgaaaagggcatgcctgtgacagctcgtgtggtgtttgtttttggtcctgataagaagctgaagctgtctatcctctacccagctaccactggcaggaactttgatgagattctcagggtagtcatctctctccagctgacagcagaaaaaagggttgccaccccagttgattggaaggatggggatagtgtgatggtccttccaaccatccctgaagaagaagccaaaaaacttttcccgaaaggagtcttcaccaaagagctcccatctggcaagaaatacctccgctacacaccccagccttaagtctcttggagaagctggtgctgtgagccagaggatgtcagctgccaattgtgttttcctgcagcaattccataaacacatcctggtgtcatcacagccaaggtttttaggttgctataccaatggcttattaaatgaaaatggcactaaaagtttcttgagattctttatactctctgccttcagcaatcaattccattcatacatcagcactctgctggttctgtttgaaatatg

SEQ ID NO: 4 - protein, 224 aa, Homo sapiens
  misc_feature: Incyte ID No 3200830CD1
MPGGLLLGDVAPNFEANTTVGRIRFHDFLGDSWGILFSHPRDFTPVCTTELGRAAKLAPEFAKRNVKLIALSIDSVEDHLAWSKDINAYNCEEPTEKLPFPIIDDRNRELAILLGMLDPAEKDEKGMPVTARVVFVFGPDKKLKLSILYPATTGRNFDEILRVVISLQLTAEKRVATPVDWKDGDSVMVLPTIPEEEAKKLFPKGVFTKELPSGKKYLRYTPQP

SEQ ID NO: 5 - DNA, 457 bp, Homo sapiens
  misc_feature: Incyte ID No 006512.8
atcatcgatgataggaatcgggagcttgccatcctgttgggcatgctggatccagcagagaaggatgaaaagggcatgcctgtgacagctcgtgtggtgtttgtttttggtcctgataagaagctgaagctgtctatcctctacccagctaccactggcaggaactttgatgagattctcagggatggggatagtgtgatggtccttccaaccatccctgaagaagaagccaaaaaaacttttcccgaaaggagtcttcaccaaagagctcccatctggcaagaaatacctccgctacacaccccagccttaagtctcttggagaagctggtgctgtgagccagaggatgtcagctgccaattgtgttttcctgcagcaattccataaacacatcctggtgtcatcacagccaagtttttaggtgtatacaaatggttattaaatgaagatatacga

SEQ ID NO: 6 - DNA, 2365 bp, Homo sapiens
  misc_feature: Incyte ID No 3819039CB1
cggacgcgtgggcgagccggtctttgagcgctaacgtctttctgtctccccgcggtggtgatgacggtgaaaactgaggctgctaagggcaccctcacttactccaggatgaggggcatggtggcaattctcatcgctttcatgaagcagaggaggatgggtctgaacgactttattcagaagattgccaataactcctatgcatgcaaacaccctgaagttcagtccatcttgaagatctcccaacctcaggagcctgagcttatgaatgccaacccttctcctccaccaagtccttctcagcaaatcaaccttggcccgtcgtccaatcctcatgctaaaccatctgactttcacttcttgaaagtgatcggaaagggcagttttggaaaggttcttctagcaagacacaaggcagaagaagtgttctatgcagtcaaagttttacagaagaaagcaatcctgaaaaagaaagaggagaagcatattatgtcggagcggaatgttctgttgaagaatgtgaagcaccctttcctggtgggccttcacttctctttccagactgctgacaaattgtactttgtcctagactacattaatggtggagagttgttctaccatctccagagggaacgctgcttcctggaaccacgggctcggttctatgctgctgaaatagccagtgccttgggctacctgcattcactgaacatcgtttatagagacttaaaaccagagaatattttgctagattcacagggacacattgtccttactgacttcggactctgcaaggagaacattgaacacaacagcacaacatccaccttctgtggcacgccggagtatctcgcacctgaggtgcttcataagcagccttatgacaggactgtggactggtggtgcctgggagctgtcttgtatgagatgctgtatggcctgccgcctttttatagccgaaacacagctgaaatgtacgacaacattctgaacaagcctctccagctgaaaccaaatattacaaattccgcaagacacctcctggagggcctcctgcagaaggacaggacaaagcggctcggggccaaggatgacttcatggagattaagagtcatgtcttcttctccttaattaactgggatgatctcattaataagaagattactcccccttttaacccaaatgtgagtgggcccaacgacctacggcactttgaccccgagtttaccgaagagcctgtccccaactccattggcaagtcccctgacagcgtcctcgtcacagccagcgtcaaggaagctgccgaggctttcctaggcttttcctatgcgcctcccacggactctttcctctgaaccctgttagggcttggttttaaaggattttatgtgtgtttccgaatgttttagttagccttttggtggagccgccagctgacaggacatcttacaagagaatttgcacatctctggaagcttagcaatcttattgcacactgttcgctggaagctttttgaagagcacattctcctcagtgagctcatgaggttttcatttttattcttccttccaacgtggtgctatctctgaaacgagcgttagagtgccgccttagacggaggcaggagtttcgttagaaagcggacgctgttctaaaaaaggtctcctgcagatctgtctgggctgtgatgacgaatattatgaaatgtgccttttctgaagagattgtgttagctccaaagcttttcctatcgcagtgtttcagttctttattttcccttgtggatatgctgtgtgaaccgtcgtgtgagtgtggtatgcctgatcacagatggattttgttataagcatcaatgtgacacttgcaggacactacaacgtgggacattgtttgtttcttccatatttggaagataaatttatgtgtagacttttttgtaagatacggttaataactaaaatttattgaaatggtcttgcaatgactcgtattcagatgcttaaagaaagcattgctgctacaaatatttctatttttagaaagggtttttatggaccaatgccccagttgtcagtcagagccgttggtgtttttcattgtttaaaatgtcacctgtaaaatgggcattatttatgtttttttttttgcattcctgataattgtatgtattgtataaagaacgtctgtacattgggttataacactagtatatttaaacttacaggcttatttgtaatgtaaaccaccattttaatgtactgtaattaacatggttataatacgtacaatccttccctcatcccatcacacaactttttttgtgtgtgataaactgattttggtttgcaataaaaccttagaaa

SEQ ID NO: 7 - protein, 431 aa, Homo sapiens
  misc_feature: Incyte ID No 3819039CD1
MTVKTEAAKGTLTYSRMRGMVAILIAFMKQRRMGLNDFIQKIANNSYACKHPEVQSILKISQPQEPELMNANPSPPPSPSQQINLGPSSNPHAKPSDFHFLKVIGKGSFGKVLLARHKAEEVFYAVKVLQKKAILKKKEEKHIMSERNVLLKNVKHPFLVGLHFSFQTADKLYFVLDYINGGELFYHLQRERCFLEPRARFYAAEIASALGYLHSLNIVYRDLKPENILLDSQGHIVLTDFGLCKENIEHNSTTSTFCGTPEYLAPEVLHKQPYDRTVDWWCLGAVLYEMLYGLPPFYSRNTAEMYDNILNKPLQLKPNITNSARHLLEGLLQKDRTKRLGAKDDFMEIKSHVFFSLINWDDLINKKITPPFNPNVSGPNDLRHFDPEFTEEPVPNSIGKSPDSVLVTASVKEAAEAFLGFSYAPPTDSFL

SEQ ID NO: 8 - DNA, 4000 bp, Homo sapiens
  misc_feature: Incyte ID No 330923.5
tgtaaatgaccacttgccaacatttactcgtacttcttatgtgacatcagtggaagaaaatacagttgatgtggaaatcttacgagttactgttgaggataaggacttagtgaatactgctaactggagagctaattataccattttaaagggcaatgaaaatggcaattttaaaattgtaacagatgccaaaaccaatgaaggagttctttgtgtagttaagcctttgaattatgaagaaaagcaacagatgatcttgcaaattggtgtagttaatgaagctccattttccagagaggctagtccaagatcagccatgagcacagcaacagttactgttaatgtagaagatcaggatgagggccctgagtgtaaccctccaatacagactgttcgcatgaaagaaaatgcagaagtgggaacaacaagcaatggatataaagcatatgncccagaaacaagaagtagcagtggcataaggtataagaaattaactgatccaacagggtgggtcaccattgatgaaaatacaggatcaatcaaagttttcagaagcctggatagagaggcagagaccatcaaaaatggcatatataatattacagtccttgcatcagaccaaggagggagaacatgtacggggacactgggcattatacttcaagacgtgaatgataacagcccattcatacctaaaaagacagtgatcatctgcaaacccaccatgtcatctgcggagattgttgcggttgatcctgatgagcctatccatggcccaccctttgactttagtctggagagttctacttcagaagtacagagaatgtggagactgaaagcaattaatgatacagcagcacgtctttcctatcagaatgatcctccatttggctcatatgtagtacctataacagtgagagatagacttggcatgtctagtgtcacttcattggatgttacactgtgtgactgcattaccgaaaatgactgcacacatcgtgtagatccaaggattggcggtggaggagtacaacttggaaagtgggccatccttgcaatattgttgggcatagcattgctcttttgcatcctgtttacgctggtctgtggggcttctgggacgtctaaacaaccaaaagtaattcctgatgatttagcccagcagaacctaattgtatcaaacacagaagctcctggagatgacaaagtgtattctgcgaatggcttcacaacccaaactgtgggcgcttctgctcagggagtttgtggcaccgtgggatcaggaatcaaaaacggaggtcaggagaccatcgaaatggtgaaaggaggacaccagacctcggaatcctgccggggggctggccaccatcacaccctggactcctgcaggggaggacacacggaggtggacaactgcagatacacttactcggagtggcacagttttactcagccccgtcttggtgaaaaagtgtatctgtgtaatcaagatgaaaatcacaagcatgcccaagactatgtcctgacatataactatgaaggaagaggatcggtggctgggtctgtaggttgttgcagtgaacgacaagaagaagatgggcttgaatttttggataatttggagcccaaatttaggacactagcagaagcatgcatgaagagatgagtgtgttctaataagtctctgaaagccagtggctttatgacttttaaaaaaaattacaaaccaagaattttttaaagcagaagatgctatttgtgggggtttttctctcattatttggatggaatctctttggtcaaatgcacatttacagagagacactataaacaagtacacaaatttttcaatttttacatatttttaaattacttatcttctatccaaggaggtctacagagaaattaaagtctgccttatttgttacatttgggtataatgacaacagccaatttatagtgcaataaaatgtaattaattcaagtccttattatagactatttgaagcacaacctaatggaaaattgtagagaccttgctttaacattatctccagttaattaagtgttcatgtggtgcttggaaactgttgttttcctgaacatctaaagtgtgtagactgcattcttgctattattttattcttgtaatgtgaccttttcactgtgcaaagggagatttctagccaggcattgactattacaatttcattttggtggagtttagttttaggttttattgtatataaaatcctgcactgaatctgtgtctcctctgttacctacttttgccagtgaaatttaagttttaaaatactttcagaatgtatttttactactgcaagtttttggtctttaaaatgtcaagtagcatctctctctttctctctgtctctttctgtttctctctccagtttttttttttttaatttccatatgggctaaagaatccaaatattttaaaaatctgtctctcttttcttctctcataaagtgaattattccttttttttgttttatgtaagtgtatatattcttagtttttcttgaaatcattgtaatgttaactttgttgtttcaaatatcttggtgattgcttcattatctcttcaacaaaaaaaacctttaattttgccattgaaactgtagaactatgccatgcttttattagaagcagtgctctgtgttaacaacaagaatggtgtaattagaattgggatgtggatatttactgtatgacaacacatttacagttctgtaatgcaaggatgcagtttaaaaatgtgaagtagtgatggtttttgaaataagctttaaaatatagggatcttgaaggctccctggggtaactattttataacttagataaaatggctagtcatatctgtgtgtttgtaaagttatttttttaatattttaagattacaattttaacaaatgtagaaatgagccaaactatttaaattttaaaacagtaaaacaaaatgaaacttaatagctcacaaaattccagtccatgtttcatgacttattttagtcaatgaattttctatttatactaaacatatggacattttaaatgtgtttctaatatttttgattatctataatgtgcctgtcttcaattcacaagattgggttataacaattatttgccagattaacactagggaattatttgataaccagcttatcttatcagtagttttattgctgatcaggcaaaaatagttttccaaagttatttttaataaagtatatacaaaattcttatatattactagtcatgataaagtaaattaagcagtttttaaaacttagtgtgagtttgttcatcacaggtctgatatgagtttaagggatttcgcactccctgaatcagagaagtaagaccccttccttagattcctgttatacattttttaaaatgtagagtttgttttggagacattttcagtgcattgttattgccatatttatataatatgactattctaaaggctgtgaggccatggggtattggttaagttgcttgcttttgctttgtccattttcatcattttaaaatgggggataataacagaacttgtttcctagggccattgtaagtcacttgaataaaaaatagttttgaagcatgagagtcatacagagcggtccacctaaaaggcactcctgataataataaatgattttaaacaagaaaaaaaaaaaaaaaaaaaaaaaaaaaaaaaaaaaaaaaaaaaaaaaaaaaaaaaaaaaaaaaaaaaaaaaaaaaaaaaanaaaaaaaaaanaaangggggggccgttttgggggttccaagtttaggtaggggtgaatggggggtaatggtttttttttggggtcccctaatttaaattaattggccgtggttttaaaaggtggtgattgggaaaaccctggggttacccaatttaatggctttgaagaaaatccccctttggccagttggggtaatagcgaaggggcccgaaccg

SEQ ID NO: 9 - DNA, 3474 bp, Homo sapiens
  misc_feature: Incyte ID No 234630.57
aaaaactgcagccaacttccgaggcagcctcattgcccagcggaccccagcctctgcccaggttcggtccgccatcctcgtcccgtcctccgccggcccctgccccgcgcccagggatcctccagctcctttcgcccgcgccctccgttcgctccggacaccatggacaagttttggtggcacgcagcctggggactctgcctcgtgccgctgagcctggcgcagatcgatttgaatataacctgccgctttgcaggtgtattccacgtggagaaaaatggtcgctacagcatctctcggacggaggccgctgacctctgcaaggctttcaatagcaccttgcccacaatggcccagatggagaaagctctgagcatcggatttgagacctgcaggtatgggttcatagaagggcacgtggtgattccccggatccaccccaactccatctgtgcagcaaacaacacaggggtgtacatcctcacatccaacacctcccagtatgacacatattgcttcaatgcttcagctccacctgaagaagattgtacatcagtcacagacctgcccaatgcctttgatggaccaattaccataactattgttaaccgtgatggcacccgctatgtccagaaaggagaatacagaacgaatcctgaagacatctaccccagcaaccctactgatgatgacgtgagcagcggctcctccagtgaaaggagcagcacttcaggaggttacatcttttacaccttttctactgtacaccccatcccagacgaagacagtccctggatcaccgacagcacagacagaatccctgctaccagtacgtcttcaaataccatctcagcaggctgggagccaaatgaagaaaatgaagatgaaagagacagacacctcagtttttctggatcaggcattgatgatgatgaagattttatctccagcaccatttcaaccacaccacgggcttttgaccacacaaaacagaaccaggactggacccagtggaacccaagccattcaaatccggaagtgctacttcagacaaccacaaggatgactgatgtagacagaaatggcaccactgcttatgaaggaaactggaacccagaagcacaccctcccctcattcaccatgagcatcatgaggaagaagagaccccacattctacaagcacaatccaggcaactcctagtagtacaacggaagaaacagctacccagaaggaacagtggtttggcaacagatggcatgagggatatcgccaaacacccaaagaagactcccattcgacaacagggacagctgcagcctcagctcataccagccatccaatgcaaggaaggacaacaccaagcccagaggacagttcctggactgatttcttcaacccaatctcacaccccatgggacgaggtcatcaagcaggaagaaggatggatatggactccagtcatagtacaacgcttcagcctactgcaaatccaaacacaggtttggtggaagatttggacaggacaggacctctttcaatgacaacgcagcagagtaattctcagagcttctctacatcacatgaaggcttggaagaagataaagaccatccaacaacttctactctgacatcaagcaataggaatgatgtcacaggtggaagaagagacccaaatcattctgaaggctcaactactttactggaaggttatacctctcattacccacacacgaaggaaagcaggaccttcatcccagtgacctcagctaagactgggtcctttggagttactgcagttactgttggagattccaactctaatgtcaatcgttccttatcaggagaccaagacacattccaccccagtggggggtcccataccactcatggatctgaatcagatggacactcacatgggagtcaagaaggtggagcaaacacaacctctggtcctataaggacaccccaaattccagaatggctgatcatcttggcatccctcttggccttggctttgattcttgcagtttgcattgcagtcaacagtcgaagaaggtgtgggcagaagaaaaagctagtgatcaacagtggcaatggagctgtggaggacagaaagccaagtggactcaacggagaggccagcaagtctcaggaaatggtgcatttggtgaacaaggagtcgtcagaaactccagaccagtttatgacagctgatgagacaaggaacctgcagaatgtggacatgaagattggggtgtaacacctacaccattatcttggaaagaaacaaccgttggaaacataaccattacagggagctgggacacttaacagatgcaatgtgctactgattgtttcattgcgaatcttttttagcataaaattttctactctttttgttttttgtgttttgttctttaaagtcaggtccaatttgtaaaaacagcattgctttctgaaattagggcccaattaataatcagcaagaatttgatcgttccagttcccacttggaggcctttcatccctcgggtgtgctatggatggcttctaacaaaaactacacatatgtattcctgatcgccaacctttcccccaccagctaaggacatttcccagggttaatagggcctggtccctgggaggaaatttgaatgggtccattttgcccttccatagcctaatccctgggcattgctttccactgaggttgggggttggggtgtactagttacacatcttcaacagaccccctctagaaatttttcagatgcttctgggagacacccaaagggtgaagctatttatctgtagtaaactatttatctgtgtttttgaaatattaaaccctggatcagtcctttgatcagtataattttttaaagttactttgtcagaggcacaaaagggtttaaactgattcataataaatatctgtacttcttcgatcttcaccttttgtgctgtgattcttcagtttctaaaccagcactgtctgggtccctacaatgtatcaggaagagctgagaatggtaaggagactcttctaagtcttcatctcagagaccctgagttcccactcagacccactcagccaaatctcatggaagaccaaggagggcagcactgtttttgttttttgttttttgtttttttttttggacactgtccaaaggttttccatcctgtcctggaatcagagttggaagctgaggagcttcagcctcttttatggtttaatggccacctgttctctcctgtgaaaggcttggcaaagtcacattaagttggcatgacctgttatccctggggccctatttcatagaggctggccctattagtgatttccaaaaacaatatggaagtgcctttggatgtcttacaataagagaagaagccaagggaattgaaagagatgggcaaaggggaaggatgatgccatgtagatcctgttggacatttttatggctgt

SEQ ID NO: 10 - DNA, 1736 bp, Homo sapiens
  misc_feature: Incyte ID No 611514CB1
ggaacagcggcctctgacaccagcacagcaaacccgccgggatcaaagtgtaccagtcggcagcatggctacgaaatgtgggaattgtggacccggctactccacccctctggaggccatgaaaggacccagggaagagatcgtctacctgccctgcatttaccgaaacacaggcactgaggccccagattatctggccactgtggatgttgaccccaagtctccccagtattgccaggtcatccaccggctgcccatgcccaacctgaaggacgagctgcatcactcaggatggaacacctgcagcagctgcttcggtgatagcaccaagtcgcgcaccaagctggtgctgcccagtctcatctcctctcgcatctatgtggtggacgtgggctctgagccccgggccccaaagctgcacaaggtcattgagcccaaggacatccatgccaagtgcgaactggcctttctccacaccagccactgcctggccagcggggaagtgatgatcagctccctgggagacgtcaagggcaatggcaaagggggttttgtgctgctggatggggagacgttcgaggtgaaggggacatgggagagacctgggggtgctgcaccgttgggctatgacttctggtaccagcctcgacacaatgtcatgatcagcactgagtgggcagctcccaatgtcttacgagatggcttcaaccccgctgatgtggaggctggactgtacgggagccacttatatgtatgggactggcagcgccatgagattgtgcagaccctgtctctaaaagatgggcttattcccttggagatccgcttcctgcacaacccagacgctgcccaaggctttgtgggctgcgcactcagctccaccatccagcgcttctacaagaacgagggaggtacatggtcagtggagaaggtgatccaggtgccccccaagaaagtgaagggctggctgctgcccgaaatgccaggcctgatcaccgacatcctgctctccctggacgaccgcttcctctacttcagcaactggctgcatggggacctgaggcagtatgacatctctgacccacagagaccccgcctcacaggacagctcttcctcggaggcagcattgttaagggaggccctgtgcaagtgctggaggacgaggaactaaagtcccagccagagcccctagtggtcaagggaaaacgggtggctggaggccctcagatgatccagctcagcctggatgggaagcgcctctacatcaccacgtcgctgtacagtgcctgggacaagcagttttaccctgatctcatcagggaaggctctgtgatgctgcaggttgatgtagacacagtaaaaggagggctgaagttgaaccccaacttcctggtggacttcgggaaggagccccttggcccagcccttgcccatgagctccgctaccctgggggcgattgtagctctgacatctggatttgaactccaccctcatcacccacactccctattttgggccctcacttccttggggacctggcttcattctgctctctcttggcacccgacccttggcagcatgtaccacacagccaagctgagactgtggcaatgtgttgagtcatatacatttactgaccactgttgcttgttgctcactgtgctgcttttccatgagctcttggaggcaccaagaaataaactcgtaaccctgtccttcaaaaaaaaaaaaaaa

SEQ ID NO: 11 - protein, 472 aa, Homo sapiens
  misc_feature: Incyte ID No 611514CD1
MATKCGNCGPGYSTPLEAMKGPREEIVYLPCIYRNTGTEAPDYLATVDVDPKSPQYCQVIHRLPMPNLKDELHHSGWNTCSSCFGDSTKSRTKLVLPSLISSRIYVVDVGSEPRAPKLHKVIEPKDIHAKCELAFLHTSHCLASGEVMISSLGDVKGNGKGGFVLLDGETFEVKGTWERPGGAAPLGYDFWYQPRHNVMISTEWAAPNVLRDGFNPADVEAGLYGSHLYVWDWQRHEIVQTLSLKDGLIPLEIRFLHNPDAAQGFVGCALSSTIQRFYKNEGGTWSVEKVIQVPPKKVKGWLLPEMPGLITDILLSLDDRFLYFSNWLHGDLRQYDISDPQRPRLTGQLFLGGSIVKGGPVQVLEDEELKSQPEPLVVKGKRVAGGPQMIQLSLDGKRLYITTSLYSAWDKQFYPDLIREGSVMLQVDVDTVKGGLKLNPNFLVDFGKEPLGPALAHELRYPGGDCSSDIWI

SEQ ID NO: 12 - DNA, 570 bp, Homo sapiens
  misc_feature: Incyte ID No 2072479CB1
cgctggcccggcccacccggggcggttgtggtcgctatatataaggtggggaggccgccggcccgttcggttccgggcgttaccatcgtccgtgcgcaccgcccggcgtccagatttggcaattcttcgctgaagtcatcatgagctttttccaactcctgatgaaaaggaaggaactcattcccttggtggtgttcatgactgtggcggcgggtggagcctcatctttcgctgtgtattctctttggaaaaccgatgtgatccttgatcgaaaaaaaaatccagaaccttgggaaactgtggaccctactgtacctcaaaagcttataacaatcaaccaacaatggaaacccattgaagagttgcaaaatgtccaaagggtgaccaaatgacgagccctcgcctctttcttctgaagagtactctataaatctagtggaaacatttctgcacaaactagattctggacaccagtgtgcggaaatgcttctgctacatttttagggtttgtctacattttttgggctctggataaggaattaaaggagtgcagcaataactgcactgtct

SEQ ID NO: 13 - protein, 83 aa, Homo sapiens
  misc_feature: Incyte ID No 2072479CD1
MSFFQLLMKRKELIPLVVFMTVAAGGASSFAVYSLWKTDVILDRKKNPEPWETVDPTVPQKLITINQQWKPIEELQNVQRVTK

SEQ ID NO: 14 - DNA, 3719 bp, Homo sapiens
  misc_feature: Incyte ID No 410911.5
gccctctcgaaggcaggacagcagtggcggcagccacggtgttggatacacttgtatgaggcttcagctgtgctgcataaatttcttgaagaacctgtgagccagaagcaaaatttcaggacagggggcacacactgaacttgaaatataaagacctgaagatagtcttttctgtccaaagatggaaaacagtactactaccatttctcgggaggagcttgaagaactacaagaggcatttaataaaatagatattgacaatagtgggtatgtcagtgactatgaacttcaagacctgtttaaggaagcaagccttcctctgcctggctacaaggtgcgcgagattgtggagaaaattctatcagttgctgacagcaacaaagatggcaaaatcagttttgaagagtttgtgtcactaatgcaagaattaaaaagcaaagatatcagcaaaacattccgaaaaataattaacaagagggaagggattactgctattggaggaacttcaactatttccagtgagggcacacagcattcttattcagaggaagaaaaagtggcttttgttaactggataaacaaagccctggagaatgaccctgactgtaagcatcttatacccatgaatcccaatgatgatagtcttttcaagtcacttgcagatggcatccttctttgcaaaatgatcaacttatctgaaccagatacaattgatgaaagagccatcaataagaaaaagctcacgccattcactatttctgaaaatttaaacctagctctgaattctgcctcagccattggttgtacagtggtcaacattggtgcatcagatctcaaagaaggaaaacctcacttggtcttgggacttctctggcagatcatcaaagttggcctttttgctgatattgagatttccaggaatgaagctctgattgcattgttaaatgaaggtgaggaactagaggagctgatgaagctttctcccgaggaattactgctgcgatgggtgaactaccatctgaccaatgcaggatggcataccatcagcaacttcagccaagacattaaggactcgagagcctattttcatctgcttaatcagattgcccctaaaggtggggaagatggacctgccattgccattgacctttcaggaattaatgagacaaatgacctgaagcgtgctggactcatgcttcaagaagcagataaactgggctgcaaacagtttgttactcctgcagatgtggtttcaggcaatcctaaacttaatttagcttttgtagctaatttgtttaacacatacccatgcctgcacaagccgaataataatgacatcgatatgaatttactggaaggagagagcaaggaagagagaacatttcggaactggatgaattccttgggagtcaacccatacattaatcatttgtacagtgaccttgcagatgctttagtgatctttcagctctatgagatgatccgagtgccagtcaactggagccatgtcaacaaacctccttatcctgcccttggagggaacatgaagaagattgaaaactgtaactatgcagtggaacttgggaagaacaaggccaaattctccttggttggcattgctgggcaggacctaaatgaagggaattcaacacttaccctggcattggtatggcagctgatgagaaggtacacattgaatgtgttatcggatcttggagagggtgaaaaagtaaatgatgaaattataattaaatgggtcaatcagactcttaaaagtgcaaacaaaaagacttctatttccagcttcaaggataaatctataagcacaagtttacctgtcctagatttaatagatgccattgcaccaaatgcagttcgtcaagaaatgatcaggagagaaaacttatctgatgaggacaagctgaacaatgctaaatacgccatttcagttgctcgaaagatcggtgcccggatatatgcattacctgatgacctcgtagaagtgaaaccaaagatggttatgacggtgtttgcatgcttaatgggaaaaggactgaacagaataaaataatcatttcatatgattttctgccacattaaacatattgtatgcctcacagtttacaggattctgaaatgtagtgggtgtaaaaccagagattatttgtatgctcaaaatagttatatattcattaatgaattcaatatcctgttcatactagttagagctggtcagcctttttgggtaacacagttaatttaccaactgatacagataatagaatatattcataatcaagctgatacttcatgattaaattatttttgttgcttaaaagtcgtattagacaagactaaatcattcttttttatggttcaaaaaagatgaatacaaacgtttttgcaggttctgctgtgaaatgtggtttgatttttttggtgtgttaattttgatcataaatgcattcatactcataatccagtttaatccttttatttgcttcctccaactatttaaagtggtccaaaaacacttttctgtaagtttctatactgtctaaaaccttatggtgaccagaattgtttattaatatcaaacttttttatatatgagaactaattcttgaataaaccccaaagttcactctcttgtttaagtagcagcagctttttacttaaaatttaattttaactacattgatactttacacatcctagtttggtaacacagctttaactatgtcatgcaacatatatatgttggtaggatgttattagagagatatgtgtgcatatatatttttttgcacctgaatcacccagcttttcataagtggtatgtttaattggtcattcagccaaccatcagtattttccccccacaacatgtgtaacacttttcagtctgtggatatctgatacattaagatttctttttataagtattcattttgaatgtgcatatagttatttgaccccttccaaatacttgtagccaaacattggctagaacatcccaagatatgctgacactgtcctgttagcttcatattatacttgctagtttaggtctctatagaagccctatataatttagaatatgcccactgaatatctttaatagaaagtaacataaagctagtattcaatgtagagtattttcatatgtttttcacagcccgttacaaattggcaatgtttggttaatgtttgtattacttggaaatcgctacagcttggactatttttttctaaatttttagcattagtccatttctgctgctaacaattgaatccagaaatctactttctccatcttccactgttagtgccagtgagcaatactgttgtgcaacaaaaatgtcactttatctcagtgtgaatgagtagtctaaattccctttctaccattgatttaaatatatatattggtaagagagactgcccatgtgtttagaatagaattttttaaatgaaatgatcaacaggtggaatttgaaatatattcttctacaaaagagatttctttcccttttatattttgatgattgttttcttaagattaagatatgttcttgctcttttataagattatttaaattatgtttccctctgattttttttcaccattgtatttactaagttattggatttacatgaaatctggcactttagggtgttctttttctcacagagtatatttaataaaaatgctgtgt

SEQ ID NO: 15 - DNA, 4368 bp, Homo sapiens
  misc_feature: Incyte ID No 1285632CB1
gcaggcggcggggagaaagactctctcacctggtcttgcggctgtggccaccgccggccaggggtgtggagggcgtgctgccggagacgtccgccgggctctgcagttccgccgggggtcgggcagctatggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccagctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgccaacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctggattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagttgaggaagaggatggcaagactgcaactcaaccactgttgaaaaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataaatgttctatacagtggacagccctccagaatggtacttcagtgcctagtgtagtaactgaaatcttcaatgacacattaacatcacaatggcgaatggtgacttttctttcacgatttcattaatttgaaagcacacaggaaagttgctccattgataacgtgtatggagacttcggttttagtcaattccatatctcaatcttaatggtgattcttctctgttgaactgaagtttgtgagagtagttttcctttgctacttgaatagcaataaaagcgtgttaactttttgattgatgaaagaagtacaaaaagcctttagccttgaggtgccttctgaaattaaccaaatttcatccatatatcctcttttataaacttatagaatgtcaaactttgccttcaactgtttttatttctagtctcttccactttaaaacaaaatgaacactgcttgtcttcttccattgaccatttagtgttgagtactgtatgtgttttgttaattctataaaggtatctgttagatattaaaggtgagaattagggcaggttaatcaaaaatggggaaggggaaatggtaaccaaaaaaaaaaaaaaa

SEQ ID NO: 16 - protein, 1212 aa, Homo sapiens
  misc_feature: Incyte ID No 1285632CD1
MEPRPTAPSSGAPGLAGVGETPSAAALAAARVELPGTAVPSVPEDAAPASRDGGGVRDEGPAAAGDGLGRPLGPTPSQSRFQVDLVSENAGRAAAAAAAAAAAAAAAGAGAGAKQTPADGEASGESEPAKGSEEAKGRFRVNFVDPAASSSAEDSLSDAAGVGVDGPNVSFQNGGDTVLSEGSSLHSGGGGGSGHHQHYYYDTHTNTYYLRTFGHNTMDAVPRIDHYRHTAAQLGEKLLRPSLAELHDELEKEPFEDGFANGEESTPTRDAVVTYTAESKGVVKFGWIKGVLVRCMLNIWGVMLFIRLSWIVGQAGIGLSVLVIMMATVVTTITGLSTSAIATNGFVRGGGAYYLISRSLGPEFGGAIGLIFAFANAVAVAMYVVGFAETVVELLKEHSILMIDEINDIRIIGAITVVILLGISVAGMEWEAKAQIVLLVILLLAIGDFVIGTFIPLESKKPKGFFGYKSEIFNENFGPDFREEETFFSVFAIFFPAATGILAGANISGDLADPQSAIPKGTLLAILITTLVYVGIAVSVGSCVVRDATGNVNDTIVTELTNCTSAACKLNFDFSSCESSPCSYGLMNNFQVMSMVSGFTPLISAGIFSATLSSALASLVSAPKIFQALCKDNIYPAFQMFAKGYGKNNEPLRGYILTFLIALGFILIAELNVIAPIISNFFLASYALINFSVFHASLAKSPGWRPGFKYYNMWISLLGAILCCIVMFVINWWAALLTYVIVLGLYIYVTYKKPDVNWGSSTQALTYLNALQHSIRLSGVEDHVKNFRPQCLVMTGAPNSRPALLHLVHDFTKNVGLMICGHVHMGPRRQAMKEMSIDQAKYQRWLIKNKMKAFYAPVHADDLREGAQYLMQAAGLGRMKPNTLVLGFKKDWLQADMRDVDMYINLFHDAFDIQYGVVVIRLKEGLDISHLQGQEELLSSQEKSPGTKDVVVSVEYSKKSDLDTSKPLSEKPITHKVEEEDGKTATQPLLKKESKGPIVPLNVADQKLLEASTQFQKKQGKNTIDVWWLFDDGGLTLLIPYLLTTKKKWKDCKIRVFIGGKINRIDHDRRAMATLLSKFRIDFSDIMVLGDINTKPKKENIIAFEEIIEPYRLHEDDKEQDIADKMKEDEPWRITDNELELYKTKTYRQIRLNELLKEHSSTANIIVMSLPVARKGAVSSALYMAWLEALSKDLPPILLVRGNHQSVLTFYS

SEQ ID NO: 17 - DNA, 735 bp, Homo sapiens
  misc_feature: Incyte ID No 474322.36
gtgcagaacaaacaagacggcctggggatacaactctggagtcctctgagaggttggatgggagagcatgtctgtgtgtctcagagccaccaaggaggagcaggggagcgacggccggggcagaagttgagaccaccagcagaggagctaggccagtccatctgcatttgtcacccaagaactcttaccatgaagaccctcctactgttggcagtgatcatgatctttggcctactgcaggcccatgggaatttggtgaatttccacagaatgatcaagttgacgacaggaaaggaagccgcactcagttatgggcttctatggctgccactgtggcgtgggtggcagaggatcccccaaggatgcaacgggatcgctgtgtgtcacntccatgactgttgctacaaacgtctggagaaacgtggatgtggcaccaaatttctgagctacaagtttagcaactcggggagcagaatcacctgtgcaaaacaggactcctgcagaagtcaactgtgtgagtgtgataaggctgctgccacctgttttgctagaaacaagacgacctacaataaaaattccttggccatgcagaaagcatccctcacccatcctagaggccaggcaggagcccttctatacccacccagaatgagacatccagcagatttccagccttctactgctctcctccacctcaactccgtgcttaaccaaagaagctgtactccggggggt

SEQ ID NO: 18 - DNA, 1868 bp, Homo sapiens
  misc_feature: Incyte ID No 3040213CB1
gtcccagtcagtccggaggctgcggctgcagaagtaccgcctgcggagtaactgcaaagatgctgtccgtgcgcgttgctgcggccgtggtccgcgcccttcctcggcgggccggactggtctccagaaatgctttgggttcatctttcattgctgcaaggaacttccatgcctctaacactcatcttcaaaagactgggactgctgagatgtcctctattcttgaagagcgtattcttggagctgatacctctgttgatcttgaagaaactgggcgtgtcttaagtattggtgatggtattgcccgcgtacatgggctgaggaatgttcaagcagaagaaatggtagagttttcttcaggcttaaagggtatgtccttgaacttggaacctgacaatgttggtgttgtcgtgtttggaaatgataaactaattaaggaaggagatatagtgaagaggacaggagccattgtggacgttccagttggtgaggagctgttgggtcgtgtagttgatgcccttggtaatgctattgatggaaagggtccaattggttccaagacgcgtaggcgagttggtctgaaagcccccggtatcattcctcgaatttcagtgcgggaaccaatgcagactggcattaaggctgtggatagcttggtgccaattggtcgtggtcagcgtgaactgattattggtgaccgacagactgggaaaacctcaattgctattgacacaatcattaaccagaaacgtttcaatgatggatctgatgaaaagaagaagctgtactgtatttatgttgctattggtcaaaagagatccactgttgcccagttggtgaagagacttacagatgcagatgccatgaagtacaccattgtggtgtcggctacggcctcggatgctgccccacttcagtacctggctccttactctggctgttccatgggagagtattttagagacaatggcaaacatgctttgatcatctatgacgacttatccaaacaggctgttgcttaccgtcagatgtctctgttgctccgccgaccccctggtcgtgaggcctatcctggtgatgtgttctacctacactcccggttgctggagagagcagccaaaatgaacgatgcttttggtggtggctccttgactgctttgccagtcatagaaacacaggctggtgatgtgtctgcttacattccaacaaatgtcatttccatcactgacggacagatcttcttggaaacagaattgttctacaaaggtatccgccctgcaattaacgttggtctgtctgtatctcgtgtcggatccgctgcccaaaccagggctatgaagcaggtagcaggtaccatgaagctggaattggctcagtatcgtgaggttgctgcttttgcccagttcggttctgacctcgatgctgccactcaacaacttttgagtcgtggcgtgcgtctaactgagttgctgaagcaaggacagtattctcccatggctattgaagaacaagtggctgttatctatgcgggtgtaaggggatatcttgataaactggagcccagcaagattacaaagtttgagaatgctttcttgtctcatgtcgtcagccagcaccaagccttgttgggcactatcagggctgatggaaagatctcagaacaatcagatgcaaagctgaaagagattgtaacaaatttcttggctggatttgaagcttaaactcctgtggattcacatcaaataccagttcagttttgtcattgttctagtaaattagttccatttgtaaaagggttactctcatactccttatgtacagaaatcacatgaaaaataaaggttccataatgcaaaaaaaaaaaaaaa

SEQ ID NO: 19 - protein, 553 aa, Homo sapiens
  misc_feature: Incyte ID No 3040213CD1
MLSVRVAAAVVRALPRRAGLVSRNALGSSFIAARNFHASNTHLQKTGTAEMSSILEERILGADTSVDLEETGRVLSIGDGIARVHGLRNVQAEEMVEFSSGLKGMSLNLEPDNVGVVVFGNDKLIKEGDIVKRTGAIVDVPVGEELLGRVVDALGNAIDGKGPIGSKTRRRVGLKAPGIIPRISVREPMQTGIKAVDSLVPIGRGQRELIIGDRQTGKTSIAIDTIINQKRFNDGSDEKKKLYCIYVAIGQKRSTVAQLVKRLTDADAMKYTIVVSATASDAAPLQYLAPYSGCSMGEYFRDNGKHALIIYDDLSKQAVAYRQMSLLLRRPPGREAYPGDVFYLHSRLLERAAKMNDAFGGGSLTALPVIETQAGDVSAYIPTNVISITDGQIFLETELFYKGIRPAINVGLSVSRVGSAAQTRAMKQVAGTMKLELAQYREVAAFAQFGSDLDAATQQLLSRGVRLTELLKQGQYSPMAIEEQVAVIYAGVRGYLDKLEPSKITKFENAFLSHVVSQHQALLGTIRADGKISEQSDAKLKEIVTNFLAGFEA

SEQ ID NO: 20 - DNA, 528 bp, Homo sapiens
  misc_feature: Incyte ID No 1282225CB1
ggagccccctataaaacagcctacagtggacagtctggtcggcagagccgcaggtcagtcgtgaagagggagctctattgccaccatgagtttctccggcaagtaccaactgcagagccaggaaaactttgaagccttcatgaaggcaatcggtctgccggaagagctcatccagaaggggaaggatatcaagggggtgtcggaaatcgtgcagaatgggaagcacttcaagttcaccatcaccgctgggtccaaagtgatccaaaacgaattcacggtgggggaggaatgtgagctggagacaatgacaggggagaaagtcaagacagtggttcagttggaaggtgacaataaactggtgacagctttcaaaaacatcaagtctgtgaccgaactcaacggcgacataatcaccaataccatgacattgggtgacattgtcttcaagagaatcagcaagagaatttaaacaagtctgcatttcatattattttagtgtgtaaaattaatgtaataaagtgaactttg

SEQ ID NO: 21 - protein, 127 aa, Homo sapiens
  misc_feature: Incyte ID No 1282225CD1
MSFSGKYQLQSQENFEAFMKAIGLPEELIQKGKDIKGVSEIVQNGKHFKFTITAGSKVIQNEFTVGEECELETMTGEKVKTVVQLEGDNKLVTAFKNIKSVTELNGDIITNTMTLGDIVFKRISKRI

SEQ ID NO: 22 - DNA, 2486 bp, Homo sapiens
  misc_feature: Incyte ID No 991163CB1
gggcgggccgggccgagtaggcgcgagctaagcaggaggcggaggcggaggcggagggcgaggggcggggagcgccgcctggagcgcggcaggtcatattgaacattccagatacctatcattactcgatgctgttgataacagcaagatggctttgaactcagggtcaccacctgctattggaccttactatgaaaaccatggataccaaccggaaaacccctatcccgcacagcccactgtggtccccactgtctacgaggtgcatccggctcagtactacccgtcccccgtgccccagtacgccccgagggtcctgacgcaggcttccaaccccgtcgtctgcacgcagcccaaatccccatccgggacagtgtgcacctcaaagactaagaaagcactgtgcatcaccttgaccctggggaccttcctcgtgggagctgcgctggccgctggcctactctggaagttcatgggcagcaagtgctccaactctgggatagagtgcgactcctcaggtacctgcatcaacccctctaactggtgtgatggcgtgtcacactgccccggcggggaggacgagaatcggtgtgttcgcctctacggaccaaacttcatccttcaggtgtactcatctcagaggaagtcctggcaccctgtgtgccaagacgactggaacgagaactacgggcgggcggcctgcagggacatgggctataagaataatttttactctagccaaggaatagtggatgacagcggatccaccagctttatgaaactgaacacaagtgccggcaatgtcgatatctataaaaaactgtaccacagtgatgcctgttcttcaaaagcagtggtttctttacgctgtatagcctgcggggtcaacttgaactcaagccgccagagcaggattgtgggcggcgagagcgcgctcccgggggcctggccctggcaggtcagcctgcacgtccagaacgtccacgtgtgcggaggctccatcatcacccccgagtggaccgtgacagccgcccactgcgtggaaaaacctcttaacaatccatggcattggacggcatttgcggggattttgagacaatctttcatgttctatggagccggataccaagtagaaaaagtgatttctcatccaaattatgactccaagaccaagaacaatgacattgcgctgatgaagctgcagaagcctctgactttcaacgacctagtgaaaccagtgtgtctgcccaacccaggcatgatgctgcagccagaacagctctgctggatttccgggtggggggccaccgaggagaaagggaagacctcagaagtgctgaacgctgccaaggtgcttctcattgagacacagagatgcaacagcagatatgtctatgacaacctgatcacaccagccatgatctgtgccggcttcctgcaggggaacgtcgattcttgccagggtgacagtggagggcctctggtcacttcgaagaacaatatctggtggctgataggggatacaagctggggttctggctgtgccaaagcttacagaccaggagtgtacgggaatgtgatggtattcacggactggatttatcgacaaatgagggcagacggctaatccacatggtcttcgtccttgacgtcgttttacaagaaaacaatggggctggttttgcttccccgtgcatgatttactcttagagatgattcagaggtcacttcatttttattaaacagtgaacttgtctggctttggcactctctgccattctgtgcaggctgcagtggctcccctgcccagcctgctctccctaaccccttgtccgcaaggggtgatggccggctggttgtgggcactggcggtcaagtgtggaggagaggggtggaggctgccccattgagatcttcctgctgagtcctttccaggggccaattttggatgagcatggagctgtcacctctcagctgctggatgacttgagatgaaaaaggagagacatggaaagggagacagccaggtggcacctgcagcggctgccctctggggccacttggtagtgtccccagcctacctctccacaaggggattttgctgatgggttcttagagccttagcagccctggatggtggccagaaataaagggaccagcccttcatgggtggtgacgtggtagtcacttgtaaggggaacagaaacatttttgttcttatggggtgagaatatagacagtgcccttggtgcgagggaagcaattgaaaaggaacttgccctgagcactcctggtgcaggtctccacctgcacattgggtggggctcctgggagggagactcagccttcctcctcatcctccctgaccctgctcctagcaccctggagagtgcacatgccccttggtcctggcagggcgccaagtctggcaccatgttggcctcttcaggcctgctagtcactggaaattgaggtccatgggggaa

SEQ ID NO: 23 - protein, 384 aa, Homo sapiens
  misc_feature: Incyte ID No 991163CD1
MGSKCSNSGIECDSSGTCINPSNWCDGVSHCPGGEDENRCVRLYGPNFILQVYSSQRKSWHPVCQDDWNENYGRAACRDMGYKNNFYSSQGIVDDSGSTSFMKLNTSAGNVDIYKKLYHSDACSSKAVVSLRCIACGVNLNSSRQSRIVGGESALPGAWPWQVSLHVQNVHVCGGSIITPEWTVTAAHCVEKPLNNPWHWTAFAGILRQSFMFYGAGYQVEKVISHPNYDSKTKNNDIALMKLQKPLTFNDLVKPVCLPNPGMMLQPEQLCWISGWGATEEKGKTSEVLNAAKVLLIETQRCNSRYVYDNLITPAMICAGFLQGNVDSCQGDSGGPLVTSKNNIWWLIGDTSWGSGCAKAYRPGVYGNVMVFTDWIYRQMRADG

SEQ ID NO: 24 - DNA, 1001 bp, Homo sapiens
  misc_feature: Incyte ID No 3220207CB1
atgattacgaattcgagctcgtacccccccagaatgttgacagtcgctctcctagcccttctctgtgcctcagcctctggcaatgccattcaggccaggtcttcctcctatagtggagagtatggaagtggtggtggaaagcgattctctcattctggcaaccagttggacggccccatcaccgccctccgggtccgagtcaacacatactacatcgtaggtcttcaggtgcgctatggcaaggtgtggagcgactatgtgggtggtcgcaacggagacctggaggagatctttctgcaccctggggaatcagtgatccaggtttctgggaagtacaagtggtacctgaagaagctggtatttgtgacagacaagggccgctatctgtcttttgggaaagacagtggcacaagtttcaatgccgtccccttgcaccccaacaccgtgctccgcttcatcagtggccggtctggttctctcatcgatgccattggcctgcactgggatgtttaccccactagctgcagcagatgctgagcctcctctccttggcaggggcactgtgatgaggagtaagaactcccttatcactaacccccatccaaatggnnnnnnnnnnnnnnnnnnnnnnnnnnnnnnnnnnnnnnntcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcagggggatacgcaggaaagaacatgtgagcaaaaggccagcaaa

SEQ ID NO: 25 - protein, 178 aa, Homo sapiens
  misc_feature: Incyte ID No 3220207CD1
MITNSSSYPPRMLTVALLALLCASASGNAIQARSSSYSGEYGSGGGKRFSHSGNQLDGPITALRVRVNTYYIVGLQVRYGKVWSDYVGGRNGDLEEIFLHPGESVIQVSGKYKWYLKKLVFVTDKGRYLSFGKDSGTSFNAVPLHPNTVLRFISGRSGSLIDAIGLHWDVYPTSCSRC

SEQ ID NO: 26 - DNA, 2129 bp, Homo sapiens
  misc_feature: Incyte ID No 203309.2
ggcgccagtgtgctggaaagtgaagctacaggtgctccctcctggaatctccaatggatttcagtcgcagaagcttccacagaagcctgagctcctccttgcaggcccctgtagtcagtacagtgggcatgcagcgcctcgggacgacacccagcgtttatgggggtgctggaggccggggcatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatcaatttgtaggtaatgttcctgaaaattgcaatacatttcaattatactaaacctcacaaagtagaggaatccatgtaaattgcaaataaaccactttctaattttttcctgtttctgaattgtaaaaccccctttgggagtccctggtttcttattgagccaatttctgggttaatcttattgatttttcagcatcagtacaactctacaacctttgagctatatctgctttttcccattgcttccactgccttttaaaactcaacacagctttttgaataatttgagagtcaaattcaatcacaaatgctgagcagaataagagtgaagtacactatacttaaaatggaaatagattaaaaacaacattactgaaacccttctcaaggcaaaatgtgtctccttttgataataagctgcatatactatcaggtcctctctttctttatatggtgaacatatatttttaatgaaatgtctctcatttttttaataacaga

SEQ ID NO: 27 - DNA, 880 bp, Homo sapiens
  misc_feature: Incyte ID No 998971.1
gaaaaaaaaaatccctgtgaacaacttacagcaaaaatgaaacaaatgaaaaaaaagcttcgtgtactacaaaaggaactatcagaaccaaaagaaataaaatcatagagaatcaaaaagttaaaagagaacaagagctctgcagtgtgaggtatgacatactagtatataggatacttttagtactagctgacttaccttctgaggtttaactagagaaagaaatctctgtcttgtagagtatcaaattcatttaaataatacaagttcttaactgtgaatacatctcctgataattaaatgcatatttatttaaatcacaattttaatggctgcatagaaggccattatttggaaacccaattatttacttaacaaattaattttttatatttaatttttttgtgttataataagtgctgcaaggcataactgcatgtaaatctttttctatcattctaattattgacttggaataaattattcaatataaaaatatttggttaaattataggaagttttaagaagttctttgttcattacttctaaattgttctcaagaaaatttatattcatttatagttcaacaaagagagtgtgaaacggccattcctctacccccaataatcattttcctttaattatacacttgtaatcttaatatgcatggagtataaagaaaaatatagagtaatttatgactagtatattcaacatctctctctctcctacataaataaaatcaattcagaattctatgttaaaaacacttattatttttattttttaattaaatataatattctgtcttattctaaaagagatttaaaatttgttgataaaatataaaataatcaacaagatacatttaattattact

SEQ ID NO: 28 - DNA, 4101 bp, Homo sapiens
  misc_feature: Incyte ID No 333076.1
gtcctcgccgcgcccgtaggtcccggcagccgggccccgcctccttcggagtccgagcgatgggcggggaaagggacaggcaggtatagctctgtcggcgacgcggtgtccacctcagtcaggccacggtggaagacgcgtgccgcgagcgcctggttgcctgcagcggcccggacccgagaggaagctgaaccatctatctccagaaatgtcttcagaaagtaaagagcaacataacgtttcaaccaagagactcagctgaaggaaatgacagttatccatctgggatccatctggaacttcaaagggaatcaagtactgacttcaagcaatttgagaccaatgatcaatgcagaccttatcataggatccttattgagcgtcaagagaaatcagatacaaacttcaaggagtttgttattaaaaagctgcagaagaattgccagtgcagtccagccaaagccaaaaatatgattttaggtttccttcctgttttgcagtggctcccaaaatacgacctaaagaaaaacattttaggggatgtgatgtcaggcttgattgtgggcatattattggtgccccagtccattgcttattccctgctggctggccaagaacctgtctatggtctgtacacatctttttttgccagcatcatttattttctcttgggtacctcccgtcacatctctgtgggcatttttggagtactgtgccttatgattggtgagacagttgaccgagaactacagaaagctggctatgacaatgcccatagtgctccttccttaggaatggtttcaaatgggagcacattattaaatcatacatcagacaggatatgtgacaaaagttgctatgcaattatggttggcagcactgtaacctttatagctggagtttatcaggtagcgatgggcttctttcaagtgggttttgtttctgtctacctctcagatgccttgctgagtggatttgtcactggtgcctccttcactattcttacatctcaggccaagtatcttcttgggctcaaccttcctcggactaatggtgtgggctcactcatcactaccgggagtacatgtcttcagaaacatccataagaccaatctctgtgatcttatcaccagaccttttgtgccttttaggttctttatgccaaccaaagaactcaagtgaacacttcaaatccaagcttaaggcaccagattcctattagaacgtggttgttgttgtagcagacacattagcctctcattttggaaaactacatgaaaattataattctagtattgctggacatattcccactgggtttatgccagcccaaagtaccagaatggaacctaattcctagtgtggctgtagatgcaactagctatttccatcattggttttgctatcactgtatcactttctgagatgtttgccaagaaacatggttacacagtcaaagcaaaccaggaaatgtatgccattggcttttgtaatatcatcccttccttcttccactgttttactactagtgcagctcttgcaaagacattggttaaagaatcaacaggctgccatactcagctttctggtgtggtaacagacctggttcttttcgttggtcctcctagtaatagctcctttgttctattcccttcaaaaaagtgtccttggtgtgatcacaattgtaaatctacggggagcccttcgtaaatttagggatcttcccaaaatgtggagtattagtagaatggatacagcttatctggctttgttactatgctgtcctctgcactgctaagtactgaaataggcctacttgttggggtttgtttttctatattttgtgtcatcctccgcactcagaagccaaagagttcactgcttggcttggtggaagagtctgaggtctttgaatctgtgtctgcttacaagaaccttcagactaagccaggcatcaagattttccgctttgtagcccctctctactacataaacaaagaatgctgttaaatctgctttatacaaacaaactgtcaacccaatcttaataaaggtggcttggaagaaggcagcaaagagaaagatcaaagacaaagtagtgactcttggtggaatccaggatgaaatgtcagtgcaactttcccatgatcccttggagctgcatactatagtgattgactgcagtgcaattcaatttttagatacagcagggatccacacactgaaagaagttcgcagagattatgaagccattggaatccaggttctgctggctcagtgcaatcccactgtgagggattccctaaccaacggagaatattgcaaaaaggaagaagaaaaccttctcttctatagtgtgtatgaagcgatggcttttgcagaagtatctaaaaatcagaaaggagtatgtgttcccaatggtctgagtcttagtagtgattaattgagaaggtagatagaagaatgtctagccaataggttaaaatttcaagtgtccaacatttcccagttccacagtgggaaattttgcacacttgaaattttaaccaagtggctagatattattcctcctttgaagctaatggcatttgtatatacacactgcagcagagcttgtagctggacagagtcaaaaagaagaaaatacggtttcaggctttcttgcagatatgaagtattcttggaatgcaataagtatgtattgaactgtactgtaaagtagctccaaaacttaattactctcctgttttaggggttatacatttgggactgtgcattctccaagagatgaagcggtgaagttggggatttacattgggaagtgctgtagacttctttatgtggcccagtggaagagagggaaaagaatgttgcacctgctctaagtaccataggtcaagaggcttctggatcacaaaagtcataaccaagacaggtttgttcttgtagttttctatccccagtctttgctccccagatggcagtagtttttagtaggaaagtgccattcctgtccttaaggcacagtctcatcagaagtttaatacctgggcaggtttataacatcctgagagccagcctgacattagacagaataccctttgtaatacattggaaatttttactcatgcctttttgtttaggataaataggtaagcacaaagagctcttcaaaatcagaaaaaacaataggagtccttccttgtctttcctgtgatctctgtccttgtttctgagactttctctaccattaagctctattttaggtttcagttattctagtttgtttcccatggaattctgtcctaaaactggtgtttttgtcagtgacagtccttgcccagtcagcaatttcctaacagcattttaaatgagtttgatgtacagtaaatattgatgacaatgacagcttttaactcttcaagtcacctaaagctattatgcaggaggatttagaagtcacattcataaaacccaagtgctatgggtgtattattcatgatagctggcccacaggtcatgaattgaggaggaatttgctttcaaaaagcaagaatgtccaacactgaaagtttatagttttatatttggaccttgaaaggtaagaaaaaaccaggttctccaaagttaggaatagggaactaatttatgaaacagccatcttaaaaaaaaaaaaaagtaaactgcaaaagtacaaaatcatttttcaatctgttcccagtttctaaacaattttaaatatttatgagaagcaaaccctatgtgtagggcatctgtgggagtgggatgcttttagacatattttaagtatgtacatgtttaatatgtttttttaaaatggcatatatattttattatatctatattatcctatatagatatttgtaacttagctttattggttagctccataagctggcagtggttggcttttctggtgggtagagctctcccattgggtgacttggaaaatacctttccattatcacaacaaagcagtggtcagtagaacgtctagatttctgtcttataggtgattcgtgcttataggtgattataatcaagtgtaggcttcctgaatttggacatcttttagaactg

SEQ ID NO: 29 - DNA, 2918 bp, Homo sapiens
  misc_feature: Incyte ID No 989613CB1
cttgcagcaatggcttggattagatccacttgcattctcttttttaccttgctttttgcccacatagcagctgtaccgattaagtatcttcctgaagaaaatgtacatgatgcagattttggtgaacagaaggatatttcagaaatcaatttagctgcaggcttggacctctttcaaggggacatcctcttgcagaaatccagaaatggcctgagagacccaaacaccaggtggacgttccccattccttacatcttggctgataatttggggctgaatgctaaaggagccattctgtatgcctttgagatgttccgtctcaagtcctgtgtggatttcaagccctatgaaggagagagctcatatatcatatttcaacagtttgatgggtgctggtctgaggttggtgaccaacatgtgggacagaacatttccattggccaaggatgtgcctataaggccatcatagaacacgagatcctgcatgctttgggattttaccacgagcagtcaaggacggaccgggatgattatgtgaacatctggtgggaccaaattctttcaggttaccagcacaactttgacacctatgatgatagcttaatcacagacctcaatacaccctatgattatgagtctttgatgcactaccagcctttctcatttaacaagaatgcaagtgttcccaccatcacagccaagatccctgagtttaactccattatcggacaacgcctggatttcagtgccattgatttagagaggctgaaccgaatgtacaattgcaccacaactcacactcttttggaccactgtacttttgagaaggcaaacatctgtggaatgattcagggcaccagagatgacactgactgggcccatcaggacagtgctcaggctggagaagtggatcacaccttgttgggacaatgcacaggtgccggctacttcatgcagttcagcaccagctcggggtccgcggaagaggcagccctactggagtctcggattctttacccaaagaggaagcagcagtgcctgcaatttttctataaaatgacgggaagtccttcagacagactcgttgtctgggtcaggagggatgacagcacaggcaatgttcgcaagttggtgaaggtgcagacttttcaaggagatgatgaccacaattggaaaattgcccatgtggtgctcaaagaggaacagaagtttcgctaccttttccagggcacaaaaggcgaccctcagaactcaactgggggaatttacctagatgacatcactctgacagaaaccccctgccccacaggggtctggacagtccggaatttctcccaagtccttgagaacaccagcaaaggggacaagcttcagagccctcgattctacaattcggagggatatggttttgggttaactttatacccaaatagcagagaaagctctggttacttgagacttgcttttcatgtgtgcagtggggagaacgatgctatcctggagtggccggtagaaaacagacaggtgataattaccatccttgaccaggagcctgatgtccagaacaggatgtcctcaagcatggtgttcactacctcgaagtcgcacacatctccagcgataaatgacactgtcatctgggacaggccgtccagggtgggaacctatcatacagactgtaattgttttagaagcatcgacttgggctggagtggtttcatttcccaccaaatgctgaaaaggaggagtttcctgaaaaatgatgacctcatcatatttgtggactttgaagatatcacccacctcagccagactgaagttcccactaaaggcaaaagactgagcccccaaggcctcattctccaaggccaggagcagcaggtctccgaagaaggttcgggaaaggccatgttagaggaagccctacctgtcagcctgagccaggggcagcccagccgacagaagcggtcggtggagaacacaggccccctggaggaccataactggccacagtacttcagagacccatgtgacccaaacccttgccaaaatgacggcatctgtgtgaacgtgaaggggatggcgagctgcaggtgcatctctggacatgctttcttctacacgggggagcgctgtcaggccgtgcaggtgcacggcagtgtcctgggcatggtgatcggaggcacggctggcgtgatcttcttgaccttctccatcatcgccatcctttcccaaaggccaaggaagtgacctgcctgctggcattggccagaccacagcagcacctcctccatgcaggccttaactttcccatgttcaatgcagtttggggcagcttttttatcagccttgctttggataggacctccaaggactaaggcctccagccccatgtgtgacccttgtcatctctctgccccacataattatgttactttgctatgtgctcctaatgtatctagtgtgtcctgtgacaacactcatcacacttcattgtaaatcacttgttttattgactgtctttcctatagactgtaagctccatgagggcaggcacatgttgttctcattgaccgtgctggccccagtgcctagatgcatggctggcacattgttggcactcaacaatggttgaatgaataaaacaataaatgaatgaataactaagatatagaaactctcatttatattgcagattgaatatatatgatgaaattcttatgttgaatatgttagaatcaaatactcatttttcattagatacagtagtgtcatcactcttttaagatcttgttaaagatttcaaataaaggtacttctgggcagccaggctgcacagcatttgctttcctctgagattctaagagaaggcctttaataaatttaataaatattgagttagcaaaaaaaaaaaaaaa

SEQ ID NO: 30 - protein, 746 aa, Homo sapiens
  misc_feature: Incyte ID No 989613CD1
MAWIRSTCILFFTLLFAHIAAVPIKYLPEENVHDADFGEQKDISEINLAAGLDLFQGDILLQKSRNGLRDPNTRWTFPIPYILADNLGLNAKGAILYAFEMFRLKSCVDFKPYEGESSYIIFQQFDGCWSEVGDQHVGQNISIGQGCAYKAIIEHEILHALGFYHEQSRTDRDDYVNIWWDQILSGYQHNFDTYDDSLITDLNTPYDYESLMHYQPFSFNKNASVPTITAKIPEFNSIIGQRLDFSAIDLERLNRMYNCTTTHTLLDHCTFEKANICGMIQGTRDDTDWAHQDSAQAGEVDHTLLGQCTGAGYFMQFSTSSGSAEEAALLESRILYPKRKQQCLQFFYKMTGSPSDRLVVWVRRDDSTGNVRKLVKVQTFQGDDDHNWKIAHVVLKEEQKFRYLFQGTKGDPQNSTGGIYLDDITLTETPCPTGVWTVRNFSQVLENTSKGDKLQSPRFYNSEGYGFGLTLYPNSRESSGYLRLAFHVCSGENDAILEWPVENRQVIITILDQEPDVQNRMSSSMVFTTSKSHTSPAINDTVIWDRPSRVGTYHTDCNCFRSIDLGWSGFISHQMLKRRSFLKNDDLIIFVDFEDITHLSQTEVPTKGKRLSPQGLILQGQEQQVSEEGSGKAMLEEALPVSLSQGQPSRQKRSVENTGPLEDHNWPQYFRDPCDPNPCQNDGICVNVKGMASCRCISGHAFFYTGERCQAVQVHGSVLGMVIGGTAGVIFLTFSIIAILSQRPRK

SEQ ID NO: 31 - DNA, 1142 bp, Homo sapiens
  misc_feature: Incyte ID No 2921920CB1
ggagctccgagtgtccacaggaagggaactatcagctcctggcatctgtaaggatgctgtccatgctgaggacaatgaccagactctgcttcctgttattcttctctgtggccaccagtgggtgcagtgcagcagcagcctcttctcttgagatgctctcgagggaattcgaaacctgtgccttctccttttcttccctgcctagaagctgcaaagaaatcaaggaacgctgccatagtgcaggtgatggcctgtattttctccgcaccaagaatggtgttgtctaccagaccttctgtgacatgacttctgggggtggcggctggaccctggtggccagcgtgcacgagaatgacatgcatgggaagtgcacggtgggtgatcgctggtccagtcagcagggcaacaaagcagactacccagagggggatggcaactgggccaactacaacacctttggatctgcagaggcggccacgagcgatgactacaagaaccctggctactacgacatccaggccaaggacctgggcatctggcatgtgcccaacaagtcccccatgcagcattggagaaacagcgccctgctgaggtaccgcaccaacactggcttcctccagagactgggacataatctgtttggcatctaccagaaatacccagtgaaatacagatcagggaaatgttggaatgacaatggcccagccatacctgtggtctatgactttggtgatgctaagaagactgcatcttattactcaccgtatggtcaacgggaatttgttgcaggattcgttcagttccgggtgtttaataacgagagagcagccaacgccctttgtgctgggataaaagttactggctgtaacactgagcatcactgcatcggtggaggagggttcttcccacagggcaaaccccgtcagtgtggggacttctccgcctttgactgggatggatatggaactcacgttaagagcagctgcagtcgggagataacggaggcggctgtactcttgttctatagatgagacagagctctgcggtgtcagggcgagaacccatcttccaaccccggctatttggagacggaaaaactggaattctaacaaggaggagaggagactaaatcacatcaattc

SEQ ID NO: 32 - protein, 325 aa, Homo sapiens
  misc_feature: Incyte ID No 2921920CD1
MLSMLRTMTRLCFLLFFSVATSGCSAAAASSLEMLSREFETCAFSFSSLPRSCKEIKERCHSAGDGLYFLRTKNGVVYQTFCDMTSGGGGWTLVASVHENDMHGKCTVGDRWSSQQGNKADYPEGDGNWANYNTFGSAEAATSDDYKNPGYYDIQAKDLGIWHVPNKSPMQHWRNSALLRYRTNTGFLQRLGHNLFGIYQKYPVKYRSGKCWNDNGPAIPVVYDFGDAKKTASYYSPYGQREFVAGFVQFRVFNNERAANALCAGIKVTGCNTEHHCIGGGGFFPQGKPRQCGDFSAFDWDGYGTHVKSSCSREITEAAVLLFYR

SEQ ID NO: 33 - DNA, 2966 bp, Homo sapiens
  misc_feature: Incyte ID No 997080.1
gcagatcagtgtgtgagggaactgccatcatgaggtctgacaagtcagctttggtatttctgctcctgcagctcttctgtgttggctgtggattctgtgggaaagtcctggtgtggccctgtgacatgagccattggcttaatgtcaaggtcattctagaagagctcatagtgagaggccatgaggtaacagtattgactcactcaaagccttcgttaattgactacaggaagccttctgcattgaaatttgaggtggtccatatgccacaggacagaacagaagaaaatgaaatatttgttgacctagctctgaatgtcttgccaggcttatcaacctggcaatcagttataaaattaaatgatttttttgttgaaataagaggaactttaaaaatgatgtgtgagagctttatctacaatcagacgcttatgaagaagctacaggaaaccaactacgatgtaatgcttatagaccctgtgattccctgtggagacctgatggctgagttgcttgcagtcccttttgtgctcacacttagaatttctgtaggaggcaatatggagcgaagctgtgggaaacttccagctccactttcctatgtacctgtgcctatgacaggactaacagacagaatgacctttctggaaagagtaaaaaattcaatgctttcagttttgttccacttctggattcaggattacgactatcatttttgggaagagttttatagtaaggcattaggaaggcccactacattatgtgagactgtgggaaaagctgagatatggctaatacgaacatattgggattttgaatttcctcaaccataccaacctaactttgagtttgttggaggattgcactgtaaacctgccaaagctttgcctaaggaaatggaaaattttgtccagagttcaggggaagatggtattgtggtgttttctctggggtcactgtttcaaaatgttacagaagaaaaggctaatatcattgcttcagcccttgcccagatcccacagaaggtgttatggaggtacaaaggaaaaaaaccatccacattaggagccaatactcggctgtatgattggataccccagaatgatcttcttggtcatcccaaaaccaaagcttttatcactcatggtggaatgaatgggatctatgaagctatttaccatggggtccctatggtgggagttcccatatttggtgatcagcttgataacatagctcacatgaaggccaaaggagcagctgtagaaataaacttcaaaactatgacaagcgaagatttactgagggctttgagaacagtcattaccgattcctcttataaagagaatgctatgagattatcaagaattcaccatgatcaacctgtaaagcccctagatcgagcagtcttctggatcgagtttgtcatgcgccacaaaggagccaagcacctgcgatcagctgcccatgacctcacctggttccagcactactctatagatgtgattgggttcctgctgacctgtgtggcaactgctatattcttgttcacaaaatgttttttattttcctgtcaaaaatttaataaaactagaaagatagaaaagagggaatagatctttccaaattcaagaaagacctgatggggtaatcctgttaattccagccacatagaatttggtgaaaaccttgctattttcatattatctattctgttattttatcttagctatatagcctagaattccacgatcatgaggttgtgagtatatctcattctttcgttgtattttcctaggtgtctttactctcttctctcactttgtgacacaaggacatgaatacatctaaattttcctatttctgatatgactgttttgatgatgtcattacttctataaccttaagtgatagggtgacatgcaatatgattattcctggtgtgcgcccaaacacatggatataaagaggtaaaaaacttaaaattcacaaaattcagtaaaccacacaaatcaggtaagtgttctatgagattagctggctatgagaaacataatgatgtttctttttcaatttaaataagcctttctacatagccagcatcagtgatctcagaaaataaattgctaataatgatgacatggcattatgcttagaaaagtttgctgtatttccatagacctcatctagatgtcatggcctacatttctgccatcactcaaccaatacttttttctgttttcttgatgataaaaagacctttctcatgattgccatcaaataacaaaagaaactattttttttctcacatagagaacatgtcagtaagatattcaaggtgaacagatatttttgggattagtaactatttgaaatatgtggtgataattactgagtttataaaatttatttgatagtacacttaaagaagatttatatgtttattctttaaaaatgatgaatactcataattcttatctctataatcaaaagtataatttactgtagaaaaataaagagatgcttgttctgaaagtaagatcagtgaactgcttttcagtctcaatctttgagaattgtaaattcatcaaataattgcttacatagtaaaaatttaaggtattagaaaacctgcataacaaatagtattatatattaaatattttgatatgtaaagctctacacaaagctaaatatagtgtaataatgtttacactagtaagcaaatatgttaatcttctcatttttttactgtcatataatcttagtgatatgcctattaatagttttaaataaataaattggcttatctggctttttgaaaattttgaaattcttacagatgttgattaggtatatctacaaattaatttcaattttaaaatgatgatataaaaataaatataagtatttttcttgtgtatgtatacaataaatataaataaaattgtttactgttttgaaagttt

SEQ ID NO: 34 - DNA, 2574 bp, Homo sapiens
  misc_feature: Incyte ID No 5517972CB1
gcgcctcccacgccggccgccgccgacgtgatcgctcgggcgcgccgggcgtggttgggggaaggggttgtgccgcgcgacggtctgcgtgctgtgcccactcaaaaggttccgggcgcgcaggacggaagacgcagtgctcgccactcccactgagattgagagacgcggcaaggaggcagcctgtggaggaactgggtaggatttaggaacgcaccgtgcacatgcttggtggtcttgttaagtggaaactgctgctttagagtttgtttggaaggtccgggtgactcatcccaacatttacatccttaattgttaaagcgctgcctccgagcgcacgcatcctgagatcctgagcctttggttaagaccgagctctattaagctgaaaagataaaaactctccagatgtcttccagtaatgtcgaagtttttatcccagtgtcacaaggaaacaccaatggcttccccgcgacagcttccaatgacctgaaggcatttactgaaggagctgtgttaagttttcataacatctgctatcgagtaaaactgaagagtggctttctaccttgtcgaaaaccagttgagaaagaaatattatcgaatatcaatgggatcatgaaacctggtctcaacgccatcctgggacccacaggtggaggcaaatcttcgttattagatgtcttagctgcaaggaaagatccaagtggattatctggagatgttctgataaatggagcaccgcgacctgccaatttcaaatgtaattcaggttacgtggtacaagatgatgttgtgatgggcactctgacggtgagagaaaacttacagttctcagcagctcttcggcttgcaacaactatgacgaatcatgaaaaaaacgaacggattaacagggtcattcaagagttaggtctggataaagtggcagactccaaggttggaactcagtttatccgtggtgtgtctggaggagaaagaaaaaggactagtataggaatggagcttatcactgatccttccatcttgttcttggatgagcctacaactggcttagactcaagcacagcaaatgctgtccttttgctcctgaaaaggatgtctaagcagggacgaacaatcatcttctccattcatcagcctcgatattccatcttcaagttgtttgatagcctcaccttattggcctcaggaagacttatgttccacgggcctgctcaggaggccttgggatactttgaatcagctggttatcactgtgaggcctataataaccctgcagacttcttcttggacatcattaatggagattccactgctgtggcattaaacagagaagaagactttaaagccacagagatcatagagccttccaagcaggataagccactcatagaaaaattagcggagatttatgtcaactcctccttctacaaagagacaaaagctgaattacatcaactttccgggggtgagaagaagaagaagatcacagtcttcaaggagatcagctacaccacctccttctgtcatcaactcagatgggtttccaagcgttcattcaaaaacttgctgggtaatccccaggcctctatagctcagatcattgtcacagtcgtactgggactggttataggtgccatttactttgggctaaaaaatgattctactggaatccagaacagagctggggttctcttcttcctgacgaccaaccagtgtttcagcagtgtttcagccgtggaactctttgtggtagagaagaagctcttcatacatgaatacatcagcggatactacagagtgtcatcttatttccttggaaaactgttatctgatttattacccatgaggatgttaccaagtattatatttacctgtatagtgtacttcatgttaggattgaagccaaaggcagatgccttcttcgttatgatgtttacccttatgatggtggcttattcagccagttccatggcactggccatagcagcaggtcagagtgtggtttctgtagcaacacttctcatgaccatctgttttgtgtttatgatgattttttcaggtctgttggtcaatctcacaaccattgcatcttggctgtcatggcttcagtacttcagcattccacgatatggatttacggctttgcagcataatgaatttttgggacaaaacttctgcccaggactcaatgcaacaggaaacaatccttgtaactatgcaacatgtactggcgaagaatatttggtaaagcagggcatcgatctctcaccctggggcttgtggaagaatcacgtggccttggcttgtatgattgttattttcctcacaattgcctacctgaaattgttatttcttaaaaaatattcttaaatttccccttaattcagtatgatttatcctcacataaaaaagaagcactttgattgaagtattcaatcaagtttttttgttgttttctgttcccttgccatcacactgttgcacagcagcaattgttttaaagagatacatttttagaaatcacaacaaactgaattaaacatgaaagaacccaagaaaaaaaaaaaa

SEQ ID NO: 35 - protein, 655 aa, Homo sapiens
  misc_feature: Incyte ID No 5517972CD1
MSSSNVEVFIPVSQGNTNGFPATASNDLKAFTEGAVLSFHNICYRVKLKSGFLPCRKPVEKEILSNINGIMKPGLNAILGPTGGGKSSLLDVLAARKDPSGLSGDVLINGAPRPANFKCNSGYVVQDDVVMGTLTVRENLQFSAALRLATTMTNHEKNERINRVIQELGLDKVADSKVGTQFIRGVSGGERKRTSIGMELITDPSILFLDEPTTGLDSSTANAVLLLLKRMSKQGRTIIFSIHQPRYSIFKLFDSLTLLASGRLMFHGPAQEALGYFESAGYHCEAYNNPADFFLDIINGDSTAVALNREEDFKATEIIEPSKQDKPLIEKLAEIYVNSSFYKETKAELHQLSGGEKKKKITVFKEISYTTSFCHQLRWVSKRSFKNLLGNPQASIAQIIVTVVLGLVIGAIYFGLKNDSTGIQNRAGVLFFLTTNQCFSSVSAVELFVVEKKLFIHEYISGYYRVSSYFLGKLLSDLLPMRMLPSIIFTCIVYFMLGLKPKADAFFVMMFTLMMVAYSASSMALAIAAGQSVVSVATLLMTICFVFMMIFSGLLVNLTTIASWLSWLQYFSIPRYGFTALQHNEFLGQNFCPGLNATGNNPCNYATCTGEEYLVKQGIDLSPWGLWKNHVALACMIVIFLTIAYLKLLFLKKYS

SEQ ID NO: 36 - DNA, 3405 bp, Homo sapiens
  misc_feature: Incyte ID No 1397781.7
ttgtttcccccactcttccctcctgtggaggagaaaaagtagagggttaacaacgccaggcagggcaccagggcccacagggcgaggacgagcagcatccacacgcagccggagggcagactgcagggagcagagctggcccagcgatcctgggggaggccgcgccagagacgcagccgcgctcccaccacccacacccaccgcgccctgcgttcgcctcttctccgggagccagtcccgcgccaccgccgccgcccaggccatcgccaccctccgcagccatgtccaccaggtccgtgtcctcgtcctcctaccgcaggatgttcggcggcccgggcaccgcgagccggccgagctccagccggagctacgtgactacgtccacccgcacctacagcctgggcagcgcgctgcgccccagcaccagccgcagcctctacgcctcgtccccgggcggcgtgtatgccacgcgctcctctgccgtgcgcctgcggagcagcgtgcccggggtgcggctcctgcaggactcggtggacttctcgctggccgacgccatcaacaccgagttcaagaacacccgcaccaacgagaaggtggagctgcaggagctgaatgaccgcttcgccaactacatcgacaaggtgcgcttcctggagcagcagaataagatcctgctggccgagctcgagcagctcaagggccaaggcaagtcgcgcctgggggacctctacgaggaggagatgcgggagctgcgccggcaggtggaccagctaaccaacgacaaagcccgcgtcgaggtggagcgcgacaacctggccgaggacatcatgcgcctccgggagaaattgcaggaggagatgcttcagagagaggaagccgaaaacaccctgcaatctttcagacaggatgttgacaatgcgtctctggcacgtcttgaccttgaacgcaaagtggaatctttgcaagaagagattgcctttttgaagaaactccacgaagaggaaatccaggagctgcaggctcagattcaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgtgaaatggaagagaactttgccgttgaagctgctaactaccaagacactattggccgcctgcaggatgagattcagaatatgaaggaggaaatggctcgtcaccttcgtgaataccaagacctgctcaatgttaagatggcccttgacattgagattgccacctacaggaagctgctggaaggcgaggagagcaggatttctctgcctcttccaaacttttcctccctgaacctgagggaaactaatctggattcactccctctggttgatacccactcaaaaaggacacttctgattaagacggttgaaactagagatggacaggttatcaacgaaacttctcagcatcacgatgaccttgaataaaaattgcacacactcagtgcagcaatatattaccagcaagaataaaaaagaaatccatatcttaaagaaacagctttcaagtgcctttctgcagtttttcaggagcgcaagatagatttggaataggaataagctctagttcttaacaaccgacactcctacaagatttagaaaaaagtttacaacataatctagtttacagaaaaatcttgtgctagaatactttttaaaaggtattttgaataccattaaaactgcttttttttttccagcaagtatccaaccaacttggttctgcttcaataaatctttggaaaaacaaagcagttttaatggtattcaaaataccttttaaaaagtattctagcacaagatttttctgtaaactagattatgttgtaaacttttttctaaatcttgtaggagtgtcggttgttaagaactagagcttattcctattccaaatctatcttgcgctcctgaaaaactgcagaaaggcacttgaaagctgtttctttaagatatggatttcttttttattcttgctggtaatatattgctgcactgagtgtgtgcaatttttattcaaggtcatcgtgatgctgagaagtttcgttgataacctgtttaaaaaaaaaaaccaaaataaatataatgccacatgccaaagactaaattatcctatatatcacggcaaataaaaatcctgctaaatatatgtaatttattgtgcaaatcagaaggttatgttgcttctgcatgagtgaaaaactagaaggggcagacacacaagaccctcttccactacaggcaatataaggatccagtggaatgttgtaacttacaaaaagaaatttcttaaaaaactgtgtttataaaagatggtataatagttgtacacaatacagccaagtttaaaagcaattaagacatattaaaatgtgcagcaggtacattttaaatttatgtgcaagggaagatttcatgatgactacaataaattgcaactatttctttcctggcatagggaggtaataagaaactaaatgatcgtatggcacatgcttgtattatatagatgggtttaggaatctataaagtatggaggtaggaagacaccatatgtccaggatcaaaacattcctcatattgaggtagtctagtgaagctgtttcatgtagctgctttaggaagtggtttaaggaagcttactcccacttcaaattaagcaccaaagcaaatcactaattctggagcacaggaagactgctatctcaacttcacatccaggagccaaataattagttggctgtgtgtactgctggaagaagaagcctattctttttttagtaggcatgaatgaaacctgaacctctcaaattagccagctgtatctgagattcagattcactgattttaaaatatatcaactcctgtctgagattaccctattttttccttaaaagataccaacctgtccatctctagtttcaaccgtcttaatcagaagtgtcctttttgagtgggtatcaaccagagggagtgaatccagattagtttctaaataaacaaacaggccaaaaatgagtaaaaacgagttttttcttggaaaaaccaagtaaccattttactgaaacaattatgatttgtgggggagaggcaattgtgtaaagtactactcgccaaataattttagggtagtctctgtttggaaacccaggcc

SEQ ID NO: 37 - DNA, 273 bp, Homo sapiens
  misc_feature: Incyte ID No 236655.3
ctctgggaagtggactgtggtttttccagaggaactcagttaagaaatcgagagtggattagactcccagttccaccaaacctatgagccttccactgtggatgggggccgtgatcctgatggtcacattgctttaacccagcagggcttcggccaggggctttccacttgaggatagcagcttcactaggctggccggccagctccacatctgactgggttcttacttctcagccagtacctacccctattgcggtcctccagctcatcttt

SEQ ID NO: 38 - DNA, 2333 bp, Homo sapiens
  misc_feature: Incyte ID No 345275.4
gctgcgcgcgggcgagctgccgcggagcacccggcaggggctgacagcatggcctcgcccgacccgcccgccaccagctacgccccgtccgacgtgccctcgggggtcgcgctgttcctcaccatccctttcgccttcttcctgcccgagctgatatttgggttcttggtctggaccatggtagccgccacccacatagtataccccttgctgcaaggatgggtgatgtatgtctcgctcacctcgtttctcatctccttgatgttcctgttgtcttacttgtttggattttacaaaagatttgaatcctggagagttctggacagcctgtaccacgggaccactggcatcctgtacatgagcgctgccgtcctacaagtacatgccacgattgtttctgagaaactgctggacccaagaatttactacattaattcggcagcctcgttcttcgccttcatcgccacgctgctctacattctccatgccttcagcatctattaccactgatgcacaggcgccaggccaagggggaaatgctctttgaaagctccaattattggtccccaaaagcagcttccaacgtttgccatctggatgacaaacggaagatccactaaaacgtccacgggattaacagaacgtccttgcagactgagcgatgacaccacactttgtttggacatttaaattcactctgctgaataggaggaagcttttctttttcctgggaaaacaactgtctcttggaattatctgaccatgaacttgctcttctagacaactcacatcaaagccctcactccactaatggagaatcctagccccactaatgccaagtctgtttggggattttgcctcagctatgggcttccctagagtaggtctaggggaatactcagtctgatctttttnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnntgattctcttaaaattgaagaggtgctgccaagggccttcagatctaacgcagatgcatagaccttgttcctggtacttgttcagcctgtgctggggagccgtggtcccgagttccctgggaggctgacagggtcaagccaccctgcccaccaccctcccacttcccctcccctttcctctccagcattaggattcaagggaaatctgcatgaagccaattttgagggtagacgtgtggggaaaataaatcattatacagtaagacctggggcttgaggggtggggaatggggagggaagggcatagcctgctcctccatgagtctgacatctcggaaactgagcagctgccggacgcctgggtcaggaatccaagaccccacctcttaaggactggttcctcagaaagcaccctcagggaaaaaggtgaaaacattacatccgtggattctcctgccacaaccgcattggaagaaaaggctgccgcaacatctcagcgaggagtgaaggacccatgtcccaggaaccgcgctgcgccacctgcactcacccccctcacattctcttaagcacccggtggccctccgaggcctggcggaatggtggtgcccacggggttgggcaagggctcaccaggacctcaacgggcaaagttgtgcacactaaaatatcaaatcaaggtgcttggttttaaagtaaatgtttttctaaagaaagctgtgttcttctgttgacccagacgaatagggcacagccctgtaactgcacgtgccttctgtcattgggaatgaaataaattattacgagaaagggacttgtcctaactggtttgaggccttacagttttgtatctacatttttcccctcctggggtttgcggggacagggacagaactacaggagtcatgggaaagaaaattctggcttcactactgctcactgctcactttctgatcactctgatacttttttttttntttttttttgcaacctgataccttgaaaagcttctatgtgtctctccttttgttgcctggcagctgtctaggatgatcactgattactatttactaagtagccacatgcaaataaaagttgtttggtaaaatga

SEQ ID NO: 39 - DNA, 1448 bp, Homo sapiens
  misc_feature: Incyte ID No 124600CB1
gcccgccccagcagtggctgcaccatgcacgtgaacggcaaagtggcgctggtgaccggcgcggctcagggcataggcagagcctttgcagaggcgctgctgcttaagggcgccaaggtagcgctggtggattggaatcttgaagcaggtgtacagtgtaaagctgccctggatgagcagtttgaacctcagaagactctgttcatccagtgcgatgtggctgaccagcaacaactgagagacacttttagaaaagttgtagaccactttggaagactggacattttggtcaataatgctggagtgaataatgagaaaaactgggaaaaaactctgcaaattaatttggtttctgttatcagtggaacctatcttggtttggattacatgagtaagcaaaatggaggtgaaggcggcatcattatcaatatgtcatctttagcaggactcatgcccgttgcacagcagccggtttattgtgcttcaaagcatggcatagttggattcacacgctcagcagcgttggctgctaatcttatgaacagtggtgtgagactgaatgccatttgtccaggctttgttaacacagccatccttgaatcaattgaaaaagaagaaaacatgggacaatatatagaatataaggatcatatcaaggatatgattaaatactatggaattttggacccaccattgattgccaatggattgataacactcattgaagatgatgctttaaatggtgctattatgaagatcacaacttctaagggaattcattttcaagactatgatacaactccatttcaagcaaaaacccaatgaacagcttatgtgttagccatagctgaaaataagcacaaatagcttatattcagatcctatcttcatttgaatatagcttttaaatgaaatgttacagaatgaagttttccttcatgcacttggtgataaacgtcttctaaattttcagttaagtatatggataaaaagttatgaactattaaaaatgtgatgtggaccaaaggctaggttgtaatcttgatagtctaaaaatgatcacaacaaatgattttcaaggaatattcactattctgccttccagaaagtgtatttatatctgggcttcaaaaatatcaatgttcttcagaacatcactttaanggagatactcgactcgctatctaactcaccccagatgttacacactcacattcacgttcatactttcaaagaggaaagtcatcaacaatgacacttttccacactaatcatctccattttctcccactcccactggacaactcaggcttcaagggttcttaggtcgatttttcgcggcaggacagactccttcaaggccccatatccggctaactctcgttttgcaaatcagaacacactcatcacatcggccaaaaccaaccccttctgaaacgacacttcct

SEQ ID NO: 40 - protein, 266 aa, Homo sapiens [sequence omitted]

SEQ ID NO: 41 - DNA, 743 bp, Homo sapiens
  misc_feature: Incyte ID No 978410.7
cagcccggttcctaacagaccacagaccccacaccaggtctatctcatttggtctcagagctgtgaatcagccagcaatattttagttgcaaatcactgaaaacccaactcaaagtgacttaagtcagaaagaaattttatgaattcaggtaattaaaaagtccagaagtatctgcctttaggcacagctggatccaagggcacaaatgatgtcatcaggctccagttattctccatctcccagctcagctttttctgtctgtaagcctgattttcaggaaggctctttcctagtgatggagatgaccaccatcagctccaggcttctatcctgctaacccagtaacccagtgggaagagatttacttattccaataattccaagtggagagtgtcattgacccgtttggggtctcatctctacttctaggggaatgaaacactctgagtggccaggcctgtgtcatgtgctaattcctagagccagggaaataaggtctgaggattcaggatggggtgaaaggtggttgcttaaaggaaaatgaaatacaattagcagaataaggggaaacgagtggtctgctctgctcgggcaaaacaagagatgcccattactgtgagggacccttgaagtctggactcttaaatgggtttttgctgatttcctgggtgcatgctaggatgatggggcttgatgcagtagggaagagacgatgtaaaaataataaacaatatataccttc

SEQ ID NO: 42 - DNA, 830 bp, Homo sapiens
  misc_feature: Incyte ID No 1401116.1
gtggtgcctcagccatggcctggacctttctcctcctcggcctcctctctcactgcacagactctgcgacctccctcgtgttgactcagccgccctcggtgtcggtggccccaggaaagacggccaccattatttgtgggggacacaacgttggaagttatagtgtccactggtaccaacagaagcccggccaggcccctgtcttggtcatctataacgatgacgaccggccctcagggatctctgagcgattctccggctccaattctggggacacggccaccctgaccatcagcagggtcgaagtcggggatgaggccgactattactgtcacttgtggcatagtgctagtaatcctccttatgtcctcgggactgggacgacggtcaccgtcctcggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataggttcccaactctaaccccacccacgggagcctggagctgcaggatcccaggggaggggtctctctccccatcccaagtcatccagcccttctccctgcactcatcaaacca

SEQ ID NO: 43 - DNA, 2147 bp, Homo sapiens
  misc_feature: Incyte ID No 2921009CB1
cggctcgagggattctgctaatacctatcactgtaggtgctgaagggaaacagatgaagaacatgacctcaaggagcttcctgtcaatgagaagaccaagctgacgcctggcaaagatattaaagaggagcctgaaactgttccttggacatcttatgaatgtcagaaaataccttttggagggttagaagatcaggggacatggttgttcacatttgctgccacggaacaccgccagtcttcacttggaaacagaatcacgccttgtgaagagatcatccctaagcaggagagaagctactaaaggattgtgtcctcctccaccttccctgtgctcggtctccacctgtctcccattctgtgacgatggttcaatggaaggggctctgccagctgcattacttgtgggctctgggctgctatatgctgctggccactgtggctctgaaactttctttcaggttgaagtgtgactctgaccacttgggtctggagtccagggaatctcaaagccagtactgtaggaatatcttgtataatttcctgaaacttccagcaaagaggtctatcaactgttcaggggtcacccgaggggaccaagaggcagtgcttcaggctattctgaataacctggaggtcaagaagaagcgagagcctttcacagacacccactacctctccctcaccagagactgtgagcacttcaaggctgaaaggaagttcatacagttcccactgagcaaagaagaggtggagttccctattgcatactctatggtgattcatgagaagattgaaaactttgaaaggctactgcgagctgtgtatgcccctcagaacatatactgtgtccatgtggatgagaagtccccagaaactttcaaagaggcggtcaaagcaattatttcttgcttcccaaatgtcttcatagccagtaagctggttcgggtggtttatgcctcctggtccagggtgcaagctgacctcaactgcatggaagacttgctccagagctcagtgccgtggaaatacttcctgaatacatgtgggacggactttcctataaagagcaatgcagagatggtccaggctctcaagatgttgaatgggaggaatagcatggagtcagaggtacctcctaagcacaaagaaacccgctggaaatatcactttgaggtagtgagagacacattacacctaaccaacaagaagaaggatcctcccccttataatttaactatgtttacagggaatgcgtacattgtggcttcccgagatttcgtccaacatgttttgaagaaccctaaatcccaacaactgattgaatgggtaaaagacacttatagcccagatgaacacctctgggccacccttcagcgtgcacggtggatgcctggctctgttcccaaccaccccaagtacgacatctcagacatgacttctattgccaggctggtcaagtggcagggtcatgagggagacatcgataagggtgctccttatgctccctgctctggaatccaccagcgggctatctgcgtttatggggctggggacttgaattggatgcttcaaaaccatcacctgttggccaacaagtttgacccaaaggtagatgataatgctcttcagtgcttagaagaatacctacgttataaggccatctatgggactgaactttgagacacactatgagagcgttgctacctgtggggcaagagcatgtacaaacatgctcagaacttgctgggacagtgtgggtgggagaccagggctttgcaattcgtggcatcctttaggataagagggctgctattagattgtgggtaagtagatcttttgccttgcaaattgctgcctgggtgaatgctgcttgttctctcacccctaaccctagtagttcctccactaactttctcactaagtgagaatgagaactgctgtgatagggagagtgaaggagggatatgtggtagagcacttgatttcagttgaatgcctgctggtagcttttccattctgtggagctgccgttcctaataattccaggtttggtagcgtggaggagaactttgatggaaagagaaccttcccttctgtactgttaacttaaaaataaatagctcctgattcaaagtaaaaaaaaa

SEQ ID NO: 44 - protein, 438 aa, Homo sapiens
  misc_feature: Incyte ID No 2921009CD1
MVQWKGLCQLHYLWALGCYMLLATVALKLSFRLKCDSDHLGLESRESQSQYCRNILYNFLKLPAKRSINCSGVTRGDQEAVLQAILNNLEVKKKREPFTDTHYLSLTRDCEHFKAERKFIQFPLSKEEVEFPIAYSMVIHEKIENFERLLRAVYAPQNIYCVHVDEKSPETFKEAVKAIISCFPNVFIASKLVRVVYASWSRVQADLNCMEDLLQSSVPWKYFLNTCGTDFPIKSNAEMVQALKMLNGRNSMESEVPPKHKETRWKYHFEVVRDTLHLTNKKKDPPPYNLTMFTGNAYIVASRDFVQHVLKNPKSQQLIEWVKDTYSPDEHLWATLQRARWMPGSVPNHPKYDISDMTSIARLVKWQGHEGDIDKGAPYAPCSGIHQRAICVYGAGDLNWMLQNHHLLANKFDPKVDDNALQCLEEYLRYKAIYGTEL

SEQ ID NO: 45 - DNA, 2150 bp, Homo sapiens
  misc_feature: Incyte ID No 255115.4
ggcacgaggaaagaaacaacaactggaaaagaagcattgcataagaccaggatgtctctgaaatggatgtcagtctttctgctgatgcagctcagttgttactttagctctgggagttgtggaaaggtgctggtgtggcccacagaatacagccattggataaatatgaagacaatcctggaagagcttgttcagaggggtcatgaggtgattgtgttgacatcttcggcttctattcttgtcaatgccagtaaatcatctgctattaaattagaagtttatcctacatctttaactaaaaatgatttggaagatttttttatgaaaatgttcgatagatggacatatagtatttcaaaaaatacattttggtcatatttttcacaactacaagaattgtgttgggaatattctgactataatataaagctctgtgaagatgcagttttgaacaagaaacttatgagaaaactacaagagtcaaaatttgatgtccttctggcagatgccgttaatccctgtggtgagctgctggctgaactacttaacataccctttctgtacagtctccgcttctctgttggctacacagttgagaagaatggtggaggatttctgttccctccttcctatgtacctgttgttatgtcagaattaagtgatcaaatgattttcatggagaggataaaaaatatgatatatatgctttattttgacttttggtttcaagcatatgatctgaagaagtgggaccagttttatagtgaagttctaggaagacccactacattatttgagacaatggggaaagctgaaatgtggctcattcgaacctattgggattttgaatttcctcgcccattcttaccaaatgttgattttgttggaggacttcactgtaaaccagccaaacccttgcctaaggaaatggaagagtttgtgcagagctctggagaaaatggtattgtggtgttttctctggggtcgatgatcagtaacatgtcagaagaaagtgccaacatgatgcatcagccctgcccagatcccacaaaaggttctatggagatttgatggcaagaagccaaatactttaggttccaatactcgactgtacaagtggttaccccagaatgaccttcttggtcatcccaaaaccaaagcttttataactcatggtggaaccaatggcatctatgaggcaatctaccatgggatccctatggtgggcattcccttgtttgcggatcaacatgataacattgctcacatgaaagccaagggagcagccctcagtgtggacatcaggaccatgtcaagtagagatttgctcaatgcattgaagtcagtcattaatgaccctatctataaagagaatatcatgaaattatcaagaattcatcatgatcaaccggtgaagcccctggatcgagcagtcttctggattgagtttgtcatgcgccataaaggagccaagcaccttcgggtcgcagcccacaacctcacctggatccagtaccactctttggatgtgatagcattcctgctggcctgcgtggcaactatgatatttatgatcacaaaatgttgcctgttttgtttccgaaagcttgccaaaacaggaaagaagaagaaaagggattagttatatcaaaagcctgaagtggaatgaccaaaagatgggactcctcctttattccagcatggagggttttaaatggaggatttcctttttcctgcgacaaaacgtcttttcacaacttaccctgttaagtcaaaatttattttccaggaatttaatatgtactttagttggaattattctatgtcaatgatttttaagctatgaaaaataataatataaaaccttatgggcttatattgaaatttattattctaatccaaaagttaccccacacaaaagttactgagcttccttatgtttcacacattgtatttgatcacaaaacattaacaactccactcatagtatcaacattgttttgcaaatactcagaatattttggcttcattttgagcagaatttttgtttttaattttgccaatgaaatcttcaataattaaaaaananaaaanannnaaaaaaaaanananaannaacnanngnaaaaaaaaaangggcggccgccgac

SEQ ID NO: 46 - DNA, 764 bp, Homo sapiens
  misc_feature: Incyte ID No 1213592.1
gctgggcctcaggaagcagcatcggaggtgcctcagccatggcatggatccctctcttcctcggcgtccttgcttactgcacaggttccgtggcctcttatgacctgactcagtcacccttagtgtccgtgtccccaggacagacagccaacatcacctgctctgcaggatcaattggggagtacatttgcttcttggtatcagcagaagccaggtcagtcccctgtgttggtcatctatcaggataacaagcggccctcagggatctctgagcgattctctggctccaactctgggaacactgccactctgaccatcaccgagacccaggctacggatgaggctgactattattgtcaggcgtgggacagcagggcattggggtgttcggcggagggaccaagctgaccgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaaccaaagcaaccaaccaaggtacgggggccagcagctatctgagcctgacgcctgagccagtgggaagtcccacagaagctacagctgccaggtcanggcatgaaggggagcaccgtgggaggaaggacagtggccccttacaagaatgtttcataggtttcttcaacc

SEQ ID NO: 47 - DNA, 1764 bp, Homo sapiens
  misc_feature: Incyte ID No 1376382CB1
ctcaggcctgacggtccgagtggagctgcgggacagcccgaacctccaggtcagccccgcggccctccatggcgctggtgcgcgcactcgtctgctgcctgctgactgcctggcactgccgctccggcctcgggctgcccgtggcgcccgcaggcggcaggaatcctcctccggcgataggacagttttggcatgtgactgacttacacttagaccctacttaccacatcacagatgaccacacaaaagtgtgtgcttcatctaaaggtgcaaatgcctccaaccctggcccttttggagatgttctgtgtgattctccatatcaacttattttgtcagcatttgattttattaaaaattctggacaagaagcatctttcatgatatggacaggggatagcccacctcatgttcctgtacctgaactctcaacagacactgttataaatgtgatcactaatatgacaaccaccatccagagtctctttccaaatctccaggttttccctgcgctgggtaatcatgactattggccacaggatcaactgcctgtagtcaccagtaaagtgtacaatgcagtagcaaacctctggaaaccatggctagatgaagaagctattagtactttaaggaaaggtggtttttattcacagaaagttacaactaatccaaaccttaggatcatcagtctaaacacaaacttgtactacggcccaaatataatgacactgaacaagactgacccagccaaccagtttgaatggctagaaagtacattgaacaactctcagcagaataaggagaaggtgtatatcatagcacatgttccagtggggtatctgccatcttcacagaacatcacagcaatgagagaatactataatgagaaattgatagatatttttcaaaaatacagtgatgtcattgcaggacaattttatggacacactcacagagacagcattatggttctttcagataaaaaaggaagtccagtaaattctttgtttgtggctcctgctgttacaccagtgaagagtgttttagaaaaacagaccaacaatcctggtatcagactgtttcagtatgatcctcgtgattataaattattggatatgttgcagtattacttgaatctgacagaggcgaatctaaagggagagtccatctggaagctggagtatatcctgacccagacctacgacattgaagatttgcagccggaaagtttatatggattagctaaacaatttacaatcctagacagtaagcagtttataaaatactacaattacttctttgtgagttatgacagcagtgtaacatgtgataagacatgtaaggcctttcagatttgtgcaattatgaatcttgataatatttcctatgcagattgcctcaaacagctttatataaagcacaattactagtatttcacagtttttgctaatagaaaatgctgattctgattctgagatcaatttgtgggaattttacataaatctttgttaattactgagtgggcaagtagacttcctgtctttgctttctttttttttttctttttgatgccttaatgtagatatctttatcattctgaattgtattatatatttaaagtgctcattaatagaatgatggatgtaaattggatgtaaatattcagtttatataattatatctaatttgtacccttgttgaaattgtcatttatacaataaagcgaattctttatctctaaatatgaaaaaaaaaaaaaaaagg

SEQ ID NO: 48 - protein, 453 aa, Homo sapiens
  misc_feature: Incyte ID No 1376382CD1
MALVRALVCCLLTAWHCRSGLGLPVAPAGGRNPPPAIGQFWHVTDLHLDPTYHITDDHTKVCASSKGANASNPGPFGDVLCDSPYQLILSAFDFIKNSGQEASFMIWTGDSPPHVPVPELSTDTVINVITNMTTTIQSLFPNLQVFPALGNHDYWPQDQLPVVTSKVYNAVANLWKPWLDEEAISTLRKGGFYSQKVTTNPNLRIISLNTNLYYGPNIMTLNKTDPANQFEWLESTLNNSQQNKEKVYIIAHVPVGYLPSSQNITAMREYYNEKLIDIFQKYSDVIAGQFYGHTHRDSIMVLSDKKGSPVNSLFVAPAVTPVKSVLEKQTNNPGIRLFQYDPRDYKLLDMLQYYLNLTEANLKGESIWKLEYILTQTYDIEDLQPESLYGLAKQFTILDSKQFIKYYNYFFVSYDSSVTCDKTCKAFQICAIMNLDNISYADCLKQLYIKHNY

SEQ ID NO: 49 - DNA, 2107 bp, Homo sapiens
  misc_feature: Incyte ID No 2264641CB1
tccaagctattgtcctgcccatggcttcccatctcaggacgctctctggccgctatcatcccagcagtggagttcagcccactactctgaaccagccgcaggtggctgctatgggactgaagccatgaatggtgccggccctggccccgccgcagccgccccggtcccagtcccggtcccggtcccggactggcggcagttctgcgagctgcatgcgcaggcggccgccgtggactttgcgcacaagttctgccgtttcctgcgggacaacccagcttacgacacgcccgacgccggcgcctccttctcccgccacttcgccgccaacttcctggacgtcttcggcgaggaggtgcgccgcgtgctggtggctgggccgacgactcggggcgcggccgtgagcgcagaggccatggagccggagctcgcggacacctctgcactcaaggcggcgtcctacggccactcgcggagctcggaggacgtgtccacgcacgcggccaccaaggcccgcgttcgcaagggcttctcgctgcgcaacatgagcctgtgcgtggtggacggcgtgcgcgacatgtggcaccggcgcgcctcgcccgagcccgacgcggcagctgccccgcgcaccgccgagccccgcgacaagtggacgcggcgcctgaggctgtcgcggacgctggctgccaaggtggagctggtggacattcaacgcgagggggcgctgcgcttcatggtggccgacgacgcggccgcgggctccgggggctcggctcagtggcagaagtgccgcctgctcctgcgcagggctgtggccgaggaacgcttccgcctggagttcttcgtgccgcccaaagcctccaggcccaaggtcagcatcccactgtcagccatcattgaggtccgcaccaccatgcccctggaaatgccagagaaggataacacattcgtcctcaaggtagagaatggagccgaatacatcttggagaccatcgactctctgcagaagcactcgtgggtagctgacatccagggctgcgtggaccccggtgacagtgaggaagacaccgagctctcctgtacccgaggaggctgtctggccagccgcgtggcctcctgcagctgtgagctcctgactgatgcagtcgacctgccccgccccccagagacgacagccgtgggtgcagtggtgacagccccccacagccgaggtcgagatgccgtcagagaatccctgatccacgtcccgctagagacctttctgcagaccctggaatccccgggcggcagcggcagtgacagcaataacacaggggaacagggtgcagagacggatcccgaggctgaacccgagctggagctatccgactacccatggttccacgggacactgtcccgggtcaaggctgctcaactggttctggcaggggggccccggaaccacggcctcttcgtgatccgccaaagtgagactcggcctggggagtacgtgctgaccttcaacttccagggcaaggccaagcacctgcgcctgtccctgaacggccacggccagtgtcacgtacagcatctgtggttccagtctgtgcttgacatgctccgccacttccacacacaccccatcccactggagtcagggggctcggccgacatcacccttcgcagctatgtgcgggcccaggaccccccaccagagccgggccccacgccccctgccgcgcccgcgtccccggcctgctggagcgactcgcccggccagcactacttctccagcctcgccgcggccgcctgcccgcctgcctcgccctccgacgccgccggcgcctcctcgtcttccgcctcgtcgtcctctgccgcgtcggggcccgcccccccgcgccccgtcgagggccagctcagcgcgcggagccgcagcaacagcgccgagcgcctgctggaggccgtggccgccaccgccgccgaggagcccccggaggccgcgcccggccgcgcgcgcgccgtggagaaccagtactccttctactagcccgcggcgccgcccgggtgggacacgccaagctcttcagtgaagacacgatgttattaaaagcctgttttagggactgcaaaa

SEQ ID NO: 50 - protein, 632 aa, Homo sapiens
  misc_feature: Incyte ID No 2264641CD1
MNGAGPGPAAAAPVPVPVPVPDWRQFCELHAQAAAVDFAHKFCRFLRDNPAYDTPDAGASFSRHFAANFLDVFGEEVRRVLVAGPTTRGAAVSAEAMEPELADTSALKAASYGHSRSSEDVSTHAATKARVRKGFSLRNMSLCVVDGVRDMWHRRASPEPDAAAAPRTAEPRDKWTRRLRLSRTLAAKVELVDIQREGALRFMVADDAAAGSGGSAQWQKCRLLLRRAVAEERFRLEFFVPPKASRPKVSIPLSAIIEVRTTMPLEMPEKDNTFVLKVENGAEYILETIDSLQKHSWVADIQGCVDPGDSEEDTELSCTRGGCLASRVASCSCELLTDAVDLPRPPETTAVGAVVTAPHSRGRDAVRESLIHVPLETFLQTLESPGGSGSDSNNTGEQGAETDPEAEPELELSDYPWFHGTLSRVKAAQLVLAGGPRNHGLFVIRQSETRPGEYVLTFNFQGKAKHLRLSLNGHGQCHVQHLWFQSVLDMLRHFHTHPIPLESGGSADITLRSYVRAQDPPPEPGPTPPAAPASPACWSDSPGQHYFSSLAAAACPPASPSDAAGASSSSASSSSAASGPAPPRPVEGQLSARSRSNSAERLLEAVAATAAEEPPEAAPGRARAVENQYSFY

SEQ ID NO: 51 - DNA, 1863 bp, Homo sapiens
  misc_feature: Incyte ID No 237547CB1
cctgaagctacaggtgctccctcctggaatctccaatggatttcagtcgcagaagcttccacagaagcctgagctcctccttgcaggcccctgtagtcagtacagtgggcatgcagcgcctcgggacgacacccagcgtttatgggggtgctggaggccggggtatccgcatctccaactccagacacacggtgaactatgggagcgatctcacaggcggcggggacctgtttgttggcaatgagaaaatggccatgcagaacctaaatgaccgtctagcgagctacctagaaaaggtgcggaccctggagcagtccaactccaaacttgaagtgcaaatcaagcagtggtacgaaaccaacgccccgagggctggtcgcgactacagtgcatattacagacaaattgaagagctgcgaagtcagattaaggatgctcaactgcaaaatgctcggtgtgtcctgcaaattgataatgctaaactggctgctgaggacttcagactgaagtatgagactgagagaggaatacgtctaacagtggaagctgatctccaaggcctgaataaggtctttgatgacctaaccctacataaaacagatttggagattcaaattgaagaactgaataaagacctagctctcctcaaaaaggagcatcaggaggaagtcgatggcctacacaagcatctgggcaacactgtcaatgtggaggttgatgctgctccaggcctgaaccttggcgtcatcatgaatgaaatgaggcagaagtatgaagtcatggcccagaagaaccttcaagaggccaaagaacagtttgagagacagactgcagttctgcagcaacaggtcacagtgaatactgaagaattaaaaggaactgaggttcaactaacggagctgagacgcacctcccagagccttgagatagaactccagtcccatctcagcatgaaagagtctttggagcacactctagaggagaccaaggcccgttacagcagccagttagccaacctccagtcgctgttgagctctctggaggcccaactgatgcagattcggagtaacatggaacgccagaacaacgaataccatatccttcttgacataaagactcgacttgaacaggaaattgctacttaccgccgccttctggaaggagaagacgtaaaaactacagaatatcagttaagcaccctggaagagagagatataaagaaaaccaggaagattaagacagtcgtgcaagaagtagtggatggcaaggtcgtgtcatctgaagtcaaagaggtggaagaaaatatctaaatagctaccagaaggagatgctgctgaggttttgaaagaaatttggctataatcttatctttgctccctgcaagaaatcagccataagaaagcactattaatactctgcagtgattagaaggggtggggtggcgggaatcctatttatcagactctgtaattgaatataaatgttttactcagaggagctgcaaattgcctgcaaaaatgaaatccagtgagcactagaatatttaaaacatcattactgccatctttatcatgaagcacatcaattacaagctgtagaccacctaatatcaatttgtaggtaatgttcctgaaaattgcaatacatttcaattatactaaacctcacaaagtagaggaatccatgtaaattgcaaataaaccactttctaattttttcctgtttctgaattgtaaaaccccctttgggagtccctggtttcttattgagccaatttctgggttaatcttattgatttttcagcatcagtacaactctacaacctttgagctatatctgctttttcccattgcttccactgcc

SEQ ID NO: 52 - protein, 424 aa, Homo sapiens
  misc_feature: Incyte ID No 237547CD1
MDFSRRSFHRSLSSSLQAPVVSTVGMQRLGTTPSVYGGAGGRGIRISNSRHTVNYGSDLTGGGDLFVGNEKMAMQNLNDRLASYLEKVRTLEQSNSKLEVQIKQWYETNAPRAGRDYSAYYRQIEELRSQIKDAQLQNARCVLQIDNAKLAAEDFRLKYETERGIRLTVEADLQGLNKVFDDLTLHKTDLEIQIEELNKDLALLKKEHQEEVDGLHKHLGNTVNVEVDAAPGLNLGVIMNEMRQKYEVMAQKNLQEAKEQFERQTAVLQQQVTVNTEELKGTEVQLTELRRTSQSLEIELQSHLSMKESLEHTLEETKARYSSQLANLQSLLSSLEAQLMQIRSNMERQNNEYHILLDIKTRLEQEIATYRRLLEGEDVKTTEYQLSTLEERDIKKTRKIKTVVQEVVDGKVVSSEVKEVEENI

SEQ ID NO: 53 - DNA, 3169 bp, Homo sapiens
  misc_feature: Incyte ID No 2771481CB1
gccaggaataactagagaggaacaatggggttattcagaggttttgttttcctcttagttctgtgcctgctgcaccagtcaaatacttccttcattaagctgaataataatggctttgaagatattgtcattgttatagatcctagtgtgccagaagatgaaaaaataattgaacaaatagaggatatggtgactacagcttctacgtacctgtttgaagccacagaaaaaagattttttttcaaaaatgtatctatattaattcctgagaattggaaggaaaatcctcagtacaaaaggccaaaacatgaaaaccataaacatgctgatgttatagttgcaccacctacactcccaggtagagatgaaccatacaccaagcagttcacagaatgtggagagaaaggcgaatacattcacttcacccctgaccttctacttggaaaaaaacaaaatgaatatggaccaccaggcaaactgtttgtccatgagtgggctcacctccggtggggagtgtttgatgagtacaatgaagatcagcctttctaccgtgctaagtcaaaaaaaatcgaagcaacaaggtgttccgcaggtatctctggtagaaatagagtttataagtgtcaaggaggcagctgtcttagtagagcatgcagaattgattctacaacaaaactgtatggaaaagattgtcaattctttcctgataaagtacaaacagaaaaagcatccataatgtttatgcaaagtattgattctgttgttgaattttgtaacgaaaaaacccataatcaagaagctccaagcctacaaaacataaagtgcaattttagaagtacatgggaggtgattagcaattctgaggattttaaaaacaccatacccatggtgacaccacctcctccacctgtcttctcattgctgaagatcagtcaaagaattgtgtgcttagttcttgataagtctggaagcatggggggtaaggaccgcctaaatcgaatgaatcaagcagcaaaacatttcctgctgcagactgttgaaaatggatcctgggtggggatggttcactttgatagtactgccactattgtaaataagctaatccaaataaaaagcagtgatgaaagaaacacactcatggcaggattacctacatatcctctgggaggaacttccatctgctctggaattaaatatgcatttcaggtgattggagagctacattcccaactcgatggatccgaagtactgctgctgactgatggggaggataacactgcaagttcttgtattgatgaagtgaaacaaagtggggccattgttcattttattgctttgggaagagctgctgatgaagcagtaatagagatgagcaagataacaggaggaagtcatttttatgtttcagatgaagctcagaacaatggcctcattgatgcttttggggctcttacatcaggaaatactgatctctcccagaagtcccttcagctcgaaagtaagggattaacactgaatagtaatgcctggatgaacgacactgtcataattgatagtacagtgggaaaggacacgttctttctcatcacatggaacagtctgcctcccagtatttctctctgggatcccagtggaacaataatggaaaatttcacagtggatgcaacttccaaaatggcctatctcagtattccaggaactgcaaaggtgggcacttgggcatacaatcttcaagccaaagcgaacccagaaacattaactattacagtaacttctcgagcagcaaattcttctgtgcctccaatcacagtgaatgctaaaatgaataaggacgtaaacagtttccccagcccaatgattgtttacgcagaaattctacaaggatatgtacctgttcttggagccaatgtgactgctttcattgaatcacagaatggacatacagaagttttggaacttttggataatggtgcaggcgctgattctttcaagaatgatggagtctactccaggtattttacagcatatacagaaaatggcagatatagcttaaaagttcgggctcatggaggagcaaacactgccaggctaaaattacggcctccactgaatagagccgcgtacataccaggctgggtagtgaacggggaaattgaagcaaacccgccaagacctgaaattgatgaggatactcagaccaccttggaggatttcagccgaacagcatccggaggtgcatttgtggtatcacaagtcccaagccttcccttgcctgaccaatacccaccaagtcaaatcacagaccttgatgccacagttcatgaggataagattattcttacatggacagcaccaggagataattttgatgttggaaaagttcaacgttatatcataagaataagtgcaagtattcttgatctaagagacagttttgatgatgctcttcaagtaaatactactgatctgtcaccaaaggaggccaactccaaggaaagctttgcatttaaaccagaaaatatctcagaagaaaatgcaacccacatatttattgccattaaaagtatagataaaagcaatttgacatcaaaagtatccaacattgcacaagtaactttgtttatccctcaagcaaatcctgatgacattgatcctactcctactcctactcctactcctgataaaagtcataattctggagttaatatttctacgctggtattgtctgtgattgggtctgttgtaattgttaactttattttaagtaccaccatttgaaccttaacgaagaaaaaaatcttcaagtagacctagaagagagttttaaaaaacaaaacaatgtaagtaaaggatatttctgaatcttaaaattcatcccatgtgtgatcataaactcataaaaataattttaagatgtcggaaaaggatactttgattaaataaaaacactcatggatatgtaaaaactgtcaagattaaaatttaatagtttcatttatttgttattttatttgtaagaaatagtgatgaacaaagatcctttttcatactgatacctggttgtatattatttgatgcaacagttttctgaaatgatatttcaaattgcatcaagaaattaaaatcatctatctgagtagtcaaaatacaagtaaaggagagcaaataa

SEQ ID NO: 54 - protein, 917 aa, Homo sapiens
  misc_feature: Incyte ID No 2771481CD1
MGLFRGFVFLLVLCLLHQSNTSFIKLNNNGFEDIVIVIDPSVPEDEKIIEQIEDMVTTASTYLFEATEKRFFFKNVSILIPENWKENPQYKRPKHENHKHADVIVAPPTLPGRDEPYTKQFTECGEKGEYIHFTPDLLLGKKQNEYGPPGKLFVHEWAHLRWGVFDEYNEDQPFYRAKSKKIEATRCSAGISGRNRVYKCQGGSCLSRACRIDSTTKLYGKDCQFFPDKVQTEKASIMFMQSIDSVVEFCNEKTHNQEAPSLQNIKCNFRSTWEVISNSEDFKNTIPMVTPPPPPVFSLLKISQRIVCLVLDKSGSMGGKDRLNRMNQAAKHFLLQTVENGSWVGMVHFDSTATIVNKLIQIKSSDERNTLMAGLPTYPLGGTSICSGIKYAFQVIGELHSQLDGSEVLLLTDGEDNTASSCIDEVKQSGAIVHFIALGRAADEAVIEMSKITGGSHFYVSDEAQNNGLIDAFGALTSGNTDLSQKSLQLESKGLTLNSNAWMNDTVIIDSTVGKDTFFLITWNSLPPSISLWDPSGTIMENFTVDATSKMAYLSIPGTAKVGTWAYNLQAKANPETLTITVTSRAANSSVPPITVNAKMNKDVNSFPSPMIVYAEILQGYVPVLGANVTAFIESQNGHTEVLELLDNGAGADSFKNDGVYSRYFTAYTENGRYSLKVRAHGGANTARLKLRPPLNRAAYIPGWVVNGEIEANPPRPEIDEDTQTTLEDFSRTASGGAFVVSQVPSLPLPDQYPPSQITDLDATVHEDKIILTWTAPGDNFDVGKVQRYIIRISASILDLRDSFDDALQVNTTDLSPKEANSKESFAFKPENISEENATHIFIAIKSIDKSNLTSKVSNIAQVTLFIPQANPDDIDPTPTPTPTPDKSHNSGVNISTLVLSVIGSVVIVNFILSTTI

SEQ ID NO: 55 - DNA, 846 bp, Homo sapiens
  misc_feature: Incyte ID No 1400916.1
cagagtctgggaatctcaccatggcctggacccctctcctgctccccctcctcactttctgcacagtctctgaggcctcctatgagctgacacagccaccctcggtgtccgtgtccccaggacaaacggccaatatcacctgctctggagacgcattgccaaataaatatgcatattggttccagcagaagtcagggcaggcccctttgctggtcatctatgaggacattagacgacattccgggatccctgagagattttctgggtccagctcagggacaatggccacattgaccatcagcggggcccaggtggacgatgaagctgtctactattgttactcaacagacaacagtggaaattacaaaaggctgttcggcggagggaccaggctgaccgtcctaggccagcccagggctgccccctcggtcactctgttcccgccctcctctgaggagcttcaagccaacagggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaaactacagctgccagatcacgcatgaagggagcaccgtggagaagacagtggcccctacagaatgttcataggttctaaaccctcaccccccccacgggagcctagagctgcaggatcccaggggaggggtctctcctcccaccccaaggcatcaagcccttctccctgcactcaataaaccctcaatgaata

SEQ ID NO: 56 - DNA, 1769 bp, Homo sapiens
  misc_feature: Incyte ID No 253986.11
cttctcctccagcacaggaactaggaactacggagagagaagccaagggagaggaggaggaggaaactaacgattccctgcccacccccacacccagcaccaccaacaggtggggcaagcttgccgagaaaacgcagaggggcatcctgtgagcagcaaacacatctgagcctggaaaagacgcagagaagtaaaagatcaaagtctgattggcaccggctcccattccggctccagcctccaatccgacccccatttcggctgcagcctcggacctagctccggccctcggtctatccggttgcatcctccctccctgttccggatcttatcttgcgccagcgcctactccaggatcccgtagccagacctcaagccatggctggtcccttctcccgtctgctgtccgcccgcccgggactcaggctcctggctttggccggagcggggtctctagccgctgggtttctgctccgaccggaacctgtacgagctgccagtgaacgacggaggctgtatcccccgagcgctgagtacccagacctccgaaagcacaacaactgcatggccagtcacctgaccccagcagtctatgcacggctctgcgacaagaccacacccactggttggacgctagatcagtgtatccagactggcgtggacaaccctggccaccccttcatcaagactgtgggcatggtggctggagatgaggagacctatgaggtatttgctgacctgtttgaccctgtgatccaagagcgacacaatggatatgacccccggacaatgaagcacaccacggatctagatgccagtaaaatccgttctggctactttgatgagaggtatgtattgtcctctagagtcagaactggccgaagcatccgaggactcagtctgcctccagcttgcactcgagcagagcgacgagaggtggaacgtgttgtggtggatgcactgagtggcctgaagggtgacctggctggacgttactataggctcagtgagatgacagaggctgaacagcagcagcttattgatgaccactttctgtttgataagcctgtgtccccgttgctgactgcagcaggaatggctcgagactggccagatgctcgtggaatttggcacaacaatgagaagagcttcctgatctgggtgaatgaggaggatcatacacgggtgatctccatggagaagggtggtaacatgaagagagtgtttgaaagattctgccgaggcctcaaagaggtggagagacttatccaagaacgtggctgggagttcatgtggaatgagcgtttgggatacatcttgacctgtccatctaacctgggcactggacttcgggcaggagtgcacatcaaactgcccctgctaagcaaagatagccgcttcccaaagatcctggagaacctaagactccaaaagcgtggtactggaggagtggacactgctgccacaggcggtgtctttgatatttctaatttggaccgactaggcaaatcagaggtggagctggtgcaactggtcatcgatggagtaaactatttgattgattgtgaacggcgtctggagagaggccaggatatccgcatccccacacctgtcatccacaccaagcattaactccccatcgccagctgatgactcaagattccaaggagttctgctcattctaatgatggcccattctacttgctctggacctgccctcgcatcccctgcctccatcctagtaaagactccttgctatgctgcaaaaa

SEQ ID NO: 57 - DNA, 780 bp, Homo sapiens
  misc_feature: Incyte ID No 253986.17
ctggctggacgttactataggctcagtgagatgacagaggctgaacagcagcagcttattgatgaccactttctgtttgataagcctgtgtccccgttgctgactgcagcaggaatggctcgagactggccagatgctcgtggaatttggcacaacaatgagaagagcttcctgatctgggtgaatgaggaggatcatacacgggtgatctccatggagaagggtggtaacatgaagagagtgtttgaaagattctgccgaggcctcaaagaggtggagagacttatccaagaacgtggctgggagttcatgtggaatgagcgtttgggatacatcttgacctgtccatctaacctgggcactggacttcgggcaggagtgcacatcaaactgcccctgctaagcaaagatagccgcttcccaaagatcctggagaacctaagactccaaaaacgtggtactggaggagtggacactgctgctacaggcggtgtctttgatatttctaatttggaccgactaggcaaatcagaggtggagctggtgcaactggtcatcgatggagtaaactatttgattgattgtgaacggcgtctggagagaggccaggatatccgcatccccacacctgtcatccacaccaagcattaactccccatcgccagctgatgactcaagattcccaggagttctgctcattctaatgatggcccattctacttgctctggacctgcccccgcatcccctgcctccatcctagtaaagactccttgctatgc

SEQ ID NO: 58 - DNA, 3149 bp, Homo sapiens
  misc_feature: Incyte ID No 2680109CB1
gcagcgtccagtgccctgccagtagctcctagagaggcaggggttaccaactggccagcaggctgtgtccctgaagtcagatcaacgggagagaaggaagtggctaaaacattgcacaggagaagtcggcctgagtggtgcggcgctcgggacccaccagcaatgctgctcttcgtgctcacctgcctgctggcggtcttcccagccatctccacgaagagtcccatatttggtcccgaggaggtgaatagtgtggaaggtaactcagtgtccatcacgtgctactacccacccacctctgtcaaccggcacacccggaagtactggtgccggcagggagctagaggtggctgcataaccctcatctcctcggagggctacgtctccagcaaatatgcaggcagggctaacctcaccaacttcccggagaacggcacatttgtggtgaacattgcccagctgagccaggatgactccgggcgctacaagtgtggcctgggcatcaatagccgaggcctgtcctttgatgtcagcctggaggtcagccagggtcctgggctcctaaatgacactaaagtctacacagtggacctgggcagaacggtgaccatcaactgccctttcaagactgagaatgctcaaaagaggaagtccttgtacaagcagataggcctgtaccctgtgctggtcatcgactccagtggttatgtaaatcccaactatacaggaagaatacgccttgatattcagggtactggccagttactgttcagcgttgtcatcaaccaactcaggctcagcgatgctgggcagtatctctgccaggctggggatgattccaatagtaataagaagaatgctgacctccaagtgctaaagcccgagcccgagctggtttatgaagacctgaggggctcagtgaccttccactgtgccctgggccctgaggtggcaaacgtggccaaatttctgtgccgacagagcagtggggaaaactgtgacgtggtcgtcaacaccctggggaagagggccccagcctttgagggcaggatcctgctcaacccccaggacaaggatggctcattcagtgtggtgatcacaggcctgaggaaggaggatgcagggcgctacctgtgtggagcccattcggatggtcagctgcaggaaggctcgcctatccaggcctggcaactcttcgtcaatgaggagtccacgattccccgcagccccactgtggtgaagggggtggcaggaagctctgtggccgtgctctgcccctacaaccgtaaggaaagcaaaagcatcaagtactggtgtctctgggaaggggcccagaatggccgctgccccctgctggtggacagcgaggggtgggttaaggcccagtacgagggccgcctctccctgctggaggagccaggcaacggcaccttcactgtcatcctcaaccagctcaccagccgggacgccggcttctactggtgtctgaccaacggcgatactctctggaggaccaccgtggagatcaagattatcgaaggagaaccaaacctcaaggtaccagggaatgtcacggctgtgctgggagagactctcaaggtcccctgtcactttccatgcaaattctcctcgtacgagaaatactggtgcaagtggaataacacgggctgccaggccctgcccagccaagacgaaggccccagcaaggccttcgtgaactgtgacgagaacagccggcttgtctccctgaccctgaacctggtgaccagggctgatgagggctggtactggtgtggagtgaagcagggccacttctatggagagactgcagccgtctatgtggcagttgaagagaggaaggcagcggggtcccgcgatgtcagcctagcgaaggcagacgctgctcctgatgagaaggtgctagactctggttttcgggagattgagaacaaagccattcaggatcccaggctttttgcagaggaaaaggcggtggcagatacaagagatcaagccgatgggagcagagcatctgtggattccggcagctctgaggaacaaggtggaagctccagagcgctggtctccaccctggtgcccctgggcctggtgctggcagtgggagccgtggctgtgggggtggccagagcccggcacaggaagaacgtcgaccgagtttcaatcagaagctacaggacagacattagcatgtcagacttcgagaactccagggaatttggagccaatgacaacatgggagcctcttcgatcactcaggagacatccctcggaggaaaagaagagtttgttgccaccactgagagcaccacagagaccaaagaacccaagaaggcaaaaaggtcatccaaggaggaagccgagatggcctacaaagacttcctgctccagtccagcaccgtggccgccgaggcccaggacggcccccaggaagcctagacggtgtcgccgcctgctccctgcacccatgacaatcaccttcagaatcatgtcgatcctggggccctcagctcctggggaccccactccctgctctaacacctgcctaggtttttcctactgtcctcagaggcgtgctggtcccctcctcagtgacatcaaagcctggcctaattgttcctattggggatgagggtggcatgaggaggtcccacttgcaacttctttctgttgagagaacctcaggtacggagaagaatagaggtcctcatgggtcccttgaaggaagagggaccagggtgggagagctgattgcagaaaggagagacgtgcagcgcccctctgcacccttatcatgggatgtcaacagaatttttccctccactccatccctccctcccgtccttcccctcttcttctttccttccatcaaaagatgtatttgaattcatactagaattcaggtgctttgctagatgctgtgacaggtatgcctacaacactgctcacagcctttctgaggacaccagtgaaagaagctacagctcttcttggcgtatttatactcactgagtcttaacttttcaccaggggtgctcacgtctgcccctagtgggagaggtcataaaatgtctcgagtcctaaggacttaggggtcatgtatgatgagcatacacacaggtaattataaa

SEQ ID NO: 59 - protein, 764 aa, Homo sapiens
  misc_feature: Incyte ID No 2680109CD1
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 60 - DNA, 655 bp, Homo sapiens
  misc_feature: Incyte ID No 1800311CB1
cagtaacctgccctctttaaaagtcccgccgcttccccctggcatccacaacagccacccctctctcgggcactgctgccatgaatgccttcctgctcttcgcactgtgcctccttggggcctgggccgccttggcaggaggggtcaccgtgcaggatggaaatttctccttttctctggagtcagtgaagaagctcaaagacctccaggagccccaggagcccagggttgggaaactcaggaactttgcacccatccctggtgaacctgtggttcccatcctctgtagcaacccgaactttccagaagaactcaagcctctctgcaaggagcccaatgcccaggagatacttcagaggctggaggaaatcgctgaggacccgggcacatgtgaaatctgtgcctacgctgcctgtaccggatgctaggggggcttgcccactgcctgcctcccctccgcagcagggaagctcttttctcctgcagaaagggccacccatgatactccactcccagcagctcaacctaccctggtccagtcgggaggagcagcccggggaggaactgggtgactggaggcctcgccccaacactgtccttccctgccacttcaacccccagctaataaaccagattccagagtaaaaaaaaaaaa

SEQ ID NO: 61 - protein, 115 aa, Homo sapiens
  misc_feature: Incyte ID No 1800311CD1
MNAFLLFALCLLGAWAALAGGVTVQDGNFSFSLESVKKLKDLQEPQEPRVGKLRNFAPIPGEPVVPILCSNPNFPEELKPLCKEPNAQEILQRLEEIAEDPGTCEICAYAACTGC

SEQ ID NO: 62 - DNA, 1312 bp, Homo sapiens
  misc_feature: Incyte ID No 1804734CB1
gtccctaggagataagagtatcttgcacagcaggtgcaggtttcccagcagctcaggcaagagtccgatgtttgtgccatctgatcctgatgtctggagagcagatagccatgtgtgagcctgaatttggcaatgacaaggccagggagccgagcgtgggtggcaggtggcgagtgtcctggtacgaacggtttgtgcagccatgtctggtcgaactgctgggctctgctctcttcatcttcatcgggtgcctgtcggtcattgagaatgggacggacactgggctgctgcagccggccctggcccacgggctggctttggggctcgtgattgccacgctggggaatatcagtggtggacacttcaaccctgcggtgtccctggcagccatgctgatcggaggcctcaacctggtgatgctcctcccgtactgggtctcacagctgctcggggggatgctcggggctgccttggccaaggcggtgagtcctgaggagaggttctggaatgcatctggggcggcctttgtgacagtccaggagcaggggcaggtggcaggggcgttggtggcagagatcatcctgacgacgctgctggccctggctgtatgcatgggtgccatcaatgagaagacaaagggccctctggccccgttctccatcggctttgccgtcaccgtggatatcctggctgggggccctgtgtctggaggctgcatgaatcccgcccgtgcttttggacctgcggtggtggccaaccactggaacttccactggatctactggctgggcccactcctggctggcctgcttgttggactgctcattaggtgcttcattggagatgggaagacccgcctcatcctgaaggctcggtgaagcagagctcgtgggattcctgctgctccaggtgtcctcagctcacctgtcccagactgaggacaggggagttcctgcatttcctgccagggcagaggcccagaggagcgaccccctgcttccactgcttgggcctgctttctcagatagactgactgctgaggaggctctaggttcttggaattcctttgtgctcatcagagaccccagcctggggaacacgctgcccgcactgcccagagagcagtgcaaacaccacaacacgagcgtgtttcttgagaggaatgtccccgagttggacaaggaggctgtttctgcacatcagctcatttcccgcaccccatttcttgcttgattgctttgttgggggcctggccacttccttgcttctcaagctgacaattctcactttgcaataaatagtccagtgtttc

SEQ ID NO: 63 - protein, 262 aa, Homo sapiens
  misc_feature: Incyte ID No 1804734CD1
MSGEQIAMCEPEFGNDKAREPSVGGRWRVSWYERFVQPCLVELLGSALFIFIGCLSVIENGTDTGLLQPALAHGLALGLVIATLGNISGGHFNPAVSLAAMLIGGLNLVMLLPYWVSQLLGGMLGAALAKAVSPEERFWNASGAAFVTVQEQGQVAGALVAEIILTTLLALAVCMGAINEKTKGPLAPFSIGFAVTVDILAGGPVSGGCMNPARAFGPAVVANHWNFHWIYWLGPLLAGLLVGLLIRCFIGDGKTRLILKAR

SEQ ID NO: 64 - DNA, 1556 bp, Homo sapiens
  misc_feature: Incyte ID No 3231154CB1
gggagaaggcaggggaggtggctggtttaaggggaacttgagggaagtagggaagactcctctcgggacctttggagtaggtgacacatgagcccagccccagctcacctgccaatccagctgaggagctcacctgccaatccagctgaggctgggcagaggtgggtgagaagagggaaaattgcagggacctccagttgggccaggccagaagctgctgtagctttaaccagacagctcagacctgtctggaggctgccagtgacaggttaggtttagggcagagaagaagcaagaccatggtggggaagatgtggcctgtgttgtggacactctgtgcagtcagggtgaccgtcgatgccatctctgtggaaactccgcaggacgttcttcgggcttcgcagggaaagagtgtcaccctgccctgcacctaccacacttccacctccagtcgagagggacttattcaatgggataagctcctcctcactcatacggaaagggtggtcatctggccgttttcaaacaaaaactacatccatggtgagctttataagaatcgcgtcagcatatccaacaatgctgagcagtccgatgcctccatcaccattgatcagctgaccatggctgacaacggcacctacgagtgttctgtctcgctgatgtcagacctggagggcaacaccaagtcacgtgtccgcctgttggtcctcgtgccaccctccaaaccagaatgcggcatcgagggagagaccataattgggaacaacatccagctgacctgccaatcaaaggagggctcaccaacccctcagtacagctggaagaggtacaacatcctgaatcaggagcagcccctggcccagccagcctcaggtcagcctgtctccctgaagaatatctccacagacacatcgggttactacatctgtacctccagcaatgaggaggggacgcagttctgcaacatcacggtggccgtcagatctccctccatgaacgtggccctgtatgtgggcatcgcggtgggcgtggttgcagccctcattatcattggcatcatcatctactgctgctgctgccgagggaaggacgacaacactgaagacaaggaggatgcaaggccgaaccgggaagcctatgaggagccaccagagcagctaagagaactttccagagagagggaggaggaggatgactacaggcaagaagagcagaggagcactgggcgtgaatccccggaccacctcgaccagtgacaggccagcagcagagggcggcggaggaagggttaggggttcattctcccgcttcctggcctcccttctcctttctaagccctgttctcctgtccctccatcccagacattgatggggacatttcttccccagtgtcagctgtggggaacatggctggcctggtaagggggtccctgtgctgatcctgctgacctcactgtcctgtgaagtaacccctcctggctgtgacacctggtgcgggcctggccctcactcaagaccaggctgcagcctccattcctcgtagttggcaggag

SEQ ID NO: 65 - protein, 319 aa, Homo sapiens
  misc_feature: Incyte ID No 3231154CD1
MVGKMWPVLWTLCAVRVTVDAISVETPQDVLRASQGKSVTLPCTYHTSTSSREGLIQWDKLLLTHTERVVIWPFSNKNYIHGELYKNRVSISNNAEQSDASITIDQLTMADNGTYECSVSLMSDLEGNTKSRVRLLVLVPPSKPECGIEGETIIGNNIQLTCQSKEGSPTPQYSWKRYNILNQEQPLAQPASGQPVSLKNISTDTSGYYICTSSNEEGTQFCNITVAVRSPSMNVALYVGIAVGVVAALIIIGIIIYCCCCRGKDDNTEDKEDARPNREAYEEPPEQLRELSREREEEDDYRQEEQRSTGRESPDHLDQ

SEQ ID NO: 66 - DNA, 3476 bp, Homo sapiens
  misc_feature: Incyte ID No 210095.11
aaaacagcagaggtgacagagcagccgtgctcgaagcgttcctggagcccaagctctcttccacaggtgaagacagggccagcaggagacaccatggggcacctctcagccccacttcacagagtgcgtgtaccctggcaggggcttctgctcacagcctcacttctaaccttctggaacccgcccaccactgcccagctcactactgaatccatgccattcaatgttgcagaggggaaggaggttcttctccttgtccacaatctgccccagcaactttttggctacagctggtacaaaggggaaagagtggatggcaaccgtcaaattgtaggatatgcaataggaactcaacaagctaccccagggcccgcaaacagcggtcgagagacaatataccccaatgcatccctgctgatccagaacgtcacccagaatgacacaggattctacaccctacaagtcataaagtcagatcttgtgaatgaagaagcaactggacagttccatgtatacccggagctgcccaagccctccatctccagcaacaactccaaccctgtggaggacaaggatgctgtggccttcacctgtgaacctgagactcaggacacaacctacctgtggtggataaacaatcagagcctcccggtcagtcccaggctgcagctgtccaatggcaacaggaccctcactctactcagtgtcacaaggaatgacacaggaccctatgagtgtgaaatacagaacccagtgagtgcgaaccgcagtgacccagtcaccttgaatgtcacctatggcccggacacccccaccatttccccttcagacacctattaccgtccaggggcaaacctcagcctctcctgctatgcagcctctaacccacctgcacagtactcctggcttatcaatggaacattccagcaaagcacacaagagctctttatccctaacatcactgtgaataatagtggatcctatacctgccacgccaataactcagtcactggctgcaacaggaccacagtcaagacgatcatagtcactgagctaagtccagtagtagcaaagccccaaatcaaagccagcaagaccacagtcacaggagataaggactctgtgaacctgacctgctccacaaatgacactggaatctccatccgttggttcttcaaaaaccagagtctcccgtcctcggagaggatgaagctgtcccagggcaacaccaccctcagcataaaccctgtcaagagggaggatgctgggacgtattggtgtgaggtcttcaacccaatcagtaagaaccaaagcgaccccatcatgctgaacgtaaactataatgctctaccacaagaaaatggcctctcacctggggccattgctggcattgtgattggagtagtggccctggttgctctgatagcagtagccctggcatgttttctgcatttcgggaagaccggcagggcaagcgaccagcgtgatctcacagagcacaaaccctcagtctccaaccacactcaggaccactccaatgacccaccttacaagatgaatgaagttacttattctaccctgaactttgaagcccagcaacccacacaaccaacttcagcctccccatccctaacagccacagaaataatttattcagaagtaaaaaagcagtaatgaaacctgtcctgctcactgcagtgctgatgtatttcaagtctctcaccctcatcactaggagattcctttcccctgtaggggtagaggggtggggacagaaacaactttctcctactcttccttcctaataggcatctccaggctgcctggtcactgcccctctctcagtgtcaatagatgaaagtacattgggagtctgtaggaaacccaaccttcttgtcattgaaatttggcaaagctgactttgggaaagagggaccagaacttcccctcccttccccttttcccaacctggacttgttttaaacttgcctgttcagagcactcattccttcccacccccagtcctgtcctatcactctaattcggatttgccatagccttgaggttatgtccttttccattaagtacatgtgccaggaaacaagagagagagaaagtaaaggcagtaatgccttctcctatttctccaaagccttgtgtgaactcaccaaacacaagaaaatcaaatatataaccaatagtgaaatgccacacctttgtccactgtcagggttgtctacctgtaggatcagggtctaagcaccttggtgcttagctagaataccacctaatccttctggcaagcctgtcttcagagaacccactagaagcaactaggaaaatcacttgccaaaatccaaggcaattcctgatggaaaatgcaaaagcacatatatgttttaatatctttatgggctctgttcaaggcagtgctgagagggaggggttatagcttcaggagggaaccagcttctgataaacacaatctgctaggaacttgggaaaggaatcagagagctgcccttcagcgattatttaaattattgttaaagaatacacaatttggggtattgggatttttctccttttctctgagacattccaccattttaatttttgtaactgcttatttatgtgaaaagggttatttttacttagcttagctatgtcagccaatccgattgccttaggtgaaagaaaccaccgaaatccctcaggtcccttggtcaggagcctctcaagattttttttgtcagaggctccaaatagaaaataagaaaaggttttcttcattcatggctagagctagatttaactcagtttctaggcacctcagaccaatcatcaactaccattctattccatgtttgcacctgtgcattttctgtttgcccccattcactttgtcaggaaaccttggcctctgctaaggtgtatttggtccttgagaagtgggagcaccctacagggacactatcactcatgctggtggcattgtttacagctagaaagctgcactggtgctaatgccccttggggaaatggggctgtgaggaggaggattataacttaggcctagcctcttttaacagcctctgaaatttatcttttcttctatggggtctataaatgtatcttataataaaaaggaaggacaggaggaagacaggcaaatgtacttctcacccagtcttctacacagatggaatctctttggggctaagagaaaggttttattctatattgcttacctgatctcatgttaggcctaagaggctttctccaggaggattagcttggagttctctatactcaggtacctctttcagggttttctaaccctgacacggactgtgcatactttccctcatccatgctgtgctgtgttatttaatttttcctggctaagatcatgtctgaattatgtatgaaaattattctatgtttttataataaaaataatatatcagacatcg

SEQ ID NO: 67 - DNA, 1237 bp, Homo sapiens
  misc_feature: Incyte ID No 2719813CB1
atcgctgcacccgcggcggcctcctcggtgcgcgacccccggctcagaggactctttgctgtcccgcaagatgcggatgctgctggcgctcctggccctctccgcggcgcggccatcggccagtgcagagtcacactggtgctacgaggttcaagccgagtcctccaactacccctgcttggtgccagtcaagtggggtggaaactgccagaaggaccgccagtcccccatcaacatcgtcaccaccaaggcaaaggtggacaaaaaactgggacgcttcttcttctctggctacgataagaagcaaacgtggactgtccaaaataacgggcactcagtgatgatgttgctggagaacaaggccagcatttctggaggaggactgcctgccccataccaggccaaacagttgcacctgcactggtccgacttgccatataagggctcggagcacagcctcgatggggagcactttgccatggagatgcacatagtacatgagaaagagaaggggacatcgaggaatgtgaaagaggcccaggaccctgaagacgaaattgcggtgctggcctttctggtggaggctggaacccaggtgaacgagggcttccagccactggtggaggcactgtctaatatccccaaacctgagatgagcactacgatggcagagagcagcctgttggacctgctccccaaggaggagaaactgaggcactacttccgctacctgggctcactcaccacaccgacctgcgatgagaaggtcgtctggactgtgttccgggagcccattcagcttcacagagaacagatcctggcattctctcagaagctgtactacgacaaggaacagacagtgagcatgaaggacaatgtcaggcccctgcagcagctggggcagcgcacggtgataaagtccggggccccgggtcggccgctgccctgggccctgcctgccctgctgggccccatgctggcctgcctgctggccggcttcctgcgatgatggctcacttctgcacgcagcctctctgttgcctcagctctccaagttccaggcttccggtccttagccttcccaggtgggactttaggcatgattaaaatatggacatatttttggagaaacctttctcaagtgtgtttttagccttccacaactaccccaccctgtccccctccacccacccctgttcctcctgttccagggcgggggctttaaggccaggagatt

SEQ ID NO: 68 - protein, 312 aa, Homo sapiens
  misc_feature: Incyte ID No 2719813CD1
MRMLLALLALSAARPSASAESHWCYEVQAESSNYPCLVPVKWGGNCQKDRQSPINIVTTKAKVDKKLGRFFFSGYDKKQTWTVQNNGHSVMMLLENKASISGGGLPAPYQAKQLHLHWSDLPYKGSEHSLDGEHFAMEMHIVHEKEKGTSRNVKEAQDPEDEIAVLAFLVEAGTQVNEGFQPLVEALSNIPKPEMSTTMAESSLLDLLPKEEKLRHYFRYLGSLTTPTCDEKVVWTVFREPIQLHREQILAFSQKLYYDKEQTVSMKDNVRPLQQLGQRTVIKSGAPGRPLPWALPALLGPMLACLLAGFLR

SEQ ID NO: 69 - DNA, 973 bp, Homo sapiens
  misc_feature: Incyte ID No 2886583CB1
agccgtagcgcccggctcctgcaggcgctcggcctccgctcattcctgaccccgcagtgggcgcgatggcggaggctgtactgagggtcgcccggcggcagctgagccagcgcggcgggtctggagcccccatcctcctgcggcagatgttcgagcctgtgagctgcaccttcacgtacctgctgggtgacagagagtcccgggaggccgttctgatcgacccagtcctggaaacagcgcctcgggatgcccagctgatcaaggagctggggctgcggctgctctatgctgtgaatacccactgccacgcggaccacattacaggctcggggctgctccgttccctcctccctggctgccagtctgtcatctcccgccttagtggggcccaggctgacttacacattgaggatggagactccatccgcttcgggcgcttcgcgttggagaccagggccagccctggccacaccccaggctgtgtcaccttcgtcctgaatgaccacagcatggccttcactggagatgccctgttgatccgtgggtgtgggcggacagacttccagcaaggctgtgccaagaccttgtaccactcggtccatgaaaagatcttcacacttccaggagactgtctgatctaccctgctcacgattaccatgggttcacagtgtccaccgtggaggaggagaggactctgaaccctcggctcaccctcagctgtgaggagtttgtcaaaatcatgggcaacctgaacttgcctaaacctcagcagatagactttgctgttccagccaacatgcgctgtggggtgcagacacccactgcctgatctcacttctgtcagatgctcccatccactattaatgcactaggtgggaggagagggcggcaatgacactgcacctctcctttcccaccgcattccctggagctccctaaataaaactttttttatcgtgaaaaaaaaaaaaa

SEQ ID NO: 70 - protein, 254 aa, Homo sapiens
  misc_feature: Incyte ID No 2886583CD1
MAEAVLRVARRQLSQRGGSGAPILLRQMFEPVSCTFTYLLGDRESREAVLIDPVLETAPRDAQLIKELGLRLLYAVNTHCHADHITGSGLLRSLLPGCQSVISRLSGAQADLHIEDGDSIRFGRFALETRASPGHTPGCVTFVLNDHSMAFTGDALLIRGCGRTDFQQGCAKTLYHSVHEKIFTLPGDCLIYPAHDYHGFTVSTVEEERTLNPRLTLSCEEFVKIMGNLNLPKPQQIDFAVPANMRCGVQTPTA

SEQ ID NO: 71 - DNA, 643 bp, Homo sapiens
  misc_feature: Incyte ID No 025685.3
gttttcctctccagagggatcagggtcctcttagggagtgacgggcttttcatatatttttgctgaagaatatatggaaagggtggcatttgcgtcacgtggaccaggacagtgctgaaatcagcagtgctcagaaacaatttaacatgttgaaacgacaatattctaaaatactgatgaatcttgcatcaatataattattgggtttttttctttttcctgctgtataactccttgccatgcaaactctcaagaggccaatatattcctggccatgtttgaatgagcctcttaaaataaacttagagccatgcaaatgccagcagcttaatggatttcatggaatgaaataccgtgattaactcatagctacatatcattgcataaatgggatttatcttttttctcacttattttttgctgtgaaagtcgagggcatgcaagagtttctcttccagaagccaggaggagaacgaaggtcctaatgctgtactattccaccctttggacgcctcatccaggacgcagaggactctaggtttaacattttgtacaaaacagaacctgttaatcacattaaagcacatatgtatatatcttttatttataaataaaattttaaaacaatagtttcagtatagcc

SEQ ID NO: 72 - DNA, 2879 bp, Homo sapiens
  misc_feature: Incyte ID No 1808144CB1
tgagtggatggacactgcctcttagaactagaacttagaactttatcttgaaaatgtaccactgttgcagaagctcctcacagagtatgtgtcaggcatttttaacctgctaaaggcaagaagaagtgttcaccacatagttgcaaaggtcttcaacttgccacagccaacagaaaaatcaaaatgattgaaccctttgggaatcagtatattgtggccaggccagtgtattctacaaatgcttttgaggaaaatcataaaaagacaggaagacatcataagacatttctggatcatctcaaagtgtgttgtagctgttccccacaaaaggccaagagaattgtcctctctttgttccccatagcatcttggttgccagcataccggcttaaagaatggttgctcagtgatattgtttctggtatcagcacagggattgtggccgtactacaaggtttagcatttgctctgctggtcgacattcccccagtctatgggttgtatgcatcctttttcccagccataatctaccttttcttcggcacttccagacacatatccgtgggtccgtttccgattctgagtatgatggtgggactagcagtttcaggagcagtttcaaaagcagtcccagatcgcaatgcaactactttgggattgcctaacaactcgaataattcttcactactggatgacgagagggtgagggtggcggcggcggcatcagtcacagtgctttctggaatcatccagttggcttttgggattctgcggattggatttgtagtgatatacctgtctgagtccctcatcagtggcttcactactgctgctgctgttcatgttttggtttcccaactcaaattcatttttcagttgacagtcccgtcacacactgatccagtttcaattttcaaagtactatactctgtattctcacaaatagagaagactaatattgcagacctggtgacagctctgattgtccttttggttgtatccattgttaaagaaataaatcagcgcttcaaagacaaacttccagtgcccattccaatcgaattcattatgaccgtgattgcagcaggtgtatcctacggctgtgactttaaaaacaggtttaaagtggctgtggttggggacatgaatcctggatttcagccccctattacacctgacgtggagactttccaaaacaccgtaggagattgcttcggcatcgcaatggttgcatttgcagtggccttttcagttgccagcgtctattccctcaaatacgattatccacttgatggcaatcaggagttaatagccttgggactgggtaacatagtctgtggagtattcagaggatttgctgggagtactgccctctccagatcagcagttcaggagagcacaggaggcaaaacacagattgctgggcttattggtgccatcatcgtgctgattgtcgttctagccattggatttctcctggcgcctctacaaaagtccgtcctggcagctttagcattgggaaacttaaagggaatgctgatgcagtttgctgaaataggcagattgtggcgaaaggacaaatatgattgtttaatttggatcatgaccttcatcttcaccattgtcctgggactcgggttaggcctggcagctagtgtggcatttcaactgctaaccatcgtgttcaggacccaatttccaaaatgcagcacgctggctaatattggaagaaccaacatctataagaataaaaaagattattatgatatgtatgagccagaaggagtgaaaattttcagatgtccatctcctatctactttgcaaacattggtttctttaggcggaaacttatcgatgctgttggctttagtccacttcgaattctacgcaagcgcaacaaagctttgaggaaaatccgaaaactgcagaagcaaggcttgctacaagtgacaccaaaaggatttatatgtactgttgacaccataaaagattctgacgaagagctggacaacaatcagatagaagtactggaccagccaatcaataccacagacctgcctttccacattgactggaatgatgatcttcctctcaacattgaggtccccaaaatcagcctccacagcctcattctcgacttttcagcagtgtcctttcttgatgtttcttcagtgaggggccttaaatcgattttgcaagaatttatcaggatcaaggtagatgtgtatatcgttggaactgatgatgacttcattgagaagcttaaccggtatgaattttttgatggtgaagtgaaaagctcaatatttttcttaacaatccatgatgctgttttgcatattttgatgaagaaagattacagtacttcaaagtttaatcccagtcaggaaaaagatggaaaaattgattttaccataaatacaaatggaggattacgtaatcgggtatatgaggtgccagttgaaacaaaattctaatcaacatataattcagaaggatcttcatctgactatgacataaaaacaactttatacccagaaagttattgataagttcatacattgtacgaagagtatttttgacagaatatgtttcaaactttggaacaagatggttctagcatggcatatttttcacatatctagtatgaaattatataagtattctaaattttatatcttgtagctttatcaaagggtgaaaattattttgttcatacatatttttgtagcactgacagatttccatcctagtcactaccttcatgcataggtttagcagtatagtggcgccactgttttgaatctcataatttatacaggtcatattaatatatttccattaaaaaatcagttgtacagtgaaaaaaaaaaaaaaa

SEQ ID NO: 73 - protein, 764 aa, Homo sapiens
  misc_feature: Incyte ID No 1808144CD1
MIEPFGNQYIVARPVYSTNAFEENHKKTGRHHKTFLDHLKVCCSCSPQKAKRIVLSLFPIASWLPAYRLKEWLLSDIVSGISTGIVAVLQGLAFALLVDIPPVYGLYASFFPAIIYLFFGTSRHISVGPFPILSMMVGLAVSGAVSKAVPDRNATTLGLPNNSNNSSLLDDERVRVAAAASVTVLSGIIQLAFGILRIGFVVIYLSESLISGFTTAAAVHVLVSQLKFIFQLTVPSHTDPVSIFKVLYSVFSQIEKTNIADLVTALIVLLVVSIVKEINQRFKDKLPVPIPIEFIMTVIAAGVSYGCDFKNRFKVAVVGDMNPGFQPPITPDVETFQNTVGDCFGIAMVAFAVAFSVASVYSLKYDYPLDGNQELIALGLGNIVCGVFRGFAGSTALSRSAVQESTGGKTQIAGLIGAIIVLIVVLAIGFLLAPLQKSVLAALALGNLKGMLMQFAEIGRLWRKDKYDCLIWIMTFIFTIVLGLGLGLAASVAFQLLTIVFRTQFPKCSTLANIGRTNIYKNKKDYYDMYEPEGVKIFRCPSPIYFANIGFFRRKLIDAVGFSPLRILRKRNKALRKIRKLQKQGLLQVTPKGFICTVDTIKDSDEELDNNQIEVLDQPINTTDLPFHIDWNDDLPLNIEVPKISLHSLILDFSAVSFLDVSSVRGLKSILQEFIRIKVDVYIVGTDDDFIEKLNRYEFFDGEVKSSIFFLTIHDAVLHILMKKDYSTSKFNPSQEKDGKIDFTINTNGGLRNRVYEVPVETKF

SEQ ID NO: 74 - DNA, 3503 bp, Homo sapiens
  misc_feature: Incyte ID No 201356.1
gcccttctcccaaggcaggcttaagttgagactattataggtgtctaataacctgtgacagagtaatgagtacatgcttaagatgttataattagccaacaccaacacagcaaaaaatataattccagccaaagattctggaaaatccctcagaaggagggataacaggatttgacctttaccagcgatttctgtccatatgtggatgtaaacagttctggaacgttatgcatgcagttagcgaatccttgaattatgttctggtttgtacttgtcccatccatccaaacaagagattctgcttttggtagccatctgtagaaacatttaagatgtcactagaatttacatttcatcctctctacttgggttgaggttgcctatacttgcatattgttaaaatgttttggttgctgatattcagaggaatgaaacctggaaccaaagcctaatttgccgataaaaaaactgttttcggccaggtgcagtggctcatgcctgtaatcccagcacgttgggaggccgaggcgggtggatcacctgaagtcaggagttcgagaccatcctggctaacactgtgaaaccccgtctctactaaaaatacaaagaattagcggggcatggtggcacgcgcctgtagtcccagctactcaggaggctgaggcaggagaattacttgagcccgggaggcggaggttgcagtaagcagagattgtgccactggactccagcctgggtgacagagcgagactccgtctcaaacaaacaaacaaaaaactgttttcatttgctctcttgaccaaaggataggactttagttctttaagcattattttaaacactatattgatacaaaaatatcttgcttactctaaactttagagtctaaatgaagctttttctcagtacaagattctgagtatcataaaatggttatttaattgaaacgtagtgtggtatactcttgatggttagaactcttacagccttatttatttttaagtttgttacagccaaagggttggagtgtgccagtgcacaggtagactaaggaaaacattatagaggagtgaagagaacagaccattgaaaagactattatctgaccagcggaggcagaaaagagaggaacccagttgaataggatccaatccctggttagcctctacacaataatagggagacaaggattaggagccatacctcccagagcaaggtatctttctagagcaaatttctctttctagaaggggagggtcacagggtcacagattcaccaaagctgaaagggctgaggagctcatggtagcctgggttgacctactctggagcacggtgtcttccttctaaactgagtgactgtagtactatctgtgcctctgatggtaataaaactgacaagatgtctaatttttttttaagtaggaccaaaggaaaacaagatttagatagtctgactttgcttttgaacaacagacattgcaagtcaaaattgttgtcaaatttacatatggtaaatgatgaactttaaaaatgtgtccaggtgttagatgagttcattagactcttttaatgctaatggctagtacgtttaaacaaaacagcagttctctgctgcaatattcccattgaccacttaaatgaccataagtggtcatttaagaacatgttagggttagccctgatctgaatataaaagtgagaaaagggctacagtgcatttcttggtaacttaaactgagtcttgaagttataatgatccattcgagttctgtgatccttattgttcttaattgtgtttctctacgtattgttacagatgagccatacgtttctttgtatcaatgtagacatgacttcagatacctctgaggacctacccagcagtctaggaccctgggccaagtgctgggactatggtactaaatccagtagatgggctgtgtagcaactctcccagggaacacactagggtacttagggaggtgctttgtggagcatgttgaagctttgagatctgagcaggaggcagtgatgtccctggtctattcagggaaagatttcagtgtgaaatggtaaacatccaattgacaggatttagattttgcttagtttttctgctttttaatgtttctatcccccatctcagtgttttctttatccatcccagtgatgccttatttgaaactgggcttaaactgcaaaaagaatgaagttggatttaggaagctgttagatcattgagtggtgttgagagtgaagtttcactagcagggaagtttccttgagcctaaaataaaaagaaaaaattaaaaagaatcagtttttttaattaaaaaaatagaaagctgttaggctcctaattcgtggggtttttttttgtaaaaacagtttagataatcctgaatgcaatcattaacttggttgctaattacaagaatgaaaattataatggaaaaggacaaaataatataccagctggtttgttattatagtccgtgtattaaaatactattgaaatacgttaaaggtaaatttttaaggtttaaaaaaaatttagtaacttacagggatggagaatttagatgtcagaggtggggagatttatttttataaggtaatttttatcctgataaggacttaaaaaaaagttttgcaactgaaattttaaagtaaacatgttaagtacagttaaaaagtaagcattgtagtaaatagtggattctctggtgtgtattttttatctcagtgttgaaaattggaaaagaatggactgaagtctaaaaactggaataatgaaggacactaaatgcctttattgtagatactatgtttgtaagtctatagctaagcaacttaagccaaaaaggtctttcaactgaagctttaatcaacttattttggagatgttctcttcccttatctcatgcgtcatccctaaaataataagatacatgggatcaaatagcccttgccttttcaacacaaatcagttggaaaattatggtttgagtcctgttgctgccatggcttctgtttctcagaaatgagtgtgtatgaacataccaatctatgtaataggctacctttttttgtcttctttggaactttgtacacaaaccaagacaatatcagggtgacaggtgaatgaacttaaattctcagtcttgtctattcaccaaaaaagtatactgcctgttttttctttaattattcaaggttgatgacttttaggaacatgttttatactgtattttttaattaaagcaagtgccttgatgtaattccatgtaaatcattgcttaaccctcttatgggatgaggatgagttattaatgtattgcagcctactggaaaggagggggagttggttaatagcagatacttttcttctagaagcttatgttttatgctgtttattatgtaagatcctgtatgtgtgttgagatttagaggtttcatttgttttgtctgctaataaattgttactctaataatacatt

SEQ ID NO: 75 - DNA, 1575 bp, Homo sapiens
  misc_feature: Incyte ID No 978178.7
ctgaaggtaaaaaaacctaaaataggtcctactcttccaaccattaccatgagtctatcgaaaattaggtctcagaccagaacagatcaaaatatttagtgctctctagggaaaaagcttttgagataaagtggaatttcttatttctaagaagtaggaaaagagtgttttgtttggtctctacaaattatagttatttggtttcagcctgaacaaccaccacaagcaaatactggcaggcagagtctctacaggagtggtcaaaaaagtcagttttcatttccttccaagccctagtttggctgatccatccacagaaaagcagaacaaaaggcaagggattactgcacccttaagcatccaagtggctcattcggctcaggggttatttttagcaatctggctcacactgagggcttcctgctctattcagcaaatgtgcaaatggatgctgcaggaccgtaacagtagacattgatgaaggttgtgatcaatgattagactctcaagcctgttgtgtttttgatctagccatgcctttcaaccactgtggtgaacatttagctctgaataggctcatcttcttcatatgcacattctatttgtagatgttgctatgaggacagattcatcaaaaatgactgacgtggagtctggggtcgccaattttgcatcttcagcaagggcaggccgccggaatgccttaccagacatccagagttcagctgccacagacggaacctcagatttgcccctcaaactggaggctctctccgtgaaggaagatgcaaaagagaaagatgaaaaaacaacacaagaccaattggaaaagcctcaaaatgaagaaaaatgaaggctcataatctatcaagagtgctgaatttctgcatgttgaaagacttagtggttctgttttcttgagacatttaatctggtggtaactgtggtaacattgcagccctaagcagcatgtgtatattagataattgtgttgtgatgctactcactttgattgcaatgatgatgtccaaggtaagctattaaaaggcaggttacttccaaatcgcactgaaggaaaaggttaagaataatacatgatcacagaaatgcataccactgtctgtaaacccaacaaaattcactgttctcttttggatttatttagcctgatgtatttttaattcaatttttatggtgatgggcaaatcattcttggtaaatgtaaatcaaacatgattgatttaaaacttcatggaatttgtagaaaattatggacatttttggtgagaaagaacaatagtcaaaactcacatggatagagtgtgtttgttttttgccaaaaatgccccagactttttcccaaacctcaaaaacgtcttggaaaaattgtaaaagtttgataacagaaacatctttaggatatttttgtctgacgtattttgcttctagtatgtgcctactgtgatttttttcatgtggaaaatgcaaaatttgtaacaaaatggttatatggaacatgcctattaaatgaattttactatcttccctaactttgggtctgtggtatggtgtgtg

SEQ ID NO: 76 - DNA, 2222 bp, Homo sapiens
  misc_feature: Incyte ID No 237563.31
catttccgcattgaaggggctgctccgaatggagggggaggggaggtgtttaggagaaagtaggggctgtgggtgtcgggagccggctgacgggtggacaagggggggttagcagctgggctgcgaccgttagggaggggctcaaggtgtgcatgtgtgagggaagagagagagagagaagggcgcctcagaggtgactttcagcctgcgagccttcttcccggggcgccataaacgcccccaatttcccagctgctaaaggaagaggaagatcttagcaaagcaatgtctcaagatggtgcttctcagttccaagaagtcattcggcaagagctagaattatctgtgaagaaggaactagaaaaaatactcaccacagcatcatcacatgaatttgagcacaccaaaaaagacctggatggatttcggaagctatttcatagatttttgcaagaaaaggggccttctgtggattggggaaaaatccagagaccccctgaagattcgattcaaccctatgaaaagataaaggccaggggcttgcctgataatatatcttccgtgttgaacaaactagtggtggtgaaactcaatggtggtttgggaaccagcatgggctgcaaaggccctaaaagtctgattggtgtgaggaatgagaatacctttctggatctgactgttcagcaaattgaacatttgaataaaacctacaatacagatgttcctcttgttttaatgaactcttttaacacggatgaagataccaaaaaaatactacagaagtacaatcattgtcgtgtgaaaatctacactttcaatcaaagcaggtacccgaggattaataaagaatctttacttcctgtagcaaaggacgtgtcttactcaggggaaaatacagaagcttggtaccctccaggtcatggtgatatttacgccagtttctacaactctggattgcttgatacctttataggagaaggcaaagagtatatttttgtgtctaacatagataatctgggtgccacagtggatctgtatattcttaatcatctaatgaacccacccaatggaaaacgctgtgaatttgtcatggaagtcacaaataaaacacgtgcagatgtaaagggcgggacactcactcaatatgaaggcaaactgagactggtggaaattgctcaagtgccaaaagcacatgtagacgagttcaagtctgtatcaaagttcaaaatatttaatacaaacaacctatggatttctcttgcagcagttaaaagactgcaggagcaaaatgccattgacatggaaatcattgtgaatgcaaagactttggatggaggcctgaatgtcattcaattagaaactgcagtaggggctgccatcaaaagttttgagaattctctaggtattaatgtgccaaggagccgttttctgcctgtcaaaaccacatcagatctcttgctggtgatgtcaaacctctatagtcttaatgcaggatctctgacaatgagtgaaaagcgggaatttcctacagtgcccttggttaaattaggcagttcttttacgaaggttcaagattatctaagaagatttgaaagtataccagatatgcttgaattggatcacctcacagtttcaggagatgtgacatttggaaaaaatgtttcattaaagggaacggttatcatcattgcaaatcatggtgacagaattgatatcccacctggagcagtattagagaacaagattgtgtctggaaaccttcgcatcttggaccactgaaatgaaaaatactgtggacacttaaataatgggctagtttcttacaatgaaatgttctctaggattctaaaataggcaggtactttactatgttactgtaccctgcagtgttgatttttaaaatagagttttctgcagtatgcttttagtctaagaaaagcacagatggagcaatactttccttctttgaagagaatcccaaaagttagttcatcttaaagtgcaatattgtttaatcttaaaactgggcaactttggaagaacttttaacagaagcctcaatgatgatcactttgaattgcttgtgatttcaaaaataaagcagtgaagcaatacttgtgtacactggtactttataatgctaactataaactggtttattgttgttagacagttactatattagttggaagatttgccctttaagtncacactggctagtt

SEQ ID NO: 77 - DNA, 2842 bp, Homo sapiens
  misc_feature: Incyte ID No 1100412.5
gctaagaggacaagatgaggcccggcctctcatttctcctagcccttctgttcttccttggccaagctgcaggggatttgggggatgtgggacctccaattcccagccccggcttcagctctttcccaggtgtttgactccaagctccagcttcagctccagctccaggtcgggctccagctccagccgcagcttaggcagcggaggttctgtgtcccagttgttttccaatttcaccggctccgtggatgaccgtgggacctgccagtgctctgtttccctgccagacaccacctttcccgtggacagagtggaacgcttggaattcacagctcatgttctttctcagaagtttgagaaagaactttccaaagtgagggaatatgtccaattaattagtttgtatgaaaagaaactgttaaacctaactgtccgaattgacatcatgggagaaggatacatttcttacactgaactggacttcgagctgataaggtagaagtgaaggagatggaaaaactggtcatacagctgaaggagagttttggtggaagctcagaaattgttgaccagctggaggtggagataagaaatatgactctcttggtagagaagcttgagacactagacaaaaacaatgtccttgccattcgccgagaaatcgtggctctgaagaccaagctgaaagagtgtgaggcctctaaagatcaaaacacccctgtcgtccaccctcctcccactccagggagctgtggtcatggtggtgtggtgaacatcagcaaaccgtctgtggttcagctcaactggagagggttttcttatctatatggtgcttggggtagggattactctccccagcatccaaacaaaggactgtattgggtggcgccattgaatacagatgggagactgttggagtattatatactgtacaacacactggatgatttgctattgtatataaatgctcgagagttgcggatcacctatggccaaggtagtggtacagcagtttacaacaacaacatgtacgtcaacatgtacacaccgggaatattgccagagttaacctgaccaccaacacgattgctgtgactcaaactctccctaatgctgcctataataaccgcttttcatatgctaatgttgcttggcaagcatattgactttgctgtggatgagaatggattgtgggttatttattcaactgaagccagcactggttaacatggtgattagtaaactcaatgacaccacacttcaggtgctaaacacttggtataccaagcagtataaaccatctgcttctaacgccttcatggtatgtggggttctgtatgccacccgtactatgaacaccagaacagaagagattttttactattatgacacaaacacagggaaagagggcaaactagacattgtaatgcataagatgcaggaaaaagtgcagagcattaactataacccttttgaccagaaactttatgtctataacgatggttaccttctgaattatgatctttctgtcttgcagaagccccagtaagctgtttaggagttagggtgaaagagaaaatgtttgttgaaaaaatagtcttctccacttacttagatatctgcagatatctatagtaagtggagaagactattttttcaacaaacattttctctttcaccctaactcctaaacagcttactggggcttctgcaagacagaaagatcataattcagaaggtaaccatcgttatagacataaagtttctggtcaaaagggttatagttaatgctctgcactttttcctgcatcttatgcattacaatgtctagtttgccctctttccctgtgtttgtgtcataatagtaaaaaatctcttctgtttggcgtctagggattctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaattgtacatggcaaataaatcccagaaggatctgtagatgaggcacctgctttttcttttctctcattgtccaccttactaaaagtcagtagaatcttctacctcatagcttccttccaaaggcagctcagaagattagaaccagacttactaaccaattccaccccccaccaacccccttctactgcctactttaaaaaaattaatagttttctatggaactgatctaagattagaaaaattaattttctttaatttcattatgaacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaaatgctttactcccccttttaaaataaatgattaaaatgtgctttgaaaaaagtc

SEQ ID NO: 78 - DNA, 2892 bp, Homo sapiens
  misc_feature: Incyte ID No 1100412.4
ggggaaatcaccactgggcactataagaagcccctgggctctctgcagagccagcggctccagctaagaggacaagatgaggcccggcctctcatttctcctagcccttctgttcttccttggccaagctgcaggggatttgggggatgtgggacctccaattcccagccccggcttcagctctttcccaggtgttgactccagctccagcttcagctccagctccaggtcgggctccagctccagccgcagcttaggcagcggaggttctgtgtcccagttgttttccaatttcaccggctccgtggatgaccgtgggacctgccagtgctctgtttccctgccagacaccacctttcccgtggacagagtggaacgcttggaattcacagctcatgttctttctcagaagtttgagaaagaactttccaaagtgagggaatatgtccaattaattagtgtgtatgaaaagaaactgttaaacctaactgtccgaattgacatcatggagaaggataccatttcttacactgaactggacttcgagctgatcaaggtagaagtgaaggagatggaaaaactggtcatacagctgaaggagagttttggtggaagctcagaaattgttgaccagctggaggtggagataagaaatatgactctcttggtagagaagcttgagacactagacaaaaacaatgtccttgccattcgccgagaaatcgtggctctgaagaccaagctgaaagagtgtgaggcctctaaagatcaaaacacccctgtcgtccaccctcctcccactccagggagctgtggtcatggtggtgtggtgaacatcagcaaaccgtctgtggttcagctcaactggagagggttttcttatctatatggtgcttggggtagggattactctccccagcatccaaacaaaggactgtattgggtggcgccattgaatacagatgggagactgttggagtattatagactgtacaacacactggatgatttgctattgtatataaatgctcgagagttgcggatcacctatggccaaggtagtggtacagcagtttacaacaacaacatgtacgtcaacatgtacaacaccgggaatattgccagagttaacctgaccaccaacacgattgctgtgactcaaactctccctaatgctgcctataataaccgcttttcatatgctaatgttgcttggcaagatattgactttgctgtggatgagaatggattgtgggttatttattcaactgaagccagcactggtaacatggtgattagtaaactcaatgacaccacacttcaggtgctaaacacttggtataccaagcagtataaaccatctgcttctaacgccttcatggtatgtggggttctgtatgccacccgtactatgaacaccagaacagaagagattttttactattatgacacaaacacagggaaagagggcaaactagacattgtaatgcataagatgcaggaaaaagtgcagagcattaactataacccttttgaccagaaactttatgtctataacgatggttaccttctgaattatgatctttctgtcttgcagaagccccagtaagctgtttaggagttagggtgaaagagaaaatgtttgttgaaaaaatagtcttctccacttacttagatatctgcaggggtgtctaaaagtgtgttcattttgcagcaatgtttaggtgcatagttctaccacactagagatctaggacatttgtcttgatttggtgagttctcttgggaatcatctgcctcttcaggcgcattttgcaataaagtctgtctagggtgggattgtcagaggtctaggggcactgtgggcctagtgaagcctactgtgaggaggcttcactagaagccttaaattaggaattaaggaacttaaaactcagtatggcgtctagggattctttgtacaggaaatattgcccaatgactagtcctcatccatgtagcaccactaattcttccatgcctggaagaaacctggggacttagttaggtagattaatatctggagctcctcgagggaccaaatctccaacttttttttcccctcactagcacctggaatgatgctttgtatgtggcagataagtaaatttggcatgcttatatattctacatctgtaaagtgctgagttttatggagagaggcctttttatgcattaaattgtacatggcaaataaatcccagaaggatctgtagatgaggcacctgctttttcttttctctcattgtccaccttactaaaagtcagtagaatcttctacctcataacttccttccaaaggcagctcagaagattagaaccagacttactaaccaattccaccccccaccaacccccttctactgcctactttaaaaaaattaatagttttctatggaactgatctaagattagaaaaattaattttctttaatttcattatggacttttatttacatgactctaagactataagaaaatctgatggcagtgacaaagtgctagcatttattgttatctaataaagaccttggagcatatgtgcaacttatgagtgtatcagttgttgcatgtaatttttgcctttgtttaagcctggaacttgtaagaaaatgaaaatttaatttttttttctaggacgagctatagaaaagctattgagagtatctagttaatcagtgcagtagttggaaaccttgctggtgtatgtgatgtgcttctgtgcttttgaatgactttatcatctagtctttgtctatttttcctttgatgttcaagtcctagtctataggattggcagtttaaatgctttactcccccttttaaaataaatgattaaaatgtgctttgaat

SEQ ID NO: 79 - DNA, 1244 bp, Homo sapiens
  misc_feature: Incyte ID No 2101663CB1
cagtcctcaggtgcaacccctgcgtggtctctgtggcagccttctctcattcagagcttgcacagttgcagttagttattccaggtattatttttgttttcagaaaaagaaaactcagtagaagataatggcaagtccagactggggatatgatgacaaaaatggtcctgaacaatggagcaagctgtatcccattgccaatggaaataaccagtcccctgttgatattaaaaccagtgaaaccaaacatgacacctctctgaaacctattagtgtctcctacaacccagccacagccaaagaaattatcaatgtggggcattccttccatgtaaattttgaggacaacgataaccgatcagtgctgaaaggtggtcctttctctgacagctacaggctctttcagttccattttcactggggcagtacaaatgagcatggttcagaacatacagtggatggagtcaaatattctgccgagcttcacgtagctcactggaattctgcaaagtactccagccttgctgaagctgcctcaaaggctgatggtttggcagttattggtgttttgatgaaggttggtgaggccaacccaaagctgcagaaagtacttgatgccctccaagcaattaaaaccaagggcaaacgagccccattcacaaattttgacccctctactctccttccttcatccctggatttctggacctaccctggctctctgactcatcctcctctttatgagagtgtaacttggatcatctgtaaggagagcatcagtgtcagctcagagcagctggcacaattccgcagccttctatcaaatgttgaaggtgataacgctgtccccatgcagcacaacaaccgcccaacccaacctctgaagggcagaacagtgagagcttcattttgatgattctgagaagaaacttgtccttcctcaagaacacagccctgcttctgacataatccagtaaaataataatttttaagaaataaatttatttcaatattagcaagacagcatgccttcaaatcaatctgtaaaactaagaaacttaaattttagttcttactgcttaattcaaataataattagtaagctagcaaatagtaatctgtaagcataagcttatgcttaaattcaagtttagtttgaggaattctttaaaattacaactaagtgatttgtatgtctatttttttcagtttatttgaaccaataaaataattttatctctttc

SEQ ID NO: 80 - protein, 261 aa, Homo sapiens
  misc_feature: Incyte ID No 2101663CD1
MASPDWGYDDKNGPEQWSKLYPIANGNNQSPVDIKTSETKHDTSLKPISVSYNPATAKEIINVGHSFHVNFEDNDNRSVLKGGPFSDSYRLFQFHFHWGSTNEHGSEHTVDGVKYSAELHVAHWNSAKYSSLAEAASKADGLAVIGVLMKVGEANPKLQKVLDALQAIKTKGKRAPFTNFDPSTLLPSSLDFWTYPGSLTHPPLYESVTWIICKESISVSSEQLAQFRSLLSNVEGDNAVPMQHNNRPTQPLKGRTVRASF

SEQ ID NO: 81 - DNA, 1317 bp, Homo sapiens
  misc_feature: Incyte ID No 611082CB1
aaggaacaaagtaagtccattgatacgttcttgcctatctctcctccaaatcaatgggcacaaactgtggctggtctacctgtgtgggttctgttctctagattggagggatgaagacaagttcttgactctatgttgaggccagttgaaaaatgagggagaataaaaccatgaacgaaacaagaaagaaacaaaacagaagaggaatgaaaaagacataatgatgtcatccaagccaacaagccatgctgaagtaaatgaaaccatacccaacccttacccaccaagcagctttatggctcctggatttcaacagcctctgggttcaatcaacttagaaaaccaagctcagggtgctcagcgtgctcagccctacggcatcacatctccgggaatctttgctagcagtcaaccgggtcaaggaaatatacaaatgataaatccaagtgtgggaacagcagtaatgaactttaaagaagaagcaaaggcactaggggtgatccagatcatggttggattgatgcacattggttttggaattgttttgtgtttaatatccttctcttttagagaagtattaggttttgcctctactgctgttattggtggatacccattctggggtggcctttcttttattatctctggctctctctctgtgtcagcatccaaggagctttcccgttgtctggtgaaaggcagcctgggaatgaacattgttagttctatcttggccttcattggagtgattctgctgctggtggatatgtgcatcaatggggtagctggccaagactactgggccgtgctttctggaaaaggcatttcagccacgctgatgatcttctccctcttggagttcttcgtagcttgtgccacagcccattttgccaaccaagcaaacaccacaaccaatatgtctgtcctggttattccaaatatgtatgaaagcaaccctgtgacaccagcgtcttcttcagctcctcccagatgcaacaactactcagctaatgcccctaaatagtaaaagaaaaaggggtatcagtctaatctcatggagaaaaactacttgcaaaaacttcttaagaagatgtcttttattgtctacaatgatttctagtctttaaaaactgtgtttgagatttgtttttaggttggtcgctaatgatggctgtatctcccttcactgtctcttcctacattaccactactacatgctggcaaaggtgaaggatcagaggactgaaaaatgattctgcaactctcttaaagttagaaatgtttctgttcatattactttttccttaataaaatgtc

SEQ ID NO: 82 - protein, 267 aa, Homo sapiens
  misc_feature: Incyte ID No 611082CD1
MMSSKPTSHAEVNETIPNPYPPSSFMAPGFQQPLGSINLENQAQGAQRAQPYGITSPGIFASSQPGQGNIQMINPSVGTAVMNFKEEAKALGVIQIMVGLMHIGFGIVLCLISFSFREVLGFASTAVIGGYPFWGGLSFIISGSLSVSASKELSRCLVKGSLGMNIVSSILAFIGVILLLVDMCINGVAGQDYWAVLSGKGISATLMIFSLLEFFVACATAHFANQANTTTNMSVLVIPNMYESNPVTPASSSAPPRCNNYSANAPK

SEQ ID NO: 83 - DNA, 1889 bp, Homo sapiens
  misc_feature: Incyte ID No 255002.4
caaatgagtgctgttaaagttcctccaggaaacttcagcagagaaaaacatttgcttcacatctcatcaaatcttctgcatcaagccacatcatgttaaacaaccttctgctgttctcccttcagataagtctcataggaaccactcttggtgggaatgttttgatttggccaatggaaggtagtcattggctaaatgttaagataattatagatgagctcattaaaaaggngcataatgtgnntgtcctagttgcctctggtgcacttttcatcacaccaacctctaacccatctctgacatttgaaatatataaggtgccctttggcaaagaaagaatagaaggagtaattaaggacttcgttttgacatggctggaaaatagaccatctccttcaaccatttggagattctatcaggagatggccaaagtaatcaaggacttccacatggtgtctcaggagatctgtgatggcgttcttaaaaaccaacagctgatggcaaagctaaagaaaagcaagtttgaagtcctggtgtctgatccagtatttccttgtggcgatatagtagctttaaaacttggaattccatttatgtactccttgaggttttctccagcctcaacagtggaaaagcactgtgggaaggtaccataccctccttcctatgttcctgctgttttatcagaactcaccgaccaaatgtctttcactgacagaataagaaatttcatctcctaccacctacaggactacatgtttgaaactctttggaaatcatgggattcatactatagtaaagctttaggaagacccactacgttatgtgagactatggggaaagctgaaatttggttaatccgaacatattgggattttgaatttcctcgtccatacttacctaattttgagtttgttggaggattgcactgcaaacctgccaaacctttacctaaggaaatggaagaatttatccagagctcaggtaaaaatggtgttgtggtgttttctctgggatcaatggtcaaaaaccttacagaagaaaaggccaatcttattgcctcagcccttgcccagattccacagaaggttttatggagatacaaaggaaagaaaccagccacattaggaaacaatactcagctctttgattggataccccagaatgatcttcttggacatcccaaaaccaaagcttttatcactcatggtggaactaatgggatctacgaagctatttaccacggagtccctatggtgggagttcccatgtttgctgatcagcctgataacattgctcacatgaaggccaaaggagcagctgtggaagtgaacctaaacacaagtgacaagtgtggatttgcttagcgctttgagaacagtcattaatgaaccttcttataaagagaatgctatgaggttatcaagaattcaccatgatcaacctgtaaagcccctggatcgagcagtcttctggatcgagtttgtcatgcgccacaaaggagccaagcaccttcgggttgcagcccatgacctcacctggttccagtaccactctttggatgtaattgggttcttgctggtctgtgtgacaacggctatatttttggtcatacaatgttgtttgttttcctgtcaaaaatttggtaagataggaaagaagaaaaaaagagaataggtcaagaaaaagaggaaatatatatatttttaagtttggcaaaatcctgagtagtgtagtcctattaattccagacaaaaggagtttaacaaaaacacgtctcccatcctgtttccaaattttctatttctctacctgcgataagcctactgataaagcctagattttggcatgattattattaacttgtgagttatagtcttctatttttc

SEQ ID NO: 84 - DNA, 3591 bp, Homo sapiens
  misc_feature: Incyte ID No 1092257.2
cgctcccacccgcccgtggcccgcgcccatggccgcgcgcgctccacacaactcaccggagtccgcgccctgcgccgccgaccagttcgcagctccgcgccacggcagccagtctcacctggcggcaccgcccgcccaccgccccggccacagcccctgcgcccacggcagcactcgaggcgaccgcgacagtggtgggggacgctgctgagtggaagagagcgcagcccggccaccggacctacttactcgccttgctgattgtctatttttgcgtttacaacttttctaagaacttttgtatacaaaggaactttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataaccgctggcgggaggagctctcccacatgaagcgacttcccccagtgcttcccggccgcccctatgacctggcggcggcgaccgtggccacagacctggagagcggcggagccggtgcggcttgcggcggtagcaacctggcgcccctacctcggagagagaccgaggagttcaacgatctcctggacctggactttattctctccaattcgctgacccatcctccggagtcagtggccgccaccgtgtcctcgtcagcgtcagcctcctcttcgtcgtcgccgtcgagcagcggccctgccagcgcgccctccacctgcagcttcacctatccgatccgggccgggaacgacccgggcgtggcgccgggcggcacgggcggaggcctcctctatggcagggagtccgctccccctccgacggctcccttcaacctggcggacatcaacgacgtgagcccctcgggcggcttcgtggccgagctcctgcggccagaattggacccggtgtacattccgccgcagcagccgcagccgccaggtggcgggctgatgggcaagttcgtgctgaaggcgtcgctgagcgcccctggcagcgagtacggcagcccgtcggtcatcagcgtcagcaaaggcagccctgacggcagccacccggtggtggtggcgccctacaacggcgggccgccgcgcacgtgccccaagatcaagcaggaggcggtctcttcgtgcacccacttgggcgctggaccccctctcagcaatggccaccggccggctgcacacgacttccccctggggcggcagctccccagcaggactaccccgaccctgggtcttgaggaagtgctgagcagcagggactgtcaccctgccctgccgcttcctcccggcttccatccccacccggggcccaattacccatccttcctgcccgatcagatgcagccgcaagtcccgccgctccattaccaagagctcatgccacccggttcctgcatgccagaggagcccaagccaaagaggggaagacgatcgtggccccggaaaaggaccgccacccacacttgtgattacgcgggctgcggcaaaacctacacaaagagttcccatctcaaggcacacctgcgaacccacacaggtgagaaaccttaccactgtgactgggacggctgtggatggaaattcgcccgctcagatgaactgaccaggcactaccgtaaacacacggggcaccgcccgttccagtgccaaaaatgcgaccgagcattttccaggtcggaccacctcgccttacacatgaagaggcatttttaaatcccagacagtggatatgacccacactgccagaagagaattcagtattttttacttttcacactgtcttcccgatgagggaaggagcccagccagaaagcactacaatcatggtcaagttcccaactgagtcatcttgtgagtggataatcaggaaaaatgaggaatccaaaagacaaaaatcaaagaacagatggggtctgtgactggatcttctatcattccaattctaaatccgacttgaatattcctggacttacaaaatgccaagggggtgactggaagttgtggatatcagggtataaattatatccgtgagttgggggagggaagaccagaattcccttgaattgtgtattgatgcaatataagcataaaagatcaccttgtattctctttaccttctaaaagccattattatgatgttagaagaagaggaagaaattcaggtacagaaaacatgtttaaatagcctaaatgatggtgcttggtgagtcttggttctaaaggtaccaaacaaggaagccaaagttttcaaactgctgcatactttgacaaggaaaatctatatttgtcttccgatcaacatttatgacctaagtcaggtaatatacctggtttacttctttagcatttttatgcagacagtctgttatgcactgtggtttcagatgtgcaataatttgtacaatggtttattcccaagtatgccttaagcagaacaaatgtgtttttctatatagttccttgccttaataaatatgtaatataaatttaagcaaacgtctattttgtatatttgtaaactacaaagtaaaatgaacattttgtggagtttgtattttgcatactcaaggtgagaattaagttttaaataaactacatacattgcaaggcataaaccttaactgtacatgttagacaaataaatagacccatataaacttcaccccttttcgaagcaacactttcatcaccctcagaaaattcacttgtctttggtcctttttctcatgcagagattatcttgttgattttttttcaccataggattattttgccctcactttcccactctttccttaagtttaaattctgagggccacagctcccatatccatccaatatcactttttggaatgatgcttctataagcttctttcttaagaggccttgagtatcatgagaaaagatatctggctggatggctactgtctctgcactcttcacattccagggctctttcctttgctccaaaaaggtgatcaggtccggcttagagacagcgagacccagagagaccaggtttccgtagttctctaacatcacatccccatataaaagcttctgatctgagtccaggtgttcccactcctcccgagagaattctatggccacatccctgaatgtcaacagtccctcaactgaagggacaggagcaggtgcccagccagtcagatgttcagtgacctcaaagagggttcccgccgcttccgaccgcccctctccctcttctggggacccccgacctggcacattcaccatttcccagcttccgggatgtcctggcgtctgagctacggatctcccggtgcctgccggtcacagagtaacagaggttctgtctattctcagaacgcgaagccgccttggcctcccagatggaaggactggg

SEQ ID NO: 85 - DNA, 3115 bp, Homo sapiens
  misc_feature: Incyte ID No 1102315.3
cgagcgggatccaaacttccggtgcctgcagagctcggagcggcggaggcagagaccgaggctgcaccggcagaggctgcggggcggacgcgcgggccggcgcagccatggtgaagattagcttccagcccgccgtggctggcatcaagggcgacaaggctgacaaggcgtcggcgtcggcccctgcgccggcctcggccaccgagatcctgctgacgccggctagggaggagcagcccccacaacatcgatccaagagggggagctcagtgggcggcgtgtgctacctgtcgatgggcatggtcgtgctgctcatgggcctcgtgttcgcctctgtctacatctacagatacttctttcttgcacagctggcccgagataacttcttccgctgtggtgtgctgtatgaggactccctgtcctcccaggtccggactcagatggagctggaagaggatgtgaaaatctacctcgacgagaactacgagcgcatcaacgtgcctgtgccccagtttggcggcggtgaccctgcagacatcatccatgacttccagcggggtctgactgcgtaccatgatatctccctggacaagtgctatgtcatcgaactcaacaccaccattgtgctgccccctcgcaacttctgggagctcctcatgaacgtgaagagggggacctacctgccgcagacgtacatcatccaggaggagatggtggtcacggagcatgtcagtgacaaggaggccctggggtccttcatctaccacctgtgcaacgggaaagacacctaccggctccggcgccgggcaacgcggaggcggatcaacaagcgtggggccaagaactgcaatgccatccgccacttcgagaacaccttcgtggtggagacgctcatctgcggggtggtgtgaggccctcctcccccagaaccccctgccgtgttcctcttttcttctttccggctgctctctggccctcctccttccccctgcttagcttgtactttggacgcgtttctatagaggtgacatgtctctccattcctctccaaccctgcccacctccctgtaccagagctgtgatctctcggtggggggcccatctctgctgacctgggtgtggcggagggagaggcgatgctgcaaagtgttttctgtgtcccactgtcttgaagctgggcctgccaaagcctgggcccacagctgcaccggcagcccaaggggaaggaccggttgggggagccgggcatgtgaggccctgggcaaggggatggggctgtgggggcggggcggcatgggcttcagaagtatctgcacaattagaaaagtcctcagaagctttttcttggagggtacactttcttcactgtccctattcctagacctggggcttgagctgaggatgggacgatgtgcccagggagggacccaccagagcacaagagaaggtggctacctgggggtgtcccagggactctgtcagtgccttcagcccaccagcaggagcttggagtttggggagtggggatgagtccgtcaagcacaactgttctctgagtggaaccaaagaagcaaggagctaggacccccagtcctgccccccaggagcacaagcagggtcccctcagtcaaggcagtgggatgggcggctgaggaacggggcaggcaaggtcactgctcagtcacgtccacgggggacgagccgtgggttctgctgagtaggtggagctcattgctttctccaagcttggaactgttttgaaagataacacagagggaaagggagagccacctggtacttgtccaccctgcctcctctgttctgaaattccatccccctcagcttaggggaatgcacctttttccctttccttctcacttttgcatgtttttactgatcattcgatatgctaaccgttctcagccctgagccttggagaggagggctgtaacgccttcagtcagtctctggggatgaaactcttaaatgctttgtatattttctcaattagatctcttttcagaagtgtctatagaacaataaaaatcttttttttcagaagtaaaagatttttattgttctatagacacttctgaaaagagatctaattgagaaaatatacaaagcatttaagagtttcatccccagagactgactgaaggcgttacagccctcctctccaaggctcagggctgagaacggttagcatatcgaatgatcagtaaaaacatgcaaaagtgagaaggaaagggaaaaaggtgcattcccctaagctgagggggatggaatttcagaacagaggaggcagggtggacaagtaccaggtggctctccctttccctctgtgttatctttcaaaacagttccaagcttggagaaagcaatgagctccacctactcagcagaacccacggctcgtcccccgtggacgtgactgagcagtgaccttgcctgccccgttcctcagccgcccatcccactgccttgactgaggggacactgcttgtgctcctggggggcaggactgggggtcctagctccttgcttctttggttccactcagagaacagttgtgcttgacggactcatccccactccccaaactccaagctcctgctggtgggctgaaggcactgacagagtccctgggacacccccaggtagccaccttctcttgtgctctggtgggtccctccctgggcacatcgtcccntcctcngctcaagccccaggtctaggaatagggacagtgaagaaagtgtaccctccaagaaaaagcttctgaggacttttctaattgtgcagatacttctgaagcccatgccgccccgcccccacagccccatccccttgcccagggcctcacagaaaacactttgcagcatcgcctctccctccgccacacccaggtcagcagagatgggccccccaccgagagatcacagctctggtacagggaggtgggcagggttggagaggaatggagagacatgtcacctctatagaaacgcgtccaaagtacaagctaagcagggggaaggaggagggccagagagcagccggaaagaagaaaagaggaacacggcagggggtntctgggggaggagggcctcac

SEQ ID NO: 86 - DNA, 1571 bp, Homo sapiens
  misc_feature: Incyte ID No 1543330CB1
tgccaccgctgcccgagcccgagtggttcactgcactgtgaaaacagattccagacgccgggaactcacgcctccaatcccagacgctatgtccagcaaaggctccgtggttctggcctacagtggcggcctggacacctcgtgcatcctcgtgtggctgaaggaacaaggctatgacgtcattgcctatctggccaacattggccagaaggaagacttcgaggaagccaggaagaaggcactgaagcttggggccaaaaaggtgttcattgaggatgtcagcagggagtttgtggaggagttcatctggccggccatccagtccagcgcactgtatgaggaccgctacctcctgggcacctctcttgccaggccctgcatcgcccgcaaacaagtggaaatcgcccagcgggagggggccaagtatgtgtcccacggcgccacaggaaaggggaacgatcaggtccggtttgagctcagctgctactcactggccccccagataaaggtcattgctccctggaggatgcctgaattctacaaccggttcaagggccgcaatgacctgatggagtacgcaaagcaacatgggattcccatcccggtcactcccaagaacccgtggagcatggatgagaacctcatgcacatcagctacgaggctggaatcctggagaaccccaagaaccaagcgcctccaggtctctacacgaagacccaggacccagccaaagcccccaacacccctgacattctcgagatcgagttcaaaaaaggggtccctgtgaaggtgaccaacgtcaaggatggcaccacccaccagacctccttggagctcttcatgtacctgaacgaagtcgcgggcaagcatggcgtgggccgtattgacatcgtggagaaccgcttcattggaatgaagtcccgaggtatctacgagaccccagcaggcaccatcctttaccatgctcatttagacatcgaggccttcaccatggaccgggaagtgcgcaaaatcaaacaaggcctgggcttgaaatttgctgagctggtgtataccggtttctggcacagccctgagtgtgaatttgtccgccactgcatcgccaagtcccaggagcgagtggaagggaaagtgcaggtgtccgtcctcaagggccaggtgtacatcctcggccgggagtccccactgtctctctacaatgaggagctggtgagcatgaacgtgcagggtgattatgagccaactgatgccaccgggttcatcaacatcaattccctcaggctgaaggaatatcatcgtctccagagcaaggtcactgccaaatagacccgtgtacaatgaggagctggggcctcctcaatttgcagatcccccaagtacaggcgctaattgttgtgataatttgtaattgtgacttgttctccccggctggcagcgtagtggggctgccaggccccagctttgttccctggtccccctgaagcctgcaaacgttgtcatcgaagggaagggtggggggcagctgcggtggggagctataaaaatgacaattaaaagagaaaaaaaaaaa

SEQ ID NO: 87 - protein, 412 aa, Homo sapiens
  misc_feature: Incyte ID No 1543330CD1
MSSKGSVVLAYSGGLDTSCILVWLKEQGYDVIAYLANIGQKEDFEEARKKALKLGAKKVFIEDVSREFVEEFIWPAIQSSALYEDRYLLGTSLARPCIARKQVEIAQREGAKYVSHGATGKGNDQVRFELSCYSLAPQIKVIAPWRMPEFYNRFKGRNDLMEYAKQHGIPIPVTPKNPWSMDENLMHISYEAGILENPKNQAPPGLYTKTQDPAKAPNTPDILEIEFKKGVPVKVTNVKDGTTHQTSLELFMYLNEVAGKHGVGRIDIVENRFIGMKSRGIYETPAGTILYHAHLDIEAFTMDREVRKIKQGLGLKFAELVYTGFWHSPECEFVRHCIAKSQERVEGKVQVSVLKGQVYILGRESPLSLYNEELVSMNVQGDYEPTDATGFININSLRLKEYHRLQSKVTAK

SEQ ID NO: 88 - DNA, 3152 bp, Homo sapiens
  misc_feature: Incyte ID No 232992.1
taacaggatctgctgggccttgacccaggactttccagcttctagaaccatgaaaaatttctgttgttcctagcagcccaaacagaattagaaccattaatttctatttctcctttagcttaacactggactttagttttaatcttaactgagagtggtcctgctagtttctttgctttacctgtttttaaaacactttgagctaagaatttttcacccaattatcacacatgattagaaaacaagatgcttaaagtgaaaaaagacacccccacctttaccctacttgatatccaggtccttttcctccaagaatatttctagctgcttctggttttgctagtttatttcaaaaattacaattgtttgagtcaaatctatacttgatattcattgttgggtttctttaggtcattctgctgttctctcagtttcccaggtgttttgtgtgtcttccttgtgctattaattttcctgtggccttgttggtcagatccacagaggtaaatgcaagtgtttttccttgcttcagaacatgtgctgtaatcactatatcttctcctaattttgcaggtgacatgtacgttatgttcatatcgacactgactccgggtgctcccctttccgtgcatagcagagccattgttgatatgttatctactaatgtggctgtcaaaccgccgtggagagtgcctattgcattggtatgctcttcttctactttcatttcacaaatcactttcccaggagcagcagagacaagagtaatctttcccaaaactctctcaaaattgcgagccttggtcatggcctttatcacctcccgcagagactgagtcatgctggtcatcgtggacggttttccagcctttgggctttgcgcaagaacgaacttcggaagagctggaaagtccagaagttagtgaaagaaggaggacacaaagggcgaagagcccgcacccttctccgcccctaggagcgcaagaggccgggagtcaggcgagctggcagccccggtggtccgcgatttgctcactgactttcctcgcacccttgtgacgaagcgcgcatgcgcgttcgcacccaccgccccggcggctcccttgcggcgcagctgcaccagttttccgagagcggagcgcatttccccgccgggcggttgatgcgggangccatgacacaggcgcctgcgcagagccgcgcgttggcctcctgttccgcttaaaggggcggtgcagaggcggcaggaagatggagttggggagttgcctggagggcgggagggaggcggcggaggaagagggcgagcctgaggtgaaaaagcggcgacttctgtgtgtggagtttgcctcggtcgcaagctgcgatgccgcagtggctcagtgcttcctggccgagaacgactgggagatggaaagggctctgaactcctacttcgagcctccggtggaggagagcgccttggaacgccgacctgaaaccatctctgagcccaagacctatgttgacctaaccaatgaagaaacaactgattccaccacttctaaaatcagcccatctgaagatactcagcaagaaaatggcagcatgttctctctcattacctggaatattgatggattagatctaaacaatctgtcagagagggctcgaggggtgtgttcctacttagctttgtacagcccagatgtgatatttctacaggaagttattcccccatattatagctacctaaagaagagatcaagtaattatgagattattacaggtcatgaagaaggatatttcacagctataatgttgaagaaatcaagagtgaaattaaaaagccaagagattattccttttccaagtaccaaaatgatgagaaaccttttatgtgtgcatgtgaatgtgtcaggaaatgagctttgccttatgacatcccatttggagagcaccagagggcatgctgcggaacgaatgaatcagttaaaaatggttttaaagaaaatgcaagaggctccagagtcagctacagttatatttgcaggagatacaaatctaagggatcgagaggttaccagatgtggtggtttacccaacaacattgtggatgtctgggagtttttgggcaaacctaaacattgccagtatacatgggatacacaaatgaactctaatcttggaataactgctgcttgtaaacttcgttttgatcgaatatttttcagagcagcagcagaagagggacacattattccccgaagtttggaccttcttggattagaaaaactggactgtggtagatttcctagtgatcactggggtcttctgtgcaacttagatataatattgtaaaatgcttttcaagtgtgggttttgccctgattgttgcaaatacaatttccaccttctggaaaggtaggtttgctgtggaggaaataatgtactagatcattgtcacagaaaaaccaactatgatttatggttgtgttttcagaattcaacattaaagattaatgtttatttaaacgaacacattcctgcattcaggatgtgaggccatttaataaaaagggcacaaagcctgtcagagttttcaacggtgcttatagctgccagctggattccaaacaggtaccccattgtctctgagctaatgtttatatttttccattcaggcaccgaaatagttaatatttaaaataagtcttcaaaagaaaacataagagattattgagttcttgggactggatcctttatttcataagttcagatcatcttaaatgaaaatgccatgattatctgcagttaagtagatgacagctattctacatcagacttgatttttgtcagctaattacataattggtaagctataattgaaaccttatggcttaaaattccttaactcctttttgattcatgtttgtagtcatgttgtcaacagaggcaaagttaagcttgatgatggttaaaatcggtttgatagcaccatgggacatttttctaacaaaaataaatgcatgaagagacatagccttttagttttgctaattgtgaaatggaaatgctttacaggaagtaaatgcaaattacttttaagtgtgctttaaagaaaaatattttccccacaagagaaatttaaataaagaattttatttgtttat

SEQ ID NO: 89 - DNA, 1239 bp, Homo sapiens
  misc_feature: Incyte ID No 1281620CB1
cttatcccgctgcacgccccagcctcgctggcttgctgtaaacaaggaacccgggggagtgcggagggagaaagagcttctcccaggcctcaccctactgcacacaccgacttccttcctcacgcctgtctctctttcctggttcccacccgtgccaggtgacacgcagagctgaagccatggttcatcaggtgctctaccgggcgctggtctccaccaagtggctggcggagtccatcaggactggcaagctggggcccggcctgcgggtgctggacgcgtcctggtactcaccaggcacccgagaggcccgcaaggagtacctcgagcgccacgtacccggcgcctctttctttgacatagaagagtgccgggacacggcgtcgccctacgagatgatgctgcccagcgaggctggcttcgccgagtatgtgggccgcctgggcatcagcaaccacacgcacgtggtggtgtatgatggtgaacacctgggcagcttctatgctccccgggtctggtggatgttccgtgtgtttggccaccgcaccgtatcagtgctcaatggtggcttccggaactggctgaaggagggccacccggtgacatccgagccctcacgcccagaaccggccgtcttcaaagccacactggaccgctccctgctcaagacctacgagcaggtgctggagaaccttgaatctaagaggttccagctggtggattcaaggtctcaagggcggttcctgggcaccgagccggagccggatgcagtaggactggactcgggccatatccgtggtgccgtcaacatgcctttcatggacttcctgactgaggatggcttcgagaagggcccagaagagctccgtgctctgttccagaccaagaaggtggatctctcgcagcctctcattgccacgtgccgcaagggagtcaccgcctgccacgtggccttggctgcctacctctgcggcaagcctgatgtggccgtgtacgatggctcctggtccgagtggtttcgccgggcccccccagagagccgtgtgtcccagggaaagtctgagaaggcctgagccgtgacctcttctgcttactgtaactgcggccggtttagtgaccccatgacttacagccggttcttacctcttaggtgaaggagatgacatgtttttagaattgctgtgcaaggctcaccctctctctgtcaacactggaataaactttgccttttctgaaaaa

SEQ ID NO: 90 - protein, 297 aa, Homo sapiens
  misc_feature: Incyte ID No 1281620CD1
MVHQVLYRALVSTKWLAESIRTGKLGPGLRVLDASWYSPGTREARKEYLERHVPGASFFDIEECRDTASPYEMMLPSEAGFAEYVGRLGISNHTHVVVYDGEHLGSFYAPRVWWMFRVFGHRTVSVLNGGFRNWLKEGHPVTSEPSRPEPAVFKATLDRSLLKTYEQVLENLESKRFQLVDSRSQGRFLGTEPEPDAVGLDSGHIRGAVNMPFMDFLTEDGFEKGPEELRALFQTKKVDLSQPLIATCRKGVTACHVALAAYLCGKPDVAVYDGSWSEWFRRAPPESRVSQGKSEKA

SEQ ID NO: 91 - DNA, 1860 bp, Homo sapiens
  misc_feature: Incyte ID No 343502.10
ggcagatggcagggtcctttgcaggagaggcagcggcgaaagctgcccttaggaggcagcgaggaggtgaaaccagagagcaacaagtaaatgcagcactagtgggtgggattgaggtgtgccctggtgcataaatagagactcagctgtgctggcacactcagaagcttggaccgcatcctagccgccgactcacacaaggcaggtgggtgaggaaatccagagttgccatggagaaaattccagtgtcagcattcttgctccttgtggccctctcctacactctggccagagataccacagtcaaacctggagccaaaaaggacacaaaggactctcgacccaaactgccccagaccctctccagaggttggggtgaccaactcatctggactcagacatatgaagaagctctatataaatccaagacaagcaacaaacccttgatgattattcatcacttggatgagtgcccacacagtcaagctttaaagaaagtgtttgctgaaaataaagaaatccagaaattggcagagcagtttgtcctcctcaatctggtttatgaaacaactgacaaacacctttctcctgatggccagtatgtccccaggattatgtttgttgacccatctctgacagttagagccgatatcactggaagatattcaaatcgtctctatgcttacgaacctgcagatacagctctgttgcttgacaacatgaagaaagctctcaagttgctgaagactgaattgtaaagaaaaaaaatctccaagcccttctgtctgtcaggccttgagacttgaaaccagaagaagtgtgagaagactggctagtgtggaagcatagtgaacacactgattaggttatggtttaatgttacaacaactattttttaagaaaaacaagttttagaaatttggtttcaagtgtacatgtgtgaaaacaatattgtatactaccatagtgagccatgattttctaaaaaaaaaaataaatgttttgggggtgttctgttttctccaacttggtctttcacagtggttcgtttaccaaataggattaaacacacacaaaatgctcaaggaagggacaagacaaaaccaaaactagttcaaatgatgaagaccaaagaccaagttatcatctcaccacaccacaggttctcactagatgactgtaagtagacacgagcttaatcaacagaagtatcaagccatgtgctttagcataaaagaatatttagaaaaacatcccaagaaaatcacatcactacctagagtcaactctggccaggaactctaaggtacacactttcatttagtaattaaattttagtcagattttgcccaacctaatgctctcagggaaagcctctggcaagtagctttctccttcagaggtctaatttagtagaaaggtcatccaaagaacatctgcactcctgaacacaccctgaagaaatcctgggaattgaccttgtaatcgatttgtctgtcaaggtcctaaagtactggagtgaaataaattcagccaacatgtgactaattggaagaagagcaaagggtggtgacgtgttgatgaggcagatggagatcagaggttactagggtttaggaaacgtgaaaggctgtggcatcagggtaggggagcattctgcctaacagaaattagaattgtgtgttaatgtcttcactctatacttaatctcacattcattaatatatggaattcctctactgcccagcccctcctgatttctttggcccctggactatggtgctgtatataatgctttgcagtatctgttgcttgtcttgattaacttttttggataaaaccttttttgaacagaaaaaa

SEQ ID NO: 92 - DNA, 1711 bp, Homo sapiens
  misc_feature: Incyte ID No 1635966CB1
tttgatacgggagttcctccttgctctcgcccctactctttctggtgttagatcgagctaccctctaaaagcagtttagagtggtaaaaaaaaaaaaaaaacacaccaaacgctcgcagccacaaaagggatgaaatttcttctggacatcctcctgcttctcccgttactgatcgtctgctccctagagtccttcgtgaagctttttattcctaagaggagaaaatcagtcaccggcgaaatcgtgctgattacaggagctgggcatggaattgtgagactgactgcctatgaatttgctaaacttaaaagcaagctggttctctgggatataaataagcatggactggaggaaacagctgccaaatgcaagggactgggtgccaaggttcatacctttgtggtagactgcagcaaccgagaagatatttacagctctgcaaagaaggtgaaggcagaaattggagatgttagtattttagtaaataatgctggtgtagtctatacatcagatttgtttgctacacaagatcctcagattgaaaagacttttgaagttaatgtacttgcacatttctggactacaaaggcatttcttcctgcaatgacgaagaataaccatggccatattgtcactgtggcttcggcagctggacatgtctcggtccccttcttactggcttactgttcaagcaagtttgctgctgttggatttcataaaactttgacagatgaactggctgccttacaaataactggagtcaaaacaacatgtctgtgtcctaatttcgtaaacactggcttcatcaaaaatccaagtacaagtttgggacccactctggaacctgaggaagtggtaaacaggctgatgcatgggattctgactgagcagaagatgatttttattccatcttctatagcttttttaacaacattggaaaggatccttcctgagcgtttcctggcagttttaaaacgaaaaatcagtgttaagtttgatgcagttattggatataaaatgaaagcgcaataagcacctagttttctgaaaactgatttaccaggtttaggttgatgtcatctaatagtgccagaattttaatgtttgaacttctgttttttctaattatccccatttcttcaatatcatttttgaggctttggcagtcttcatttactaccacttgttctttagccaaaagctgattacatatgatataaacagagaaatacctttagaggtgactttaaggaaaatgaagaaaaagaaccaaaatgactttattaaaataatttccaagattatttgtggctcacctgaaggctttgcaaaatttgtaccataaccgtttatttaacatatatttttatttttgattgcacttaaattttgtataatttgtgtttctttttctgttctacataaaatcagaaacttcaagctctctaaataaaatgaaggactatatctagtggtatttcacaatgaatatcatgaactctcaatgggtaggtttcatcctacccattgccactctgtttcctgagagatacctcacattccaatgccaaacatttctgcacagggaagctagaggtggatacacgtgttgcaagtataaaagcatcactgggatttaaggagaattgagagaatgtacccacaaatggcagcaataataaatggatcacacttaaaaaaaaaaaaaaa

SEQ ID NO: 93 - protein, 300 aa, Homo sapiens
  misc_feature: Incyte ID No 1635966CD1
MKFLLDILLLLPLLIVCSLESFVKLFIPKRRKSVTGEIVLITGAGHGIVRLTAYEFAKLKSKLVLWDINKHGLEETAAKCKGLGAKVHTFVVDCSNREDIYSSAKKVKAEIGDVSILVNNAGVVYTSDLFATQDPQIEKTFEVNVLAHFWTTKAFLPAMTKNNHGHIVTVASAAGHVSVPFLLAYCSSKFAAVGFHKTLTDELAALQITGVKTTCLCPNFVNTGFIKNPSTSLGPTLEPEEVVNRLMHGILTEQKMIFIPSSIAFLTTLERILPERFLAVLKRKISVKFDAVIGYKMKAQ

SEQ ID NO: 94 - DNA, 2361 bp, Homo sapiens
  misc_feature: Incyte ID No 2054053CB1
taggccccaaattctgccttttaaccccttaagtgatttggaagtattcactttaacattggttttaatcctacttttacttttttgaagtaccctatgatgagaggacctgatcctgctgctgctccatatagtactcagaaatcatctgttctacctctttatgaaaatacttttcaggagctccaggtaatgaggcggagcctgaatttgtttagaacacaaatgatggatttagaattggcaatgctgcgtcagcaaaccatggtttatcatcatatgactgaggaggagaggtttgaagttgatcagctccagggtttgagaaattcagtccgaatggaacttcaggacctggaactgcagctggaggagcgcctgctgggcctggaggagcagcttcgtgctgtgcgcatgccttcacccttccgctcctccgcactcatgggaatgtgtggcagtagaagcgctgataacttgtcatgcccttctccattgaatgtaatggaaccagtcactgaactgatgcaggagcagtcatacctgaagtctgaattgggcctgggacttggagaaatgggatttgaaattcctcctggagaaagctcagaatctgttttttcccaagcaacatcagaatcatcttctgtatgttctggtccctctcatgctaacagaagaactggagtaccttctactgcctcagtgggcaaatccaaaaccccattagtggcaaggaagaaagtgttccgagcatcggtggctctaacgccaacagctccttctagaacaggctctgtgcagacacctccagatttggaaagttctgaggaagttgatgcagctgaaggagccccagaagttgtaggacctaaatctgaagtggaagaagggcatggaaaactcccatcaatgccagctgctgaggaaatgcataaaaatgtggagcaagatgagttgcagcaagtcatacgggagattaaagagtctattgttggggaaatcagacgggaaattgtaagtggacttttggcagcagtatcttcaagtaaagcgtctaattctaagcaagattatcattaaacagaaattataggttggcatggatcctattagctgtgtaatactggaattatcaatgatatgcactggtggaggtgttatttgtgctttagaagatacttgctgttgagctgggctactgtatacagtgtacaatgtgtatttcttcaaccatatattttaaaaagacgtacatagaaacttaggcactttgctatttcttttctaaactatcaaaaactctagcagtttgaaaagcctaatatttatttgtatgtcagtatttttcatttgattccctattagaattaattttaaaacttgaagacttccagacttatccaacttataaataacatatttcttcagactaacatcttaaaacactgacctctatgaggtatttactgtgcaataactgattcatttttttcagagcttgaagcatccaatgatttttccctccactgctgttaattaatgtcacttccaagaagaaaaactgttctgttgtaaaaaatataattgctcttaattcttggggaggttactaatagcagtaggatagaattttatgaggttacctacaactacttaatgtacttacactgtaagccttgttgctttacccaagacaaatgtaattttatcattgcttatgtagtatttttcttttggaaatgtgccttatgttaaacactatgtacttttactttttgcattgtccagacttctttattagatggagatgtttctttttctgtcttctagactaaatagagtatcatccaaataatggggcctatgacttgaatgaatagaaatgaataagctggtgtttgttttttcaaaatggaagtaatttagatttgttctcctcatacataaaatgattttagttcagttttaaccagtgaaaactttgtttttatgaaaaaaaaggaaaatggtttcccatttggttttatatgtgttaaataaatgtgtaaagtaaccaccaaatgttattagaatttttcttctagcatttataattttttcaactcctattgtgtttctttgtgtgtgatattttaatcaaaagtggttgagttgttaacagtgttctttgaaagaatctctaaaaggcttataaatgtttgaaatatcacacaaaggctgatttctaaaatatatatatattaaaacaataaagtatttattttgcctaaagtgttttagtggtttcttaaactgcaacatgaagattttgaattagatttgataggtaacaaatgaggtccaaaagaattgcagcat

SEQ ID NO: 95 - protein, 247 aa, Homo sapiens [sequence omitted]

SEQ ID NO: 96 - DNA, 3098 bp, Homo sapiens
  misc_feature: Incyte ID No 096954.5
cctggaggccctgggggtggaggggcagccggcagcgggcacggtgcccgcccttgcccagcctggtatcctctttctccctcctcctcctctggactttgtttcctgatcccaggtggggctggggggagggggcacacctgcctcccctgggtggggcctctgttccctggcaacctggcgggcagggcggagctgggaggcctctgtgcccatcgaggagtcagagtggaggctgcagactgtggagccgggagccggcagtaagcccagaggtctccaccccacgggaggaaggctgaggccaagaccccggaagagatggaccgcgtgaccagataccccatcctgggcatccctcaggcacaccgtggcaccggcctggtgctggatggagacaccagctacacataccatctggtgtgcatgggccccgaggccagcggctggggccaggatgagccgcagacatggcccactgaccacagggcccagcagggcgtgcagaggcagggggtgtcctacagcgtgcatgcctacactggccagccgtccccacgggggctccactcggagaacagggaggatgagggttggcaggtttaccgcctgggcgccagggatgcccaccagggacgtccaacatgggcactccgcccagaggacggggaggacaaggagatgaagacctaccgcctggatgctggggacgctgaccccaggaggctgtgtgacctggagcgggagcgctgggccgtcatccagggccaggcagtcaggaagagcagcaccgtggccacgctccagggcactcctgaccacggagaccccaggacccccggcccacctcggtccacgcccctggaggagaacgtggttgacagggagcagattgacttcctggcagcgagacagcagttcctgagtctggagcaggcgaacaagggggcccctcatagctccccggccagggggacccctgcaggcacaaccccaggggccagccaggcccccaaggccttcaacaagccccacctggccaacgggcacgtggttcccatcaagccccaggtgaagggggtggtcagggaagagaacaaggtgcgtgctgtgcccacctgggccagtgtccaagttgtggatgaccctggctccttggcctcagtggagtccccggggacccccaaggagacgcccatcgagcgggagatccgtctggctcaggagcgtgaggcagacctgcgagagcagagggggcttcggcaggcaaccgaccaccaggagctggtggaaatccccaccaggccgctgctgaccaagctgagcctgatcacagccccacggcgggagagagggcgcccgtccctctacgtgcagcgggacatagtacaggagacacagcgtgaggaagaccaccggcgggagggcctgcacgtgggccgggcgtccacacccgactgggtctcggagggtccccagcccggactccggagagccctcagctcagattccatcctcagcccggccccagatgcccgtgcggccgacccagctccagaagtgaggaaggtgaaccgcatcccacctgatgcctaccagccgtacctgagccccgggaccccccagctagaattctcagccttcggagcattcggcaagcccagcagtctctccacagcggaggccaaggctgcgacttcaccaaaggccacgatgtccccgaggcatctctcagaatcctctggaaaacccctgagcacaaagcaagaggcatcgaagccccctcggggatgcccgcaagccaacaggggtgtcgtgcggtgggagtacttccgcctgcgtcctctgcggttcagggccccagacgagccccagcaggcccaagtcccccatgtctggggctgggaggtggctggggcccctgcactgaggctgcagaagtcccagtcatctgatctgctggaaagggagagggagagtgtcctgcgccgggagcaagaggtggcagaggagcggagaaatgctctcttcccagaggtcttctccccaacgccagatgagaactctgaccagaactccaggagctcctcccaggcatccggcatcacgggcagttactcggtgtctgagtctcccttcttcagccccatccacctacactcaaacgtggcgtggacagtggaagatccagtggacagtgctcctcccgggcagagaaagaaggagcaatggtacgctggcatcaacccctcggacggtatcaactcagaggtcctggaagccatacgggtgacccgtcacaagaacgccatggcagagcgctgggaatcccgcatctacgccagtgaggaggatgactgagcctcgggatggggcgcccaccccctgccctgccctgaccctcgtgggaactgccaagaccatcgccaagcccccaccctaggaaatgggtcctaggtccaggatccaagaaccacagctcatctgccaacaatcccaccatgggcacatttgggactgttgggtttttcgtttccgtttctatcttcctttagaaatgtttctgcctttggggtctaaagcttttggggatgaaatgggacccctgctgattctttctgcttctaagactttgccaaatgccctgggtctaagaaagaaagagacccgctcctccactttcaggtgtaatttgcttccgctagtctgagggcagagggaccggtcaaagagggtggcacagatcgcagcaccttgaggggctgcgggtctgagggaggagacactcagctcctccctctgagaagtcccaagctgagaggggagacctgcccctttccaaccctgggaaaccatccagtctgagggaggaggccaaactcccagtgctgggggtccctgtgcagccctcaaacccttcaccttggtgcacccagccacacctggtggacacaaagctctcacatcgataggatcccatgaggatggtccccttcacctgggagaaaagtgacccagtttaggagctggaggggggtctttgtcccccacccccaaactgccctgaaataaacctggagtgagctgccagaaaaaaa

SEQ ID NO: 97 - DNA, 860 bp, Homo sapiens
  misc_feature: Incyte ID No 1422432CB1
agagcaaagactggatgcatttcctgagaacaaccatcactgtaaagcactttacaaatccaaagacaacccccggcaaaaactcaaaatgaaactccctctcgcagagcacaattccaattcgctctaaaaacattacaagttagttcatgtcatgccagatagctgaaggcagctcacaagttcttaaggccaggaatgccangtgtctgctatgcacagctggccctggccctgagcctgaatgacagcaaaggtgacgcagatgtgggtgccctgctcctgcccagcagcagtgcttggtggaggctgaggccctgcacaggcaccctcactgctgaccttgagcctctctctcctctcaagaggctgccagtgggacattttctcggccctgccagcccccaggaggaaggtgggtctgaatctagcaccatgacggaactagagacagccatgggcatgatcatagacgtcttttcccgatattcgggcagcgagggcagcacgcagaccctgaccaagggggagctcaaggtgctgatggagaaggagctaccaggcttcctgcagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatgaaaaaaaaaa

SEQ ID NO: 98 - protein, 95 aa, Homo sapiens
  misc_feature: Incyte ID No 1422432CD1
MTELETAMGMIIDVFSRYSGSEGSTQTLTKGELKVLMEKELPGFLQSGKDKDAVDKLLKDLDANGDAQVDFSEFIVFVAAITSACHKYFEKAGLK

SEQ ID NO: 99 - DNA, 1498 bp, Homo sapiens
  misc_feature: Incyte ID No 409895.2
agctaatgtgttacattagaatcacctcggggaggccctgggtgcccttctcagccctccctccggaggctgctgaagcccagcaaagccggagtcagagaacaatgtccgcctgagggcagggctgggctgggctggccttctggccctatctgctccgtgcccaacccagcgccccgcacagtcggagctttgtaaatacgaggtgactgtctgcctacaaactttgtaaacatcacttgaaatggccgcagggtattgcgacatggccataccactatttgtttgctattgaatttgtacttccctgccttacttttgctattgcaaaccatgctgtcactaaggtcttcatgcacacagttgtgtcttggtcagatgatatgtttctaccaattttaattgtgtttctttccacctgggacacacagctctctgggccccagggctgggtcatcagcacaccctgctgctgctgttcagatctgcatcctggtcccgcttggtcccacagtgagaacgctttgctatcacatgggcaggctctgagagccctgccggcctggccttctcaaagaagacctgagagcttgggacccaagcagagaggaagaacagggctcagggtgcttgctccatgctcgctccacacctggggctcaaccctggctttccccggctccctgtgtgacttcagggcaggtcccttgggccctctgggccttatcatcttcatctgtaacagggcgatgcctctgccgtgtctggtggtgttgaggagttcctgtttgtgtaagcagctagttcagtgccagcacgagatgggaggcccatgaagttagcagtgcacaaaaaatagagcaaagactggatgcatttcctgagaacaaccatcactgtaaagcactttacaaatccaaagacaacccccggcaaaaactcaaaatgaaactccctctcgcagagcacaattccaattcgctctaaaaacattacaagttagttcatgtcatgccagatagctgaaggcagctcacaagttcttaaggccaggaatgccatgtgtctgctatgcacagctggccctggccctgagcctgaatgacagcaaaggtgacgcagatgtgggtgccctgctcctgcccagcagcagtgcttggtggaggctgaggccctgcacaggcaccctcactgctgaccttgagcctctctctcctctagagtggaaaagacaaggatgccgtggataaattgctcaaggacctggacgccaatggagatgcccaggtggacttcagtgagttcatcgtgttcgtggctgcaatcacgtctgcctgtcacaagtactttgagaaggcaggactcaaatgatgccctggagatgtcacagattcctggcagagccatggtcccaggcttcccaaaagtgtttgttggcaattattcccctaggctgagcctgctcatgtacctctgattaataaatgcttatgaaatg

SEQ ID NO: 100 - DNA, 1138 bp, Homo sapiens
  misc_feature: Incyte ID No 4874364CB1
gccagaaccaccaccgtttcccatggttactctacagcagcttcactgcctgaaaatgaaaactgaacaaagggattaaattgtgagaacaactgtcaatctattatatatttgtacaatctggtattaaaatgctttccccaaaagatattctgcgaaaagatctgaagttggtccatggttatcccatgacctgtgcttttgcgagcaactgggaaaaaattgaacagttccatagcagaccagatgacattgtgatagccacttatcctaaatcaggtactacttgggttagtgaaattatagacatgattctaaatgatggagatattgaaaaatgtaagcgaggttttattactgaaaaagttccaatgttggaaatgactctccctggattaagaacatcaggtatagaacaattggagaagaatccatcaccccggattgtgaaaacacatctaccgactgatcttcttcctaaatctttctgggaaaacaattgcaagatgatttatctggctcgtaatgccaaggatgtttcagtctcatattaccattttgacttaatgaataatttacagccttttcctggtacctgggaagaatatctggagaaattcttaactggaaaagtggcctatggttcctggtttactcatgttaaaaactggtggaagaaaaaggaaggacacccaatactttttttgtactatgaagatatgaaagagaatccaaaggaggaaatcaagaagatcattagatttctagagaagaacctgaatgatgagatcttggataggatcatccatcacacctcatttgaagtgatgaaggacaatcctttggtaaattatacacatctaccaactacagtgatggatcatagcaaatccccctttatgcgtaaagggacggctggtgactggaagaattacttcaccgtggcccaaaatgagaaatttgatgctatttatgagacagaaatgtccaaaactgcacttcaattccgcacagagatttaaagtgtctaaatcacaaatctgagaaatagagattgtctgtagttgattgaaacgagggcagttatgaattgatttgggcaatcaaatgaatttataaaggagaataatatgccttt

SEQ ID NO: 101 - protein, 296 aa, Homo sapiens
  misc_feature: Incyte ID No 4874364CD1
MLSPKDILRKDLKLVHGYPMTCAFASNWEKIEQFHSRPDDIVIATYPKSGTTWVSEIIDMILNDGDIEKCKRGFITEKVPMLEMTLPGLRTSGIEQLEKNPSPRIVKTHLPTDLLPKSFWENNCKMIYLARNAKDVSVSYYHFDLMNNLQPFPGTWEEYLEKFLTGKVAYGSWFTHVKNWWKKKEGHPILFLYYEDMKENPKEEIKKIIRFLEKNLNDEILDRIIHHTSFEVMKDNPLVNYTHLPTTVMDHSKSPFMRKGTAGDWKNYFTVAQNEKFDAIYETEMSKTALQFRTEI

SEQ ID NO: 102 - DNA, 507 bp, Homo sapiens
  misc_feature: Incyte ID No 239568.4
cagtcctcaggtgcaaccccctgcgtggtcctctgtggcagccttctctcattcagagctaaaaagaaaactcagtagaagataatggcaagtccagactggggatatgatgacaaaaatggtcctgaacaatggagcaagctgtatcccattgccaatggaaataaccagtcccctgttgatattaaaaccagtgaaaccaaacatgacacctctctgaaacctattagtgtctcctacaacccagccacagccaaagaaattatcaatgtggggcattccttccatgtaaattttgaggacaacgataaccgatcagtgctgaaaggtggtcctttctctgacagctacaggctctttcagttccattttcactggggcagtacaaatgagcatggttcagaacatacagtggatggagtcaaatattctgccgagcttcacgtagctcactggaattctgcaaagtactccagccttgctgaagctgcctcnaaggctgatggt

SEQ ID NO: 103 - DNA, 494 bp, Homo sapiens
  misc_feature: Incyte ID No 255041.1
acaacttgcatgtgattaggttcctgctggcctatgtgtcaactgtgatcttcatcatcacaagatgttgcctgtttcctttctgaaagtttgctaaaacaggaaagaaggagaaaagggattagttacataaaaggcttgaagctggaatgaccaaaagatagaattccttcagttaattccagcatgaagaggttgatacagaggatttccttcctcctgtgacaaaacatatttttataacttacactgttaggtcaaaatttattttccagagatttagtacctatttaaaagttagaaatattcttagtcaaatacttaaaaacaaggaanaataaaaaataatgtaaaaccatatgggtttatgtgaaatacattattctattcacaagtggcacatgcacaaaaattactgagctgtgtttcacactttatacattgacccaatattaagncatccgttcacaatattaataccattttgcaaatac

SEQ ID NO: 104 - DNA, 2147 bp, Homo sapiens
  misc_feature: Incyte ID No 2555628CB1
gtgtttaggagaaagtaggggctgtgggtgtcgggagccggctgacgggtggacaagggggggttagcagctgggctgcgaccgttagggaggggctcaaggtgtgcatgtgtgagggaagagagagagagagaagggcgcctcagaggtgactttcagcctgcgagccttcttcccggggcgccataaacgcccccaatttcccagctgctaaaggaagaggaagatcttagcaaagcaatgtctcaagatggtgcttctcagttccaagaagtcattcggcaagagctagaattatctgtgaagaaggaactagaaaaaatactcaccacagcatcatcacatgaatttgagcacaccaaaaaagacctggatggatttcggaagctatttcatagatttttgcaagaaaaggggccttctgtggattggggaaaaatccagagaccccctgaagattcgattcaaccctatgaaaagataaaggccaggggcttgcctgataatatatcttccgtgttgaacaaactagtggtggtgaaactcaatggtggtttgggaaccagcatgggctgcaaaggccctaaaagtctgattggtgtgaggaatgagaatacctttctggatctgactgttcagcaaattgaacatttgaataaaacctacaatacagatgttcctcttgttttaatgaactcttttaacacggatgaagataccaaaaaaatactacagaagtacaatcattgtcgtgtgaaaatctacactttcaatcaaagcaggtacccgaggattaataaagaatctttacttcctgtagcaaaggacgtgtcttactcaggggaaaatacagaagcttggtaccctccaggtcatggtgatatttacgccagtttctacaactctggattgcttgatacctttataggagaaggcaaagagtatatttttgtgtctaacatagataatctgggtgccacagtggatctgtatattcttaatcatctaatgaacccacccaatggaaaacgctgtgaatttgtcatggaagtcacaaataaaacacgtgcagatgtaaagggcgggacactcactcaatatgaaggcaaactgagactggtggaaattgctcaagtgccaaaagcacatgtagacgagttcaagtctgtatcaaagttcaaaatatttaatacaaacaacctatggatttctcttgcagcagttaaaagactgcaggagcaaaatgccattgacatggaaatcattgtgaatgcaaagactttggatggaggcctgaatgtcattcaattagaaactgcagtaggggctgccatcaaaagttttgagaattctctaggtattaatgtgccaaggagccgttttctgcctgtcaaaaccacatcagatctcttgctggtgatgtcaaacctctatagtcttaatgcaggatctctgacaatgagtgaaaagcgggaatttcctacagtgcccttggttaaattaggcagttcttttacgaaggttcaagattatctaagaagatttgaaagtataccagatatgcttgaattggatcacctcacagtttcaggagatgtgacatttggaaaaaatgtttcattaaagggaacggttatcatcattgcaaatcatggtgacagaattgatatcccacctggagcagtattagagaacaagattgtgtctggaaaccttcgcatcttggaccactgaaatgaaaaatactgtggacacttaaataatgggctagtttcttacaatgaaatgttctctaggattctaaaataggcaggtactttactatgttactgtaccctgcagtgttgatttttaaaatagagttttctgcagtatgcttttagtctaagaaaagcacagatggagcaatactttccttctttgaagagaatcccaaaagttagttcatcttaaagtgcaatattgtttaatcttaaaactgggcaactttggaagaacttttaacagaagcctcaatgatgatcactttgaattgcttgtgatttcaaaaataaagcagtgaagcaatacttgtgtacactggtactttataatgctaactataaactggtttattgttgttagacagttactatattagttggaag

SEQ ID NO: 105 - protein, 497 aa, Homo sapiens
  misc_feature: Incyte ID No 2555628CD1
MSQDGASQFQEVIRQELELSVKKELEKILTTASSHEFEHTKKDLDGFRKLFHRFLQEKGPSVDWGKIQRPPEDSIQPYEKIKARGLPDNISSVLNKLVVVKLNGGLGTSMGCKGPKSLIGVRNENTFLDLTVQQIEHLNKTYNTDVPLVLMNSFNTDEDTKKILQKYNHCRVKIYTFNQSRYPRINKESLLPVAKDVSYSGENTEAWYPPGHGDIYASFYNSGLLDTFIGEGKEYIFVSNIDNLGATVDLYILNHLMNPPNGKRCEFVMEVTNKTRADVKGGTLTQYEGKLRLVEIAQVPKAHVDEFKSVSKFKIFNTNNLWISLAAVKRLQEQNAIDMEIIVNAKTLDGGLNVIQLETAVGAAIKSFENSLGINVPRSRFLPVKTTSDLLLVMSNLYSLNAGSLTMSEKREFPTVPLVKLGSSFTKVQDYLRRFESIPDMLELDHLTVSGDVTFGKNVSLKGTVIIIANHGDRIDIPPGAVLENKIVSGNLRILDH

SEQ ID NO: 106 - DNA, 706 bp, Homo sapiens
  misc_feature: Incyte ID No 255803.1
caaaggccagagagtttccctggaggctgccatcgcagatgcctagcagtgtggggagttggctgttaaggatgccaatgccaagctctccgagctggaggctgccctgcagcgggccaagcaggacatggcgtanagctgcatgagtaccaggaatgacgaacatcaggctggccctggacatcaagatcaccatctacaggaagctgctggagggcgaggagagccagttggagtctgggatgcagaacatgagtatccatacgaagaccaccagcggctatgcaggtgatctgagctcggcctatgtgggcctcacaactcgtgacctcagctatggcctgggctccagctttggctctggcacaggcttcagctccttcaggtgcaccagctccaccagggccgtggttgtgaagaagatcgagacccgcgatgggaagctggtgtctgagtcctctgacttcctgcccaagtgaacaactgtggcagctcctcctagtctgcccctcccgtggctgccacagactccaggagggaggctgctgtgtggggaagcacagacaacaggagaccccccacctgaggctcaacccacccgtgggggagtttactgtctggggaccccccttgcccatgcttccagctagaaaacaattgtggtttttttttgggtccaaaataaaacctcagttagttccaccac

SEQ ID NO: 107 - DNA, 1589 bp, Homo sapiens
  misc_feature: Incyte ID No 900341CB1
gatagctgcctgatccaaggcccagcagcttctgccttccggatccccaccgacggacggaggcagcccacctgcaggaccggggcgctgtcggtccacgtggccgctctggagcaggttggagttggcagcgggatgcgctgcacggacaggcgcggcggacagccccgggggcagcggcggggtcctgtctgggagaggacagggttgcggcgggcggaacggtgtctccttcacttcgccctccagctgctggagctgcagcccgaccgcgagcgtgccaagcggcttcagcagctagcggagcggatggcggcggcccccctcaggacaccaccagattcccctcttcccgcggcctcgccatggcgacccacggacagacttgcgcgcgtccaatgtgtattcctccatcatatgctgaccttggcaaagctgccagagatattttcaacaaaggatttggttttgggttggtgaaactggatgtgaaaacaaagtcttgcagtggcgtggaattttcaacgtccggttcatctaatacagacactggtaaagttactgggaccttggagaccaaatacaagtggtgtgagtatggtctgactttcacagaaaagtggaacactgataacactctgggaacagaaatcgcaattgaagaccagatttgtcaaggtttgaaactgacatttgatactaccttctcaccaaacacaggaaagaaaagtggtaaaatcaagtcttcttacaagagggagtgtataaaccttggttgtgatgttgactttgattttgctggacctgcaatccatggttcagctgtctttggttatgagggctggcttgctggctaccagatgacctttgacagtgccaaatcaaagctgacaaggaataactttgcagtgggctacaggactggggacttccagctacacactaatgtcaatgatgggacagaatttggaggatcaatttatcagaaagtttgtgaagatcttgacacttcagtaaaccttgcttggacatcaggtaccaactgcactcgttttggcattgcagctaaatatcagttggatcccactgcttccatttctgcaaaagtcaacaactctagcttaattggagtaggctatactcagactctgaggcctggtgtgaagcttacactctctgctctggtagatgggaagagcattaatgctggaggccacaaggttgggctcgccctggagttggaggcttaatccagctgaaagaaacctttgggaatggatatcagaagatttggccttaatatatttccattgtgaccagcagcaggcttttttccccccagaagatgatcaaaacaaaggatgatctcaacaagagctgtattttaagtatttagacagttctttgttagctggtttctagttggttatctagttaccaatgctgcagtcctgcagtcacctatacattatttaaatgtatttaactgttaaatgcgctacccaccaataatgaaatagacctttatgaaaactgtgcaattgtgtgcatgtttgtttttatgttcctttagaaaacattgactgttt

SEQ ID NO: 108 - protein, 294 aa, Homo sapiens
  misc_feature: Incyte ID No 900341CD1
MATHGQTCARPMCIPPSYADLGKAARDIFNKGFGFGLVKLDVKTKSCSGVEFSTSGSSNTDTGKVTGTLETKYKWCEYGLTFTEKWNTDNTLGTEIAIEDQICQGLKLTFDTTFSPNTGKKSGKIKSSYKRECINLGCDVDFDFAGPAIHGSAVFGYEGWLAGYQMTFDSAKSKLTRNNFAVGYRTGDFQLHTNVNDGTEFGGSIYQKVCEDLDTSVNLAWTSGTNCTRFGIAAKYQLDPTASISAKVNNSSLIGVGYTQTLRPGVKLTLSALVDGKSINAGGHKVGLALELEA

SEQ ID NO: 109 - DNA, 1870 bp, Homo sapiens
  misc_feature: Incyte ID No 273879CB1
actcccctccgaggggtctgaccacgcttgggccgagtcatacgcccacgcgtccgggacctcctgccctcaggtgatccatccacctcggccagtcaaagtgctgggattacaggcatgagccattgcacccagccgatactactatatccccattttacagatgagcacatgggcaaattgagggtaaggcactgacccatgatcatacagctgagaagtggcaaaggcaggatttgaacctagaacctctggctccacacactagtaatctaaaccactctccctacaatacaacatacgtggtaaagatgtgtggtgggcacgcaatcaacgtaggtcccttcacagttgctgggagaggcaggaatttgcagttcctccgcgttctcctcctccgctgcccacctgtcctgggtcattcctgcagcctgccctgccctgcctggtctcaccctccctctgccaacagaagtctgggcagggttttatgggctctgataaggccctggcagggccgaagttcatgagcacttcctctttgcaggagggcgtaggggaggggacccaggtgatttgggtcctggctggtcaccagggaagctggcaagggaagggagactagggtgcgctctaggagaagccgacagcctgagagtcccagaagaggagccctgtggaccctcccctgccagccactcccttaccctgggtataagagccaccaccgcctgccatccgccaccatctcccactcctgcagctcttctcacaggaccagccactagcgcagcctcgagcgatggcctatgtccccgcaccgggctaccagcccacctacaacccgacgctgccttactaccagcccatcccgggcgggctcaacgtgggaatgtctgtttacatccaaggagtggccagcgagcacatgaagcggttcttcgtgaactttgtggttgggcaggatccgggctcagacgtcgccttccacttcaatccgcggtttgacggctgggacaaggtggtcttcaacacgttgcagggcgggaagtggggcagcgaggagaggaagaggagcatgcccttcaaaaagggtgccgcctttgagctggtcttcatagtcctggctgagcactacaaggtggtggtaaatggaaatcccttctatgagtacgggcaccggcttcccctacagatggtcacccacctgcaagtggatggggatctgcaacttcaatcaatcaacttcatcggaggccagcccctccggccccagggacccccgatgatgccaccttaccctggtcccggacattgccatcaacagctgaacagcctgcccaccatggaaggacccccaaccttcaacccgcctgtgccatatttcgggaggctgcaaggagggctcacagctcgaagaaccatcatcatcaagggctatgtgcctcccacaggcaagagctttgctatcaacttcaaggtgggctcctcaggggacatagctctgcacattaatccccgcatgggcaacggtaccgtggtccggaacagccttctgaatggctcgtggggatccgaggagaagaagatcacccacaacccatttggtcccggacagttctttgatctgtccattcgctgtggcttggatcgcttcaaggtttacgccaatggccagcacctctttgactttgcccatcgcctctcggccttccagagggtggacacattggaaatccagggtgatgtcaccttgtcctatgtccagatctaatctattcctggggccataactcatgggaaaacagaattatcccctaggactcctttctaagcccctaataaaatgtctgagggtgaaaaaaaaaaa

SEQ ID NO: 110 - protein, 323 aa, Homo sapiens
  misc_feature: Incyte ID No 273879CD1
MAYVPAPGYQPTYNPTLPYYQPIPGGLNVGMSVYIQGVASEHMKRFFVNFVVGQDPGSDVAFHFNPRFDGWDKVVFNTLQGGKWGSEERKRSMPFKKGAAFELVFIVLAEHYKVVVNGNPFYEYGHRLPLQMVTHLQVDGDLQLQSINFIGGQPLRPQGPPMMPPYPGPGHCHQQLNSLPTMEGPPTFNPPVPYFGRLQGGLTARRTIIIKGYVPPTGKSFAINFKVGSSGDIALHINPRMGNGTVVRNSLLNGSWGSEEKKITHNPFGPGQFFDLSIRCGLDRFKVYANGQHLFDFAHRLSAFQRVDTLEIQGDVTLSYVQI

SEQ ID NO: 111 - DNA, 1137 bp, Homo sapiens
  misc_feature: Incyte ID No 141804.1
atatggcggaggcttcttttggaagttcgagcccagttgggtctttgtcttctgaggatcatgattttgaccccactgctgagatgttggtccatgactatgatgatgaaagaactcttgaagaagaggaaatgatggatgagggtaaaaacttcagtttcagaaattgaagacttagaaaaggaaggaaccatgcctctagaagatttactggcattctatggctatgaacctacaattccagcagttgcaaattccagtgcaaatagttccccaagtgaactggcagatgaactaccagacatgacactagacaaagaggaaatagcaaaagacctgttgtcaggtgatgacgaggaaactcagtcttctgcggatgatctgacgccatctgtgacttcccatgaaacttctgatttcttccctaggcctttacgatcaaatactgcatgtgatggtgataaggaatcagaggttgaagatgttgaaacagacagtggtaattcacctgaagatttgaggaaggaaataatgattggtttacaatatcaggcagagattcccccttatcttggagagtacgatggtaatgagaaagtatatgaaaacgaagaccagttactttggtgtcctgatgtggttttggagagcaaagttaaggaataccttgttgagacttcattaaggactggcagtgaaaaaataatggataggatttctgcaggaacacacacaagggacaatgaacaggcattatatgaacttctcaagtgtaaccacaatataaaggaagcaatcgaaagatactgctgcaatggaaaggcctctcaagaaggaatgactgcatggacggaagaagaatgccgaagctttgaacatgcactcatgctttttggaaaagattttcatcttatacagaagaataaggtaaattaggcagattagtacagataaattactagttacagtgaatgtaagcaactaatttataagtcacttaagtaggaaaaaagaacaacttactaatgttcccttgccaatagcatntcagatatcattctaacatgcactaatttgctcagagatgccacagcctgcttatggaatggctgatagtatattcacatatcttttcac

SEQ ID NO: 112 - DNA, 1450 bp, Homo sapiens
  misc_feature: Incyte ID No 2512879CB1
gatgcacttgagcagggaagaaatccacaaggactcaccagtctcctggtctgcagagaagacagaatcaacatgagcacagcaggaaaagtaatcaaatgcaaagcagctgtgctatgggagttaaagaaacccttttccattgaggaggtggaggttgcacctcctaaggcccatgaagttcgtattaagatggtggctgtaggaatctgtggcacagatgaccacgtggttagtggtaccatggtgaccccacttcctgtgattttaggccatgaggcagccggcatcgtggagagtgttggagaaggggtgactacagtcaaaccaggtgataaagtcatcccactcgctattcctcagtgtggaaaatgcagaatttgtaaaaacccggagagcaactactgcttgaaaaacgatgtaagcaatcctcaggggaccctgcaggatggcaccagcaggttcacctgcaggaggaagcccatccaccacttccttggcatcagcaccttctcacagtacacagtggtggatgaaaatgcagtagccaaaattgatgcagcctcgcctctagagaaagtctgtctcattggctgtggattttcaactggttatgggtctgcagtcaatgttgccaaggtcaccccaggctctacctgtgctgtgtttggcctgggaggggtcggcctatctgctattatgggctgtaaagcagctggggcagccagaatcattgcggtggacatcaacaaggacaaatttgcaaaggccaaagagttgggtgccactgaatgcatcaaccctcaagactacaagaaacccatccaggaggtgctaaaggaaatgactgatggaggtgtggatttttcatttgaagtcatcggtcggcttgacaccatgatggcttccctgttatgttgtcatgaggcatgtggcacaagtgtcatcgtaggggtacctcctgattcccaaaacctctcaatgaaccctatgctgctactgactggacgtacctggaagggagctattcttggtggctttaaaagtaaagaatgtgtcccaaaacttgtggctgattttatggctaagaagttttcattggatgcattaataacccatgttttaccttttgaaaaaataaatgaaggatttgacctgcttcactctgggaaaagtatccgtaccattctgatgttttgagacaatacagatgttttcccttgtggcagtcttcagcctcctctaccctacatgatctggagcaacagctgggaaatatcattaattctgctcatcacagattttatcaataaattacatttgggggctttccaaagaaatggaaattgatgtaaaattatttttcaagcaaatgtttaaaatccaaatgagaactaaataaagtgttgaacatcagctggggaattgaagcctataaaccttccttcta

SEQ ID NO: 113 - protein, 375 aa, Homo sapiens
  misc_feature: Incyte ID No 2512879CD1
MSTAGKVIKCKAAVLWELKKPFSIEEVEVAPPKAHEVRIKMVAVGICGTDDHVVSGTMVTPLPVILGHEAAGIVESVGEGVTTVKPGDKVIPLAIPQCGKCRICKNPESNYCLKNDVSNPQGTLQDGTSRFTCRRKPIHHFLGISTFSQYTVVDENAVAKIDAASPLEKVCLIGCGFSTGYGSAVNVAKVTPGSTCAVFGLGGVGLSAIMGCKAAGAARIIAVDINKDKFAKAKELGATECINPQDYKKPIQEVLKEMTDGGVDFSFEVIGRLDTMMASLLCCHEACGTSVIVGVPPDSQNLSMNPMLLLTGRTWKGAILGGFKSKECVPKLVADFMAKKFSLDALITHVLPFEKINEGFDLLHSGKSIRTILMF

SEQ ID NO: 114 - DNA, 583 bp, Homo sapiens
  misc_feature: Incyte ID No 2685676CB1
ccctgtaggtgctgtccttgctgtcctgctctgtgacactctcctgggagtcacccgattggagggcgttatccaccttccactgtactttggcctctctgggatagaagttattcagcaggcacacaacagaggcagttccagatttcaactgctcatcagatggcgggaagatgaagacagatggtgcagccacagttcgtttgatctccagctcgagccgctgcgtgttttcctcttgatcgggaactcctgcttctccttgcctcgaaatggaccccaactgctcctgctcgcctgttggctcctgtgcctgtgccggctcctgcaaatgcaaagagtgcaaatgcacctcctgcaagaagagctgctgctcctgctgccctgtgggctgtgccaagtgtgcccagggctgcatctgcaaaggggcatcagagaagtgcagctgctgtgcctgatgtccggacagccctgctcgaagatatagaaagagtgacctgcacaaacttggaattttttttccatacaaccctgccccatctactgtattttttttaatgaaatatgtgaatgataatagtca

SEQ ID NO: 115 - protein, 61 aa, Homo sapiens
  misc_feature: Incyte ID No 2685676CD1
MDPNCSCSPVGSCACAGSCKCKECKCTSCKKSCCSCCPVGCAKCAQGCICKGASEKCSCCA

SEQ ID NO: 116 - DNA, 1759 bp, Homo sapiens
  misc_feature: Incyte ID No 2742913CB1
cacactgacgaggccatgattgaatttaggtgacctatagacgcgctgtaactacgctcggaattcggctcgaggtcacctcctccccttgtcgcctaggtccacccgagccccctcccccgggccgcccacgagcacgaagttggcgggagcctataaaagctggtgccggcgcgacccgcggacacacagtgcaggcgcccaagccgccgccgccagatcggtgccgattcctgccctgccccgaccgccagcgcgaccatgtcccatcactgggggtacggcaaacacaacggacctgagcactggcataaggacttccccattgccaagggagagcgccagtcccctgttgacatcgacactcatacagccaagtatgacccttccctgaagcccctgtctgtttcctatgatcaagcaacttccctgaggatcctcaacaatggtcatgctttcaacgtggagtttgatgactctcaggacaaagcagtgctcaagggaggacccctggatggcacttacagattgattcagtttcactttcactggggttcacttgatggacaaggttcagagcatactgtggataaaaagaaatatgctgcagaacttcacttggttcactggaacaccaaatatggggattttgggaaagctgtgcagcaacctgatggactggccgttctaggtatttttttgaaggttggcagcgctaaaccgggccttcagaaagttgttgatgtgctggattccattaaaacaaagggcaagagtgctgacttcactaacttcgatcctcgtggcctccttcctgaatccttggattactggacctacccaggctcactgaccacccctcctcttctggaatgtgtgacctggattgtgctcaaggaacccatcagcgtcagcagcgagcaggtgttgaaattccgtaaacttaacttcaatggggagggtgaacccgaagaactgatggtggacaactggcgcccagctcagccactgaagaacaggcaaatcaaagcttccttcaaataagatggtcccatagtctgtatccaaataatgaatcttcgggtgtttccctttagctaagcacagatctaccttggtgatttggaccctggttgctttgtgtctagttttctagacccttcatctcttacttgatagacttactaataaaatgtgaagactagaccaattgtcatgcttgacacaactgctgtggctggttggtgctttgtttatggtagtagtttttctgtaacacagaatataggataagaaataagaataaagtaccttgactttgttcacagcatgtagggtgatgagcactcacaattgttgactaaaatgctgcttttaaaacataggaaagtagaatggttgagtgcaaatccatagcacaagataaattgagctagttaaggcaaatcaggtaaaatagtcatgattctatgtaatgtaaaccagaaaaaataaatgttcatgatttcaagatgttatattaaagaaaaactttaaaaattattatatatttatagcaaagttatcttaaatatgaattctgttgtaatttaatgacttttgaattacagagatataaatgaagtattatctgtaaaaattgttataattagagttgtgatacagagtatatttccattcagacaatatatcataacttaataaatattgtattttagatatattctctaataaaattcagaattctaaaaaaaaaaaa

SEQ ID NO: 117 - protein, 260 aa, Homo sapiens
  misc_feature: Incyte ID No 2742913CD1
MSHHWGYGKHNGPEHWHKDFPIAKGERQSPVDIDTHTAKYDPSLKPLSVSYDQATSLRILNNGHAFNVEFDDSQDKAVLKGGPLDGTYRLIQFHFHWGSLDGQGSEHTVDKKKYAAELHLVHWNTKYGDFGKAVQQPDGLAVLGIFLKVGSAKPGLQKVVDVLDSIKTKGKSADFTNFDPRGLLPESLDYWTYPGSLTTPPLLECVTWIVLKEPISVSSEQVLKFRKLNFNGEGEPEELMVDNWRPAQPLKNRQIKASFK

SEQ ID NO: 118 - DNA, 508 bp, Homo sapiens
  misc_feature: Incyte ID No 429183.1
gccggctctcctcgccctctagcagcttcccgtaggtgggcgatctcgatctcgatgtccagggccagcttgacgttcatcagctcctagtactcacgcagctgccgcgccatgtcctgcttggccggctggagggcggcctccagctcggacagcttggcgttggcacccttaactgcccagctcctcaggctgctcggcatctgtgatggcggcctccagggaagccctctggcctttgaggcactcagtctcagcctggagnccactgatgttccagttcatctcgnaggtctcagtctttgtatgctgcacgtcatccccgtgcttnncagacagcgtctggagctcctcanacttgatctggtacatgctctcaacctcagcccagctgcggttagcgatctcctagtactgcgccttgagctcagcgatgactctatgtccagggagcggctgttgtccatggncagctccacagacgtgtccgagatctgggtctgcagct

SEQ ID NO: 119 - DNA, 442 bp, Homo sapiens
  misc_feature: Incyte ID No 2757583CB1
cctgactatcaaagcagcggccggctgttggggtccaccacgccttccacctgccccactgcttcttcgcttctctcttggaaagtccagtctctcctcggcttgcaatggaccccaactgctcctgcgccgctggtgtctcctgcacctgcgctggttcctgcaagtgcaaagagtgcaaatgcacctcctgcaagaagagctgctgctcctgctgccccgtgggctgtagcaagtgtgcccagggctgtgtttgcaaaggggcgtcagagaagtgcagctgctgcgactgatgccaggacaacctttctcccagatgtaaacagagagacatgtacaaacctggattttttttttataccaccttgacccatttgctacattccttttcctgtgaaatatgtgagtgataattaaacactttagacctgaaaaaaaaaaa

SEQ ID NO: 120 - protein, 61 aa, Homo sapiens
  misc_feature: Incyte ID No 2757583CD1
MDPNCSCAAGVSCTCAGSCKCKECKCTSCKKSCCSCCPVGCSKCAQGCVCKGASEKCSCCD

SEQ ID NO: 121 - DNA, 3686 bp, Homo sapiens
  misc_feature: Incyte ID No 1344279CB1
gaaaggaggagtccagtgctggaggggcagggacggcggcggcgcacgtcggaacccgccaggtccagggtccaggttccagcgcccggcggcccaggcaccccccgagcccagctccacacaccgttcctggatctcctctccccaggcggagcgtgcccctgcccagtccagtgaccttcgcctgttggagccctggttaatttttgcccagtctgcctgttgtggggctcctcccctttggggatataagcccggcctggggctgctccgttctctgcctggcctgaggctccctgagccgcctccccaccatcaccatggccaagggcttctatatttccaagtccctgggcatcctggggatcctcctgggcgtggcagccgtgtgcacaatcatcgcactgtcagtggtgtactcccaggagaagaacaagaacgccaacagctcccccgtggcctccaccaccccgtccgcctcagccaccaccaaccccgcctcggccaccaccttggaccaaagtaaagcgtggaatcgttaccgcctccccaacacgctgaaacccgattcctaccaggtgacgctgagaccgtacctcacccccaatgacaggggcctgtacgtttttaagggctccagcaccgtccgtttcacctgcaaggaggccactgacgtcatcatcatccacagcaagaagctcaactacaccctcagccaggggcacagggtggtcctgcgtggtgtgggaggctcccagccccccgacattgacaagactgagctggtggagcccaccgagtacctggtggtgcacctcaagggctccctggtgaaggacagccagtatgagatggacagcgagttcgagggggagttggcagatgacctggcgggcttctaccgcagcgagtacatggagggcaatgtcagaaaggtggtggccactacacagatgcaggctgcagatgcccggaagtccttcccatgcttcgatgagccggccatgaaggccgagttcaacatcacgcttatccaccccaaggacctgacagccctgtccaacatgcttcccaaaggtcccagcaccccacttccagaagaccccaactggaatgtcactgagttccacaccacgcccaagatgtccacgtacttgctggccttcattgtcagtgagttcgactacgtggagaagcaggcatccaatggtgtcttgatccggatctgggcccggcccagtgccattgcggcgggccacggcgattatgccctgaacgtgacaggccccatccttaacttctttgctggtcattatgacacaccctacccactcccaaaatcagaccagattggcctgccagacttcaacgccggcgccatggagaactggggactggtgacctaccgggagaactccctgctgttcgaccccctgtcctcctccagcagcaacaaggagcgggtggtcactgtgattgctcatgagctggcccaccagtggttcgggaacctggtgaccatagagtggtggaatgacctgtggctgaacgagggcttcgcctcctacgtggagtacctgggtgctgactatgcggagcccacctggaacttgaaagacctcatggtgctgaatgatgtgtaccgcgtgatggcagtggatgcactggcctcctcccacccgctgtccacacccgcctcggagatcaacacgccggcccagatcagtgagctgtttgacgccatctcctacagcaagggcgcctcagtcctcaggatgctctccagcttcctgtccgaggacgtattcaagcagggcctggcgtcctacctccacacctttgcctaccagaacaccatctacctgaacctgtgggaccacctgcaggaggctgtgaacaaccggtccatccaactccccaccaccgtgcgggacatcatgaaccgctggaccctgcagatgggcttcccggtcatcacggtggataccagcacggggaccctttcccaggagcacttcctccttgaccccgattccaatgttacccgcccctcagaattcaactacgtgtggattgtgcccatcacatccatcagagatggcagacagcagcaggactactggctgatagatgtaagagcccagaacgatctcttcagcacatcaggcaatgagtgggtcctgctgaacctcaatgtgacgggctattaccgggtgaactacgacgaagagaactggaggaagattcagactcagctgcagagagaccactcggccatccctgtcatcaatcgggcacagatcattaatgacgccttcaacctggccagtgcccataaggtccctgtcactctggcgctgaacaacaccctcttcctgattgaagagagacagtacatgccctgggaggccgccctgagcagcctgagctacttcaagctcaagctcatgtttgaccgctccgaggtctatggccccatgaagaactacctgaagaagcaggtcacacccctcttcattcacttcagaaataataccaacaactggagggagatcccagaaaacctgatggaccagtacagcgaggttaatgccatcagcaccgcctgctccaacggagttccagagtgtgaggagatggtctctggccttttcaagcagtggatggagaaccccaataataacccgatccaccccaacctgcggtccactgtctactgcaacgctatcgcccagggcggggaggaggagtgggacttcgcctgggagcagttccgaaatgccacactggtcaatgaggctgacaagctccgggcagccctggcctgcagcaaagagttgtggatcctgaacaggtacctgagctacaccctgaacccggacttaatccggaagcaggacgccacctctaccatcatcagcattaccaacaacgtcattgggcaaggtctggtctgggactttgtccagagcaactggaagaagctttttaacgattatggtggtggctcgttctccttctccaacctcatccaggcagtgacacgacgattctccaccgagtatgagctgcagcagctggagcagttcaagaaggacaacgaggaaacaggcttcggctcaggcacccgggccctggagcaagccctggagaagacgaaagccaacatcaagtgggtgaaggagaacaaggaggtggtgctccagtggttcacagaaaacagcaaatagtccccagcccttgaagtcacccggccccgatgcaaggtgcccacatgtgtccatcccagcggctggtgcagggcctccattcctggagcccgaggcaccagtgtcctcccctcaaggacaaagtctccagcccacgttctctctgcctgtgagccagtctagttcctgatgacccaggctgcctgagcacctcccagcccctgcccctcatgccaaccccgccctaggcctggcatggcacctgtcgcccagtgccctggggctgatctcagggaagcccagctccagggccagatgagcagaagctctcgatggacaatgaacggccttgctgggggccgccctgtaccctctttcacctttccctaaagaccctaaatctgaggaatcaacagggcagcagatctgtatatttttttctaagagaaaatgtaaataaatgatttctagatgaga

SEQ ID NO: 122 - protein, 969 aa, Homo sapiens
  misc_feature: Incyte ID No 1344279CD1
MAKGFYISKSLGILGILLGVAAVCTIIALSVVYSQEKNKNANSSPVASTTPSASATTNPASATTLDQSKAWNRYRLPNTLKPDSYQVTLRPYLTPNDRGLYVFKGSSTVRFTCKEATDVIIIHSKKLNYTLSQGHRVVLRGVGGSQPPDIDKTELVEPTEYLVVHLKGSLVKDSQYEMDSEFEGELADDLAGFYRSEYMEGNVRKVVATTQMQAADARKSFPCFDEPAMKAEFNITLIHPKDLTALSNMLPKGPSTPLPEDPNWNVTEFHTTPKMSTYLLAFIVSEFDYVEKQASNGVLIRIWARPSAIAAGHGDYALNVTGPILNFFAGHYDTPYPLPKSDQIGLPDFNAGAMENWGLVTYRENSLLFDPLSSSSSNKERVVTVIAHELAHQWFGNLVTIEWWNDLWLNEGFASYVEYLGADYAEPTWNLKDLMVLNDVYRVMAVDALASSHPLSTPASEINTPAQISELFDAISYSKGASVLRMLSSFLSEDVFKQGLASYLHTFAYQNTIYLNLWDHLQEAVNNRSIQLPTTVRDIMNRWTLQMGFPVITVDTSTGTLSQEHFLLDPDSNVTRPSEFNYVWIVPITSIRDGRQQQDYWLIDVRAQNDLFSTSGNEWVLLNLNVTGYYRVNYDEENWRKIQTQLQRDHSAIPVINRAQIINDAFNLASAHKVPVTLALNNTLFLIEERQYMPWEAALSSLSYFKLKLMFDRSEVYGPMKNYLKKQVTPLFIHFRNNTNNWREIPENLMDQYSEVNAISTACSNGVPECEEMVSGLFKQWMENPNNNPIHPNLRSTVYCNAIAQGGEEEWDFAWEQFRNATLVNEADKLRAALACSKELWILNRYLSYTLNPDLIRKQDATSTIISITNNVIGQGLVWDFVQSNWKKLFNDYGGGSFSFSNLIQAVTRRFSTEYELQQLEQFKKDNEETGFGSGTRALEQALEKTKANIKWVKENKEVVLQWFTENSK

SEQ ID NO: 123 - DNA, 836 bp, Homo sapiens
  misc_feature: Incyte ID No 1329472.2
ggagcccagctgtgctgtgggctcaggaggcagagctctgggaatctcaccatggcctggacccctctcctgctccccctcctcactttctgcacagtctctgaggcctcctatgagctgacacagccaccctcggtgtcagtgtccccaggacaaacggccaggatcacctgctctggagatacattgccaaaaaactctgcttattggtaccagcagaagtcaggccaggccccggtgctggtcatctatgaggacaccaaacgaccctccgagatccctgagagattctctggctccagctcagggacaatgccaccttgactatcagtggggcccagtggaggatgaagctgactactactgttactcaacagacagggtgttcggcggagggaccaaggtgaccgtcctaggtcagcccaaggctgccccctcggtcactctgttcccaccctcctctgaggagcttcaagccnacaaggccacactggtgtgtctcataagtgacttctacccgtgagccaccgcgcccagcccattgtattttcttaacagacagatattgtcttcttgactatcagtggggcccaggtggaggatgaagctgactactactgttactcaacagacagggtgttcggcggagggaccaaggtgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccac

SEQ ID NO: 124 - DNA, 684 bp, Homo sapiens
  misc_feature: Incyte ID No 474457.35
atttgttgcctgtgtcaaggggcagtgcttcagtcggggtggagctgcttaaaaggcctgggatcacaccctttgggaacacatccaagcttaagacggtgaggtcagcttcacattctcaggaactctccttctttgggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaagtgagaaatagagatgaagtgttgt

SEQ ID NO: 125 - DNA, 644 bp, Homo sapiens
  misc_feature: Incyte ID No 474457.45
taagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactctaaaaaaaaaaa

SEQ ID NO: 126 - DNA, 1115 bp, Homo sapiens
  misc_feature: Incyte ID No 898779CB1
ttagccaggtgtgttggcacattcctgtaatcccagctactcaggaggctgaaacaggagaatcgcttgaacccgggaggtggaggttgcagtgagccgagatcacaccactgcactccagcctgggcgacagagcaagacttcatctcaaaaaaaaaaaaaaagggcggcgctcctcgccagcagccgtccggagccagccaacgagcggaaaatggcagacaatttttcgctccatgatgcgttatctgggtctggaaacccaaaccctcaaggatggcctggcgcatgggggaaccagcctgctggggcagggggctacccaggggcttcctatcctggggcctaccccgggcaggcacccccaggggcttatcctggacaggcacctccaggcgcctaccatggagcacctggagcttatcccggagcacctgcacctggagtctacccagggccacccagcggccctggggcctacccatcttctggacagccaagtgcccccggagcctaccctgccactggcccctatggcgcccctgctgggccactgattgtgccttataacctgcctttgcctgggggagtggtgcctcgcatgctgataacaattctgggcacggtgaagcccaatgcaaacagaattgctttagatttccaaagagggaatgatgttgccttccactttaacccacgcttcaatgagaacaacaggagagtcattgtttgcaatacaaagctggataataactggggaagggaagaaagacagtcggttttcccatttgaaagtgggaaaccattcaaaatacaagtactggttgaacctgaccacttcaaggttgcagtgaatgatgctcacttgttgcagtacaatcatcgggttaaaaaactcaatgaaatcagcaaactgggaatttctggtgacatagacctcaccagtgcttcatataccatgatataatctgaaaggggcagattaaaaaaaaaaaaagaatctaaaccttacatgtgtaaaggtttcatgttcactgtgagtgaaaatttttacattcatcaatatccctcttgtaagtcatctacttaataaatattacagtgaaaaaaaaaaa

SEQ ID NO: 127 - protein, 250 aa, Homo sapiens
  misc_feature: Incyte ID No 898779CD1
MADNFSLHDALSGSGNPNPQGWPGAWGNQPAGAGGYPGASYPGAYPGQAPPGAYPGQAPPGAYHGAPGAYPGAPAPGVYPGPPSGPGAYPSSGQPSAPGAYPATGPYGAPAGPLIVPYNLPLPGGVVPRMLITILGTVKPNANRIALDFQRGNDVAFHFNPRFNENNRRVIVCNTKLDNNWGREERQSVFPFESGKPFKIQVLVEPDHFKVAVNDAHLLQYNHRVKKLNEISKLGISGDIDLTSASYTMI

SEQ ID NO: 128 - DNA, 2528 bp, Homo sapiens
  misc_feature: Incyte ID No 1843408CB1
ctatcgagccgattcggctcgaggggaacacaaacttgctggcgggaagagcccggaaagaaacctgtggatctcccttcgagatcatccaaagagaagaaaggtaagtagagctttgcatttacattttgaaaaattacaaagatgaactcatttttttcctgagagcaataactatttggcaatgcacagccctggcaaaaggccgtcgaatgtgtttgaaggtgtctgttgttgtttttgttcaggacgcttgcgttttctatttcaggtgacctcacattcgtgccccttagcagcactctgcagaaatgcctcctcagctgcaaaacggcctgaacctctcggccaaagttgtccagggaagcctggacagcctaccccaggcagtgagggagtttctcgagaataacgctgagctgtgtcagcctgatcacatccacatctgtgacggctctgaggaggagaatgggcggcttctgggccagatggaggaagagggcatcctcaggcggctgaagaagtatgacaactgctggttggctctcactgaccccagggatgtggccaggatcgaaagcaagacggttatcgtcacccaagagcaaagagacacagtgcccatccccaaaacaggcctcagccagctcggtcgctggatgtcagaggaggattttgagaaagcgttcaatgccaggttcccagggtgcatgaaaggtcgcaccatgtacgtcatcccattcagcatggggccgctgggctcacctctgtcgaagatcggcatcgagctgacggattcgccctacgtggtggccagcatgcggatcatgacgcggatgggcacgcccgtcctggaagcactgggcgatggggagtttgtcaaatgcctccattctgtggggtgccctctgcctttacaaaagcctttggtcaacaactggccctgcaacccggagctgacgctcatcgcccacctgcctgaccgcagagagatcatctcctttggcagtgggtacggcgggaactcgctgctcgggaagaagtgctttgctctcaggatggccagccggctggccaaggaggaagggtggctggcagagcacatgctgattctgggtataaccaaccctgagggtgagaagaagtacctggcggccgcatttcccagcgcctgcgggaagaccaacctggccatgatgaaccccagcctccccgggtggaaggttgagtgcgtcggggatgacattgcctggatgaagtttgacgcacaaggtcatttaagggccatcaacccagaaaatggctttttcggtgtcgctcctgggacttcagtgaagaccaaccccaatgccatcaagaccatccagaagaacacaatctttaccaatgtggccgagaccagcgacgggggcgtttactgggaaggcattgatgagccgctagcttcaggcgtcaccatcacgtcctggaagaataaggagtggagctcagaggatggggaaccttgtgcccaccccaactcgaggttctgcacccctgccagccagtgccccatcattgatgctgcctgggagtctccggaaggtgttcccattgaaggcattatctttggaggccgtagacctgctggtgtccctctagtctatgaagctctcagctggcaacatggagtctttgtgggggcggccatgagatcagaggccacagcggctgcagaacataaaggcaaaatcatcatgcatgacccctttgccatgcggcccttctttggctacaacttcggcaaatacctggcccactggcttagcatggcccagcacccagcagccaaactgcccaagatcttccatgtcaactggttccggaaggacaaggaaggcaaattcctctggccaggctttggagagaactccagggtgctggagtggatgttcaaccggatcgatggaaaagccagcaccaagctcacgcccataggctacatccccaaggaggatgccctgaacctgaaaggcctggggcacatcaacatgatggagcttttcagcatctccaaggaattctgggagaaggaggtggaagacatcgagaagtatctggaggatcaagtcaatgccgacctcccctgtgaaatcgagagagagatccttgccttgaagcaaagaataagccagatgtaatcagggcctgagaataagccagatgtaatcagggcctgagtgctttacctttaaaatcattaaattaaaatccataaggtgcagtaggagcaagagagggcaagtgttcccaaattgacgccaccataataatcatcaccacaccgtgagcagatctgaaaggcacactttgatttttttaaggataagaaccacagaacactgggtagtagctaatgaaattgagaagggaaatcttagcatgcctccaaaaattcacatccaatgcatagtttgttcaaatttaaggttactcaggcattgatcttttcagtgttttttcactttagctatgtggattagctagaa

SEQ ID NO: 129 - protein, 622 aa, Homo sapiens [sequence omitted]

SEQ ID NO: 130 - DNA, 757 bp, Homo sapiens
  misc_feature: Incyte ID No 351241.1
atgactcaaaccccttaaagaacacagaacaaaggcaccactcaccccatttgggggtgttctgttttctttgtggagtttcaagagtcatgggcagattcttcttaggtctaaagttctgctttcctgtattacattacctcacctctttggcttttgtgagtagcagagattaccttgtactgtgagaggattttaccttgatgtgtgtactggcggatgagagctacaaagttaaagctgactgaggacagtttacaggaagcagtcttcactgttttgttttttccacctaggaagttgtttaggatcctaattctaattcagaggtgcattctacagagtcttctccattgcctttcctctcaaaattaatcttgattggcttctctgggcatttgcgtgaggaactgaaactcattttcatagataaatgagagaatgagtttcctcagctccctaaagggcattttgcttctcccagctgaaaggctcccctggattactaggggctaagtgggagtgtctagtgggttgacccccagcaacgtgcagcagccctacagggaatccccaacaaaattagttttaaaaggcttgtccaagaaatgaatataggagctggtcattccatgctttgagccctcctggaggtgctagacctctggagacaaaaatgactcagtggataacacgctatggagtcctgccaataacccagcacacttcaacccatcccactaaaccctaggccttt

SEQ ID NO: 131 - DNA, 3036 bp, Homo sapiens
  misc_feature: Incyte ID No 413348.40
ggtaaacaacaggactataaatatcagagtgtgctgctgtggctttgtggagctgccagagtaaagcaaagagaaaggaagcaggcccgttggaagtggttgtgacaaccccagcaatgtggagaagcctggggcttgccctggctctctgtctcctcccatcgggaggaacagagagccaggaccaaagctccttatgtaagcaacccccagcctggagcataagagatcaagatccaatgctaaactccaatggttcagtgactgtggttgctcttcttcaagccagctgatacctgtgcatactgcaggcatctaaattagaagacctgcgagtaaaactgaagaaagaaggatattctaatatttcttatattgttgttaatcatcaaggaatctcttctcgattaaaatacacacatcttaagaataaggtttcagagcatattcctgtttatcaacaagaagaaaaccaaacagatgtctggactcttttaaatggaagcaaagatgacttcctcatatatgatagatgtggccgtcttgtatatcatcttggtttgcctttttccttcctaactttcccatatgtagaagaagccattaagattgcttactgtgaaaagaaatgtggaaactgctctctcacgactctcaaagatgaagacttttgtaaacgtgtatctttggctactgtggataaaacagttgaaactccatcgcctcattaccatcatgagcatcatcacaatcatggacatcagcaccttggcagcagtgagctttcagagaatcagcaaccaggagcaccaaatgctcctactcatcctgctcctccaggccttcatcaccaccataagcacaagggtcagcataggcagggtcacccagagaaccgagatatgccagcaagtgaagatttacaagatttacaaaagaagctctgtcgaaagagatgtataaatcaattactctgtaaattgcccacagattcagagttggctcctaggagctgatgctgccattgtcgacatctgatatttgaaaaaacagggtctgcaatcacctgatagtgtaaagaaaacctcccatctttatgtagctgacagggacttcgggcagaggagaacataactgaatcttgtcagtgacgtttgcctccagctgcctgacaaataagtcagcagcttatacccacagaagccagtgccagttgacgctgaaagaatcaggcaaaaaagtgagaatgaccttcaaactaaatatttaaaataggacatactccccaatttagtctagacacaatttcatttccagcatttttataaactaccaaattagtgaaccaaaaatagaaatcagatttgtgcaaacatggagaaatctactgaattggcttccagattttaaattttatgtcatagaaatattgactcaaaccatattttttatgatggagcaactgaaaggtgattgcagcttttggttaatatgtctttttttttctttttccagtgttctatttgctttaatgagaatagaaacgtaaactatgacctaggggtttctgttggataattagcagtttagaatggaggaagaacaacaaagacatgctttccatttttttctttacttatctctcaaaacaatattactttgtcttttcaatcttctacttttaactaataaaataagtggattttgtattttaagatccagaaatattcacgtgttaagtatttcctggatcttaaaatacttaacacgtgaatattttgctaaaaaagcatatataactattttaaatatccatttatcttttgtatatctaagactcatcctgatttttactatcacacatgaataaagcctttgtatctttctttctctaatgttgtatcatactcttctaaaacttgagtggctgtcttaaaagatataaggggaaagataatattgtctgtctctatattgcttagtaagtatttccatagtcaatgatggtttaataggtaaaccaaaccctataaacctgacctcctttatggttaatactatttaagcaagaatgcagtacagaattggatacagtacggatttgtccaaataaattcaataaaaaccttaaagctgacttcgtttgttatgtaggctgtatgcatatattgaaaacagaagtgaaactttcgattgcttttaaataaataccaactaatgaatttactgctaagctcaaaaccgtctacgcttaaggtgagaatctttgccaacagaaaggcgttgctctaaatcgcgaatggtattacgaagaattgcaatctccttgttttttttcttcttgaaaatgctgaagtttccttctgtagatactttgtggcaaaacagtagaatcctgatatgattttgatttagtctgaactcttgctagtttggcatcaaactgaatcaaattgaaaagaaagttattgaaactctagacaattagaaacaattgtttctataattataatccatgttatcctttgacatgtaatctgtaattcaggtgtaaattacagtatagacaatagaaaaatctttctttaagaaataatttttcagaaagagcaaagaacagaaaaacaagattaaccctgattgcacggtaaaattacctggagaggttttttttttctttgtttctaaagtaccaaatgcctgggcactactttagagattctggtgtgcagctagcgtcatctttttctcaaaacatcacgtgattctatagtgcagacagggtttagaatctagtttaacttaaatataggtaaaaatatttttacgttaaaatgagtgtattataaagaaaatgtaataaacagtcaaggaagaaaacctcacgtagctttctgcaggaactttgaggctgaacagtagaatttgaatacgatcttgtcgatatagtttgaacccttgctggcttcaattaaactgaatcaggttaaaaaagttctcataggccattcttaacaggccatttttttttgtttt

SEQ ID NO: 132 - DNA, 4440 bp, Homo sapiens
  misc_feature: Incyte ID No 983354.2
tctgtggtgccttgggtcaacgaccaggatgtccctttctgtccagactgtgggaataagttcagcatccggaaccgccgccaccactgccgcctctgcgggtctattatgtgcaagaagtgtatggagctcatcagccttcccttggcaaaaattacgactttgcatggagaaagttgaccagaaagctccagaatacatcaggatggcagcatcattaaatgctggggagacaacctacagtctggaacatgccagtgaccttcgagtggaagtgcagaaagtgtatgagctgatagacgctttaagtaagaagatcttaaccttgggcttgaaccaggaccctccaccacatccaagcaatttgcggctgcagagaatgatcagatactcagctacactttttttcagttcctcaaactgttctttgatgtcactgccaaccaaagaacagtttgaggaactgaaaaagaaaaggaaggaggaaatggagaggaagagggccgtggagagacaagctgccctggagtcccagcgaaggcttgaggaaaggcagagtggcctggcttctcgagcggccaacggggaggtggcatctctccgcaggggccctgcccccttgagaaaggctgagggctggctcccactgtcaggaggtcaggggcagagtgaggactcagacccgctcctccagcagatccacaacatcacatcattcatcaggcaggccaaggccgcgggccgcatggatgaagtgcgcactctgcaggagaacctgcggcagctgcaggacgagtatgaccagcagcagacagagaaggccatcgagctgtcccggaggcaggctgaggaggaggacctgcagcgggaacagctgcagatgttgcgtgaacgggagttggaacgagaaagggagcagtttcgggtggcatccctgcacacacggactcggtccctggacttcagagaaatcggcccttttcagctggagcccagcagagagcctcgcacccaccttgcttatgctttggatctaggctcttccccagttccaagcagcacagctcccaagaccccttcacttagctcaactcaacccaccagagtgtggtctgggcccccagccgttggccaggagcgcttaccccagagcagcatgccacagcaacatgaggggccctccttaaacccctttgatgaggaagacctctccagccccatggaagaggccactactggtcctcctgctgcaggggtttccttagacccttcagcccgcatcctgaaagagtacaatcctttcgaggaagaggacgaagaggaggaagcagtggcagggaatccattcattcagccagacagcccagctcctaaccccttcagtgaggaagacgaacatccccagcagaggctctcaagccctctggttcctggtaacccctttgaggaacccacctgtatcaacccctttgagatggacagtgacagtgggccagaggctgaggagcccatagaggaagagctcctcctgcagcagatcgataacatcaaggcatacatctttgatgccaagcagtgcggccgcctggatgaggtagaggtgctgacagagaatctgcgggagctgaagcacaccctggccaagcagaaggggggcactgactgaccagcagtggagagggcacctttgggcccaggggtctggcaggagccagtggagcaggacagagggcaggcaggatggatggggaaggtggcagggtgagaactcagatgcacacaggtgaggggcaggaatctgctgttttgtgttgcgcactttgaggtatttccactacagttgaataataaaatagaaactagaacagggagaatcagcattcagttgctgcttttcctgtttattattactatcttttgtaatcggaggtttaccccttttgaagggactttacatttttactaccgagatataactaaatgcagctctgttgggcccagggcagaaatggctgctgtgtacctcttgggtccatttgctactgcctagtcttggttccttatgcagtattatagggcagcctttttagagcccttcctttagccaagacagagaagatagattccactgagctctattctgctctgacagaagtccatccctagtaggctgtgagttccatttcacctggggccgcctctcccctgctctgcacttcctgtctgtacaatagaagggggaggtgctgctatgaaggggagagtttagacccaggagagcccagcacctctttttaaggtggggtgatgggaatatttcaccagggtctattttctcagtttaagttcttttttgtctctttcaggaagttaagctcccaaagtgctgggaatacaggcgtgagccactgtgcccgccctgatgagatattttattaccaatgttagtattgagaaactgaaatgtttgaagaagcacaacccaggatcgtgctggtagcaccacagtacttaaactattggtcaattaaggccagaaagggaaattgttaatttagctctggtgctttggtttacaggaacataactcttaactgacatctgacatcatgatagccatatgtgctcagctctgggtagagtttctgcagttactcatctgaactaatgaacaataactgaccactagtcactttatgccgtgtaactagctctaggccatactttcactggttactggtgtgaaagctgaaattcattttgttacattctggtgaagacccctcttgataatgggaatgttttaactctcttgatgaaaaaataatctgtatttgtgttgatgttcacatttctgtagcacatttcttatccttttggttgaatgaaaagatcttgtataggggtgtggagatggggagtgggtagaagtgtgtgaaggacgctttgcatttgggatctgttcacaaacagccatatgagtgtgttaatgaatgtcagccagttaccaaccctgctggttgttatgggttgttttgagaagttggcaaccaggcatctaagatgttgcctggtacaggcctcttttcttccctgaggcccatgacatttctctgttactctagagggtttctcagatggccagtaggctcctccctgcttagggtctcatttctctgaaaagaggatgaactgaaaagcaggtagttccagaagctaattgctgctttccatcataattatttttcttgtgagaacatttctcttttaattagctagtgattttgattaagactaattcactaaacatacccttccctcaaatcacctcaggtagcaatctgtaagtaactaaaagcattgaaaaacacacaagaaacatttttaaaaactatttttaaaggcctggccgggtgcagtggctcactcctgtaatcccagcactttgggaggccgagacgggtggatcacctgaggtcaggaatttgagactagcatggccaacatggtgaaaccccgtgtctactaaaaatacaaaaattagctgggcgtggtggcagacgcctgtaatcccagctactcagggaggctgaggcacgagaatcgcttgaacctgggaggcggaggttgcagtgagccaagattgcgccactgcactccaacctgggtgacaagagcaagactccgtctcacaaaaaacaaacaaaactgactgattgaatatactaaaccaaactaaaatcatattcttttgattaagtttatccatggctgtattcttctatgaattcttccatttaattcttccacgattatcttctcctgtaaatacatcacaggagttagaattctctacccatcagctgtaccatgtcgcagaattcatgcaggcacaaagttggagttacagagatgggttgacagcaggcaaacttggcctatgtattataaccacaacttcaagttcttacctcatgtgaatattcaccctttctttagtcttccaaggcaaacagccccgtctcatcaccagatgagcaaggtcttgatatggcatagcagatctccctagacacagatcatgagaaaagatggaagagacttagggattcaggcatcagatgaagttggcttttcccttttatgccttgtttgtatttaccctgtctaatacactaaggatacttactcattgtacttgcagctcaatatgtctttgctgttcagatactaaaatgtacctctgagtcattgtgagctgtgtggtaggttggacattggcatagttggtgatgggactcaaaatgaaaaggtggtctctttaccaggtcacagactgtagcagattgtgcttgttatctgacaatgactgtcactttgagggtcgttgatttgcatgcactactctggggccttgtattggagccttttttaaaaaaa

SEQ ID NO: 133 - DNA, 1456 bp, Homo sapiens
  misc_feature: Incyte ID No 235845.20
gccaggtggaggcctgctgaggcagtggttgtggggatcggtctccaggcagcagggggcagcagggtcaaggagaggctaactggccacgggtggggccagcaggcgggcagaaggaggctttaaagcgcctaccctgcctgcaggtgagcagtggtgtgtgagagccaggcgtccctctgcctgcccactcagtggcaacacccgggagctgttttgtcctttgtggagcctcagcagttccctctttcagaactcactgccaagagccctgaacaggagccaccatgcagtgcttcagcttcattaagaccatgatgatcctcttcaatttgctcatctttctgtgtggtgcagccctgttggcagtgggcatctgggtgtcaatcgatggggcatcctttctgaagatcttcgggccactgtcgtccagtgccatgcagtttgtcaacgtgggctacttcctcatcgcagccggcgttgtggtctttgctcttggtttcctgggctgctatggtgctaagactgagagcaagtgtgccctcgtgacgttcttcttcatcctcctcctcatcttcattgctgaggttgcagctgctgtggtcgccttggtgtacaccacaatggctgagcacttcctgacgttgctggtagtgcctgccatcaagaaagattatggttcccaggaagacttcactcaagtgtggaacaccaccatgaaagggctcaagtgctgtggcttcaccaactatacggattttgaggactcaccctacttcaaagagaacagtgcctttcccccattctgttgcaatgacaacgtcaccaacacagccaatgaaacctgcaccaagcaaaaggctcacgaccaaaaagtagagggttgcttcaatcagcttttgtatgacatccgaactaatgcagtcaccgtgggtggtgtggcagctggaattgggggcctcgagctggctgccatgattgtgtccatgtatctgtactgcaatctacaataagtccacttctgcctctgccactactgctgccacatgggaactgtgaagaggcaccctggcaagcagcagtgattgggggaggggacaggatctaacaatgtcacttgggccagaatggacctgccctttctgctccagacttggggctagatagggaccactccttttaggcgatgcctgactttccttccattggtgggtggatgggtggggggcattccagagcctctaaggtagccagttctgttgcccattcccccagtctattaaacccttgatatgccccctaggcctagtggtgatcccagtgctctactgggggatgagagaaaggcattttatagcctgggcataagtgaaatcagcagagcctctgggtggatgtgtagaaggcacttcaaaatgcataaacctgttacaatgttaaaaaaaaaacaaaaagg

SEQ ID NO: 134 - DNA, 1398 bp, Homo sapiens
  misc_feature: Incyte ID No 266360.18
ggggcggagaggcctggcgcacagggcgagggcggctgcggcgcagtctggcagcatggcgtacccggggcatcctggcgccggcggcgggtactacccaggcgggtatggaggggctcccggagggcctgcgtttcccggacaaactcaggatccgctgtatggttactttgctgctgtagctggacaggatgggcagatagatgctgatgaattgcagagatgtctgacacagtctggcattgctggaggatacaaaccttttaacctggagacttgccggcttatggtttcaatgctggatagagatatgtctggcacaatgggtttcaatgaatttaaagaactctgggctgtactgaatggctggagacaacactttatcagttttgacactgacaggagtggaacagtagacccacaagaattgcagaaggccctgacaacaatgggatttaggttgagtccccaggctgtgaattcaattgcaaaacgatacagcaccaatggaaagatcaccttcgacgactacatcgcctgctgcgtcaaactgagggctcttacagacagctttcgaagacgggatactgctcagcaaggtgttgtgaatttcccatatgatgatttcattcaatgtgtcatgagtgtttaaatcaagaggaagctgcatgaatgtaatcaacattccaactggagctctcctttgcttgtcctctttgccttcggtaatatgtataaacttacatcacgactttctcttaacagctgttgtaaagtttattactttatgtacaactgaagttttgttttagttttgataataaattctttggaactttaataagatctagtctgttacaccatttagaactttcctgagccattatcagtcatgccttattttcttgctaaaactctatgtaaatttaagtatgcaaaatgtttaagtcacattatttatttttcattgtgagacactaaaaactgttaatcagactacagctgttatctttcctctcctacaaagaatactccacacataaaaacttaggtaaatgacatagacgcacttgggtgaaataaaacaacaaaaaaggtaatccagtaatccacgtcaggattcaccttagaagtttagcacacgcccttcaaaacctgttgaataatttgattggcaaatactatctgtcaccaagtccctttttgtcatctatttaaacctttgttaactctccttaaaaatcttgtacattataagcttaactatataaaaagaaaattgatagaataaggctaagggggtatatggatatattaacgatgtttagtttggatgagtgagatctagatgacttatgattgcttagatcagtggtgtccgagttccataggcttgcc

SEQ ID NO: 135 - DNA, 694 bp, Homo sapiens
  misc_feature: Incyte ID No 266360.15
cggcgcagctctgcagcatggcgtaacccggggcatcctggcgccggcggcgggtaactacccaggcggggtaagtngctgactgcgcgggatcgcggcccangcggctccggggcgctaagaggccaccttggagatgctgcggccagcgggtatggaggggctcccggagggcctgcgtttcccggacaaactcaggatccgctgtatggttactttgctgctgtagctggacaggatgggcagatagatgctgatgaattgcagagatgtctgacacagtctggcattgctggaggatacaaacgatttaggttgagtccccaggctgtgaattcaattgcaaaacgatacagcaccaatggaaagatcaccttcgacgactacatcgcctgctgcgtcaaactgagggctcttacagacagctttcgaagacgggatactgctcagcaaggtgttgtgaatttcccatatgatgatttcattcaatgtgtcatgagtgtttaaatcaagaggaagctgcatgaatgtaatcaacattccaactggagctctcctttgcttgtcctctttgccttcggtaatatgtataaacttacatcacgactttctcttaacagctgttgtaaagtttattactttatgtacaactgaagtttggttttagttttgataataaattctttggaacttt

SEQ ID NO: 136 - DNA, 406 bp, Homo sapiens
  misc_feature: Incyte ID No 1310030.1
gagaagacaggactcaggacaatctccagcatggcctggtcccctctcttcctcaccctcatcactcactgtgcagggtcctgggcccagtctgtgctgactcagccaccctcggtgtctgggacccccggacagagggtcaccatctcctgttctggcagcagctccaacatcggaagtctactgtgactggtatcaacaattcccaggaatggcccccagacttctcatttatagggattatcagcggccctcaggggtccctgaccgattctactggctccaggtctggcaccgcagcctccctggccatcagtggtctccagcctgaagatgaggctgattactattgtgcacctgggataccagcctgggtgttcatgtgctgttcggcggagggaccaaa

SEQ ID NO: 137 - DNA, 1380 bp, Homo sapiens
  misc_feature: Incyte ID No 2804864CB1
cgcacagggcgagggcggctgcggcgcagtctgcagcatggcgtacccggggcatcctggcgccggcggcgggtactacccaggcgggtatggaggggctcccggagggcctgcgtttcccggacaaactcaggatccgctgtatggttactttgctgctgtagctggacaggatgggcagatagatgctgatgaattgcagagatgtctgacacagtctggcattgctggaggatacaaaccttttaacctggagacttgccggcttatggtttcaatgctggatagagatatgtctggcacaatgggtttcaatgaatttaaagaactctgggctgtactgaatggctggagacaacactttatcagttttgacactgacaggagtggaacagtagacccacaagaattgcagaaggccctgacaacaatgggatttaggttgagtccccaggctgtgaattcaattgcaaaacgatacagcaccaatggaaagatcaccttcgacgactacatcgcctgctgcgtcaaactgagggctcttacagacagctttcgaagacgggatactgctcagcaaggtgttgtgaatttcccatatgatgatttcattcaatgtgtcatgagtgtttaaatcaagaggaagctgcatgaatgtaatcaacattccaactggagctctcctttgcttgtcctctttgccttcggtaatatgtataaacttacatcacgactttctcttaacagctgttgtaaagtttattactttatgtacaactgaagttttgttttagttttgataataaattctttggaactttaataagatctagtctgttacaccatttagaactttcctgagccattatcagtcatgccttattttcttgctaaaactctatgtaaatttaagtatgcaaaatgtttaagtcacattatttatttttcattgtgagacactaaaaactgttaatcagactacagctgttatctttcctctcctacaaagaatactccacacataaaaacttaggtaaatgacatagacgcacttgggtgaaataaaacaacaaaaaaggtaatccagtaatccacgtcaggattcaccttagaagtttagcacacgcccttcaaaacctgttgaataatttgattggcaaatactatctgtcaccaagtccctttttgtcatctatttaaacctttgttaactctccttaaaaatcttgtacattataagcttaactatataaaaagaaaattgatagaataaggctaagggggtatatggatatattaacgatgtttagtttggatgagtgagatctagatgacttatgattgcttagatcagtggtgtccgagttccataggcttgcc

SEQ ID NO: 138 - protein, 198 aa, Homo sapiens
  misc_feature: Incyte ID No 2804864CD1
MAYPGHPGAGGGYYPGGYGGAPGGPAFPGQTQDPLYGYFAAVAGQDGQIDADELQRCLTQSGIAGGYKPFNLETCRLMVSMLDRDMSGTMGFNEFKELWAVLNGWRQHFISFDTDRSGTVDPQELQKALTTMGFRLSPQAVNSIAKRYSTNGKITFDDYIACCVKLRALTDSFRRRDTAQQGVVNFPYDDFIQCVMSV

SEQ ID NO: 139 - DNA, 1527 bp, Homo sapiens
  misc_feature: Incyte ID No 349615.7
gcaggggttcgtttgtgatatggtagtacgatgcctgtaattttgatattttaaattattttatatcaaaataatttcttttaaaatttatttttaaaatgactgcctttaaaaatattcatgctcctaccctcacccccttgcaggaacttgcatcaacaatacttcagtcatgatgtttaaaaaggggagctttgaaattggaggaaccatacatccagttgcaattaagtataaccctcagttcggtgatgcattttggaacagtagtaaatacaacatggtgagctacctgcttcgaatgatgaccagctgggccatcgtctgtgacgtgtggtacatgccccccatgaccagagaggaaggagaagatgcagtccagtttgctaacagggttaagtctgctattgctatacaaggaggcctgactgaacttccctgggatggaggactaaagagagcaaaggtgaaggacatctttaaggaagagcagcagaaaaattacagcaagatgattgtgggcaatggatctctcagctaagaggacggatgacagcctttagatctagaactagcccttagaaatggaatggcttttttgttttgttttgttttattgttttgtttttattattgttaatcttttctacagaatgattgtctctacctctttatgccagaggcagaacctacaggtgccctttttggcttttgttgttgttgtaacattagccccatggattgtaaggtggtttactgagttaaaacagattctgcttttgtaaaatgatggcatcactgtggactgaatgaaatatttgtatagaaaaaagtgcttgaaaagtgtgtttggaactcatcgatagggtaattctccaaaaatgcccaaactctctttctgtaattagccttgccactttcttcagtcacttaaatggtgagattacacatcagtgcaagatgaccattatggttatggtctactgcaaggttgaaaggaaaaatggaggattgtatttaggaaaagggacaactttgtggccacctgctctgaaagtcaaaaggaaatgtaaattagtgtcattagtgtgttggaagagaaatactattcagtaagcttcgccaaagaaaagtgagtcaaagttaatgtgtgtgtgcatttatatgtaggcagctcgtagaccacattttagccagcaactggtaacaaagagcttagttttccttgtttgaatgctgtagatctgtacctagtacccctcccatctactgatttgtttgtttttgtaaccaaacacattttcagatagaaggagccttaaaaaaaaaaaaatcacattgagtaacttcagtatgaatgaatgagagtgtgtggagctacccctcaccctccacccctttgggctttttattcccgaattttcccagtctcttaaacagaaaaatgactgatataattatcttttggaaactgagccttaattttttttagagggggaaataagtttccccaactcacacagcata

SEQ ID NO: 140 - DNA, 1114 bp, Homo sapiens
  misc_feature: Incyte ID No 632664CB1
gccgcctctgccgccgcggacttcccgaacctcttcagccgcccggagccgctcccggagcccggccgtagaggctgcaatcgcagccggtgagcccgcagcccgcgccccgagcccgccgccgcccttcgagggcgccccaggccgcgccatggtgaaggtgacgttcaactccgctctggcccagaaggaggccaagaaggacgagcccaagagcggcgaggaggcgctcatcatcccccccgacgccgtcgcggtggactgcaaggacccagatgatgtggtaccagttggccaaagaagagcctggtgttggtgcatgtgctttggactagcatttatgcttgcaggtgttattctaggaggagcatacttgtacaaatattttgcacttcaaccagatgacgtgtactactgtggaataaagtacatcaaagatgatgtcatcttaaatgagccctctgcagatgccccagctgctctctaccagacaattgaagaaaatattaaaatctttgaagaagaagaagttgaatttatcagtgtgcctgtcccagagtttgcagatagtgatcctgccaacattgttcatgactttaacaagaaacttacagcctatttagatcttaacctggataagtgctatgtgatccctctgaacacttccattgttatgccacccagaaacctactggagttacttattaacatcaaggctggaacctatttgcctcagtcctatctgattcatgagcacatggttattactgatcgcattgaaaacattgatcacctgggtttctttatttatcgactgtgtcatgacaaggaaacttacaaactgcaacgcagagaaactattaaaggtattcagaaacgtgaagccagcaattgtttcgcaattcggcattttgaaaacaaatttgccgtggaaactttaatttgttcttgaacagtcaagaaaaacattattgaggaaaattaatatcacagcataaccccaccctttacattttgtgcagtgattattttttaaagtcttctttcatgtaagtagcaaacagggctttactatcttttcatctcattaattcaattaaaaccattaccttaa

SEQ ID NO: 141 - protein, 266 aa, Homo sapiens
  misc_feature: Incyte ID No 632664CD1
MVKVTFNSALAQKEAKKDEPKSGEEALIIPPDAVAVDCKDPDDVVPVGQRRAWCWCMCFGLAFMLAGVILGGAYLYKYFALQPDDVYYCGIKYIKDDVILNEPSADAPAALYQTIEENIKIFEEEEVEFISVPVPEFADSDPANIVHDFNKKLTAYLDLNLDKCYVIPLNTSIVMPPRNLLELLINIKAGTYLPQSYLIHEHMVITDRIENIDHLGFFIYRLCHDKETYKLQRRETIKGIQKREASNCFAIRHFENKFAVETLICS

SEQ ID NO: 142 - DNA, 1030 bp, Homo sapiens
  misc_feature: Incyte ID No 995929.22
gttgcgacatgcagtgcgccggaggaactgtgctctttgaggccgacgctaggggcccggaagggaaactgcgaggcgaaggtgaccggggaccgagcatttcagatctgctcggtagacctggtgcaccaccaccatgttggctgcaaggctggtgtgtctccggacactaccttctagggttttccacccagctttcaccaaggcctcccctgttgtgaagaattccatcacgaagaatcaatggctgttaacacctagcagggaatatgccaccaaaacaagaattgggatccggcgtgggagaactggccaagaactcaaagaggcagcattggaaccatcgatggaaaaaatatttaaaattgatcagatgggaagatggtttgttgctggaggggctgctgttggtcttggagcattgtgctactatggcttgggactgtctaatgagattggagctattgaaaaggctgtaatttggcctcagtatgtcaaggatagaattcattccacctatatgtacttagcagggagtattggtttaacagctttgtctgccatagcaatcagcagaacgcctgttctcatgaacttcatgatgagaggctcttgggtgacaattggtgtgacctttgcagccatggttggagctggaatgctggtacgatcaataccatatgaccagagcccaggcccaaagcatcttgcttggttgctacattctggtgtgatgggtgcagtggtggctcctctgacaatattagggggtcctcttctcatcagagctgcatggtacacagctggcattgtgggaggcctctccactgtggccatgtgtgcgcccagtgaaaagtttctgaacatgggtgcacccctgggagtgggcctgggtctcgtctttgtgtcctcattgggatctatgtttcttccacctaccaccgtggctggtgccactctttactcagtggcaatgtacggtggattagttcttttcagcatgttccttctgtatgatacccagaaagta

SEQ ID NO: 143 - DNA, 2386 bp, Homo sapiens
  misc_feature: Incyte ID No 995929.27
gtgaccggggaccgagcatttcagatctgctcggtagacctggtgcaccaccaccatgttggctgcaaggctggtgtgtctccggacactaccttctagggttttccacccagctttcaccaaggcctcccctgttgtgaagaattccatcacgaagaatcaatggctgttaacacctagcagggaatatgccaccaaaacaagaattgggatccggcgtgggagaactggccaagaactcaaagaggcagcattggaaccatcgatggaaaaaatatttaaaattgatcagatgggaagatggtttgttgctggaggggctgctgttggtcttgggtgacaattggtgtgacctttgcagccatggttggagctggaatgctggtacgatcaataccatatgaccagagcccaggcccaaagcatcttgcttggttgctacattctggtgtgatgggtgcagtggtggctcctctgacaatattagggggtcctcttctcatcagagctgcatggtacacagctggcattgtgggaggcctctccactgtggccatgtgtgcgcccagtgaaaagtttctgaacatgggtgcacccctgggagtgggcctgggtctcgtctttgtgtcctcattgggatctatgtttcttccacctaccaccgtggctggtgccactctttactcagtggcaatgtacggtggattagttcttttcagcatgttccttctgtatgatacccagaaagtaatcaagcgtgcagaagtatcaccaatgtatggagttcaaaaatatgatcccattaactcgatgctgagtatctacatggatacattaaatatatttatgcgagttgcaactatgctggcaactggaggcaacagaaagaaatgaagtgactcagcttctggcttctctgctacatcaaatatcttgtttaatggggcagatatgcattaaatagtttgtacaagcagctttcgttgaagtttagaagataagaaacatgtcatcatatttaaatgttccggtaatgtgatgcctcaggtctgcctttttttctggagaataaatgcagtaatcctctcccaaataagcacacacattttcaattctcatgtttgagtgattttaaaatgttttggtgaatgtgaaaactaaagtttgtgtcatgagaatgtaagtcttttttctactttaaaatttagtaggttcactgagtaactaaaatttagcaaacctgtgtttgcatatttttttggagtgcagaatattgtaattaatgtcataagtgatttggagctttggtaaagggaccagagagaaggagtcacctgcagtcttttgtttttttaaatacttagaacttagcacttgtgttattgattagtgaggcgccagtaagaaacatctgggtatttggaaacaagtggtcattgttacattcatctgctgaacttaacaaaactgttcatcctgaaacaggcacaggtgatgcattctcctgctgttgcttctcagtgctctctttccaatatagatgtggtcatgtttgacttgtacagaatgttaatcatacagagaatccttgatggaattatatatgtgtgttttacttttgaatgttacaaaaggaaataactttaaaactattctcaagagaaaatattcaaagcatgaaatatgttgctttttccagaatacaaacagtatactcatgattgctaagtgtttttttatttttgcatatttattgaactgtctaattgaatacagcttgctcttgtcacctcttcaagctttcaagcctttatagaaaagcttctttgtggcttacactggaaattatgaaagcagtttttctcctaagacttttggtttctcgcattgcctctcagactaatcantaaaaagcaaagcaaaacagaactagttctgtcttaatgaaatatatcaacccaaaagtgtaatgaggaaaatgcttcattagtttcccctagcagacttttacttctcttacactgctacaccattacattcttgagacatttgtaagttctttgatacagaagagttatatttagaagtctttaatgaagggaaagaagtctgtatgatgactgaggtgattgctctttttgacaaatgtttattacagtaatgcagaaatactgttaatcatctcaaagtacataggaggcttatttctgaaacaaaggtttcagccagatgttgattagtctaatttttccaagtgaatcatatgcatgccatttgaatagggatttcttcaagtnagcaacttagtttcttgccagtatagtatagtgcagtgtgataagtacatgatacttgtagctagactacctctatttgaatctcaact

SEQ ID NO: 144 - DNA, 1212 bp, Homo sapiens
  misc_feature: Incyte ID No 1397029.1
atccatgctaaaggtaaacaaactgcaacttatatctgcaatttattttggtatagacaagaggtatgccagtagcacactggtggcttcagaagaaattctcaacacctagctcgccagagagtctatgtatgggattgaacaatctgtaaactaaaggatcctaatcatgaaaataagtatgataaattataagtccctaattggcactgttgtttatattagcctcctggatcatttttacagttttccagaactccacaaaggtttggtctgctctaaacttatccatctccctccattactggaacaactccacaaagtccttattccctaaaacaccactgatatcattaaagccactaacagagactgaactcagaataaaggaaatcatagagaaactagatcagcagatcccacccagacctttcacccacgtgaacaccaccaccagcgccacacatagcacagccaccatcctcaaccctcgagatacgtactgcaggggagaccagctgcacatcctgctggaggtgagggaccacttgggacgcaggaagcaatatggcggggatttcctgagggccaggatgtcttccccagcgctgatggcaggtgcttcaggaaaggtgactgacttcaacaacggcacctacctggtcagcttcactctgttctgggagggccaggtctctctgtctctgctgctcatccaccccagtgaagggggtgtcagctctctggagtgcaaggaaccaaggctattgacagggtgatcttcactggccagtttgtcaatggcacttccccaagtccactctgaatgtgggcctgatcttaaaacaccaaatgctgaattgtgccagtacctggacaacagagaccaagaaggcttctactgtgtgaggcctcaacacatgccctgtgctgcactcactcacatgtattctaagaacaagaaagtttcttatcttagcaaacaagaaaagagcctctttgaaaggtaaaaataattacttcttgagactacctgtgcaaatattgtgatttggcctatatactgatccaaagaaaagtcttgtgagtgtattaattttgggtgtctttagtaagagcctttggggaaaggatctgtgaattcatttagagacagtgcccattctctagtaatccacaaactttttgaacatttaattcttatcaatggga

SEQ ID NO: 145 - DNA, 841 bp, Homo sapiens
  misc_feature: Incyte ID No 403560.1
ttatcttaggggacggagaaaagagcctctttgaaaggtcaaatgtgggtgtagagattatggaaaaattcaatacaattagtgtctccaaatgcaacacaagaaacagttgcaatgaaagagaaatgcaagtttggaatgacatccacaatccccagtgggcatgtctggagaaacacatggaatcctgtctcctgtagtttggctacagtcaaaatgaaggaatgcctgagaggaaaactcatatacctaatgggagattccacgatccgccagtggatggaatacttcaaagccagtatcaacacactgaagtcagtggatctgcatgaatctggaaaattgcaacaccagcttgctgtggatttggataggaacatcaacatccagtggcaaaaacattgttatcccttgataggatcaatgacctattcagtcaaagagatggagtacctcacccgggccattgacagaactggaggagaaaaaaatactgtcattgttatttccctgggccagcatttcagaccctttcccattgatgtttttatccgaagggccctcaatgtccacaaagccattcagcatcttcttctgagaagcccagacactatggttatcatcaaaacagaaaacatcagggagatgtacaatgatgcagaaagatttagtgactttcatggttacattcaatatctcatcataaaggacattttccaggatctcagtgtgagtatcattgatgcctgggatataacaattgcatatggcacaaataatgtacacccacctcaacatgtagtcggaaatcagattaatatattattaaactatatttgtt

SEQ ID NO: 146 - DNA, 1480 bp, Homo sapiens
  misc_feature: Incyte ID No 1329606.3
tctgtgacctcctatgtgctgaatcagccaccttcggtgtcagtggccccaggacagacggccagacttacctgtgggggaaataatattgaaagtaagagtgtccactggtaccagcagaagccaggccaggnccctgtcctggtcgtctatcttgatgacgaccggccctcagggatccctgaccgattctctggntccaactntgggaacacggccaccctgaccatcagcaggtcgaagtcggggatgagccgactatttctgtcacgtgtgggatggcgttggggattctcctggtgctttcttcggacctgggacctgggtcagcgtcctaggtcagcccaaggccaaccccactgtcactctgttcccgccctcctctgaggagctccaagccaacaaggccacactagtgtgtctgatcagtgacttctacccgggagctgtgacagtggcctggaaggcagatggcagccccgtcaaggcgggagtggagaccaccaaaccctccaaacagagcaacaacaagtacgcggccagcagctacctgagcctgacgcccgagcagtggaagtcccacagaagctacagctgccaggtcacgcatgaagggagcaccgtggagaagacagtggcccctacagaatgttcataggttcccaactctaaccccacccacgggagcctggagctgcaggatcccaggggaggggttagagttgggaacctataacattctgtaggggccactgtcttctccacggtgctcccttcatgcgtgacctggcagctgtagcttctgtgggacttccactgctcgggcgtcaggctcaggtagctgctggccgcgtacttgttgttgctctgtttggagggtttggtggtctccactcccgccttgacggggctgccatctgccttccaggccactgtcacagctcccgggtagaagtcactgatcagacacactagtgtggccttgttggcttggagctcctcagaggagggcgggaacagagtgacagtggggttggccttgggctgacctaggacggagaccttggtcccagttccgaagaccccctcgagattgctactggacttatatgagctgcagtaataaacagcctcgtcctcagtctgcagtccagagatggtcagggtggccgtgtggccagactgggagccagagaagcgagttgaaacccctgagggccgatcagagacagcataaatcaagagtttgggggctttgcctgggtgttgttggtaccaggagacatttttataagcaccaatgacactgctggctccagtgcaggagatggcgatcgactgtccacgagacccagacacggaggcaggctgagtcagggcagactgggcccaggaccctgtgccctgagtgaggagggtgaggaatagcagagcccaggccatggtggagacgtcccgagagcgctgcctcctggagcgt

SEQ ID NO: 147 - DNA, 532 bp, Homo sapiens
  misc_feature: Incyte ID No 1092257.12
tttttaaaaaagacgcttccaagttatatttaatccaaagaagaaggatctcggccaatttggggttttgggttttggcttcgtttcttctcttcgttgactttggggttcaggtgccccagctgcttcgggctgccgaggaccttctgggcccccacattaatgaggcagccacctggcgagtctgacatggctgtcagcgacgcgctgctcccatctttctccacgttcgcgtctggcccggcgggaagggagaagacactgcgtcaagcaggtgccccgaataacagctcatgccacccggttcctgcatgcccagangagcccaagccaaagaggggaagacgatcgtggccccggaaaaggaccgncacccacacttgtgattacgcgggctgcggcaaaaccttagacaaagagttcccatctcaaggcacaccntgcgaaccccacacaggtgaggaaccttaccactgtgactngggacggcngtggatgggaaattcgcccgctcaagatgaaactngaccag

SEQ ID NO: 148 - DNA, 1853 bp, Homo sapiens
  misc_feature: Incyte ID No 474322.38
gatatgtggctgcatggatggcacaactgtgataaagaccatgggagcaggtcatggtggagggtggggaggagcagttctatttccagcatgttgagtttaggggcctccagcacccagggaggggtccagcaggcagctgtctatacaaatgcagctcaggggagaattcaggactgggacacagattcagaagccagcagcagagacctgagaggtgggtgtgatcactcatttgctgtttaaaggcccagaaaggagacagagaagggatggacagagagggagaaggggaactgagcgagaaggtcaaggagtcagtaaggaaatggttagcaagggccaagtgaacagggagtcctccatgaaaagggccaacaaggctcccctggatgttgaggcagaaacgcatgagggactcaggggaagctgtttccatggagtcgggagggcaaagccagattagaccaggtgggggctgatgggaaggcaaataaagacaggaggcaaagacaacattctggagaaagtttggccctgaaggggaggagaggtgggtggcacttggaaggctttgcttggtgtccccagacagctgactcatgagtgggatttggaaaaagcgtggactcctgcccatggcctgagtcctttaagatcagaaattatgtctcccatcatggcctctccatagaggcatgtatcttcagcaggcgttaggtcacaagccacatgatgccaagctgacagtggcttgcataatggggatatgtgactgtcgcataactagaattctggaagagtgcagtgccaggcttggggcagctttccagccatgtcattaagaatccagccttctcctggccttcagctatgccacgtggccagtgtctacacctgggatgtcaagagacaggctgcaggtccaccctcctggcctcatactatggaagaggctttccttttgggcatctctctttttggagggaggaaatagatcgttcccagaagcccccagcagacttccccttgttgctcattggttggaacaaggttacatgatacacaaagaccaatcactgcaaaggaaaaagggatgaccctgcctggcttacaccaatcacaatctattcccagacccccgaggctagggctttgcctcctggacacatctgttagcaagaggaagagattatggctgttaggaaggcctttgagaaagtatcccagtgcctggctgtgtctccaccaggctggaggccagcatcccaagggcaagaattctgtctccccattggtcagaaatatctggagcgcaggtgtttgtctccaactaggagcttctggaggacagggctgtgtcttctaccccagggttcccacaagaagccactgaatattaataaactcccatcttgtgtttattttcttatgatttcagcaaaacaggactcctgcagaagtcaactgtgtgagtgtgataaggctgctgccacctgttttgctagaaacaagacgacctacaataaaaagtaccagtactattccaataaacactgcagagggagcacccctcgttgctgagtcccctcttccctggaaaccttccacccagtgctgaatttccctctctcataccctccctccctaccctaaccaagttccttggccatgcagaaagcatccctcacccatcctagaggccaggcaggagcccttctatacccacccagaatgagacatccagcagatttccagccttctactgctctcctccacctcaactccgtgcttaaccaaagaagctgtactccggggggtctcttctgaataaagcaattagcaaatcatgt

SEQ ID NO: 149 - DNA, 334 bp, Homo sapiens
  misc_feature: Incyte ID No 255002.3
tatggggaaagctgaaatttggttaatccgaacatattgggattttgaatttcctcgtccaaaattaggtaagtatggacgaggattgcactgcaaacctgccaaacctttacctaaggttttatggagatacaaaggaaagaaaccagccacattaggaaacaatactcagctctttgattggataccccagaatgatcttcttggacatcccaaaaccaaagcttttatcactcatcntggaataatgggatctacgaagctatttaccacggagtccctatggtgggagttcccatgtttgctgatcagcctgataancttgctcacaatg

SEQ ID NO: 150 - DNA, 237 bp, Homo sapiens
  misc_feature: Incyte ID No 1330137.1
ggtctttctatctcttgtactacactgaattcacccccactgaaaaagtgtctttttcatagatcgagacatgtaagcagcatcatggaggtttgaagatgccgcatttggattggatgaattccaaattctgcttgcntgctttttaatattgatatgcttatacacttacactttatgcacaaaatgtagggttataataatgttaacaggacatgatctcttataatctacttg

SEQ ID NO: 151 - DNA, 2349 bp, Homo sapiens
  misc_feature: Incyte ID No 3699582CB1
ggagcaaaggcgccatggctgtggagtcccagggcggacgcccacttgtcctgggcctgctgctgtgtgtgctgggcccagtggtgtcccatgctgggaagatactgttgatcccagtggatggcagccactggctgagcatgcttggggccatccagcagctgcagcagaggggacatgaaatagttgtcctagcacctgacgcctcgttgtacatcagagacggagcattttacaccttgaagacgtaccctgtgccattccaaagggaggatgtgaaagagtcttttgttagtctcgggcataatgtttttgagaatgattctttcctgcagcgtgtgatcaaaacatacaagaaaataaaaaaggactctgctatgcttttgtctggctgttcccacttactgcacaacaaggagctcatggcctccctggcagaaagcagctttgatgtcatgctgacggaccctttccttccttgcagccccatcgtggcccagtacctgtctctgcccactgtattcttcttgcatgcactgccatgcagcctggaatttgaggctacccagtgccccaacccattctcctacgtgcccaggcctctctcctctcattcagatcacatgaccttcctgcagcgggtgaagaacatgctcattgccttttcacagaactttctgtgcgacgtggtttattccccgtatgcaacccttgcctcagaattccttcagagagaggtgactgtccaggacctattgagctctgcatctgtctggctgtttagaagtgactttgtgaaggattaccctaggcccatcatgcccaatatggtttttgttggtggaatcaactgccttcaccaaaatccactatcccaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatag

SEQ ID NO: 152 - protein, 533 aa, Homo sapiens
  misc_feature: Incyte ID No 3699582CD1
MAVESQGGRPLVLGLLLCVLGPVVSHAGKILLIPVDGSHWLSMLGAIQQLQQRGHEIVVLAPDASLYIRDGAFYTLKTYPVPFQREDVKESFVSLGHNVFENDSFLQRVIKTYKKIKKDSAMLLSGCSHLLHNKELMASLAESSFDVMLTDPFLPCSPIVAQYLSLPTVFFLHALPCSLEFEATQCPNPFSYVPRPLSSHSDHMTFLQRVKNMLIAFSQNFLCDVVYSPYATLASEFLQREVTVQDLLSSASVWLFRSDFVKDYPRPIMPNMVFVGGINCLHQNPLSQEFEAYINASGEHGIVVFSLGSMVSEIPEKKAMAIADALGKIPQTVLWRYTGTRPSNLANNTILVKWLPQNDLLGHPMTRAFITHAGSHGVYESICNGVPMVMMPLFGDQMDNAKRMETKGAGVTLNVLEMTSEDLENALKAVINDKSYKENIMRLSSLHKDRPVEPLDLAVFWVEFVMRHKGAPHLRPAAHDLTWYQYHSLDVIGFLLAVVLTVAFITFKCCAYGYRKCLGKKGRVKKAHKSKTH

SEQ ID NO: 153 - DNA, 2385 bp, Homo sapiens
  misc_feature: Incyte ID No 344537.24
acagtcagctgtcggtggcttctgctgagatggccagaggactccaggttcccctgccgcggctggccacaggactgctgctcctcctcagtgtccagccctgggctgagagtggaaaggtgttggtggtgcccactgatggcagcccctggctcagcatgcgggaggccttgcgggagctccatgccagaggccaccaggcggtggtcctcaccccagaggtgaatatgcacatcaaagaagagaaatttttcaccctgacagcctatgctgttccatggacccagaaggaatttgatcgcgttacgctgggctacactcaagggttctttgaaacagaacatcttctgaagagatattctagaagtatggcaattatgaacaatgtatctttggcccttcataggtgttgtgtggagctactgcataatgaggccctgatcaggcacctgaatgctacttcctttgatgtggttttaacagaccccgttaacctctgcggggcggtgctggctaagtacctgtcgattcctgctgtgtttttttggaggtacattccatgtgacttagactttaagggcacacagtgtccaaatccttcctcctatattcctaagttactaacgaccaattcagaccacatgacattcctgcaaagggtcaagaacatgctctaccctctggccctgtcctacatttgccatactttttctgccccttatgcaagtcttgcctctgagctttttcagagagaggtgtcagtggtggatcttgtcagctatgcatccgtgtggctgttccgaggggactttgtgatggactaccccaggccgatcatgcccaacatggtcttcattgggggcatcaactgtgccaacgggaagccactatctcaggaatttgaagcctacattaatgcttctggagaacatggaattgtggttttctctttgggatcaatggtctcagaaattccagagaagaaagctatggcaattgctgatgctttgggcaaaatccctcagacagtcctgtggcggtacactggaacccgaccatcgaatcttgcgaacaacacgatacttgttaagtggctaccccaaaacgatctgcttggtcacccgatgacccgtgcctttatcacccatgctggttcccatggtgtttatgaaagcatatgcaatggcgttcccatggtgatgatgcccttgtttggtgatcagatggacaatgcaaagcgcatggagactaagggagctggagtgaccctgaatgttctggaaatgacttctgaagatttagaaaatgctctaaaagcagtcatcaatgacaaaagttacaaggagaacatcatgcgcctctccagccttcacaaggaccgcccggtggagccgctggacctggccgtgttctgggtggagtttgtgatgaggcacaagggcgcgccacacctgcgccccgcagcccacgacctcacctggtaccagtaccattccttggacgtgattggtttcctcttggccgtcgtgctgacagtggccttcatcacctttaaatgttgtgcttatggctaccggaaatgcttggggaaaaaagggcgagttaagaaagcccacaaatccaagacccattgagaagtgggtgggaaataaggtaaaattttgaaccattccctagtcatttccaaacttgaaaacagaatcagtgttaaattcattttattcttattaaggaaatactttgcataaattaatcagccccagagtgctttaaaaaattctcttaaataaaaataatagactcgctagtcagtaaagatatttgaatatgtatcgtgccccctccggtgtctttgatcaggatgacatgtgccatttttcagaggacgtgcagacaggctggcattctagattacttttcttactctgaaacatggcctgtttgggagtgcgggattcaaaggtggtcccaccgctgcccctactgcaaatggcagttttaatcttatcttttggcttctgcagatggttgcaattgatccttaaccaataatggtcagtcctcatctctgtcctgcttcataggtgccaccttgtgtgtttaaagaagggaagctttgtacctttagagtgtaggtgaaatgaatgaatggcttggagtgcactgagaacagcatatgatttcttgctttggggaaaaagaatgatgctatgaaattggtgggtggtgtatttgagaagataatcattgcttatgtcaaatggagctgaatttgataaaaacccaaaatacagctatgaagtgctgggcaagtttactttttttctgatgtttcctacaactaaaaataaattaataaatttatataaattctaaaaaaaaaaa

SEQ ID NO: 154 - DNA, 391 bp, Homo sapiens
  misc_feature: Incyte ID No 016124.2
agcaacagcaggcatggaccaaagcagtgaaggatgtatgaaaaagattagcagtgtgaatcttgacaaacttataaatgacttctcacagatagaaaagaaaatggtagaaaccaatggaaagaacaatatactggatattcagttggaaaaaagtaattgcctattaaaagtaatgcaagcaaaggaggtctccattaaagaagaatgtgctactcttcataatataataaaagggctacaacagaccattgaatatcaacagaatttgaaaggtgaaaatgaacaactaaaaataagtgctgatcttataaaagagaagttaaagtctcatgaacaggatataagaataatattgccaaacttgtaagtgaaatgnaaatcnnagagg

SEQ ID NO: 155 - DNA, 3607 bp, Homo sapiens
  misc_feature: Incyte ID No 104423.33
cgcacataaggtgtgaccttttcattcccgttgttatggaggtaggctctctaggaatctgggagtagtagctggggggcaagagcaaataaagagctcgagcttctgtggtctctggggagatgttcccgggaagcctgtctagagggcggagggcagctgttgagatggcgtggctccccggctcctgcgcccgcgtggctttcgcggcgggcgctgcggcccggtattggacagcctggcagggcagcgcggggccgaatccggctgccgtggctgaggctcatggatcactcttttgtggtagggccacatctgccagagcctggagtctgcgaaggccgggacccggttccccggcccacagtgggggtgtgcaaacccgagagaactgggttgcaaattcgtgaagaatcagcatcatgtttggcagctgagtattggagccaggagcctgccatgaggttttgagaacagagtgctgttttagagctggcagcagcatctcagcccaagagaaggttatattcccagaggatgtcagtcccaaggaccagtagctgccatcagtttggattctgaaaactaactggcatcaacactgggtgtagaaacatgcttgccttatgtatcagaggacatgctcagcagatccaagagatatatttggcaactttttctagaaaaggcacattgggtatcattcattacatttttgagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnccaagttgtagctttgtcccttgccatcatgcccaacaagaggttctataccttttaatgaattgactttcataaattggttatgttggtgggcaagttctttaagctggaaattgtaaattcctcctgaaatgttttttcatgcagttaccatgaactaatactacaataaaggatggtcttgggtgtcaattcttgaaaatgtgtatttgaagaaaaagtaaatttcgcctactgttgtcagatattagcagtccagatattcgctctagcttgtaaacttccctgtggtcagcctaagctcttaaccccctattctacagctttagactgaaggcaaaagctcaaagcatttaaggcctcctcaatttcggcccctgctgctctgtgctctgttccttggtgtagctttgttccagctcctgcagcattgcaccattcctaaccacaaccttaacagcatgtagggccccatactctaggccccatcttgctgtcttcctgtgttggaatcttgcctaccaaggcctgtcaagagggtcctcccccatactgtttgcccacaaggttcaagagtgattggctcccagtcttcctggactgaactacttggatctaggggttgcctgttatttagaagagatcccttaactggtagggccctttcagtaggacagtatttgggtaaggagagggtataagatgagaacctttgctaagttaggttcctttgggagcctccagtttgtttctccctctccccagccatcttgttggatctgcccaagggattggactccttcattgtcctaggtttttaaggttttaaagcccaccttctgcagtcggttttctggaatgtgttggtggattacttaagagttatggggaaagagttcttacggcctgtctaggtcagtgaagtcatactggcaagtgacaagaaggaagccaggtttgtcacttaccctgcatgctaaggacatcagagcccacatagaagtctggtagtttttcagccagcagtgggtagggaaggtggataggggatttctgaacaggactttatgcctgtatgcatgggcaccatgtctgtcctgagcactaccacatggcatcgtaggctgctctgtcaaaggatatagcgatagctggtcctatgggcagatgcctagaatcaaggtgggcctcagaaatagggctgggtctccataaggaggtaggccacgttcagtccacccctagcatagatggaactggggtctcgggcaaccaggagttacaggaattttgctaaagattcccaccttccttgagtggggacacattcctctggttggagcttgttcccctaaatgaaaaatgtaatgcagtacatgtaggggccctactgtattctttttctgggggaaagctcttggtgttcacaatggctcaggcttacagtacctccagccatgtgcccatttccacaagtctttggggaaggccatgagctgagctgagagggttctctggggcctgggatgacttaagaaccaaatggcagataatgggatagggggcagatgcagaggaccaacgggaagttttaacacaaatccccttccccagatgctatggtttgcctttctgtaggtgggtggcccaggccaatttttggcaatatccttaacccacctgataggagaagcgccctcccctgcctgccttgcccattctacccaacaaggactccctggtgaggcccttcacttccatgtcacgtctgagctgcagctacagtctccttgccctcatggtggtcttcggcaggtgccacagggccccctccagggtcctcctggcctactgtcccttcccggacctcctccccactgcctgggccctcacccagctccaggatggtgggtaggtggccctgctttggggagcgcttgcgcaggctgagcaggaagggctggggcaaggagaagatgtccacgttgttgcccaggtcaatccacgtgacattggggaacttgctgggatccttaaggatgtcagtgaggtcgcgcagcacggcccgggtcaaccggttgccattgagtgccagtgtggtgaggcggggcagggtgctgagtgctggcagcagctgcaggaccatgtcgtccgtgaggcctgtgaagcccagctccacgctgtctacctgctccccacagcgctgtaggtagctggtcacccgctccaggtctcgggaggtcagtgggattcccgacaggtccactgtgttgtctggggggcttccggccaggacagccttgaggcaggcctgtggcttccttttcaccagcccccggtgccgcctccactgggagtgggggctgaggtggtatgtcagctgccggcacagcttctccatggcgcccagtgcatcgccttccggcttgggacagtggatgtagcgtgccaggctgatgatgaggtcgtgtgtgatggggtccaccaggttccggaagctggcgtagcgatacagaacgtcgtctagcgaggtcgactccatgcccaggtcatacgagggcactccccactctcagctgtgctcctgtgtctctcctggttaagataaagttagaaacaacaaatgcc

SEQ ID NO: 156 - DNA, 2405 bp, Homo sapiens
  misc_feature: Incyte ID No 406977.2
aggccgctagggtgcggggttggggaggaggccgctagtctacgcctgtggagccgatactcagccctctgcgaccatgggctgtgctggcggcacttctgcgcagcggcgcccgcagccgcagccccctgctccggaggctggtgcaggaaataagatatgtggaacggagttatgtatcaaaacccactttgaaggaagtggtcatagtaagtgctacaagaacacccattggatcttttttaggcagcctttccttgctgccagccactaagcttggttccattgcaattcagggagccattgaaaaggcagggattccaaaagaagaagtgaaagaagcatacatgggtaatgttctacaaggaggtgaaggacaagctcctacaaggcaggcagtattgggtgcaggcttacctatttctactccatgtaccaccataaacaaagtttgtgcttcaggaatgaaagccatcatgatggcctctcaaagtcttatgtgtggacatcaggatgtgatggtggcaggtgggatggagagcatgtccaatgttccatatgtaatgaacagaggatcaacaccatatggtggggtaaagcttgaagatttgattgtaaaagacgggctaactgatgtctacaataaaattcatatgggcagctgtgctgagaatacagcaaagaagctgaatattgcacgaaatgaacaggacgcttatgctattaattcttataccagaagtaaagcagcatgggaagctgggaaatttggaaatgaagttattcctgtcacagttacagtaaaaggtcaaccagatgtagtggtgaaagaagatgaagaatataaacgtgttgattttagcaaagttccaaagctgaagacagttttccagaaagaaaatggcacagtaacagctgccaatgccagtacactgaatgatggagcagctgctctggttctcatgacggcagatgcagcgaagaggctcaatgttacaccactggcaagaatagtagcatttgctgacgctgctgtagaacctattgattttccaattgctcctgtatatgctgcatctatggttcttaaagatgtgggattgaaaaaagaagatattgcaatgtgggaagtaaatgaagcctttagtctggttgtactagcaaacattaaaatgttggagattgatccccaaaaagtgaatatcaatggaggagctgtttctctgggacatccaattgggatgtctggagccaggattgttggtcatttgactcatgccttgaagcaaggagaatacggtcttgccagtatttgcaatggaggaggaggtgcttctgccatgctaattcagaagctgtagacaacctctgctatttaaggagacaaccctatgtgaccagaaggcctgctgtaatcagtgtgactactgtgggtcagcttatattcagataagctgtttcattttttattattttctatgttaacttttaaaaatcaaaatgatgaaatcccaaaacattttgaaattaaaaataaatttcttcttctgcttttttcttggtaaccttgaaaggtttgatncatttttgnattctgagtctanacttatcgaaatatggtagaaataccaatgtgtaatattagtgncttacataagtagctagaagtttccatttgtgagaacacatttctatttttgaggattgttaaaggtcaagtgaatgctctttataggtaatttacatttagtaaattacggtaaattaaattacttctctttacagtaagagttggctattctggacaaactagcagtgcttcatataatcactcaaaccacagtgtgtgcagcagtactagaaacaagacagaagcccatgtcctcagggtctagagtgggggcaatttcttataacctcaacattcagggttgggggaggtcaagcagaaaaccctggagtttgggctctgaattactatagcagcatagagagtgggaagggaggtagaaactgatatgctgaatggatatataaaaaagggaacagatcaccacttccaatacacgacaatgcctgttcttaagcaggacagactgtaacagaagtatctcgcattgcattttatctgggaaaaaattaattaattaaaacgctacaggcaggggctatgcacactaccctcaattgcagctctgaagagggaaaactccccctcaggaagaggagggggcagtaaaggtcattgtgtacacattatctgttggcaaataatgtattgtagatcttttgaaggagcaaaatgacaatatatgggagaaatagattagattgctcttcagattcagaggaagagtagagtattttcaaaaacatctgtaatcatcaccaacttttgaggcttg

SEQ ID NO: 157 - DNA, 1760 bp, Homo sapiens
  misc_feature: Incyte ID No 3355973CB1
ctcctcaccaagcagcagcttctccgctccttctaggatctccgcctggttcggcccgcctgcctccactcctgcctccaccatgtccatcagggtgacccagaagtcctacaaggtgtccacctctggcccccgggccttcagcagccgctcctacacgagtgggcccggttcccgcatcagctcctcgagcttctcccgagtgggcagcagcaactttcgcggtggcctgggcggcggctatggtggggccagcggcatgggaggcatcaccgcagttacggtcaaccagagcctgctgagcccccttgtcctggaggtggaccccaacatccaggccgtgcgcacccaggagaaggagcagatcaagaccctcaacaacaagtttgcctccttcatagacaaggtacggttcctggagcagcagaacaagatgctggagaccaagtggagcctcctgcagcagcagaagacggctcgaagcaacatggacaacatgttcgagagctacatcaacaaccttaggcggcagctggagactctgggccaggagaagctgaagctggaggcggagcttggcaacatgcaggggctggtggaggacttcaagaacaagtatgaggatgagatcaataagcgtacagagatggagaacgaatttgtcctcatcaagaaggatgtggatgaagcttacatgaacaaggtagagctggagtctcgcctggaagggctgaccgacgagatcaacttcctcaggcagctatatgaagaggagatccgggagctgcagtcccagatctcggacacatctgtggtgctgtccatggacaacagccgctccctggacatggacagcatcattgctgaggtcaaggcacagtacgaggatattgccaaccgcagccgggctgaggctgagagcatgtaccagatcaagtatgaggagctgcagagcctggctgggaagcacggggatgacctgcggcgcacaaagactgagatctctgagatgaaccggaacatcagccggctccaggctgagattgagggcctcaaaggccagagggcttccctggaggccgccattgcagatgccgagcagcgtggagagctggccattaaggatgccaacgccaagttgtccgagctggaggccgccctgcagcgggccaagcaggacatggcgcggcagctgcgtgagtaccaggagctgatgaacgtcaagctggccctggacatcgagatcgccacctacaggaagctgctggagggcgaggagagccggctggagtctgggatgcagaacatgagtattcatacgaagaccaccagcggctatgcaggtggtctgagctcggcctatgggggcctcacaagccccggcctcagctacagcctgggctccagctttggctctggcgcgggctccagctccttcagccgcaccagctcctccagggccgtggttgtgaagaagatcgagacacgtgatgggaagctggtgtctgagtcctctgacgtcctgcccaagtgaacagctgcggcagcccctcccagcctacccctcctgcgctgccccagagcctgggaaggaggccgctatgcagggtagcactgggaacaggagacccacctgaggctcagccctagccctcagcccacctggggagtttactacctggggaccccccttgcccatgcctccagctacaaaacaattcaattgctttttttttggtccaaaataaaacctcagctag

SEQ ID NO: 158 - protein, 483 aa, Homo sapiens
  misc_feature: Incyte ID No 3355973CD1
MSIRVTQKSYKVSTSGPRAFSSRSYTSGPGSRISSSSFSRVGSSNFRGGLGGGYGGASGMGGITAVTVNQSLLSPLVLEVDPNIQAVRTQEKEQIKTLNNKFASFIDKVRFLEQQNKMLETKWSLLQQQKTARSNMDNMFESYINNLRRQLETLGQEKLKLEAELGNMQGLVEDFKNKYEDEINKRTEMENEFVLIKKDVDEAYMNKVELESRLEGLTDEINFLRQLYEEEIRELQSQISDTSVVLSMDNSRSLDMDSIIAEVKAQYEDIANRSRAEAESMYQIKYEELQSLAGKHGDDLRRTKTEISEMNRNISRLQAEIEGLKGQRASLEAAIADAEQRGELAIKDANAKLSELEAALQRAKQDMARQLREYQELMNVKLALDIEIATYRKLLEGEESRLESGMQNMSIHTKTTSGYAGGLSSAYGGLTSPGLSYSLGSSFGSGAGSSSFSRTSSSRAVVVKKIETRDGKLVSESSDVLPK

SEQ ID NO: 159 - DNA, 5427 bp, Homo sapiens
  misc_feature: Incyte ID No 406457.3
gcaggcggcggggagaaagactctctcacctggtcttgcggctgtggccaccgccggccaggggtgtggagggcgtgctgccggagacgtccgccgggctctgcagttccgccgggggtcgggcagctatggagccgcggcccacggcgccctcctccggcgccccgggactggccggggtcggggagacgccgtcagccgctgcgctggccgcagccagggtggaactgcccggcacggctgtgccctcggtgccggaggatgctgcgcccgcgagccgggacggcggcggggtccgcgatgagggccccgcggcggccggggacgggctgggcagacccttggggcccaccccgagccagagccgtttccaggtggacctggtttccgagaacgccgggcgggccgctgctgcggcggcggcggcggcggcggcagcggcggcggctggtgctggggcgggggccaagcagacccccgcggacggggaagccagcggcgagagcgagccagctaaaggcagcgaggaagccaagggccgcttccgcgtgaacttcgtggacccagctgcctcctcgtcggctgaagacagcctgtcagatgctgccggggtcggagtcgacgggcccaacgtgagcttccagaacggcggggacacggtgctgagcgagggcagcagcctgcactccggcggcggcggcggcagtgggcaccaccagcactactattatgatacccacaccaacacctactacctgcgcaccttcggccacaacaccatggacgctgtgcccaggatcgatcactaccggcacacagccgcgcagctgggcgagaagctgctccggcctagcctggcggagctccacgacgagctggaaaaggaaccttttgaggatggctttgcaaatggggaagaaagtactccaaccagagatgctgtggtcacgtatactgcagaaagtaaaggagtcgtgaagtttggctggatcaagggtgtattagtacgttgtatgttaaacatttggggtgtgatgcttttcattagattgtcatggattgtgggtcaagctggaataggtctatcagtccttgtaataatgatggccactgttgtgacaactatcacaggattgtctacttcagcaatagcaactaatggatttgtaagaggaggaggagcatattatttaatatctagaagtctagggccagaatttggtggtgcaattggtctaatcttcgcctttgcccacgctgttgcagttgctatgtatgtggttggatttgcagaaaccgtggtggagttgcttaaggaacattccatacttatgatagatgaaatcaatgatatccgaattattggagccattacagtcgtgattcttttaggtatctcagtagctggaatggagtgggaagcaaaagctcagattgttcttttggtgatcctacttcttgctattggtgatttcgtcataggaacatttatcccactggagagcaagaagccaaaagggttttttggttataaatctgaaatatttaatgagaactttgggcccgattttcgagaggaagagactttcttttctgtatttgccatcttttttcctgctgcaactggtattctggctggagcaaatatctcaggtgatcttgcagatcctcagtcagccatacccaaaggaacactcctagccattttaattactacattggtttacgtaggaattgcagtatctgtaggttcttgtgttgttcgagatgccactggaaacgttaatgacactatcgtaacagagctaacaaactgtacttctgcagcctgcaaattaaactttgatttttcatcttgtgaaagcagtccttgttcctatggcctaatgaacaacttccaggtaatgagtatggtgtcaggatttacaccactaatttctgcaggtatattttcagccactctttcttcagcattagcatccctagtgagtgctcccaaaatatttcaggctctatgtaaggacaacatctacccagctttccagatgtttgctaaaggttatgggaaaaataatgaacctcttcgtggctacatcttaacattcttaattgcacttggattcatcttaattgctgaactgaatgttattgcaccaattatctcaaacttcttccttgcatcatatgcattgatcaatttttcagtattccatgcatcacttgcaaaatctccaggatggcgtcctgcattcaaatactacaacatgtggatatcacttcttggagcaattctttgttgcatagtaatgttcgtcattaactggtgggctgcattgctaacatatgtgatagtccttgggctgtatatttatgttacctacaaaaaaccagatgtgaattggggatcctctacacaagccctgacttacctgaatgcactgcagcattcaattcgtctttctggagtggaagaccacgtgaaaaactttaggccacagtgtcttgttatgacaggtgctccaaactcacgtccagctttacttcatcttgttcatgatttcacaaaaaatgttggtttgatgatctgtggccatgtacatatgggtcctcgaagacaagccatgaaagagatgtccatcgatcaagccaaatatcagcgatggcttattaagaacaaaatgaaggcattttatgctccagtacatgcagatgacttgagagaaggtgcacagtatttgatgcaggctgctggtcttggtcgtatgaagccaaacacacttgtccttggatttaagaaagattggttgcaagcagatatgagggatgtggatatgtatataaacttatttcatgatgcttttgacatacaatatggagtagtggttattcgcctaaaagaaggtctggatatatctcatcttcaaggacaagaagaattattgtcatcacaagagaaatctcctggcaccaaggatgtggtagtaagtgtggaatatagtaaaaagtccgatttagatacttccaaaccactcagtgaaaaaccaattacacacaaagttgaggaagaggatggcaagactgcaactcaaccactgttgaaaaaagaatccaaaggccctattgtgcctttaaatgtagctgaccaaaagcttcttgaagctagtacacagtttcagaaaaaacaaggaaagaatactattgatgtctggtggctttttgatgatggaggtttgaccttattgataccttaccttctgacgaccaagaaaaaatggaaagactgtaagatcagagtattcattggtggaaagataaacagaatagaccatgaccggagagcgatggctactttgcttagcaagttccggatagacttttctgatatcatggttctaggagatatcaataccaaaccaaagaaagaaaatattatagcttttgaggaaatcattgagccatacagacttcatgaagatgataaagagcaagatattgcagataaaatgaaagaagatgaaccatggcgaataacagataatgagcttgaactttataagaccaagacataccggcagatcaggttaaatgagttattaaaggaacattcaagcacagctaatattattgtcatgagtctcccagttgcacgaaaaggtgctgtgtctagtgctctctacatggcatggttagaagctctatctaaggacctaccaccaatcctcctagttcgtgggaatcatcagagtgtccttaccttctattcataaatgttctatacagtggacagccctccagaatggtacttcagtgcctagtgtagtaactgaaatcttcaatgacacattaacatcacaatggcgaatggtgacttttctttcacgatttcattaatttgaaagcacacaggaaagttgctccattgataacgtgtatggagacttcggttttagtcaattccatatctcaatcttaatggtgattcttctctgttgaactgaagtttgtgagagtagttttcctttgctacttgaatagcaataaaagcgtgttaactttttgattgatgaaagaagtacaaaaagcctttagccttgaggtgccttctgaaattaaccaaatttcatccatatatcctcttttataaacttatagaatgtcaaactttgccttcaactgtttttatttctagtctcttccactttaaaacaaaatgaacactgcttgtcttcttccattgaccatttagtgttgagtactgtatgtgttttgttaattctataaaggtatctgttagatattaaaggtgagaattagggcaggttaatcaaaaatggggaaggggaaatggtaaccaaaaagtaaccccatggtaaggtttatatgagtatatgtgaatatagagctaggaaaaaaagcccccccaaatacctttttaacccctctgattggctattattactatatttattattatttattgaaaccttagggaagattgaagattcatcccatacttctatataccatgcttaaaaatcacgtcattctttaaacaaaaatactcaagatcatttatatttatttggagagaaaactgtcctaatttagaatttccctcaaatctgagggacttttaagaaatgctaacagatttttctggaggaaatttagacaaaacaatgtcatttagtagaatatttcagtatttaagtggaatttcagtatactgtactatcctttataagtcattaaaataatgtttcatcaaatggttaaatggaccactggtttcttagagaaatgtttttaggcttaattcattcaattgtcaagtacacttagtcttaatacactcaggtttgaacagattattctgaatattaaaatttaatccattcttaatattttaaaacttttgttaagaaaaactgccagtttgtgcttttgaaatgtctgttttgacatcatagtctagtaaaattttgacagtgcatatgtactgttactaaaagctttatatgaaattattaatgtgaagtttttcatttataattcaaggaaggatttcctgaaaacatttcaagggatttatgtctacatatttgtgtgtgtgtgtgtatatatatgtaatatgcatacacagatgcatatgtgtatatataatgaaatttatgttgctggtattttgcattttaaagtgatcaagattcattaggcaaactttggtttaagtaaacatatgttcaaaatcagattaacagatacaggtttcatagagaacaaaggtgatcatttgaagggcatgctgtaatttcacacaattttccagttcaaaaatggagaatacttcgcctaaaatactgttaagtgggttaattgatacaagtttctgtggtggaaaatttatgcaggttttcacgaatcctt

SEQ ID NO: 160 - DNA, 1145 bp, Homo sapiens
  misc_feature: Incyte ID No 2190217CB1
aggaccattatatattgctctatattctaggtcgcccactttacacttccttctcatgcacttggtcaataccacgcccgctgaccacactggcgacttccctctctgtcgcccctccgtgaagtcagacccactctgcgggccaagaaaggtgaccgggcttccttccggcttgctaagcagaggccggaagcggtggtttttagcggctctctgggtagcagggtggtgtgatagcggcagcgaggggctcggagaggtgctcggattctcgtagctgtgccgggacttaaccaccaccatgtcgagcaaaagaacaaagaccaagaccaagaagcgccctcagcgtgcaacatccaatgtgtttgctatgtttgaccagtcacagattcaggagttcaaagaggccttcaacatgattgatcagaacagagatggtttcatcgacaaggaagatttgcatgatatgcttgcttcattggggaagaatccaactgatgagtatctagatgccatgatgaatgaggctccaggccccatcaatttcaccatgttcctcaccatgtttggtgagaagttaaatggcacagatcctgaagatgtcatcagaaatgcctttgcttgctttgatgaagaagcaactggcaccatacaggaagattacttgagagagctgctgacaaccatgggggatcggtttacagatgaggaagtggatgagctgtacagagaagcacctattgataaaaaggggaatttcaattacatcgagttcacacgcatcctgaaacatggagccaaagacaaagatgactgaaataacttcaaattccagccaaacgttccttgttgccactttgggtattctgagattttctcttgcatgcccttagctttacagcttttgcatttcctgttgtatttattctcagccattttgggcatatgtatctttataatcagactggaaacgggactttctattaatatcattttcagaataaaaaatagggtaatttaacctaccagcccttctcccccaataactgtggtctatacagagtcaatatattttttcagagaaagttattcgctcgattttttctgaatcataattaaactttatgataaaataaaaaaaaaaa

SEQ ID NO: 161 - protein, 171 aa, Homo sapiens
  misc_feature: Incyte ID No 2190217CD1
MSSKRTKTKTKKRPQRATSNVFAMFDQSQIQEFKEAFNMIDQNRDGFIDKEDLHDMLASLGKNPTDEYLDAMMNEAPGPINFTMFLTMFGEKLNGTDPEDVIRNAFACFDEEATGTIQEDYLRELLTTMGDRFTDEEVDELYREAPIDKKGNFNYIEFTRILKHGAKDKDD

SEQ ID NO: 162 - DNA, 1184 bp, Homo sapiens
  misc_feature: Incyte ID No 029061.1
attagctacactttcttcactagcaagataaaataatttccacattttctagttttactttgtagaaataactctctgtaattggactgtattcaacgaaaacttagtaagttgtaattatgcctcaggtatgtttctatgcactgagtgaagagtggagataaaaatagaatttagattttcctttactttttaaataggttgttgcctcttatatatttattctatgatgcaaatgtcactatcctaattcctcagtttatgtttaacagcacacagtggcacttctatgattcaaatacatttgataacctttgaaatcaatcagaatactgcaaaattaatttttctaaaacaatgcttttatcgttatttctcctgttgaatcatcagtacaatttccaaatgaaaacacttaaaataatctcatattacaatctttctctaacagaaccatgatgtaaggacagtgataacaaatatctgacaatgatatgattatttcctcatccatggaaattttccttaataaactaaagggctattttctaaaaagccaaagcattgcttacaagaacttttcatcatgacatggatagacactcagattcatacattcaaagggaagtgtcatgtattccctttcaatccaccctattctattgtgttatcttcctaaattattttctatctacattcttcattctctttcccattgaccctatgttctgtgtgataaaaattgcgtcattggaggcttttgaaggttaagtattatgccccatttcaccattaatcaacatacaacccttctccatattttgtaattcctttcatatacagaaaaaaagatactataatttcttcaaaatgcttgatattaatgatatatgggaaaacaattattttgtgcagcaatcttcagataactgggaaaggccggggaaaaagagagatactggtggttatcaatgacccatgtataaattgtttttattatgtaagctgtcttcacaaatgtcttcttatgtatgatcattagaactgttttatatatatatgtaaaatttccacattatcgagacattactttcagcagtgaagtaatccttttttaactgccacttaatgaattcaataaaatataatttattgtattttgctataataaactattgatgact

SEQ ID NO: 163 - DNA, 2613 bp, Homo sapiens
  misc_feature: Incyte ID No 1262593.2
gccgtcgccgccatttcaagaccgtactaggtagatggtcaattagagttcccagggtttgaagcctgtaactgctgccgccgctcaagccctccagagcattgctacggctgctgcccttgtactactacctccaaatacgttcttgctggtagtggcggcagcaggaccaattacctcttttttgctctccctcgagaagctccagatggcgtcttccgtgggcaacgtggccgacagcacagaaccaacgaaacgtatgctttccttccaagggttagctgagttggcacatcgagaatatcaggcaggagattttgaggcagctgagagacactgcatgcagctctggagacaagagccagacaatactggtgtgcttttattactttcatctatacacttccagtgtcgaaggctggacagatctgctcactttagcactctggcaattaaacagaacccccttctggcagaagcttattcgaatttggggaatgtgtacaaggaaagagggcagttgcaggaggcaattgagcattatcgacatgcattgcgtctcaaacctgatttcatcgatggttatattaacctggcagccgccttggtagcagcgggtgacatggaaggggcagtacaagcttacgtctctgctcttcagtacaatcctgatttgtactgtgttcgcagtgacctggggaacctgctcaaagccctgggtcgcttggaagaagccaaggtaggtgtttgatagaacacatttaaacatcagtattatgaaaacttgtactttttgccaagtcttcaactcttcattgagctatcttcacaaaacagtcctttgaaactgaggaaaactgacggcacgaatcgcctcagaatagagcagggccaggctttggcatatctgttctaaatctgggggtaaagcaagaacctgaacattttggagcctttctgctgagctagaccatctttataacactgggctccgtcatgatcttatgtgggaataaataacattccttcaaatctgaggcttgcctgctggtgacaagcagagcgcctgtgatttggctcaagactcctatatgatgcaggtgccattgaaaatgctgctcttctaagtcctttgtggcttgtaagtggagaagaatttcatccaaatgttaccctgtaatactggcatttaaaattcttatttaaccttcctcccttcatcttcctcaccctttttacagtggaagaaaggctgttaaaatgattacaaattaataattggaacatcctgtcccttgtccccactcccttcccaagttcctttttcctcttttccaatcctagttgtctaccttcttttcttcctcatttccttcttttattcctccccaccccaaccccttaaaaaaaaggtcagaaggacaaagctggtttgtttgggaaatggactgatcgaaagaaaacttgccaaagtggaaaggtggcttttagcattctgtgtttccaaataatgaatttgaacaccaggttgggttaattaaagcttttggtataatttaaaattaaatttataaatgcagttgtcttgttacaagccaccttacgcaaccgcgctgcaggggtgaggagtggggagaaaccagaatgcttctgaaactcccacctgttgctctgagccccacgcgcatgctaatgcgtggagtgtatgcgcagagtagctgtctgtttgactgcttcatccagggagggagaaggcttttcagcaccatctaatgttttaaaaggcactagttttaagtgcacagctcataaattctgctgacattttggattaaccttatgtaggttgccagctaatgaattgtaattgatttcaatcttagctgataaatctaattggtaatttatagaacaaatatttgataagctcctattaattgtcaccccaccaagcggacagctaacatgaattgcacttcactgcagctttagagatcggtttaggctgagacattgcgcctgccttaggttgctgacttctttatttcagagctctggagacacctagtttgaaaaatgttattctgtttttttgtgagaacttagtaaacaagaaaatactcttgagtgaaatgcaatgtatttcttttgtaatcagtgcatttgaaaattcaagccagcatattcctagtagatggaagcaaaattaagttgtctttgtagaaaatgaagagcctttcttccagcaaaaatccctgctgtatgcaatagccctgattaaccctctcccttctgcatgtttcccatattacagacttgagactgtcctcattcccatatgtaatagacatccaaagaatttcaattgctttgttgaacttttactaatgatcttgtttttattttctctcttgtttttggtttttcaccatcgatattgtatttagaaggtttcaggtgggtgaaacctcctattccatgcgtaaggtgcctcgctgaagggagctcgaggcctggatctagggcagacacacaacctcctcctcctcttccagcaaggaacgcaccgaaaagtcacatgatgagaaatatggtaacgggtttgtaactgcc

SEQ ID NO: 164 - DNA, 713 bp, Homo sapiens
  misc_feature: Incyte ID No 1094812.1
gctaatcaccttccagcaaccttcccacaacgaaactgtattccttgaaggaacaattagaaactacttcattctgagagttgtttcccagcccccattgtaaaataatttcactttcatttcttctcctcttttctctccatgacagataatgctctaccacaagaaaatggcctctcacctggggccattgctggcattgtgattggagtagtggccctggttgctctgatagcagtagccctggcatgttttctgcatttcgggaagaccggcaggtatgatggcctttcctcttgttctgtttcctccaaggctgactgccatgcttgggagagggaaagaattctttgcctgtctctgggcctggatctcatactcctcccactaaactcctgcttctcggcactaattcccacaggtttcctcttccctggtcttcgtgctccctgtctcccattatctcttggacatgggtattccaatccccatctaatctagaggaagagactcaagtatgttcctctgtcccctagtcagggaaagcaaggtctaagaaggaaaacaaatgaaaaaatgaaagaagcagcaggagaaagcaagtcatgtgctctgttgaccacattggaagaggagggagaaatagcataggaagagaacctaagagaaagcaaaaacaaccaaagtcctcttgaacaataaatagaaaaaag

SEQ ID NO: 165 - DNA, 1636 bp, Homo sapiens
  misc_feature: Incyte ID No 2434655CB1
gatcagtgtgtgagggaactgccatcatgaggtctgacaagtcagctttggtatttctgctcctgcagctcttctgtgttggctgtggattctgtgggaaagtcctggtgtggccctgtgacatgagccattggcttaatgtcaaggtcattctagaagagctcatagtgagaggccatgaggtaacagtattgactcactcaaagccttcgttaattgactacaggaagccttctgcattgaaatttgaggtggtccatatgccacaggacagaacagaagaaaatgaaatatttgttgacctagctctgaatgtcttgccaggcttatcaacctggcaatcagttataaaattaaatgatttttttgttgaaataagaggaactttaaaaatgatgtgtgagagctttatctacaatcagacgcttatgaagaagctacaggaaaccaactacgatgtaatgcttatagaccctgtgattccctgtggagacctgatggctgagttgcttgcagtcccttttgtgctcacacttagaatttctgtaggaggcaatatggagcgaagctgtgggaaacttccagctccactttcctatgtacctgtgcctatgacaggactaacagacagaatgacctttctggaaagagtaaaaaattcaatgctttcagttttgttccacttctggattcaggattacgactatcatttttgggaagagttttatagtaaggcattaggaaggcccactacattatgtgagactgtgggaaaagctgagatatggctaatacgaacatattgggattttgaatttcctcaaccataccaacctaactttgagtttgttggaggattgcactgtaaacctgccaaagctttgcctaaggaaatggaaaattttgtccagagttcaggggaagatggtattgtggtgttttctctggggtcactgtttcaaaatgttacagaagaaaaggctaatatcattgcttcagcccttgcccagatcccacagaaggtgttatggaggtacaaaggaaaaaaaccatccacattaggagccaatactcggctgtatgattggataccccagaatgatcttcttggtcatcccaaaaccaaagcttttatcactcatggtggaatgaatgggatctatgaagctatttaccatggggtccctatggtgggagttcccatatttggtgatcagcttgataacatagctcacatgaaggccaaaggagcagctgtagaaataaacttcaaaactatgacaagcgaagatttactgagggctttgagaacagtcattaccgattcctcttataaagagaatgctatgagattatcaagaattcaccatgatcaacctgtaaagcccctagatcgagcagtcttctggatcgagtttgtcatgcgccacaaaggagccaagcacctgcgatcagctgcccatgacctcacctggttccagcactactctatagatgtgattgggttcctgctgacctgtgtggcaactgctatattcttgttcacaaaatgttttttattttcctgtcaaaaatttaataaaactagaaagatagaaaagagggaatagatctttccaaattcaagaaagacctg

SEQ ID NO: 166 - protein, 527 aa, Homo sapiens
  misc_feature: Incyte ID No 2434655CD1
MRSDKSALVFLLLQLFCVGCGFCGKVLVWPCDMSHWLNVKVILEELIVRGHEVTVLTHSKPSLIDYRKPSALKFEVVHMPQDRTEENEIFVDLALNVLPGLSTWQSVIKLNDFFVEIRGTLKMMCESFIYNQTLMKKLQETNYDVMLIDPVIPCGDLMAELLAVPFVLTLRISVGGNMERSCGKLPAPLSYVPVPMTGLTDRMTFLERVKNSMLSVLFHFWIQDYDYHFWEEFYSKALGRPTTLCETVGKAEIWLIRTYWDFEFPQPYQPNFEFVGGLHCKPAKALPKEMENFVQSSGEDGIVVFSLGSLFQNVTEEKANIIASALAQIPQKVLWRYKGKKPSTLGANTRLYDWIPQNDLLGHPKTKAFITHGGMNGIYEAIYHGVPMVGVPIFGDQLDNIAHMKAKGAAVEINFKTMTSEDLLRALRTVITDSSYKENAMRLSRIHHDQPVKPLDRAVFWIEFVMRHKGAKHLRSAAHDLTWFQHYSIDVIGFLLTCVATAIFLFTKCFLFSCQKFNKTRKIEKRE

SEQ ID NO: 167 - DNA, 910 bp, Homo sapiens
  misc_feature: Incyte ID No 206344.1
catgtatgcaataaaaaataaaagatacatacacaaaattctttaaatgtcccacacacaagacaaatacgtgttcaaatacatcagtctctgaagcctctgcaccactctacacgctgctccttctgactagtaatgccctcctgcccctcctgtccacgtgtcaaactcccaatcaccctttaaaaccagattgaattattttgcttctgtgaagctttccctgactatccccgggatagaataatgtttccactagtgttttgtcatttactcgctataataagaatacgaaagaacatgtatttttgaaaagtatctgtgatctctaatgagcttgtaaacatcttgaggaatagagactaagttttgcttctttgttcccccaaagagaactttattaataacatttaccatctctttagagagagggtttttcccatctctgtgagaaagctccagaatctacaaccaggaataagtgttaatgggatagaaccaatgtagagaacagcatatgatatgtgaaatgtactttattattaatacgaattcagtgggctcacagaatgaacctttttgccaaactggggggaaagcattttctgtaaaggtatctttagaaaaatatgtataatttgaaaaatggttatccaaatttaacatttgtcatataaaaggctcataaaacgtgtgtggctgtgtttctcaaaattgtggggtcaattggtcacattatgcctagacattctggttttgttgcttggggttaataatggttgtggtcttatacagaaaaggaaatctggacatcttgcccctgttattaatacacctgtcattactaataaaagtggtttgttgatatgctaaataggttgaaaaagctgtcactttgcatgaaattaac

SEQ ID NO: 168 - DNA, 1525 bp, Homo sapiens
  misc_feature: Incyte ID No 1075717.7
ggtactaagtattaataatagagccaaacaggttacatttgtatccccttatagttgcattataattaggtattaaatctttggccaaaccaggccacctctgagagtaaataggatgtttttaataactacgctgagacaaatttaaactaggactgttctaggggagctagagataaggaaaaagaagaaacagtgctgaaaacttcaaatatgtaaagaaaacatataatatttaaagcttaactttaaacatttacatatgtgatcaacatatatttatatattaataaatattcagataaaaatgtgatgaagactgaaagtgaccaaacctaagatgttgataaaatgatattcaaagtacaaataggtaaaaacatccatatatttaactacatatccaattttgtatggggctgtcatatagatatctactaaataatctaagttgaaaaacaaccaagaccatcaattacttgcttagatcttaacacagccaaacagacccctgaaccatctcattttcttccgattttttttggagagatgaaatatgagagacggagaatttatgttcaactctgatttttaaattagatttaaaacaagcttactgaaatttaaagagatctcaagatgaaagagaactagaataatggttggtttttaaaacattaataattaaacttataaaaccaatggtaaaatagtttctcactcttgacgatattttgcagtgttttaaagggataccaaaaattctgcaatagtaaaccagtgaaagagaaaaatctaatatagatgaagctttaacctcttaatactgcattttgcaaggctgttcctgcaagctctggttttataggatatgatatatttagttgaattacagactaataatctcaacaatagtttctgtattgtcaatatactaaaatcttcaaaacagcctagaagattgaaaagggcatgaaattatgcaggcttggtattagatcccagctctgctactttctctttccagtagtcactggtccacatgggtttttatcaggtatttcacagtacaccctttaaaaacagaatcctttactgtttcctcaagacacttgtgcatgttaccagtggtagacaatctgtgatcatttattgaaaatatctaatcaaacgctaattttaacacttattttcttagtttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttt

SEQ ID NO: 169 - DNA, 1174 bp, Homo sapiens
  misc_feature: Incyte ID No 1075717.1
agagtatataaaagcagtgcgccttggtgcagaagtacagagcttaggacacagagcacatcaaaagttcccaaagagggcttgctctctcttcacctgctctgttctacagcacactaccagaagacagcagaaatgaaaagcatttactttgtggctggattatttgtaatgctggtacaaggcagctggcaacgttcccttcaagacacagaggagaaatccagatcattctcagcttcccaggcagacccactcagtgatcctgatcagatgaacgaggacaagcgccattcacagggcacattcaccagtgactacagcaagtatctggactccaggcgtgcccaagattttgtgcagtggttgatgaataccaagaggaacaggaataacattgccaaacgtcacgatgaatttgagagacatgctgaagggacctttaccagtgatgtaagttcttatttggaaggccaagctgccaaggaattcattgcttggctggtgaaaggccgaggaaggcgagatttcccagaagaggtcgccattgttgaagaacttggccgcagacatgctgatggttctttctctgatgagatgaacaccattcttgataatcttgccgccagggactttataaactggttgattcagaccaaaatcactgacaggaaataactatatcactattcaagatcatcttcacaacatcacctgctagccacgtgggatgtttgaaatgttaagtcctgtaaatttaagaggtgtattctgaggccacattgctttgcatgccaataaataaattttcttttagtgttgtgtagccaaaaattacaaatggaataaagttttatcaaaatattgctaaaatatcagctttaaaatatgaaagtgctagattctgttattttcttcttattttggatgaagtaccccaacctgtttacatttagcgataaaattatttttctatgatataatttgtaaatgtaaattattccgatctgacatatctgcattataataataggagaatagaagaactggtagccacagtggtgaaattggaaagagaactttcttcctgaaacctttgtcttaaaaatactcagctttcaatgtatcaaagatacaattaaataaaattttcaagcttctttacaaaaaaaaa

SEQ ID NO: 170 - DNA, 792 bp, Homo sapiens
  misc_feature: Incyte ID No 372647.1
accacgtctggcacaaaaaaaatgtatttcttaaaaaggctctaataaaaaacatttgaaagccactgttctaggtgataatgattgtaagatctttgtgtatagttcttgctagctcagtcttattaatagtttcattgagagagaattcaacaggtatttgtttgtaagtactaacaaaaaattgtacattcaatacttatcaaacaaaagttacatgatcttattcttccactattaaatttttattttatttttaaattttgattttttggcatttcacctgcaagtctttttgtcttattagagtcacactatgtgatggtattttttctttatccacaatctcccctgactcccctgttactattatggaataatgtaaagttaagaattaattatgattacagtagttatgggtaattaggtactatgaatcaaatcttagaaatcactttcattattgtaatagtgcctcagaaaacaatttttcctctttgactttttaaattgttaatactatcataaatggcatttatgtattcatttaccaaatattgatcaaaaactactttgtgtctaccatcagaatttaaaagacaccttcctagatcatagagaaggcttactgacatggcacatacagaatggtaaacagatagctatattacacaatgagataagtgcttcaataacaatatagagctgtaagggagtgaattgaagagatactcccctattcttaaatgttcagtgaaattcctttgtcccaaagcgctagttaaacgaagc

SEQ ID NO: 171 - DNA, 986 bp, Homo sapiens
  misc_feature: Incyte ID No 148512.1
gggttccgagttcattactacaggaaaaactgttctcttctgtggcacagagaaccctgcttcaaagcagaagtagcagttccggagtccagctggctaaaactcatcccagaggataatggcaacccatgccttagaaatcgctgggctgtttcttggtggtgttggaatggtgggcacagtggctgtcactgtcatgcctcagtggagagtgtcggccttcattgaaaacaacatcgtggtttttgaaaacttctgggaaggactgtggatgaattgcgtgaggcaggctaacatcaggatgcagtgcaaaatctatgattccctgctggctctttctccggacctacaggcagccagaggactgatgtgtgctgcttccgtgatgtccttcttggctttcatgatggccatccttggcatgaaatgcaccaggtgcacgggggacaatgagaaggtgaaggctcacattctgctgacggctggaatcatcttcatcatcacgggcatggtggtgctcatccctgtgagctgggttgccaatgccatcatcagagatttctataacccaatagtgaatnttgcccaaaaacgtgngcttggagaagctctctacttaggatggaccacggcactggtgctgattgttggaggagctctgttctgctgcgttttttgttgcaacgaaaagagcagtagctacagatactcgataccttcccatcgcacaacccaaaaaagttatcacaccggaaagaagtcaccgagcgtctactccagaagtcagtatgtgtagttgtgtatgtttttttaactttactataaagccatgcaaatgacaaaaatctatattactttctcaaaatggaccccaaagaaactttgatttactgttcttaactgcctaatcttaattacaggaactgtgcatcagctatttatgattctataagctatttcagcagaatgagatattaaatccaatg

SEQ ID NO: 172 - DNA, 3290 bp, Homo sapiens
  misc_feature: Incyte ID No 2023119CB1
cggcgctctctgcgtggttcttcttctcggccgctgaaacccccgcggctgcttcctgggaaggtcgtgagtcccgctgagctgtccccggtgccgccgacccgggccgtgtgcccgtggctccagccgctgccgcctcgatctcctcgtctcccgctccgccctcccttttccctggatgaacttgcgtcctttctcttctccgccatggaattctgctccgtgcttttagccctcctgagccaaagaaaccccagacaacagatgcccatacgcagcgtatagcagtaactccccagctcggtttctgtgccgtagtttacagtatttaattttatataatatatattatttattatagcatttttgatacctcatattctgtttacacatcttgaaaggcgctcagtagttctcttactaaacaaccactactccagagaatggcaacggtgattaccagtactacagctgctaccgccgcttctggtcctttggtggactacctatggatgctcatcctgggcttcattattgcatttgtcttggcattctccgtgggagccaatgatgtagcaaattcttttggtacagctgtgggctcaggtgtagtgaccctgaagcaagcctgcatcctagctagcatctttgaaacagtgggctctgtcttactgggggccaaagtgagcgaaaccatccggaagggcttgattgacgtggagatgtacaactcgactcaagggctactgatggccggctcagtcagtgctatgtttggttctgctgtgtggcaactcgtggcttcgtttttgaagctccctatttctggaacccattgtattgttggtgcaactattggtttctccctcgtggcaaaggggcaggagggtgtcaagtggtctgaactgataaaaattgtgatgtcttggttcgtgtccccactgctttctggaattatgtctggaattttattcttcctggttcgtgcattcatcctccataaggcagatccagttcctaatggtttgcgagctttgccagttttctatgcctgcacagttggaataaacctcttttccatcatgtatactggagcaccgttgctgggctttgacaaacttcctctgtggggtaccatcctcatctcggtgggatgtgcagttttctgtgcccttatcgtctggttctttgtatgtcccaggatgaagagaaaaattgaacgagaaataaagtgtagtccttctgaaagccccttaatggaaaaaaagaatagcttgaaagaagaccatgaagaaacaaagttgtctgttggtgatattgaaaacaagcatcctgtttctgaggtagggcctgccactgtgcccctccaggctgtggtggaggagagaacagtctcattcaaacttggagatttggaggaagctccagagagagagaggcttcccagcgtggacttgaaagaggaaaccagcatagatagcaccgtgaatggtgcagtgcagttgcctaatgggaaccttgtccagttcagtcaagccgtcagcaaccaaataaactccagtggccactaccagtatcacaccgtgcataaggattccggcctgtacaaagagctactccataaattacatcttgccaaggtgggagattgcatgggagactccggtgacaaacccttaaggcgcaataatagctatacttcctataccatggcaatatgtggcatgcctctggattcattccgtgccaaagaaggtgaacagaagggcgaagaaatggagaagctgacatggcctaatgcagactccaagaagcgaattcgaatggacagttacaccagttactgcaatgctgtgtctgaccttcactcagcatctgagatagacatgagtgtcaaggcagagatgggtctaggtgacagaaaaggaagtaatggctctctagaagaatggtatgaccaggataagcctgaagtctctctcctcttccagttcctgcagatccttacagcctgctttgggtcattcgcccatggtggcaatgacgtaagcaatgccattgggcctctggttgctttatatttggtttatgacacaggagatgtttcttcaaaagtggcaacaccaatatggcttctactctatggtggtgttggtatctgtgttggtctgtgggtttggggaagaagagttatccagaccatggggaaggatctgacaccgatcacaccctctagtggcttcagtattgaactggcatctgccctcactgtggtgattgcatcaaatattggccttcccatcagtacaacacattgtaaagtgggctctgttgtgtctgttggctggctccggtccaagaaggctgttgactggcgtctctttcgtaacatttttatggcctggtttgtcacagtccccatttctggagttatcagtgctgccatcatggcaatcttcagatatgtcatcctcagaatgtgaagctgtttgagattaaaatttgtgtcaatgtttgggaccatcttaggtattcctgctcccctgaagaatgattacagtgttaacagaagactgacaagagtctttttatttgggagccagaggagggaagtgttacttgtgctataactgcttttgtgctaaatatgaattgtctcaaaattagctgtgtaaaatagcccgggttccactggctcctgctgaggtcccctttccttctgggctgtgaattcctgtacatatttctctactttttgtatcaggcttcaattccattatgttttaatgttgtctctgaagatgacttgtgatttttttttcttttttttaaaccatgaagagccgtttgacagagcatgctctgcgttgttggtttcaccagcttctgccctcacatgcacagggatttaacaacaaaaatataactacaacttcccttgtagtctcttatataagtagagtccttggtactctgccctcctgtcagtagtggcaggatctattggcatattcgggagcttcttagagggatgaggttctttgaacacagtgaaaatttaaattagtaacttttttgcaagcagtttattgactgttattgctaagaagaagtaagaaagaaaaagcctgttggcaatcttggttatttctttaagatttctggcagtgtgggatggatgaatgaagtggaatgtgaactttgggcaagttaaatgggacagccttccatgttcatttgtctacctcttaactgaataaaaaagcctacagtttttagaaaaaaaaaaa

SEQ ID NO: 173 - protein, 679 aa, Homo sapiens
  misc_feature: Incyte ID No 2023119CD1
MATVITSTTAATAASGPLVDYLWMLILGFIIAFVLAFSVGANDVANSFGTAVGSGVVTLKQACILASIFETVGSVLLGAKVSETIRKGLIDVEMYNSTQGLLMAGSVSAMFGSAVWQLVASFLKLPISGTHCIVGATIGFSLVAKGQEGVKWSELIKIVMSWFVSPLLSGIMSGILFFLVRAFILHKADPVPNGLRALPVFYACTVGINLFSIMYTGAPLLGFDKLPLWGTILISVGCAVFCALIVWFFVCPRMKRKIEREIKCSPSESPLMEKKNSLKEDHEETKLSVGDIENKHPVSEVGPATVPLQAVVEERTVSFKLGDLEEAPERERLPSVDLKEETSIDSTVNGAVQLPNGNLVQFSQAVSNQINSSGHYQYHTVHKDSGLYKELLHKLHLAKVGDCMGDSGDKPLRRNNSYTSYTMAICGMPLDSFRAKEGEQKGEEMEKLTWPNADSKKRIRMDSYTSYCNAVSDLHSASEIDMSVKAEMGLGDRKGSNGSLEEWYDQDKPEVSLLFQFLQILTACFGSFAHGGNDVSNAIGPLVALYLVYDTGDVSSKVATPIWLLLYGGVGICVGLWVWGRRVIQTMGKDLTPITPSSGFSIELASALTVVIASNIGLPISTTHCKVGSVVSVGWLRSKKAVDWRLFRNIFMAWFVTVPISGVISAAIMAIFRYVILRM

SEQ ID NO: 174 - DNA, 1708 bp, Homo sapiens
  misc_feature: Incyte ID No 1973832CB1
cgcagctctgtgtgaaggtgcagttttgccaaggagtgctaaagaacttagatgtcagtgcataaagacatactccaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcatggtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagatatacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttactgtttctgattgtatggaaatataaaagtaaatatgaaacatttaaaatataatttgttgt

SEQ ID NO: 175 - protein, 99 aa, Homo sapiens
  misc_feature: Incyte ID No 1973832CD1
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 176 - DNA, 3154 bp, Homo sapiens
  misc_feature: Incyte ID No 241888.54
gcggcggtaaatattccagtaaaaaatgacagactgactatatacatcttctttccttcccaaaaccccactaaaatgacaatcgaggaatttttttttaagtataaaactctaagaataaaaataatgggaaggtgacagcagcagataagaggttttaacaaactttcagagacagcagagcacacaagcttctaggacaagagccaggaagaaaccaccggaaggaaccatctcactgtgtgtaaacatgacttccaagctggccgtggctctcttggcagccttcctgatttctgcagctctgtgtgaaggtgcagttttgccaaggagtgctaaagaacttagatgtcagtgcataaagacatactccaaacctttccaccccaaatttatcaaagaactgagagtgattgagagtggaccacactgcgccaacacagaaattattgtaaagctttctgatggaagagagctctgtctggaccccaaggaaaactgggtgcagagggttgtggagaagtttttgaagagggctgagaattcataaaaaaattcattctctgtggtatccaagaatcagtgaagatgccagtgaaacttcaagcaaatctacttcaacacttcatgtattgtgtgggtctgttgtagggttgccagatgcaatacaagattcctggttaaatttgaatttcagtaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctgggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttattagataaatttcaatcagggtttttagattaaacaaacaaacaattgggtacccagttaaattttcatttcagataaacaacaaataattttttagtataagtacattattgtttatctgaaattttaattgaactaacaatcctagtttgatactcccagtcttgtcattgccagctgtgttggtagtgctgtgttgaattacggaataatgagttagaactattaaaacagccaaaactccacagtcaatattagtaatttcttgctggttgaaacttgtttattatgtacaaatagattcttataatattatttaaatgactgcatttttaaatacaaggctttatatttttaactttaagatgtttttatgtgctctccaaattttttttaccttgacaacaaattatattttaaatgtttcatatttacttttatatttccatacaatcagaaacatttaaaaaaatttggagagcacataaaaacatcttaaagttaaaaatataaagccttgtatttaaaaatgcagtcatttaaataatattataagaatctatttgtacataataaacaagtttcaaccagcaagaaattactaatattgactgtggagttttggctgttttaatagttctaactcattattccgtaattcaacacagcactaccaacacagctggcaatgacaagactgggagtatcaaactaggattgttagttcaattaaaatttcagataaacaataatgtacttatactaaaaaattatttgttgtttatctgaaatgaaaatttaactgggtacccaattgtttgtttgtttaatctaaaaaccctgattgaaatttatctaataaaacatcatttaatatctaaaataaaataaatttactataacatctttataactattcaatcaatgattcatcttctatttttccaaattcttgcacaaatatttgatgcttaaataaatacataaataataaataggttaataagttacacttgaaaataatttatgttatgatgaaaaaacttaaagtgcttccacatgtcctcacaacatcactgtgaggtaagatggtggctaatactttttccacttagaaataaaggagaaaccaaggcacagtggaacaaggacttgtggatcctggctagcagactagggttgccagatttaacagaaaaaaatccaggatttccagctaaatttgactttatggcaaaatttattgtcccatcatttttatgtgatgcttcaaatatcacattctagcaaacccattcaattcctgaaattaaagttcggatattctcttggcccttggcgtcaattttgctatttgtatattctcccgtgcaatatctaggaaagccttatatttaaaaattatttgttgttttttgtagattcaaataaataatactttacatataagtatgttctggatatttcatggtacaatgaaaaactattcattgtttactgaaattcaaatttaaccaggaatcttgtattgcatctggcaacacttcatgtattgtgtgggtctgttgtagggttgccagangcnatataagtatgttctggatatttcatggtacaatgaaaaactattcattgtttactgaaattcaaatttaaccaggaatcttgtattgcatctggcaaccctacaacangncccacacaanncatgaagtgttgaagtagatttgcttgaagtttcactgtgccttggtttctcctttatttctaagtggaaaaagnattagccaccatcttacctcacagtgatgttgtga

SEQ ID NO: 177 - DNA, 800 bp, Homo sapiens
  misc_feature: Incyte ID No 1736965CB1
gacgtgaaaatctgccttctcaccatgaggcttctagtcctttccagcctgctctgtatcctgcttctctgcttctccatcttctccacagaagggaagaggcgtcctgccaaggcctggtcaggcaggagaaccaggctctgctgccaccgagtccctagccccaactcaacaaacctgaaaggacatcatgtgaggctctgtaaaccatgcaagcttgagccagagccccgcctttgggtggtgcctggggcactcccacaggtgtagcactcccaaagcaagactccagacagcggagaacctcatgcctggcacctgaggtacccagcagcctcctgtctcccctttcagccttcacagcagtgagctgcaatgttggagggcttcatctcgggctgcaaggaccctgggaaagttccagaactccacgtccttgtctcaattgtgccatcaactttcagagctatcatgagccaacctcaccccacagggcctcagtcgccaccatgtgggcctctccagtgcaaaccaccgagcattccaccatgaccggtcacagctacaaatccagagaccatcaatcctgctagagtgcagggtggcaagcacccaagggtggctgaccaagactgcagagtctcctccatcttcaggtccattcagcctcctggcatttaactaccagcatccagtggtccccaaggaatcccttcctagcctcctgacatgagtctgctggaaagagcatccaaacaaacaagtaataaataaataaataaactcaaaaaaaaaaaaaa

SEQ ID NO: 178 - protein, 81 aa, Homo sapiens
  misc_feature: Incyte ID No 1736965CD1
MRLLVLSSLLCILLLCFSIFSTEGKRRPAKAWSGRRTRLCCHRVPSPNSTNLKGHHVRLCKPCKLEPEPRLWVVPGALPQV

SEQ ID NO: 179 - DNA, 1738 bp, Homo sapiens
  misc_feature: Incyte ID No 412065.17
ggaacagcggcctctgacaccagcacagcaaacccgccgggatcaaagtgtaccagtcggcagcatgggctacgaaatgtgggaattgtggacccggctactccacccctctggaggccatgaaaggacccagggaagagatcgtctacctgccctgcatttaccgaaacacaggcactgaggccccagattatctggccactgtggatgttgaccccaagtctccccagtattgccaggtcatccaccggctgcccatgcccaacctgaaggacgagctgcatcactcaggatggaacacctgcagcagctgcttcggtgatagcaccaagtcgcgcaccaagctggtgctgcccagtctcatctcctctcgcatctatgtggtggacgtgggctctgagccccgggccccaaagctgcacaaggtcattgagcccaaggacatccatgccaagtgcgaactggcctttctccacaccagccactgcctggccagcggggaagtgatgatcagctccctgggagacgtcaagggcaatggcaaagggggttttgtgctgctggatggggagacgttcgaggtgaaggggacatgggagagacctgggggtgctgcaccgttgggctatgacttctggtaccagcctcgacacaatgtcatgatcagcactgagtgggcagctcccaatgtcttacgagatggcttcaaccccgctgatgtggaggctggactgtacgggagccacttatatgtatgggactggcagcgccatgagattgtgcagaccctgtctctaaaagatgggcttattcccttggagatccgcttcctgcacaacccagacgctgcccaaggctttgtgggctgcgcactcagctccaccatccagcgcttctacaagaacgagggaggtacatggtcagtggagaaggtgatccaggtgccccccaagaaagtgaagggctggctgctgcccgaaatgccaggcctgatcaccgacatcctgctctccctggacgaccgcttcctctacttcagcaactggctgcatggggacctgaggcagtatgacatctctgacccacagagaccccgcctcacaggacagctcttcctcggaggcagcattgttaagggaggccctgtgcaagtgctggaggacgaggaactaaagtcccagccagagcccctagtggtcaagggaaaacgggtggctggaggccctcagatgatccagctcagcctggatgggaagcgcctctacatcaccacgtcgctgtacagtgcctgggacaagcagttttaccctgatctcatcagggaaggctctgtgatgctgcaggttgatgtagacacagtaaaaggagggctgaagttgaaccccaacttcctggtggacttcgggaaggagccccttggcccagcccttgcccatgagctccgctaccctgggggcgattgtagctctgacatctggatttgaactccaccctcatcacccacactccctattttgggccctcacttccttggggacctggcttcattctgctctctcttggcacccgacccttggcagcatgtaccacacagccaagctgagactgtggcaatgtgttgagtcatatacatttactgaccactgttgcttgttgctcactgtgctgcttttccatgagctcttggaggcaccaagaaataaactcgtaaccctgtccttcaaaaaaaaaaaaaagg

SEQ ID NO: 180 - DNA, 1969 bp, Homo sapiens
  misc_feature: Incyte ID No 988660.32
ccggctcgccgcgctccccaccttgcctgcgcccgcccggagccagcggttctccaagcacccagcatcctgctagacgcgccgcgcaccgacggaggggacatgggcagagcaatggtggccaggctcgggctggggctgctgctgctggcacatgctcctacccacgcagatttattccagtgaaacaacaactggaacttcaagtaactcctcccagagtacttccaactctgggttggccccaaatccaactaatgccaccaccaaggtggctggtggtgccctgcagtcaacagccagtctcttcgtggtctcactctctcttctgcatctctactcttaagagactcaggccaagaaacgtcttctaaatttccccatcttctaaacccaatccaaatggcgtctggaagtccaatgtggcaaggaaaaacaggtcttcatcgaatctactaattccacaccttttattgacacagaaaatgttgagaatcccaaatttgattgatttgaagaacatgtgagaggtttgactagatgatggatgccaatattaaatctgctggagtttcatgtacaagatgaaggagaggcaacatccaaaatagttaagacatgatttccttgaatgtggcttgagaaatatggacacttaatactaccttgaaaataagaatagaaataaaggatgggattgtggaatggagattcagttttcatttggttcattaattctataaggccataaaacaggtaatataaaaagcttccatgattctatttatatgtacatgagaaggaacttccaggtgttactgtaattcctcaacgtatgtttcgacagcactaatttaatgccgatatactctagatgaagttttacantgttgagctantgctgttctcttgggaactgaactcactttcctcctgaggcttggattgacatgcattgaccttttatgtagtaatgacatgtgccaggcaatgatgaatgagaatctacccccagatccaagcatccttgagcaactctttgattatccatattgagtcaaatggtaggcatttcctatcacctgtttccattcaacaagagcactacattcatttagctaaacggattccaaagagtagaattgcattgaccgcgactaatttcaaaatgctttttattattattattttttagacagtctcactttgtcgcccaggctggtttcgaactcctgacctcaggtgatccacccgcctcggcctcccaaagtgctgggattacaggcttgagcccccgcgcccagccatcaaaatgctttttatttctgcatatgttgaatactttttacaatttaaaaaaatgatctgttttgaaggcaaaattgcaaatcttgaaattaagaaggcaaaaatgtaaaggagtcaaaactataaatcaagtatttgggaagtgaagactggaagctaatttgcattaaattcacaaacttttatactctttctgtatatacattttttttctttaaaaaacaactatggatcagaatagccacatttagaacactttttgttatcagtcaatatttttagatagttagaacctggtcctaagcctaaaagtgggcttgattctgcagtaaatcttttacaactgcctcgacacacataaacctttttaaaaatagacgctccccgaagtctttgttcgcatggtcacacactgatgcttagatgttccagtaatctaatatggccacagtagtcttgatgaccaagtcctttttttccatctttagaaaactacatgggaacaacagatcgaacagttttgaagctacctgtgtgtgtgaatgaacactcttgctttattccagaaatgctgtacatctattttggattgtatattgtgtttgtgtatttacgcttgattcatagtaacttcttatgaaatgattgcatgacacactgt

SEQ ID NO: 181 - DNA, 919 bp, Homo sapiens
  misc_feature: Incyte ID No 1434821CB1
acggcctaggcctgnaatntacggtcatggcgtaccacanaccgccgncttaatnnccgctaaggcgcgtcccatcgccattcanntnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncgctctttaagcaaacagagcctgccctataaaatccggggctcgggcggcctctcatccctgactcggggtcgcctttggagcagagaggaggcaatggccaccatggagaacaaggtgatctgcgccctggtcctggtgtccatgctggccctcggcaccctggccgaggcccagacagagacgtgtacagtggccccccgtgaaagacagaattgtggttttcctggtgtcacgccctcccagtgtgcaaataagggctgctgtttcgacgacaccgttcgtggggtcccctggtgcttctatcctaataccatcgacgtccctccagaagaggagtgtgaattttagacacttctgcagggatctgcctgcatcctgacgcggtgccgtccccagcacggtgattagtcccagagctcggctgccacctccaccggacacctcagacacgcttctgcagctgtgcctcggctcacaacacagattgactgctctgactttgactactcaaaattggcctaaaaattaaaagagatcgatattaaaaaaaaaaaaanananaanggaaangnnancnnngaaagaganaaaaaaaagggcggcnncgactaatgaat

SEQ ID NO: 182 - protein, 84 aa, Homo sapiens
  misc_feature: Incyte ID No 1434821CD1
MATMENKVICALVLVSMLALGTLAEAQTETCTVAPRERQNCGFPGVTPSQCANKGCCFDDTVRGVPWCFYPNTIDVPPEEECEF

SEQ ID NO: 183 - DNA, 5648 bp, Homo sapiens
  misc_feature: Incyte ID No 464689.64
ggtgtggtgtcggtgtcggcagcatccccggcgccctgctgcggtcgccgcgagcctcggcctctgtctcctccccctcccgcccttacctccacgcgggaccgcccgcgccagtcaactcctcgcactttgcccctgcttggcagcggataaaagggggctgaggaaataccggacacggtcacccgttgccagctctagcctttaaattcccggctcggggacctccacgcaccgcggctagcgccgacaaccagctagcgtgcaaggcgccgcggctcagcgcgtaccggcgggcttcgaaaccgcagtcctccggcgaccccgaactccgctccggagcctcagccccctggaaagtgatcccggcatcggagagccaagatgccggcccacttgctgcaggacgatatctctagctcctataccaccaccaccaccattacagcgcctccctccaggggtcctgcagaatggaggagataagttggagacgatgcccctctacttggaagacgacattcgccctgatataaaagatgatatatatgaccccacctacaaggataaggaaggcccaagccccaaggttgaatatgtctggagaaacatcatccttatgtctctgctacacttgggagccctgtatgggatcactttgattcctacctgcaagttctacacctggctttggggggtattctactattttgtcagtgccctgggcataacagcaggagctcatcgtctgtggagccaccgctcttacaaagctcggctgcccctacggctctttctgatcattgccaacacaatggcattccagaatgatgtctatgaatgggctcgtgaccaccgtgcccaccacaagttttcagaaacacatgctgatcctcataattcccgacgtggctttttcttctctcacgtgggttggctgcttgtgcgcaaacacccagctgtcaaagagaaggggagtacgctagacttgtctgacctagaagctgagaaactggtgatgttccagaggaggtactacaaacctggcttgctgatgatgtgcttcatcctgcccacgcttgtgccctggtatttctggggtgaaacttttcaaaacagtgtgttcgttgccactttcttgcgatatgctgtggtgcttaatgccacctggctggtgaacagtgctgcccacctcttcggatatcgtccttatgacaagaacattagcccccgggagaatatcctggtttcacttggagctgtgggtgagggcttccacaactaccaccactcctttccctatgactactctgccagtgagtaccgctggcacatcaacttcaccacattcttcattgattgcatggccgccctcggtctggcctatgaccggaagaaagtctccaaggccgccatcttggccaggattaaaagaaccggagatggaaactacaagagtggctgagtttggggtccctcaggttcctttttcaaaaaccagccaggcagaggttttaatgtctgtttattaactactgaataatgctaccaggatgctaaagatgatgatgttaacccattccagtacagtattcttttaaaattcaaaagtattgaaagccaacaactctgcctttatgatgctaagctgatattatttcttctcttatcctctctctcttctaggcccattgtcctccttttcactttattgctatcgccctcctttcccttattgcctcccaggcaagcagctggtcagtctttgctcagtgtccagcttccaaagcctagacaacctttctgtagcctaaaacgaatggtctttgctccagataactctctttccttgagctgttgtgagctttgaagtaggtggcttgagctagagataaaacagaatcttctgggtagtcccctgttgattatcttcagcccaggcttttgctagatggaatggaaaagcaacttcatttgacacaaagcttctaaagcaggtaaattgtcgggggagagagttagcatgtatgaatgtaaggatgagggaagcgaagcaagaggaacctctcgccatgatcagacatacagctgcctacctaatgaggacttcaagccccaccacatagcatgcttcctttctctcctggctcggggtaaaaagtggctgcggtgtttggcaatgctaattcaatgccgcaacatatagttgaggccgaggataaagaaagacattttaagtttgtagtaaaagtggtctctgctggggaagggtttcttttcttttttctttatcacaaggagatttcttagttcatatatcaagaagtcttgaagttgggtgtttccagaattggtaaaaacagcagctcatggaattttgagtattccatgagctgctcattacagttctttcctctttctgctctgccatcttcaggatattggttcttcccctcatagtaataagatggctgtggcatttccaaacatccaaaaaaagggaaggatttaaggaggtgaagtcgggtcaaaaataaaatatatatacatatatacattgcttagaacgttaaactattagagtatttcccttccaaagagggatgtttggaaaaaactctgaaggagaggaggaattagttgggatgccaatttcctctccactgctggacatgagatggagaggctgagggacaggatctataggcagcttctaagagcgaacttcacataggaagggatctgagaacacgttgccaggggcttgagaaggttactgagtgagttattgggagtcttaataaaataaactagatattaggtccattcattaattagttccagtttctccttgaaatgagtaaaaactagaaggcttctctccacagtgttgtgccccttcactcatttttttttgaggagaagggggtctctgttaacatctagcctaaagtatacaactgcctggggggcagggttaggaatctcttcactaccctgattcttgattcctggctctaccctgtctgtcccttttctttgaccagatctttctcttccctgaacgttttcttctttccctggacaggcagcctcctttgtgtgtattcagaggcagtgatgacttgctgtccaggcagctccctcctgcacacagaatgctcagggtcactgaaccactgcttctcttttgaaagtagagctagctgccactttcacgtggcctccgcagtgtctccacctacacccctgtgctcccctgccacactgatggctcaagacaaggctggcaaaccctcccagaaacatctctggcccagaaagcctctctctccctccctctctcatgaggcacagccaagccaagcgctcatgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactccttgtccctgtagctcagtcatcaaagcagaagtcctggctttgctctataaagaattggaaatggtacactacccaaacactcagttcacttgttgagccccagtgcctggaagggaggaaggcctttcttctgtgttaattgccgtagaggctacaggggttagccctggactaaaggcatccttgtcttttgagctattcacctcagtagaaaaggatctaagggaagatcactgtagtttagttctgttgaccttgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttgggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagttttttttttttttttgggttaaaagatggttgtagcatttaaaatggaaaattttctccttggtttgctagtatcttgggtgtattctctgtaagtgtagctcaaataggtcatcatgaaaggttaaaaaagcgaggtggccatgttatgctggtggttaaggccagggcctctccaaccactgtgccactgacttgctgtgtgacctctgggcaagtcacttaacgtataaggtgcctcagttttccttctgttaaaatggggataataatactgacctacctcaaagggcagttttgaggcatgactaatgctttttagaaagcattttgggatccttcagcacaggaattctcaagacctgagtattttttataataggaatgtccaccatgaacttgatacgtccgtgtgtcccagatgctgtcattagtctatatggttctccaagaaactgaatgaatccattggagaagcggtggataactagccagacaaaatttgagaatacataaacaacgcattgccacggaaacatacagaggatgccttttctgtgattgggtgggatttttttccctttttatgtgggatatagtagttacttgtgacaagaataattttggaataatttctattaatatcaactctgaagctaattgtactaatctgagattgtggtgagcagtgacgatgaggagttgtccagggacagagacgtatatgtgactacccatactcccagaaacgccagggatgagggcgctacaggcctcaggccctcaggtactgtcagttgtcccatctgcatggactggatactcagaggtaagtaaaccaagctgtatcttccaggcttctggtttctaaacttcactgaaagaattggatgagacaggatcttccccctcggtgggattggacacccctactcacagtcatgcctgggccctcacttattgcagatctgcctgtgaggggagaatgtgcc

SEQ ID NO: 184 - DNA, 1437 bp, Homo sapiens
  misc_feature: Incyte ID No 464689.59
ttcagaggcagtgatgacttgctgtccaggcagctccctcctgcacactgatggctcaagacaaggctggcaaaccctcccagaaacatctctggcccagaaagcctctctctccctccctctctcatgaggcacagccaagccaagcgctcacgttgagccagtgggccagccacagagcaaaagagggtttattttcagtcccctctctctgggtcagaaccagagggcatgctgaatgccccctgcttacttggtgagggtgccccgcctgagtcagtgctctcagctggcagtgcaatgcttgtagaagtaggaggaaacagttctcactgggaagaagcaagggcaagaacccaagtgcctcacctcgaaaggaggccctgttccctggagtcagggtgaactgcaaagctttggctgagacctgggatttgagataccacaaaccctgctgaacacagtgtctgttcagcaaactaaccagcattccctacagcctagggcagacaatagtatagaagtctggaaaaaaacaaaaacagaatttgagaaccttggaccactcctgtccctgtagctcagtcatcaaagcagaagtctggctttgctctattaagattggaaatgtacactaccaaacactcagtccactgttgagccccagtgctggaagggaggaaggcctttcttctgtgttaattgcgtagaggctacaggggttagcctggactaaaggcatccttgtcttttgagctattcacctcagtagaaaaggatctaagggaagatcactgtagtttagttctgttgacctgtgcacctaccccttggaaatgtctgctggtatttctaattccacaggtcatcagatgcctgcttgataatatataaacaataaaaacaactttcacttcttcctattgtaatcgtgtgccatggatctgatctgtaccatgaccctacataaggctggatggcacctcaggctgagggccccaatgtatgtgtggctgtgggtgtgggtgggagtgtgtctgctgagtaaggaacacgattttcaagattctaaagctcaattcaagtgacacattaatgataaactcagatctgatcaagagtccggatttctaacagtccttgctttggggggtgtgctgacaacttagctcaggtgccttacatcttttctaatcacagtgttgcatatgagcctgccctcactccctctgcagaatccctttgcacctgagaccctactgaagtggctggtagaaaaaggggcctgagtggaggattatcagtatcacgatttgcaggattcccttctgggcttcattctggaaacttttgttagggctgcttttcttaagtgcccacatttgatggagggtggaaataatttgaatgtatttgatttataagtttttttttttt

SEQ ID NO: 185 - DNA, 3583 bp, Homo sapiens
  misc_feature: Incyte ID No 1384719.3
catcctgagttcttttgaagctgatctcaggcatcggattatttcttctgtaaatatttcagaatgtatctctccaagatgagagctcacttaaaagacaattacagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacactggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggaggccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtanatatatatacatatgtttangtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctc

SEQ ID NO: 186 - DNA, 5048 bp, Homo sapiens
  misc_feature: Incyte ID No 407463.1
ataaaaacctactgaccaaggttcgagatgctgccctgtggttggagaccttgtcagacagcagacctgccaaggcttccctctccaccacctcctccattgctgatttcttccttgccttaaccatctgcaactctgtcatggtgtccacaaccaccgagcccaggcagagggtcaccatcaaaccctcaagcaaggctcgtggggacgtccctggagaagattcagcagctcttccagaagttgaagctattgagcctcagccagtcattctcatccactgcaccctctgacacagacctcggggagagcttaggggccaacgtggccaccacagactcggatgagagagatgatgcatctgtgtgcagtggaggtgactccactgatgacggtggctacaggagcagcatgtgggaccagggcgacatcctggagtctgggtcaggcacttccttggaggaggcattggaggccccagccacagacctggccaggcctgagttctgttacgaggctgagagccctgatgaggccgccctggtgcacgctgcccatgcctacagcttcacactagtgtcccggacacctgagcaggtgactgtgcgcctgccccagggcacctgcctcaccttcagcctcctctgcaccctgggctttgactctgtcaggaagagaatgtctgtggttgtgaggcacccactgactggcgagattgttgtctacaccaagggtgctgactcggtcatcatggacctgctggaagacccagcctgcgtacctgacattaatatggaaaagaagctgagaaaaatccgagcccggacccaaaagcatctagacttgtatgcaagagatggcctgcgcacactatgcattgccaagaaggttgtaagcgaagaggacttccggagatgggccagtttccggcgtgaggctgaggcatccctcgacaaccgagatgagcttctcatggaaactgcacagcatctggagaatcaactcaccttacttggagccactgggatcgaagaccggctgcaggaaggagttccagatacgattgccactctgcgggaggctgggatccagctctgggtcctgactggagataagcaggagacagcggtcaacattgcccattcctgcagactgttaaatcagaccgacactgtttataccatcaatacagagaatcaggagacctgtgaatccatcctcaattgtgcattggaagagctaaagcaatttcgtgaactacagaagccagaccgcaagctctttggattccgcttaccttccaagacaccatccatcacctcagaagctgtggttccagaagctggacttggtcatcgatgggaagacattgaactgccatcttccaggaaagctagagaagaagtttctggaattgacccagtattgtcggtccgtcctgtgctgccgctccacgccactccagaagagtatgatagtcaagctggtgcgagacaagttcgctgcgtcatgaccctttccataggatgatggagcaaatgatgtaagcatgattcaagctgctgatattggcaattggaatatctggacaggaaggcatgcaggctgtcatgtccagcgactttgccatcacccgctttaagcatctcaagaagttgctgctcgtgcatggccactggtgttactcgcgcctggccaggatggtggtgtactacctctacaagaacgtgtgctaacgtcaacctgctcttctggtatcagttcttctgtggtttctccagctccaccatgattgattactggcagatgatattcttcaatctcttctttacctgccttgcctcctcttgtctttggagtccttgacaaagacatctctgcagaaacactcctggcattgcctgagctatacaagagtggccagaactctgagtgctataacctgtcgactttctggatttctatggtggatgcattctaccagagcctcatctgtttctttatcccttacctggcctataagggctctgatatagatgtctttacctttgggacaccaatcaacaccatctccctcaccacaatccttttgcaccaggcaatggaaatgaagacatggaccattttccacggagtcgtgctcctcggcagcttcctgatgtactttctggtatccctcctgtacaatgccacctgcgtcatctgcaacagccccaccaatccctattgggtgatggaaggccagctctcaaaccccactttctacctcgtctgctttctcacaccagttgttgctcttctcccaagatactttttcctgtctctgcaaggaacttgtgggaagtctctaatctcaaaagctcagaaaattgacaaactccccccagacaaaagaaacctggaaatccagagttggagaagcagacagaggcctgcccctgtccccgaagtggctcgaccaactcaccacccagtgtcatctatcacaggacaggacttcagtgccagcaccccaaagagctctaaccctcccaagaggaagcatgtggaagagtcagtgctccacgaacagagatgtggcacggagtgcatgagggatgactcatgctcaggggactcctcagctcaactctcatccggggagcacctgctgggacctaacaggataatggcctactcaggaggacagactgatatgtgccggtgctcaaagaggagcagccatcgccgatcccagagttcactgaccatatgaggagctgcagaaatctgtacaaactcaacagaggccacctagtcactggtccacataacccttgaccccttcttcttcatagaggaaacaatgtgccagtcttattcttttcttcaacaaccttgacttccatggaggaagtgctggccccaaggggtctgacacaaagacgggaaacccagtcggcctctagttttctgctgctctcaggcagcacatcttgcaaacagtttggagaaggaggctgtttttgttgaatcgagttctcaaatcggtttagaccaaagccattcttctgaccctctagataagcgtagcctacaacccagtgccgtaagtttccaagattcaagaagtgtatcaacccaggcaatatctcaggatatggaagtttctgggtttatttacccctcagtgcccagagttaaagtttcagaagagacttgtgcacataagggcttcatctcaagtgtattgcagtaatggctgaatcggggttaacatcccttccaggcacagcgagttggttctgctttttgcctgtaagccaaagaaaagccacatctaaaaagctactactaaaagccagaaagaaaagtggatttgaactcagtgtcacagactcttctgagtgttttagggtcacagctagtgtaagaggcatgaagaatagacatgcaaaagggaacgggtgcaccagagacccctgttttggctgacagaccatatgtcccaccagctggggaatctgacaagaggacataggtggcactctttttttaaagctatttattgtatctatttttaaataaaattgcccatcctcattcagctcttagaacaaaagcaaaaaaccctgtaaatcaggagatataagcacatctgcacccagaataggcccatatgatagggcaaccctgagcttaaacaatgacatcttcaagggtagaactaatctgaaacccccttccagcctctggaagacactggcctgcatcagttagagtcagagcaagtgtcacttcacagggaaaagaaggattatatagacttcctatccctagagtttataaatgtcaactatataaaaaaagctcaaaacagtgttaaaggaatgaacagtagaattttaataggctgtccaaagaagccaggtctgctgtgggcaagtatagcctaaccctagtcttgtaaaataagccagaaagggttactgagccaccttaagctagtacctatatagtaggcaaaaagtacagaaatagatgcaataagtgtggtgagtctttgagcctacgagtcatgccaccagccataagttgacctatcacttgagaacctcctcagcaaagatgccagaaaacattcaatcaagttggcaaatgacacagggagctggccctctgaccatcttcctggcaaacctggactggaagggccatttgcagcactgtcctggagctaatacactgtttcactgcctctgccatataatgatgccagcactagccagctggtgggtatttggaggaatcctgcatgaggattgcccaataaggggcaggtacacatacctggcaaagtgatgatgatgtgaattgtttccagtgaggggattgagtcaaaacttggatctcaggtacctcaatttttcccccaatttctggctactactaaaagccagaaagaacagaacagtggcctcaggagatctgagtttgaatccttgctctctaggatgcaggtggcttgaagcagaatgccacacctgcaagttgattagaactgcctttcttcccaggcttgacataggtattaagtcaaaattacatgaaacccagtggtaaaaaagcctctgaaagctgtaacaccctcagtaataacaaaagggatttttatttcacagctaaagggaaaataggtggagaagttaaaaaataatgtctgatcctgttcctaagttccaaactatagccaacactctgatgctgctctttttcttgtaggaccaaccgtcccagtttgcctgggactttctcatttttacagagtcccaaatcctaggaaactggagcaactggtacaactggtcacctactcttgcccctctgtaaatcaagccaactgtgaccatccaatgtgtcatcttacagggaaaagttataaccactattcccctataacataatgctaatgattgtacttagtacatttttatacttttatgatattttactgattggaaatgtcatcctttattaaaaataaacatggttttccgtagttgcctgcc

SEQ ID NO: 187 - DNA, 1273 bp, Homo sapiens
  misc_feature: Incyte ID No 522433CB1
gccgcaacctgcacagccatgcccgggcaagaactcaggacgctgaatggctctcagatgctcctggtgttgctggtgctctcgtggctgccgcatgggggcgccctgtctctggccgaggcgagccgcgcaagtttcccgggaccctcagagttgcacaccgaagactccagattccgagagttgcggaaacgctacgaggacctgctaaccaggctgcgggccaaccagagctgggaagattcgaacaccgacctcgtcccggcccctgcagtccggatactcacgccagaagtgcggctgggatccggcggccacctgcacctgcgtatctctcgggccgcccttcccgaggggctccccgaggcctcccgccttcaccgggctctgttccggctgtccccgacggcgtcaaggtcgtgggacgtgacacgacctctgcggcgtcagctcagccttgcaagaccccaggcgcccgcgctgcacctgcgactgtcgccgccgccgtcgcagtcggaccaactgctggcagaatcttcgtccgcacggccccagctggagttgcacttgcggccgcaagccgccagggggcgccgcagagcgcgtgcgcgcaacggggaccactgtccgctcgggcccgggcgttgctgccgtctgcacacggtccgcgcgtcgctggaagacctgggctgggccgattgggtgctgtcgccacgggaggtgcaagtgaccatgtgcatcggcgcgtgcccgagccagttccgggcggcaaacatgcacgcgcagatcaagacgagcctgcaccgcctgaagcccgacacggtgccagcgccctgctgcgtgcccgccagctacaatcccatggtgctcattcaaaagaccgacaccggggtgtcgctccagacctatgatgacttgttagccaaagactgccactgcatatgagcagtcctggtccttccactgtgcacctgcgcgggggaggcgacctcagttgtcctgccctgtggaatgggctcaaggttcctgagacacccgattcctgcccaaacagctgtatttatataagtctgttatttattattaatttattggggtgaccttcttggggactcgggggctggtctgatggaactgtgtatttatttaaaactctggtgataaaaataaagctgtctgaactgttaaaaaaaaaaaaaaaaaaaaaaaaaaaaatggttgcggccgcaagcttattccctttagtgagggttaattttagcttgcactgacc

SEQ ID NO: 188 - protein, 308 aa, Homo sapiens [sequence omitted]

SEQ ID NO: 189 - DNA, 1712 bp, Homo sapiens
  misc_feature: Incyte ID No 480489.5
atcgcattgcaccaggatgactctgaaatggacttcagttcttctgctgatacatctcagttgttactttagctctgggagttgtggaaaagtgctggtgtgggccgcagaatacagccattggatgaatatgaagacaatcctgaaagagcttgttcagagaggtcatgaggtgactgtactggcatcttcagcttccattctttttgatcccaatgatgcatccactcttaaatttgaagtttatcctacatctttaactaaaactgaatttgagaatatcatcatgcaacaggttaagagatggtcagacattcgaaaagatagcttttggttatatttttcacaagaacaagaaatcctgtgggaattatatgacatatttagaaacttctgtaaagatgtagtttcaaataagaaagttatgaaaaaactacaagagtcaagatttgacatcgtttttgcagatgctgtttttccctgtggtgagctgctggctgcgctacttaacatacggtttgtgtacagtctccgctttactcctggctacacaattgaaaggcacagtggaggactgattttccctccttcctacatacctattgttatgtcaaaattaagtgatcaaatgactttcatggagagggtaaaaaatatgatctatgtgctttattttgacttttggttccaaatgtctgatatgaagaagtgggatcagttttacagtgaagttttaggaagacccactaccttatttgagacaatgggaaaagctgacatatggcttatgcgaaactcctggagttttcaatttcctcatccattcttaccaaacgttgattttgttggaggattccactggcaaacctgccaaacccctacctaaggaaatggaggagtttgtacagagctctggagaaaatggtgttgtggtgttttctctggggtcagtgataagtaacatgacagcagaaagggccaatgtaattgcaacagcccttgccaagatcccacaaaaggttctgtggagatttgacgggaataaaccagatgccttaggtctcaatactcggctgtacaagtggataccccagaatgaccttctaggtcatccaaaaaccagagcttttataactcatggtggagccaatggcatctatgaggcaatctaccatgggatccctatggtgggcattccattgttttttgatcaacctgataacattgctcacatgaaggccaagggagcagctgttagattggacttcaacacaatgtcgagtacagacctgctgaatgcactgaagacagtaattaatgatcctttatataaagagaatattatgaaattatcaagaattcaacatgatcaaccagtaaagcccctggatcgagcagtcttctggattgaatttgtcatgccccacaaaggagccaaacaccttcgagttgcagcccatgacctcacctggttccagtaccactctttggatgtgattgggtttctgctggcctgtgtggcaactgtgatatttatcatcacaaagttttgtctgttttgtttctggaagtttgctagaaaagggaagaagggaaaaagagattagttatgtctgacatttgaagctggaaaaccagatagataggacaacttcagtttattccagcaagaaagaaaagattgttatgcaagatttctttcttcctgtgac

SEQ ID NO: 190 - DNA, 624 bp, Homo sapiens
  misc_feature: Incyte ID No 480489.2
aaaaaattaactttctcacctgaccttccatttctgctttaaaaatgtttgtcaatgagaaaagtccaatttaaaagccaaactatctatgataactcaaattaaaatacacaaattctctgtcaattctttgacatttattttgaattatttgatgctttaaaagcctttcatagacttgatatgtacaggcaaattaacttactttcagtgttggtatccttatttttatccttcagatataaagagaatattatgaaattatcaagaattcaacatgatcaaccagtaaagcccctggatcgagcagtcttctggattgaatttgtcatgccccacaaaggagccaaacaccttcgagttgcagcccatgacctcacctggttccagtaccactctttggatgtgattgggtttctgctggcctgtgtggcaactgtgatatttatcatcacaaagttttgtctgttttgtttctggaagtttgctagaaaagggaagaagggaaaaagagattagttatgtctgacatttgaagctggaaaaccagatagataggacaacttcagtttattccagcaagaaagaaaagattgttatgcaagatttctttcttcctgtgac

SEQ ID NO: 191 - DNA, 3111 bp, Homo sapiens
  misc_feature: Incyte ID No 1737775CB1
cggctcgaggaaatcacagggagatgtacagcaatggggccatttaagagttctgtgttcatcttgattcttcaccttctagaaggggccctgagtaattcactcattcagctgaacaacaatggctatgaaggcattgtcgttgcaatcgaccccaatgtgccagaagatgaaacactcattcaacaaataaaggacatggtgacccaggcatctctgtatctgtttgaagctacaggaaagcgattttatttcaaaaatgttgccattttgattcctgaaacatggaagacaaaggctgactatgtgagaccaaaacttgagacctacaaaaatgctgatgttctggttgctgagtctactcctccaggtaatgatgaaccctacactgagcagatgggcaactgtggagagaagggtgaaaggatccacctcactcctgatttcattgcaggaaaaaagttagctgaatatggaccacaaggtagggcatttgtccatgagtgggctcatctacgatggggagtatttgacgagtacaataatgatgagaaattctacttatccaatggaagaatacaagcagtaagatgttcagcaggtattactggtacaaatgtagtaaagaagtgtcagggaggcagctgttacaccaaaagatgcacattcaataaagtaacaggactctatgaaaaaggatgtgagtttgttctccaatcccgccagacggagaaggcttctataatgtttgcacaacatgttgattctatagttgaattctgtacagaacaaaaccacaacaaagaagctccaaacaagcaaaatcaaaaatgcaatctccgaagcacatgggaagtgatccgtgattctgaggactttaagaaaaccactcctatgacaacacagccaccaaatcccaccttctcattgctgcagattggacaaagaattgtgtgtttagtccttgacaaatctggaagcatggcgactggtaaccgcctcaatcgactgaatcaagcaggccagcttttcctgctgcagacagttgagctggggtcctgggttgggatggtgacatttgacagtgctgcccatgtacaaagtgaactcatacagataaacagtggcagtgacagggacacactcgccaaaagattacctgcagcagcttcaggagggacgtccatctgcagcgggcttcgatcggcatttactgtgattaggaagaaatatccaactgatggatctgaaattgtgctgctgacggatggggaagacaacactataagtgggtgctttaacgaggtcaaacaaagtggtgccatcatccacacagtcgctttggggccctctgcagctcaagaactagaggagctgtccaaaatgacaggaggtttacagacatatgcttcagatcaagttcagaacaatggcctcattgatgcttttggggccctttcatcaggaaatggagctgtctctcagcgctccatccagcttgagagtaagggattaaccctccagaacagccagtggatgaatggcacagtgatcgtggacagcaccgtgggaaaggacactttgtttcttatcacctggacaacgcagcctccccaaatccttctctgggatcccagtggacagaagcaaggtggctttgtagtggacaaaaacaccaaaatggcctacctccaaatcccaggcattgctaaggttggcacttggaaatacagtctgcaagcaagctcacaaaccttgaccctgactgtcacgtcccgtgcgtccaatgctaccctgcctccaattacagtgacttccaaaacgaacaaggacaccagcaaattccccagccctctggtagtttatgcaaatattcgccaaggagcctccccaattctcagggccagtgtcacagccctgattgaatcagtgaatggaaaaacagttaccttggaactactggataatggagcaggtgctgatgctactaaggatgacggtgtctactcaaggtatttcacaacttatgacacgaatggtagatacagtgtaaaagtgcgggctctgggaggagttaacgcagccagacggagagtgataccccagcagagtggagcactgtacatacctggctggattgagaatgatgaaatacaatggaatccaccaagacctgaaattaataaggatgatgttcaacacaagcaagtgtgtttcagcagaacatcctcgggaggctcatttgtggcttctgatgtcccaaatgctcccatacctgatctcttcccacctggccaaatcaccgacctgaaggcggaaattcacgggggcagtctcattaatctgacttggacagctcctggggatgattatgaccatggaacagctcacaagtatatcattcgaataagtacaagtattcttgatctcagagacaagttcaatgaatctcttcaagtgaatactactgctctcatcccaaaggaagccaactctgaggaagtctttttgtttaaaccagaaaacattacttttgaaaatggcacagatcttttcattgctattcaggctgttgataaggtcgatctgaaatcagaaatatccaacattgcacgagtatctttgtttattcctccacagactccgccagagacacctagtcctgatgaaacgtctgctccttgtcctaatattcatatcaacagcaccattcctggcattcacattttaaaaattatgtggaagtggataggagaactgcagctgtcaatagcctagggctgaatttttgtcagataaataaaataaatcattcatccttttttttgattataaaattttctaaaatgtattttagacttcctgtagggggcgatatactaaatgtatatagtacatttatactaaatgtattcctgtagggggcgatatactaaatgtattttagacttcctgtagggggcgataaaataaaatgctaaacaactgggtatacatgcataaaaactatccattcaaacccaaaaatttaataatcattgagtcttttattaatgaatttgaatactagaaagaaacagggcttgcatcaataaatggaagtatgagtgt

SEQ ID NO: 192 - protein, 914 aa, Homo sapiens [sequence omitted]

SEQ ID NO: 193 - DNA, 1714 bp, Homo sapiens
  misc_feature: Incyte ID No 088078CB1
aactggaaaacaagcattgcattgcaccaggatgtctgtgaaatggacttcagtaattttgctaatacaactgagcttttgctttagctctgggaattgtggaaaggtgctggtgtgggcagcagaatacagccattggatgaatataaagacaatcctggatgagcttattcagagaggtcatgaggtgactgtactggcatcttcagcttccattctttttgatcccaacaactcatccgctcttaaaattgaaatttatcccacatctttaactaaaactgagttggagaatttcatcatgcaacagattaagagatggtcagaccttccaaaagatacattttggttatatttttcacaagtacaggaaatcatgtcaatatttggtgacataactagaaagttctgtaaagatgtagtttcaaataagaaatttatgaaaaaagtacaagagtcaagatttgacgtcatttttgcagatgctatttttccctgtagtgagctgctggctgagctatttaacataccctttgtgtacagtctcagcttctctcctggctacacttttgaaaagcatagtggaggatttattttccctccttcctacgtacctgttgttatgtcagaattaactgatcaaatgactttcatggagagggtaaaaaatatgatctatgtgctttactttgacttttggttcgaaatatttgacatgaagaagtgggatcagttttatagtgaagttctaggaagacccactacgttatctgagacaatggggaaagctgacgtatggcttattcgaaactcctggaattttcagtttcctcatccactcttaccaaatgttgattttgttggaggactccactgcaaacctgccaaacccctgcctaaggaaatggaagactttgtacagagctctggagaaaatggtgttgtggtgttttctctggggtcaatggtcagtaacatgacagaagaaagggccaacgtaattgcatcagccctggcccagatcccacaaaaggttctgtggagatttgatgggaataaaccagataccttaggtctcaatactcggctctacaagtggataccccagaatgaccttctaggtcatccaaagaccagagcttttataactcatggtggagccaatggcatctacgaggcaatctaccatgggatccctatggtggggattccattgtttgctgatcaacctgataacattgctcacatgaaggccaggggagcagctgttagagtggacttcaacacaatgtcgagtacagacttgctgaatgcattgaagagagtaattaatgatccttcatataaagagaatgttatgaaattatcaagaattcaacatgatcaaccagtgaagcccctggatcgagcagtcttctggattgaatttgtcatgcgccacaaaggagctaaacaccttcgggttgcagcccacgacctcacctggttccagtaccactctttggatgtgattgggttcctgctggtctgtgtggcaactgtgatatttatcgtcacaaaatgttgtctgttttgtttctggaagtttgctagaaaagcaaagaagggaaaaaatgattagttatatctgagatttgaagctggaaaacctgataggtgagactacttcagtttattccagcaagaaagattgtgatgcatgatgtcgatcttc

SEQ ID NO: 194 - protein, 529 aa, Homo sapiens
  misc_feature: Incyte ID No 088078CD1
MSVKWTSVILLIQLSFCFSSGNCGKVLVWAAEYSHWMNIKTILDELIQRGHEVTVLASSASILFDPNNSSALKIEIYPTSLTKTELENFIMQQIKRWSDLPKDTFWLYFSQVQEIMSIFGDITRKFCKDVVSNKKFMKKVQESRFDVIFADAIFPCSELLAELFNIPFVYSLSFSPGYTFEKHSGGFIFPPSYVPVVMSELTDQMTFMERVKNMIYVLYFDFWFEIFDMKKWDQFYSEVLGRPTTLSETMGKADVWLIRNSWNFQFPHPLLPNVDFVGGLHCKPAKPLPKEMEDFVQSSGENGVVVFSLGSMVSNMTEERANVIASALAQIPQKVLWRFDGNKPDTLGLNTRLYKWIPQNDLLGHPKTRAFITHGGANGIYEAIYHGIPMVGIPLFADQPDNIAHMKARGAAVRVDFNTMSSTDLLNALKRVINDPSYKENVMKLSRIQHDQPVKPLDRAVFWIEFVMRHKGAKHLRVAAHDLTWFQYHSLDVIGFLLVCVATVIFIVTKCCLFCFWKFARKAKKGKND

(19) United States
(12) Patent Application Publication
(10) Pub. No.: US 2002/0160382 A1
(43) Pub. Date: Oct. 31, 2002

(54) Genes expressed in colon cancer

(76) Inventors: Amy W. Lasek, Oakland, CA (US); David A. Jones, Salt Lake City, UT (US)

Correspondence Address: INCYTE GENOMICS, INC., 3160 Porter Drive, Palo Alto, CA, 94304, US

(21) Appl. No.: 09/981,353

(22) Filed: Oct. 11, 2001

Related U.S. Application Data
(60) Provisional application No. 60/239,841, filed on Oct. 11, 2000.

(51) Int. Cl. C12Q 1/68; G01N 33/574; C07H 21/04; C12P 21/04; C12N 9/00

(52) U.S. Cl. 435/6; 435/7.23; 435/70.21; 435/69.1; 435/183; 536/23.2

(57) ABSTRACT

The present invention relates to a combination comprising a plurality of cDNAs which are differentially expressed in colon cancer, or in a precancerous condition of the colon and which may be used in their entirety or in part as to diagnose, to stage to treat or to monitor the treatment of a subject with a colon cancer.

### FIELD OF THE INVENTION

The present invention relates to a combination comprising a plurality of cDNAs which are differentially expressed in colon cancer and in premalignant conditions of the colon and which may be used entirely or in part to diagnose, to stage, to treat, or to monitor the progression or treatment of colon cancer.

### BACKGROUND OF THE INVENTION

Array technology can provide a simple way to explore the expression of a single polymorphic gene or the expression profile of a large number of related or unrelated genes. When the expression of a single gene is examined, arrays are employed to detect the expression of a specific gene or its variants. When an expression profile is examined, arrays provide a platform for examining which genes are tissue specific, carrying out housekeeping functions, parts of a signaling cascade, or specifically related to a particular genetic predisposition, condition, disease, or disorder.

The potential application of gene expression profiling is particularly relevant to improving diagnosis, prognosis, and treatment of disease. For example, both the levels and sequences expressed in tissues from subjects with colon cancer may be compared with the levels and sequences expressed in normal tissue.

Colorectal cancer is the fourth most common cancer and the second most common cause of cancer death in the United States with approximately 130,000 new cases and 55,000 deaths per year. Colon and rectal cancers share many environmental risk factors and both are found in individuals with specific genetic syndromes. (See Potter (1999) J Natl Cancer Institute 91:916-932 for a review of colorectal cancer.) Colon cancer is the only cancer that occurs with approximately equal frequency in men and women, and the five-year survival rate following diagnosis of colon cancer is around 55% in the United States (Ries et al. (1990) National Institutes of Health, DHHS Publ No. (NIH)90-2789).

Colon cancer is causally related to both genes and the environment. Several molecular pathways have been linked to the development of colon cancer, and the expression of key genes in any of these pathways may be lost by inherited or acquired mutation or by hypermethylation. There is a particular need to identify genes for which changes in expression may provide an early indicator of colon cancer or a predisposition for the development of colon cancer.

For example, it is well known that abnormal patterns of DNA methylation occur consistently in human tumors and include, simultaneously, widespread genomic hypomethylation and localized areas of increased methylation. In colon cancer in particular, it has been found that these changes occur early in tumor progression such as in premalignant polyps that precede colon cancer. Indeed, DNA methyltransferase, the enzyme that performs DNA methylation, is significantly increased in histologically normal mucosa from patients with colon cancer or the benign polyps that precede cancer, and this increase continues during the progression of colonic neoplasms (Wafik et al. (1991) Proc Natl Acad Sci USA 88:3470-3474). Increased DNA methylation occurs in G+C rich areas of genomic DNA termed “CpG islands” that are important for maintenance of an “open” transcriptional conformation around genes, and that hypermethylation of these regions results in a “closed” conformation that silences gene transcription. It has been suggested that the silencing or downregulation of differentiation genes by such abnormal methylation of CpG islands may prevent differentiation in immortalized cells (Anteguera et al. (1990) Cell 62:503-514).

Familial Adenomatous Polyposis (FAP) is a rare autosomal dominant syndrome that precedes colon cancer and is caused by an inherited mutation in the adenomatous polyposis coli (APC) gene. FAP is characterized by the early development of multiple colorectal adenomas that progress to cancer at a mean age of 44 years. The APC gene is a part of the APC-β-catenin-Tcf (T-cell factor) pathway. Impairment of this pathway results in the loss of orderly replication, adhesion, and migration of colonic epithelial cells that results in the growth of polyps. A series of other genetic changes follow activation of the APC-β-catenin-Tcf pathway and accompanies the transition from normal colonic mucosa to metastatic carcinoma. These changes include mutation of the K-Ras proto-oncogene, changes in methylation patterns, and mutation or loss of the tumor suppressor genes p53 and Smad4/DPC4. While the inheritance of a mutated APC gene is a rare event, the loss or mutation of APC and the consequent effects on the APC-β-catenin-Tcf pathway is believed to be central to the majority of colon cancers in the general population.

Hereditary nonpolyposis colorectal cancer (HNPCC) is another inherited autosomal dominant syndrome with a less well defined phenotype than FAP. HNPCC, which accounts for about 2% of colorectal cancer cases, is distinguished by the tendency to early onset of cancer and the development of other cancers, particularly those involving the endometrium, urinary tract, stomach and biliary system. HNPCC results from the mutation of one or more genes in the DNA mis-match repair (MMR) pathway. Mutations in two human MMR genes, MSH2 and MLH1, are found in a large majority of HNPCC families identified to date. The DNA MMR pathway identifies and repairs errors that result from the activity of DNA polymerase during replication. Furthermore, loss of MMR activity contributes to cancer progression through accumulation of other gene mutations and deletions, such as loss of the BAX gene which controls apoptosis, and the TGFβ receptor II gene which controls cell growth. Because of the potential for irreparable damage to DNA in an individual with a DNA MMR defect, progression to carcinoma is more rapid than usual.

Although ulcerative colitis is a minor contributor to colon cancer, affected individuals have about a 20-fold increase in risk for developing cancer. Progression is characterized by loss of the p53 gene which may occur early, appearing even in histologically normal tissue. The progression of the disease from ulcerative colitis to dysplasia/carcinoma without an intermediate polyp state suggests a high degree of mutagenic activity resulting from the exposure of proliferating cells in the colonic mucosa to the colonic contents.

Almost all colon cancers arise from cells in which the estrogen receptor (ER) gene has been silenced. The silencing of ER gene transcription is age related and linked to hypermethylation of the ER gene (Issa et al. (1994) Nature Genetics 7:536-540). Introduction of an exogenous ER gene into cultured colon carcinoma cells results in marked growth suppression. The connection between loss of the ER protein in colonic epithelial cells and the consequent development of cancer has not been established.

Clearly there are a number of genetic alterations associated with colon cancer and with the development and progression of the disease, particularly the downregulation or deletion of genes, that potentially provide early indicators of cancer development, and which may also be used to monitor disease progression or provide possible therapeutic targets. The specific genes affected in a given case of colon cancer depend on the molecular progression of the disease. Identification of additional genes associated with colon cancer and the precancerous state would provide more reliable diagnostic patterns associated with the development and progression of the disease.

The present invention provides for a composition comprising a plurality of cDNAs for use in detecting changes in expression of genes encoding proteins associated with colon cancer. Such a composition satisfies a need in the art by providing a set of differentially expressed genes which may be used entirely or in part in the diagnosis, prognosis or treatment of colon cancer.

### SUMMARY

The present invention provides a combination comprising a plurality of cDNAs and their complements which are differentially expressed in precancerous colon polyps and colon cancer and which are selected from SEQ ID NOs:1-3, 5, 6, 8-10,12, 14, 15, 17, 18, 20, 22, 24, 26-29, 31, 33, 34, 36-39, 41-43, 45-47, 49, 51, 53. 55-58, 60, 62, 64, 66, 67, 69, 71, 72, 74-79, 81, 83-86, 88, 89, 91, 92, 94, 96, 97, 99, 100, 102-104, 106, 107, 109, 111, 112, 114, 116, 118, 119, 121, 123-126, 128, 130, 131-137, 139, 140, 142-151, 153-157, 159, 160, 162-165, 167-172, 174, 176, 177, 179-181, 183-187, 189-191, and 193 as presented in the Sequence Listing. In one aspect, the combination is useful to diagnose a precancerous or cancerous condition in colon. In another aspect, the combination is immobilized on a substrate.

The invention also provides a combination comprising a subset of these cDNAs and their complements which are differentially expressed in colon cancer relative to colon polyps or normal colon tissue and which are selected from SEQ ID NOs:172, 174, 176, 177, 179-181, 183-187, 189-191, and 193. In one aspect, the combination is useful to diagnose a colon cancer or the progression of a colon disorder from colon polyps to colon cancer.

The invention further provides a high throughput method to detect differential expression of one or more of the cDNAs of the combination. The method comprises hybridizing the substrate comprising the combination with the nucleic acids of a sample, thereby forming one or more hybridization complexes, detecting the hybridization complexes, and comparing the hybridization complexes with those of a standard, wherein differences in the size and signal intensity of each hybridization complex indicates differential expression of nucleic acids in the sample. In one aspect, the sample is from a subject with colon cancer and differential expression determines an early, mid, and late stage of that disorder.

The invention further provides a high throughput method of screening a library or plurality of molecules or compounds to identify a ligand. The method comprises combining the substrate comprising the combination with a library or plurality of molecules or compounds under conditions to allow specific binding and detecting specific binding, thereby identifying a ligand. The library or plurality of molecules or compounds are selected from DNA molecules, RNA molecules, peptide nucleic acid molecules, mimetics, peptides, transcription factors, repressors, and other regulatory proteins. The invention additionally provides a method for purifying a ligand, the method comprising combining a cDNA of the invention with a sample under conditions which allow specific binding, recovering the bound cDNA, and separating the cDNA from the ligand, thereby obtaining purified ligand.

The invention still further provides an isolated cDNA selected from SEQ ID NOs:12, 41, 71, 74, 154,162, 167, 170, and 177 as presented in the Sequence Listing. The invention also provides a vector comprising the cDNA, a host cell comprising the vector, and a method for producing a protein comprising culturing the host cell under conditions for the expression of a protein and recovering the protein from the host cell culture.

The present invention provides a purified protein encoded and produced by a cDNA of the invention. The invention also provides a high-throughput method for using a protein to screen a library or a plurality of molecules or compounds to identify a ligand. The method comprises combining the protein or a portion thereof with the library or plurality of molecules or compounds under conditions to allow specific binding and detecting specific binding, thereby identifying a ligand which specifically binds the protein. A library or plurality of molecules or compounds are selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents. The invention further provides for using a protein to purify a ligand. The method comprises combining the protein or a portion thereof with a sample under conditions to allow specific binding, recovering the bound protein, and separating the protein from the ligand, thereby obtaining purified ligand. The invention still further provides a composition comprising the protein and a pharmaceutical carrier.

The invention also provides methods for using a protein to prepare and purify polyclonal and monoclonal antibodies which specifically bind the protein. The method for preparing a polyclonal antibody comprises immunizing a animal with protein under conditions to elicit an antibody response, isolating animal antibodies, attaching the protein to a substrate, contacting the substrate with isolated antibodies under conditions to allow specific binding to the protein, dissociating the antibodies from the protein, thereby obtaining purified polyclonal antibodies. The method for preparing and purifying monoclonal antibodies comprises immunizing a animal with a protein under conditions to elicit an antibody response, isolating antibody producing cells from the animal, fusing the antibody producing cells with immortalized cells in culture to form monoclonal antibody producing hybridoma cells, culturing the hybridoma cells, and isolating from culture monoclonal antibodies which specifically bind the protein.

The invention provides a purified antibody that specifically binds a protein expressed in colon cancer. The invention also provides a method for using an antibody to detect expression of a protein in a sample comprising combining the antibody with a sample under conditions which allow the formation of antibody:protein complexes and detecting complex formation, wherein complex formation indicates expression of the protein in the sample.

### DESCRIPTION OF THE SEQUENCE LISTING AND TABLES

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

The Sequence Listing is a compilation of cDNAs obtained by sequencing and extension of clone inserts. Each sequence is identified by a sequence identification number (SEQ ID NO) and by the template number (TEMPLATE ID) from which it was obtained.

Table 1 lists the differential expression of clones representing the cDNAs of the present invention that are differentially expressed in both colon polyps and colon cancer relative to normal colon tissue. Column 1 lists the Incyte cDNA Clone ID, and columns 2-11 list the differential expression values observed in colon tissue samples from patients with colon polyps (columns 2-4) and colon cancer (columns 5-11).

Table 2 lists the differential expression values of clones representing cDNAs that are differentially expressed in colon polyps and colon cancer relative to normal colon in which the expression in colon cancer is found to be statistically more significant than in colon polyps. Column 1 lists the Incyte Clone ID, columns 2-9 list the differential expression values in colon polyps (columns 2-4) and colon cancer (columns 5-9), and column 10 lists the value of the student t-test for the significance between the expression in colon polyps versus colon cancer.

Table 3 links the differentially expressed clones on a microarray with Incyte cDNA templates. Columns 1 and 2 show the SEQ ID NO and TEMPLATE ID, respectively. Column 3 shows the CLONE ID and columns 4 and 5 show the first residue (START) and last residue (STOP) encompassed by the clone on the template.

Table 4 shows Incyte nucleotide templates presented in the Sequence Listing and the corresponding protein templates encoded by these cDNAs, also presented in the sequence Listing. Columns 1 and 2 show the SEQ ID NO and the Nucleotide Template ID, respectively, and columns 3 and 4 show the corresponding SEQ ID NO and Protein Template ID, respectively.

Table 5 shows the annotation of both nucleotide and protein Template IDs of the invention to sequences in GenBank. Columns 1 and 2 show the SEQ ID NO and Template ID, respectively. Columns 3, 4, and 5 show the GenBank hit (GI Number), probability score (E-value), and functional annotation, respectively, as determined by BLAST analysis (version 1.4 using default parameters; Altschul (1993) J Mol Evol 36: 290-300; Altschul et al. (1990) J Mol Biol 215:403-410) of the cDNA against GenBank (release 116; National Center for Biotechnology Information (NCBI), Bethesda Md.).

Table 6 shows Pfam annotations of the cDNAs and proteins of the present invention. Columns 1 and 2 show the SEQ ID NO and TEMPLATE ID, respectively. Columns 3 and 4 show the first residue (START), last residue (STOP), respectively, for the segment of the cDNA or protein identified by Pfam analysis. Column 5 shows the reading frame for cDNA sequences. Columns 6 and 7 show the Pfam hit and Pfam description, respectively, corresponding to the polypeptide domain encoded by the cDNA segment or found in the protein sequence, and column 8 shows the E-value for the annotation.

Table 7 shows signal peptide and transmembrane regions predicted within the cDNAs of the present invention and in the proteins of the invention. Columns 1 and 2 show the SEQ ID NO and TEMPLATE ID, respectively. Columns 3 and 4 show the first residue (START), last residue (STOP), respectively, for the segment of the cDNA or the protein identified as a signal peptide or transmembrane region, and column 5 shows the reading frame for cDNA sequences. Column 6 identifies the polypeptide region as either a signal peptide (SP) or transmembrane (TM) domain.

### DESCRIPTION OF THE INVENTION

Definitions

“Array” refers to an ordered arrangement of at least two cDNAs on a substrate. At least one of the cDNAs represents a control or standard sequence, and the other, a cDNA of diagnostic interest. The arrangement of from about two to about 40,000 cDNAs on the substrate assures that the size and signal intensity of each labeled hybridization complex formed between a cDNA and a sample nucleic acid is individually distinguishable.

The “complement” of a nucleic acid molecule of the Sequence Listing refers to a nucleotide sequence which is completely complementary over the full length of the sequence and which will hybridize to the nucleic acid molecule under conditions of high stringency.

“cDNA” refers to a chain of nucleotides, an isolated polynucleotide, nucleic acid molecule, or any fragment or complement thereof. It may have originated recombinantly or synthetically, be double-stranded or single-stranded, coding and/or noncoding, an exon with or without an intron from a genomic DNA molecule, and purified or combined with carbohydrate, lipids, protein or inorganic elements or substances. Preferably, the cDNA is from about 400 to about 10,000 nucleotides.

The phrase “cDNA encoding a protein” refers to a nucleic acid sequence that closely aligns with sequences which encode conserved regions, motifs or domains that were identified by employing analyses well known in the art. These analyses include BLAST (Basic Local Alignment Search Tool; Altschul (1993) J Mol Evol 36: 290-300; Altschulet al. (1990) J Mol Biol 215:403-410) which provides identity within the conserved region. Brenner et al. (1998; Proc Natl Acad Sci 95:6073-6078) who analyzed BLAST for its ability to identify structural homologs by sequence identity found 30% identity is a reliable threshold for sequence alignments of at least 150 residues and 40% is a reasonable threshold for alignments of at least 70 residues (Brenner et al., page 6076, column 2).

“Derivative” refers to a cDNA or a protein that has been subjected to a chemical modification. Derivatization of a cDNA can involve substitution of a nontraditional base such as queosine or of an analog such as hypoxanthine. These substitutions are well known in the art. Derivatization of a protein involves the replacement of a hydrogen by an acetyl, acyl, alkyl, amino, formyl, or morpholino group. Derivative molecules retain the biological activities of the naturally occurring molecules but may confer advantages such as longer lifespan or enhanced activity.

“Differential expression” refers to an increased or upregulated or a decreased or downregulated expression as detected by absence, presence, or at least two-fold change in the amount of transcribed messenger RNA or translated protein in a sample.

“Disorder” refers to conditions or diseases of the colon, including colon cancer and precancerous conditions such as premalignant polyps.

“Fragment” refers to a chain of consecutive nucleotides from about 200 to about 700 base pairs in length. Fragments may be used in PCR or hybridization technologies to identify related nucleic acid molecules and in binding assays to screen for a ligand. Nucleic acids and their ligands identified in this manner are useful as therapeutics to regulate replication, transcription or translation.

A “hybridization complex” is formed between a cDNA and a nucleic acid of a sample when the purines of one molecule hydrogen bond with the pyrimidines of the complementary molecule, e.g., 5′-A-G-T-C-3′ base pairs with 3′-T-C-A-G-5′. The degree of complementarity and the use of nucleotide analogs affect the efficiency and stringency of hybridization reactions.

“Identity” as applied to sequences, refers to the quantification (usually percentage) of nucleotide or residue matches between at least two sequences aligned using a standardized algorithm such as Smith-Waterman alignment (Smith and Waterman (1981) J Mol Biol 147:195-197), CLUSTALW (Thompson et al. (1994) Nucleic Acids Res 22:4673-4680), or BLAST2 (Altschul et al. (1997) supra). BLAST2 may be used in a standardized and reproducible way to insert gaps in one of the sequences in order to optimize alignment and to achieve a more meaningful comparison between them. “Similarity” as applied to proteins uses the same algorithms but takes into account conservative substitutions of nucleotides or residues.

“Ligand” refers to any agent, molecule, or compound which will bind specifically to a complementary site on a cDNA molecule or polynucleotide, or to an epitope or a protein. Such ligands stabilize or modulate the activity of polynucleotides or proteins and may be composed of inorganic or organic substances including nucleic acids, proteins, carbohydrates, fats, and lipids.

“Oligonucleotide” refers a single stranded molecule from about 18 to about 60 nucleotides in length which may be used in hybridization or amplification technologies or in regulation of replication, transcription or translation. Substantially equivalent terms are amplimer, primer, and oligomer.

“Portion” refers to any part of a protein used for any purpose which retains at least one biological or antigenic characteristic of a native protein; but especially, to an epitope for the screening of ligands or for the production of antibodies.

“Post-translational modification” of a protein can involve lipidation, glycosylation, phosphorylation, acetylation, racemization, proteolytic cleavage, and the like. These processes may occur synthetically or biochemically. Biochemical modifications will vary by cellular location, cell type, pH, enzymatic milieu, and the like.

“Probe” refers to a cDNA that hybridizes to at least one nucleic acid molecule in a sample. Where targets are single stranded, probes are complementary single strands. Probes can be labeled with reporter molecules for use in hybridization reactions including Southern, northern, in situ, dot blot, array, and like technologies or in screening assays.

“Protein” refers to a polypeptide or any portion thereof. An “oligopeptide” is an amino acid sequence from about five residues to about 15 residues that is used as part of a fusion protein to produce an antibody.

“Purified” refers to any molecule or compound that is separated from its natural environment and is preferably 60% free, and more preferably 90% free from other components with which it is naturally associated.

“Sample” is used in its broadest sense as containing nucleic acids, proteins, antibodies, and the like. A sample may comprise a bodily fluid; the soluble fraction of a cell preparation, or an aliquot of media in which cells were grown; a chromosome, an organelle, or membrane isolated or extracted from a cell; genomic DNA, RNA, or cDNA in solution or bound to a substrate; a cell; a tissue or tissue biopsy; a tissue print; a fingerprint, buccal cells, skin, or hair; and the like.

“Specific binding” refers to a special and precise interaction between two molecules which is dependent upon their structure, particularly their molecular side groups. For example, the intercalation of a regulatory protein into the major groove of a DNA molecule, the hydrogen bonding along the backbone between two single stranded nucleic acids, or the binding between an epitope of a protein and an agonist, antagonist, or antibody.

“Substrate” refers to any rigid or semi-rigid support to which cDNAs or proteins are bound and includes membranes, filters, chips, slides, wafers, fibers, magnetic or nonmagnetic beads, gels, capillaries or other tubing, plates, polymers, and microparticles with a variety of surface forms including wells, trenches, pins, channels and pores.

“Variant” refers to molecules that are recognized variations of a cDNA or a protein encoded by the cDNA. Splice variants may be determined by BLAST score, wherein the score is at least 100, and most preferably at least 400. Allelic variants have a high percent identity to the cDNAs and may differ by about three bases per hundred bases. “Single nucleotide polymorphism” (SNP) refers to a change in a single base as a result of a substitution, insertion or deletion. The change may be conservative (purine for purine) or non-conservative (purine to pyrimidine) and may or may not result in a change in an encoded amino acid.

The Invention

The present invention provides for a combination comprising a plurality of cDNAs or their complements, SEQ ID NOs:1-3, 5, 6, 8-10,12, 14, 15, 17, 18, 20, 22, 24, 26-29, 31, 33, 34, 36-39, 41-43, 45-47, 49, 51, 53. 55-58, 60, 62, 64, 66, 67, 69, 71, 72, 74-79, 81, 83-86, 88, 89, 91, 92, 94, 96, 97, 99, 100, 102-104, 106, 107, 109, 111, 112, 114, 116, 118, 119, 121, 123-126, 128, 130, 131-137, 139, 140, 142-151, 153-157, 159, 160, 162-165, 167-172, 174, 176, 177, 179-181, 183-187, 189-191, and 193 which may be used on a substrate to diagnose, to stage, to treat or to monitor the progression or treatment of colon cancer. These cDNAs represent known and novel genes differentially expressed in colon polyps and colon cancer. SEQ ID NOs:12, 41, 71, 74,154,162, 167, 170, and 177 represent novel cDNAs associated with colon cancer. Since the novel cDNAs were identified solely by their differential expression, it is not essential to know a priori the name, structure, or function of the gene or it's encoded protein. The usefulness of the novel cDNAs exists in their immediate value as diagnostics for colon cancer.

Table 1 shows cDNA clones on an array having at least a 2-fold increase (upregulated) or decrease (downregulated, indicated by a minus sign) in at least 50% of the samples tested from patients with either colon polyps or colon cancer compared with normal colon. Column 1 shows the Incyte Clone ID and columns 2-4 show differential expression values from patients with colon polyps, while columns 5-11 show values from patients with colon cancer. These genes are useful in diagnosing a precancerous condition in colon, or the presence of colon cancer.

Table 2 shows cDNA clones on an array that are differentially expressed in colon cancer relative to colon polyps. Column 1 shows the Incyte Clone ID and columns 2-4 show the differential expression values from patients with colon polyps, while columns 5-9 show the values from patients with colon cancer. Column 10 shows the P value for a t-test comparing colon polyps with colon tumor samples. Clones were selected on the basis of a P value in the t-test of less than or equal to 0.05, indicating a confidence level of at least 95% that the gene was differentially expressed in colon tumors to a greater or lesser extent than in colon polyps. These genes are useful in diagnosing colon cancer or monitoring the progression of a colon disorder from premalignant colon polyps to colon cancer.

Tables 3 and 4 further link the differentially expressed cDNA clones to full-length genes and to proteins in the Incyte database, and Table 5 provides the annotation of these sequences to known proteins in GenBank. Tables 6 and 7 provide further identification of encoded protein sequences by Pfam and the presence of signal peptide or transmembrane regions. Of particular note in Table 5 is SEQ ID NO:72, Incyte Template ID 1808144CB 1 that is identified as a human mucosa associated mRNA (DRA; down-regulated in adenoma), g291963, a gene known to be down-regulated in colon adenomas and adenocarcinomas.

The cDNAs of the invention define a differential expression pattern for colon cancer or for a premalignant condition leading to colon cancer. Experimentally, differential expression of the cDNAs can be evaluated by methods including, but not limited to, differential display by spatial immobilization or by gel electrophoresis, genome mismatch scanning, representational discriminant analysis, clustering, transcript imaging and array technologies. These methods may be used alone or in combination.

The combination may be arranged on a substrate and hybridized with tissues from subjects with diagnosed colon disorders to identify those sequences which are differentially expressed in either colon cancer or premalignant colon polyps. This allows identification of those sequences of highest diagnostic and potential therapeutic value. In one embodiment, an additional set of cDNAs, such as cDNAs encoding signaling molecules, are arranged on the substrate with the combination. Such combinations may be useful in the elucidation of pathways which are affected in a particular disorder or to identify new, coexpressed, candidate, therapeutic molecules.

In another embodiment, the combination can be used for large scale genetic or gene expression analysis of a large number of novel, nucleic acid molecules. These samples are prepared by methods well known in the art and are from mammalian cells or tissues which are in a certain stage of development; have been treated with a known molecule or compound, such as a cytokine, growth factor, a drug, and the like; or have been extracted or biopsied from a mammal with a known or unknown condition, disorder, or disease before or after treatment. The sample nucleic acid molecules are hybridized to the combination for the purpose of defining a novel gene profile associated with that developmental stage, treatment, or disorder.

cDNAs and Their Uses

cDNAs can be prepared by a variety of synthetic or enzymatic methods well known in the art. cDNAs can be synthesized, in whole or in part, using chemical methods well known in the art (Caruthers et al. (1980) Nucleic Acids Symp Ser (7)215-233). Alternatively, cDNAs can be produced enzymatically or recombinantly, by in vitro or in vivo transcription.

Nucleotide analogs can be incorporated into cDNAs by methods well known in the art. The only requirement is that the incorporated analog must base pair with native purines or pyrimidines. For example, 2, 6-diaminopurine can substitute for adenine and form stronger bonds with thymidine than those between adenine and thymidine. A weaker pair is formed when hypoxanthine is substituted for guanine and base pairs with cytosine. Additionally, cDNAs can include nucleotides that have been derivatized chemically or enzymatically.

cDNAs can be synthesized on a substrate. Synthesis on the surface of a substrate may be accomplished using a chemical coupling procedure and a piezoelectric printing apparatus as described by Baldeschweiler et al. (PCT publication WO95/251116). Alternatively, the cDNAs can be synthesized on a substrate surface using a self-addressable electronic device that controls when reagents are added as described by Heller et al. (U.S. Pat. No. 5,605,662). cDNAs can be synthesized directly on a substrate by sequentially dispensing reagents for their synthesis on the substrate surface or by dispensing preformed DNA fragments to the substrate surface. Typical dispensers include a micropipette delivering solution to the substrate with a robotic system to control the position of the micropipette with respect to the substrate. There can be a multiplicity of dispensers so that reagents can be delivered to the reaction regions efficiently.

cDNAs can be immobilized on a substrate by covalent means such as by chemical bonding procedures or UV irradiation. In one method, a cDNA is bound to a glass surface which has been modified to contain epoxide or aldehyde groups. In another method, a cDNA is placed on a polylysine coated surface and UV cross-linked to it as described by Shalon et al. (WO95/35505). In yet another method, a cDNA is actively transported from a solution to a given position on a substrate by electrical means (Heller, supra). cDNAs do not have to be directly bound to the substrate, but rather can be bound to the substrate through a linker group. The linker groups are typically about 6 to 50 atoms long to provide exposure of the attached cDNA. Preferred linker groups include ethylene glycol oligomers, diamines, diacids and the like. Reactive groups on the substrate surface react with a terminal group of the linker to bind the linker to the substrate. The other terminus of the linker is then bound to the cDNA. Alternatively, polynucleotides, plasmids or cells can be arranged on a filter. In the latter case, cells are lysed, proteins and cellular components degraded, and the DNA is coupled to the filter by UV cross-linking.

The cDNAs may be used for a variety of purposes. For example, the combination of the invention may be used on an array. The array, in turn, can be used in high-throughput methods for detecting a related polynucleotide in a sample, screening a plurality of molecules or compounds to identify a ligand, diagnosing a colon cancer, or inhibiting or inactivating a therapeutically relevant gene related to the cDNA.

When the cDNAs of the invention are employed on a microarray, the cDNAs are arranged in an ordered fashion so that each cDNA is present at a specified location. Because the cDNAs are at specified locations on the substrate, the hybridization patterns and intensities, which together create a unique expression profile, can be interpreted in terms of expression levels of particular genes and can be correlated with a particular metabolic process, condition, disorder, disease, stage of disease, or treatment.

Hybridization

The cDNAs or fragments or complements thereof may be used in various hybridization technologies. The cDNAs may be labeled using a variety of reporter molecules by either PCR recombinant, or enzymatic techniques. For example, a commercially available vector containing the cDNA is transcribed in the presence of an appropriate polymerase, such as T7 or SP6 polymerase, and at least one labeled nucleotide. Commercial kits are available for labeling and cleanup of such cDNAs. Radioactive (Amersham Pharmacia Biotech (APB), Piscataway N.J.), fluorescent (Operon Technologies, Alameda Calif.), and chemiluminescent labeling (Promega, Madison Wis. ) are well known in the art.

A cDNA may represent the complete coding region of an mRNA or be designed or derived from unique regions of the mRNA or genomic molecule, an intron, a 3′ untranslated region, or from a conserved motif. The cDNA is at least 18 contiguous nucleotides in length and is usually single stranded. Such a cDNA may be used under hybridization conditions that allow binding only to an identical sequence, a naturally occurring molecule encoding the same protein, or an allelic variant. Discovery of related human and mammalian sequences may also be accomplished using a pool of degenerate eDNAs and appropriate hybridization conditions. Generally, a cDNA for use in Southern or northern hybridizations may be from about 400 to about 6000 nucleotides long. Such cDNAs have high binding specificity in solution-based or substrate-based hybridizations. An oligonucleotide, a fragment of the cDNA, may be used to detect a polynucleotide in a sample using PCR.

The stringency of hybridization is determined by G+C content of the cDNA, salt concentration, and temperature. In particular, stringency is increased by reducing the concentration of salt or raising the hybridization temperature. In solutions used for some membrane based hybridizations, addition of an organic solvent such as formamide allows the reaction to occur at a lower temperature. Hybridization may be performed with buffers, such as 5× saline sodium citrate (SS C) with 1% sodium dodecyl sulfate (SDS) at 60° C., that permit the formation of a hybridization complex between nucleic acid sequences that contain some mismatches. Subsequent washes are performed with buffers such as 0.2× SSC with 0.1% SDS at either 45° C. (medium stringency) or 65°-68° C. (high stringency). At high stringency, hybridization complexes will remain stable only where the nucleic acid molecules are completely complementary. In some membrane-based hybridizations, preferably 35% or most preferably 50%, formamide may be added to the hybridization solution to reduce the temperature at which hybridization is performed. Background signals may be reduced by the use of detergents such as Sarkosyl or Triton X-100 (Sigma Aldrich, St. Louis Mo.) and a blocking agent such as denatured salmon sperm DNA. Selection of components and conditions for hybridization are well known to those skilled in the art and are reviewed in Ausubel et al. (1997, Short Protocols in Molecular Biology, John Wiley & Sons, New York N.Y., Units 2.8-2.11, 3.18-3.19 and 4.6-4.9).

Dot-blot, slot-blot, low density and high density arrays are prepared and analyzed using methods known in the art. cDNAs from about 18 consecutive nucleotides to about 5000 consecutive nucleotides in length are contemplated by the invention and used in array technologies. The preferred number of cDNAs on an array is at least about 100,000, a more preferred number is at least about 40,000, an even more preferred number is at least about 10,000, and a most preferred number is at least about 600 to about 800. The array may be used to monitor the expression level of large numbers of genes simultaneously and to identify genetic variants, mutations, and SNPs. Such information may be used to determine gene function; to understand the genetic basis of a disorder; to diagnose a disorder; and to develop and monitor the activities of therapeutic agents being used to control or cure a disorder. (See, e.g., U.S. Pat. No. 5,474,796; WO95/11995; WO95/35505; U.S. Pat. No. 5,605,662; and U.S. Pat. No. 5,958,342.)

Screening and Purification Assays

A cDNA may be used to screen a library or a plurality of molecules or compounds for a ligand which specifically binds the cDNA. Ligands may be DNA molecules, RNA molecules, peptide nucleic acid molecules, peptides, proteins such as transcription factors, promoters, enhancers, repressors, and other proteins that regulate replication, transcription, or translation of the polynucleotide in the biological system. The assay involves combining the cDNA or a fragment thereof with the molecules or compounds under conditions that allow specific binding and detecting the bound cDNA to identify at least one ligand that specifically binds the cDNA.

In one embodiment, the cDNA may be incubated with a library of isolated and purified molecules or compounds and binding activity determined by methods such as a gel-retardation assay (U.S. Pat. No. 6,010,849) or a reticulocyte lysate transcriptional assay. In another embodiment, the cDNA may be incubated with nuclear extracts from biopsied and/or cultured cells and tissues. Specific binding between the cDNA and a molecule or compound in the nuclear extract is initially determined by gel shift assay. Protein binding may be confirmed by raising antibodies against the protein and adding the antibodies to the gel-retardation assay where specific binding will cause a supershift in the assay.

In another embodiment, the cDNA may be used to purify a molecule or compound using affinity chromatography methods well known in the art. In one embodiment, the cDNA is chemically reacted with cyanogen bromide groups on a polymeric resin or gel. Then a sample is passed over and reacts with or binds to the cDNA. The molecule or compound which is bound to the cDNA may be released from the cDNA by increasing the salt concentration of the flow-through medium and then collected.

The cDNA may be used to purify a ligand from a sample. A method for using a cDNA to purify a ligand would involve combining the cDNA or a fragment thereof with a sample under conditions to allow specific binding, recovering the bound cDNA, and using an appropriate agent to separate the cDNA from the purified ligand.

Protein Production and Uses

The full length cDNAs or fragment thereof may be used to produce purified proteins using recombinant DNA technologies described herein and taught in Ausubel et al. (supra; Units 16.1-16.62). One of the advantages of producing proteins by these procedures is the ability to obtain highly-enriched sources of the proteins thereby simplifying purification procedures.

The proteins may contain amino acid substitutions, deletions or insertions made on the basis of similarity in polarity, charge, solubility, hydrophobicity, hydrophilicity, and/or the amphipathic nature of the residues involved. Such substitutions may be conservative in nature when the substituted residue has structural or chemical properties similar to the original residue (e.g., replacement of leucine with isoleucine or valine) or they may be nonconservative when the replacement residue is radically different (e.g., a glycine replaced by a tryptophan). Computer programs included in LASERGENE software (DNASTAR, Madison Wis. ), MACVECTOR software (Genetics Computer Group, Madison Wis. ) and RasMol software (Roger Sayle, University of Massachusetts, Amherst Mass.) may be used to help determine which and how many amino acid residues in a particular portion of the protein may be substituted, inserted, or deleted without abolishing biological or immunological activity.

Expression of Encoded Proteins

Expression of a particular cDNA may be accomplished by cloning the cDNA into a vector and transforming this vector into a host cell. The cloning vector used for the construction of cDNA libraries in the LIFESEQ databases may also be used for expression. Such vectors usually contain a promoter and a polylinker useful for cloning, priming, and transcription. An exemplary vector may also contain the promoter for β-galactosidase, an amino-terminal methionine and the subsequent seven amino acid residues of β-galactosidase. The vector may be transformed into competent E. coli cells. Induction of the isolated bacterial strain with isopropylthiogalactoside (IPTG) using standard methods will produce a fusion protein that contains an N terminal methionine, the first seven residues of β-galactosidase, about 15 residues of linker, and the protein encoded by the cDNA.

The cDNA may be shuttled into other vectors known to be useful for expression of protein in specific hosts. Oligonucleotides containing cloning sites and fragments of DNA sufficient to hybridize to stretches at both ends of the cDNA may be chemically synthesized by standard methods. These primers may then be used to amplify the desired fragments by PCR. The fragments may be digested with appropriate restriction enzymes under standard conditions and isolated using gel electrophoresis. Alternatively, similar fragments are produced by digestion of the cDNA with appropriate restriction enzymes and filled in with chemically synthesized oligonucleotides. Fragments of the coding sequence from more than one gene may be ligated together and expressed.

Signal sequences that dictate secretion of soluble proteins are particularly desirable as component parts of a recombinant sequence. For example, a chimeric protein may be expressed that includes one or more additional purification-facilitating domains. Such domains include, but are not limited to, metal-chelating domains that allow purification on immobilized metals, protein A domains that allow purification on immobilized immunoglobulin, and the domain utilized in the FLAGS extension/affinity purification system (Immunex, Seattle Wash.). The inclusion of a cleavable-linker sequence such as ENTEROKINASEMAX (Invitrogen, San Diego Calif.) between the protein and the purification domain may also be used to recover the protein.

Suitable host cells may include, but are not limited to, mammalian cells such as Chinese Hamster Ovary (CHO) and human 293 cells, insect cells such as Sf9 cells, plant cells such as Nicotiana tabacum, yeast cells such as Saccharomyces cerevisiae, and bacteria such as E. coli. For each of these cell systems, a useful vector may also include an origin of replication and one or two selectable markers to allow selection in bacteria as well as in a transformed eukaryotic host. Vectors for use in eukaryotic host cells may require the addition of 3′ poly(A) tail if the cDNA lacks poly(A).

Additionally, the vector may contain promoters or enhancers that increase gene expression. Many promoters are known and used in the art. Most promoters are host specific and exemplary promoters include SV40 promoters for CHO cells; T7 promoters for bacterial hosts; viral promoters and enhancers for plant cells; and PGH promoters for yeast. Adenoviral vectors with the rous sarcoma virus enhancer or retroviral vectors with long terminal repeat promoters may be used to drive protein expression in mammalian cell lines. Once homogeneous cultures of recombinant cells are obtained, large quantities of secreted soluble protein may be recovered from the conditioned medium and analyzed using chromatographic methods well known in the art. An alternative method for the production of large amounts of secreted protein involves the transformation of mammalian embryos and the recovery of the recombinant protein from milk produced by transgenic cows, goats, sheep, and the like.

In addition to recombinant production, proteins or portions thereof may be produced manually, using solid-phase techniques (Stewart et al. (1969) Solid-Phase Peptide Synthesis, W H Freeman, San Francisco Calif.; Merrifield (1963) J Am Chem Soc 5:2149-2154), or using machines such as the ABI 431A peptide synthesizer (Applied Biosystems, Foster City Calif.). Proteins produced by any of the above methods may be used as pharmaceutical compositions to treat disorders associated with null or inadequate expression of the genomic sequence.

Screening and Purification Assays

A protein or a portion thereof encoded by the cDNA may be used to screen a library or a plurality of molecules or compounds for a ligand with specific binding affinity or to purify a molecule or compound from a sample. The protein or portion thereof employed in such screening may be free in solution, affixed to an abiotic or biotic substrate, or located intracellularly. For example, viable or fixed prokaryotic host cells that are stably transformed with recombinant nucleic acids that have expressed and positioned a protein on their cell surface can be used in screening assays. The cells are screened against a library or a plurality of ligands and the specificity of binding or formation of complexes between the expressed protein and the ligand may be measured. The ligands may be DNA, RNA, or PNA molecules, agonists, antagonists, antibodies, immunoglobulins, inhibitors, peptides, pharmaceutical agents, proteins, drugs, or any other test molecule or compound that specifically binds the protein. An exemplary assay involves combining the mammalian protein or a portion thereof with the molecules or compounds under conditions that allow specific binding and detecting the bound protein to identify at least one ligand that specifically binds the protein.

This invention also contemplates the use of competitive drug screening assays in which neutralizing antibodies capable of binding the protein specifically compete with a test compound capable of binding to the protein or oligopeptide or fragment thereof. One method for high throughput screening using very small assay volumes and very small amounts of test compound is described in U.S. Pat. No. 5,876,946. Molecules or compounds identified by screening may be used in a model system to evaluate their toxicity, diagnostic, or therapeutic potential.

The protein may be used to purify a ligand from a sample. A method for using a protein to purify a ligand would involve combining the protein or a portion thereof with a sample under conditions to allow specific binding, recovering the bound protein, and using an appropriate chaotropic agent to separate the protein from the purified ligand.

Production of Antibodies

A protein encoded by a cDNA of the invention may be used to produce specific antibodies. Antibodies may be produced using an oligopeptide or a portion of the protein with inherent immunological activity. Methods for producing antibodies include: 1) injecting an animal, usually goats, rabbits, or mice, with the protein, or an antigenically-effective portion or an oligopeptide thereof, to induce an immune response; 2) engineering hybridomas to produce monoclonal antibodies; 3) inducing in vivo production in the lymphocyte population; or 4) screening libraries of recombinant immunoglobulins. Recombinant immunoglobulins may be produced as taught in U.S. Pat. No. 4,816,567.

Antibodies produced using the proteins of the invention are useful for the diagnosis of prepathologic disorders as well as the diagnosis of chronic or acute diseases characterized by abnormalities in the expression, amount, or distribution of the protein. A variety of protocols for competitive binding or immunoradiometric assays using either polyclonal or monoclonal antibodies specific for proteins are well known in the art. Immunoassays typically involve the formation of complexes between a protein and its specific binding molecule or compound and the measurement of complex formation. Immunoassays may employ a two-site, monoclonal-based assay that utilizes monoclonal antibodies reactive to two noninterfering epitopes on a specific protein or a competitive binding assay (Pound (1998) Immunochemical Protocols, Humana Press, Totowa N.J.).

Immunoassay procedures may be used to quantify expression of the protein in cell cultures, in subjects with a particular disorder or in model animal systems under various conditions. Increased or decreased production of proteins as monitored by immunoassay may contribute to knowledge of the cellular activities associated with developmental pathways, engineered conditions or diseases, or treatment efficacy. The quantity of a given protein in a given tissue may be determined by performing immunoassays on freeze-thawed detergent extracts of biological samples and comparing the slope of the binding curves to binding curves generated by purified protein.

Labeling of Molecules for Assay

A wide variety of reporter molecules and conjugation techniques are known by those skilled in the art and may be used in various cDNA, polynucleotide, protein, peptide or antibody assays. Synthesis of labeled molecules may be achieved using commercial kits for incorporation of a labeled nucleotide such as ³²P-dCTP, Cy3-dCTP or Cy5-dCTP or amino acid such as ³⁵S-methionine. Polynucleotides, cDNAs, proteins, or antibodies may be directly labeled with a reporter molecule by chemical conjugation to amines, thiols and other groups present in the molecules using reagents such as BIODIPY or FITC (Molecular Probes, Eugene Oreg.).

The proteins and antibodies may be labeled for purposes of assay by joining them, either covalently or noncovalently, with a reporter molecule that provides for a detectable signal. A wide variety of labels and conjugation techniques are known and have been reported in the scientific and patent literature including, but not limited to U.S. Pat. Nos. 3,817,837; 3,850,752; 3,939,350; 3,996,345; 4,277,437; 4,275,149; and 4,366,241.

Diagnostics

The cDNAs, or fragments thereof, may be used to detect and quantify differential gene expression; absence, presence, or excess expression of mRNAs; or to monitor mRNA levels during therapeutic intervention. Disorders associated with altered or differential expression include colon cancer and premalignant colon polyps. These cDNAs can also be utilized as markers of treatment efficacy against the disorders noted above and other disorders, conditions, and diseases over a period ranging from several days to months. The diagnostic assay may use hybridization or amplification technology to compare gene expression in a biological sample from a patient to standard samples in order to detect altered or differential gene expression. Qualitative or quantitative methods for this comparison are well known in the art.

For example, the cDNA may be labeled by standard methods and added to a biological sample from a patient under conditions for hybridization complex formation. After an incubation period, the sample is washed and the amount of label (or signal) associated with hybridization complexes is quantified and compared with a standard value. If the amount of label in the patient sample is significantly altered in comparison to the standard value, then the presence of the associated condition, disease or disorder is indicated.

In order to provide a basis for the diagnosis of a condition, disease or disorder associated with gene expression, a normal or standard expression profile is established. This may be accomplished by combining a biological sample taken from normal subjects, either animal or human, with a probe under conditions for hybridization or amplification. Standard hybridization may be quantified by comparing the values obtained using normal subjects with values from an experiment in which a known amount of a substantially purified target sequence is used. Standard values obtained in this manner may be compared with values obtained from samples from patients who are symptomatic for a particular condition, disease, or disorder. Deviation from standard values toward those associated with a particular condition is used to diagnose that condition.

Such assays may also be used to evaluate the efficacy of a particular therapeutic treatment regimen in animal studies and in clinical trial or to monitor the treatment of an individual patient. Once the presence of a condition is established and a treatment protocol is initiated, diagnostic assays may be repeated on a regular basis to determine if the level of expression in the patient begins to approximate that which is observed in a normal subject. The results obtained from successive assays may be used to show the efficacy of treatment over a period ranging from several days to months.

Gene Expression Profiles

A gene expression profile comprises a plurality of cDNAs and a plurality of detectable hybridization complexes, wherein each complex is formed by hybridization of one or more probes to one or more complementary sequences in a sample. The cDNAs of the invention are used as elements on a microarray to analyze gene expression profiles. In one embodiment, the microarray is used to monitor the progression of disease. Researchers can assess and catalog the differences in gene expression between healthy and diseased tissues or cells. By analyzing changes in patterns of gene expression, disease can be diagnosed at earlier stages before the patient is symptomatic. The invention can be used to formulate a prognosis and to design a treatment regimen. The invention can also be used to monitor the efficacy of treatment. For treatments with known side effects, the micro array is employed to improve the treatment regimen. A dosage is established that causes a change in genetic expression patterns indicative of successful treatment. Expression patterns associated with the onset of undesirable side effects are avoided. This approach may be more sensitive and rapid than waiting for the patient to show inadequate improvement, or to manifest side effects, before altering the course of treatment.

In another embodiment, animal models which mimic a human disease can be used to characterize expression profiles associated with a particular condition, disorder or disease; or treatment of the condition, disorder or disease. Novel treatment regimens may be tested in these animal models using microarrays to establish and then follow expression profiles over time. In addition, microarrays may be used with cell cultures or tissues removed from animal models to rapidly to screen large numbers of candidate drug molecules, looking for ones that produce an expression profile similar to those of known therapeutic drugs, with the expectation that molecules with the same expression profile will likely have similar therapeutic effects. Thus, the invention provides the means to rapidly determine the molecular mode of action of a drug.

Assays Using Antibodies

Antibodies directed against epitopes on a protein encoded by a cDNA of the invention may be used in assays to quantify the amount of protein found in a particular human cell. Such assays include methods utilizing the antibody and a label to detect expression level under normal or disease conditions. The antibodies may be used with or without modification, and labeled by joining them, either covalently or noncovalently, with a labeling moiety.

Protocols for detecting and measuring protein expression using either polyclonal or monoclonal antibodies are well known in the art. Examples include ELISA, RIA, and fluorescent activated cell sorting (FACS). Such immunoassays typically involve the formation of complexes between the protein and its specific antibody and the measurement of such complexes. These and other assays are described in Pound (supra). The method may employ a two-site, monoclonal-based immunoassay utilizing monoclonal antibodies reactive to two non-interfering epitopes, or a competitive binding assay. (See, e.g., Coligan et al. (1997) Current Protocols in Immunology, Wiley-Interscience, New York N.Y.; Pound, supra)

Therapeutics

The cDNAs and fragments thereof can be used in gene therapy. cDNAs can be delivered ex vivo to target cells, such as cells of bone marrow. Once stable integration and transcription and or translation are confirmed, the bone marrow may be reintroduced into the subject. Expression of the protein encoded by the cDNA may correct a colon cancer or premalignant colon polyps associated with mutation of a normal sequence, reduction or loss of an endogenous target protein, or overepression of an endogenous or mutant protein. Alternatively, cDNAs may be delivered in vivo using vectors such as retrovirus, adenovirus, adeno-associated virus, herpes simplex virus, and bacterial plasmids. Non-viral methods of gene delivery include cationic liposomes, polylysine conjugates, artificial viral envelopes, and direct injection of DNA (Anderson (1998) Nature 392:25-30; Dachs et al. (1997) Oncol Res 9:313-325; Chu et al. (1998) J Mol Med 76(3-4):184-192; Weiss et al. (1999) Cell Mol Life Sci 55(3):334-358; Agrawal (1996) Antisense Therapeutics, Humana Press, Totowa N.J.; and August et al. (1997) Gene Therapy (Advances in Pharmacology, Vol. 40), Academic Press, San Diego Calif.).

In addition, expression of a particular protein can be regulated through the specific binding of a fragment of a cDNA to a genomic sequence or an mRNA which encodes the protein or directs its transcription or translation. The cDNA can be modified or derivatized to any RNA-like or DNA-like material including peptide nucleic acids, branched nucleic acids, and the like. These sequences can be produced biologically by transforming an appropriate host cell with a vector containing the sequence of interest.

Molecules which regulate the activity of the cDNA or encoded protein are useful as therapeutics for colon cancer and premalignant colon polyps. Such molecules include agonists which increase the expression or activity of the polynucleotide or encoded protein, respectively; or antagonists which decrease expression or activity of the polynucleotide or encoded protein, respectively. In one aspect, an antibody which specifically binds the protein may be used directly as an antagonist or indirectly as a delivery mechanism for bringing a pharmaceutical agent to cells or tissues which express the protein.

Additionally, any of the proteins, or their ligands, or complementary nucleic acid sequences may be administered as pharmaceutical compositions or in combination with other appropriate therapeutic agents. Selection of the appropriate agents for use in combination therapy may be made by one of ordinary skill in the art, according to conventional pharmaceutical principles. The combination of therapeutic agents may act synergistically to affect the treatment or prevention of the conditions and disorders associated with an immune response. Using this approach, one may be able to achieve therapeutic efficacy with lower dosages of each agent, thus reducing the potential for adverse side effects. Further, the therapeutic agents may be combined with pharmaceutically-acceptable carriers including excipients and auxiliaries which facilitate processing of the active compounds into preparations which can be used pharmaceutically. Further details on techniques for formulation and administration used by doctors and pharmacists may be found in the latest edition of Remington's Pharmaceutical Sciences (Mack Publishing, Easton Pa.).

Model Systems

Animal models may be used as bioassays where they exhibit a phenotypic response similar to that of humans and where exposure conditions are relevant to human exposures. Mammals are the most common models, and most infectious agent, cancer, drug, and toxicity studies are performed on rodents such as rats or mice because of low cost, availability, lifespan, reproductive potential, and abundant reference literature. Inbred and outbred rodent strains provide a convenient model for investigation of the physiological consequences of underexpression or overexpression of genes of interest and for the development of methods for diagnosis and treatment of diseases. A mammal inbred to overexpress a particular gene (for example, secreted in milk) may also serve as a convenient source of the protein expressed by that gene.

Transgenic Animal Models

Transgenic rodents that overexpress or underexpress a gene of interest may be inbred and used to model human diseases or to test therapeutic or toxic agents. (See, e.g., U.S. Pat. Nos. 5,175,383 and 5,767,337.) In some cases, the introduced gene may be activated at a specific time in a specific tissue type during fetal or postnatal development. Expression of the transgene is monitored by analysis of phenotype, of tissue-specific mRNA expression, or of serum and tissue protein levels in transgenic animals before, during, and after challenge with experimental drug therapies.

Embryonic Stem Cells

Embryonic (ES) stem cells isolated from rodent embryos retain the potential to form embryonic tissues. When ES cells such as the mouse 129/SvJ cell line are placed in a blastocyst from the C57BL/6 mouse strain, they resume normal development and contribute to tissues of the live-born animal. ES cells are preferred for use in the creation of experimental knockout and knockin animals. The method for this process is well known in the art and the steps are: the cDNA is introduced into a vector, the vector is transformed into ES cells, transformed cells are identified and microinjected into mouse cell blastocysts, blastocysts are surgically transferred to pseudopregnant dams. The resulting chimeric progeny are genotyped and bred to produce heterozygous or homozygous strains.

Knockout Analysis

In gene knockout analysis, a region of a gene is enzymatically modified to include a non-natural intervening sequence such as the neomycin phosphotransferase gene (neo; Capecchi (1989) Science 244:1288-1292). The modified gene is transformed into cultured ES cells and integrates into the endogenous genome by homologous recombination. The inserted sequence disrupts transcription and translation of the endogenous gene.

Knockin Analysis

ES cells can be used to create knockin humanized animals or transgenic animal models of human diseases. With knockin technology, a region of a human gene is injected into animal ES cells, and the human sequence integrates into the animal cell genome. Transgenic progeny or inbred lines are studied and treated with potential pharmaceutical agents to obtain information on the progression and treatment of the analogous human condition.

As described herein, the uses of the cDNAs, provided in the Sequence Listing of this application, and their encoded proteins are exemplary of known techniques and are not intended to reflect any limitation on their use in any technique that would be known to the person of average skill in the art. Furthermore, the cDNAs provided in this application may be used in molecular biology techniques that have not yet been developed, provided the new techniques rely on properties of nucleotide sequences that are currently known to the person of ordinary skill in the art, e.g., the triplet genetic code, specific base pair interactions, and the like. Likewise, reference to a method may include combining more than one method for obtaining or assembling full length cDNA sequences that will be known to those skilled in the art. It is also to be understood that this invention is not limited to the particular methodology, protocols, and reagents described, as these may vary. It is also understood that the terminology used herein is for the purpose of describing particular embodiments only, and is not intended to limit the scope of the present invention which will be limited only by the appended claims. The examples below are provided to illustrate the subject invention and are not included for the purpose of limiting the invention.

### EXAMPLES

I Construction of cDNA Libraries

RNA was purchased from Clontech Laboratories (Palo Alto Calif.) or isolated from various tissues. Some tissues were homogenized and lysed in guanidinium isothiocyanate, while others were homogenized and lysed in phenol or in a suitable mixture of denaturants, such as TRIZOL reagent (Life Technologies, Rockville Md.). The resulting lysates were centrifuged over CsCl cushions or extracted with chloroform. RNA was precipitated with either isopropanol or ethanol and sodium acetate, or by other routine methods.

Phenol extraction and precipitation of RNA were repeated as necessary to increase RNA purity. In most cases, RNA was treated with DNAse. For most libraries, poly(A) RNA was isolated using oligo d(T)-coupled paramagnetic particles (Promega), OLIGOTEX latex particles (Qiagen, Valencia Calif.), or an OLIGOTEX mRNA purification kit (Qiagen). Alternatively, poly(A) RNA was isolated directly from tissue lysates using other kits, including the POLY(A)PURE mRNA purification kit (Ambion, Austin Tex.).

In some cases, Stratagene (La Jolla Calif.) was provided with RNA and constructed the corresponding cDNA libraries. Otherwise, cDNA was synthesized and cDNA libraries were constructed with the UNIZAP vector system (Stratagene) or SUPERSCRIPT plasmid system (Life Technologies) using the recommended procedures or similar methods known in the art. (See Ausubel, supra, Units 5.1 through 6.6.) Reverse transcription was initiated using oligo d(T) or random primers. Synthetic oligonucleotide adapters were ligated to double stranded cDNA, and the cDNA was digested with the appropriate restriction enzyme or enzymes. For most libraries, the cDNA was size-selected (300-1000 bp) using SEPHACRYL S1000, SEPHAROSE CL2B, or SEPHAROSE CL4B column chromatography (APB) or preparative agarose gel electrophoresis. cDNAs were ligated into compatible restriction enzyme sites of the polylinker of the pBLUESCRIPT phagemid (Stratagene), pSPORT1 plasmid (Life Technologies), or pINCY plasmid (Incyte Genomics, Inc., Palo Alto Calif.). Recombinant plasmids were transformed into XL1-BLUE, XL1-BLUEMRF, or SOLR competent E. coli cells (Stratagene) or DH5α, DH10B, or ELECTROMAX DH10B competent E. coli cells (Life Technologies).

In some cases, libraries were superinfected with a 5× excess of the helper phage, M13K07, according to the method of Vieira et al. (1987, Methods Enzymol 153:3-11) and normalized or subtracted using a methodology adapted from Soares (1994, Proc Natl Acad Sci 91:9228-9232), Swaroop etal. (1991, Nucleic Acids Res 19:1954), and Bonaldo etal. (1996, Genome Research 6:791-806). The modified Soares normalization procedure was utilized to reduce the repetitive cloning of highly expressed high abundance cDNAs while maintaining the overall sequence complexity of the library. Modification included significantly longer hybridization times which allowed for increased gene discovery rates by biasing the normalized libraries toward those infrequently expressed low-abundance cDNAs which are poorly represented in a standard transcript image (Soares et al., supra).

II Isolation and Sequencing of cDNA Clones

Plasmids were recovered from host cells by in vivo excision using the UNIZAP vector system (Stratagene) or by cell lysis. Plasmids were purified using one of the following: the Magic or WIZARD MINIPREPS DNA purification system (Promega); the AGTC MINIPREP purification kit (Edge BioSystems, Gaithersburg Md.); the QIAWELL 8, QIAWELL 8 Plus, or QIAWELL 8 Ultra plasmid purification systems, or the REAL PREP 96 plasmid purification kit (QIAGEN). Following precipitation, plasmids were resuspended in 0.1 ml of distilled water and stored, with or without lyophilization, at 4° C.

Alternatively, plasmid DNA was amplified from host cell lysates using direct link PCR in a high-throughput format (Rao (1994) Anal Biochem 216:1-14). Host cell lysis and thermal cycling steps were carried out in a single reaction mixture. Samples were processed and stored in 384-well plates, and the concentration of amplified plasmid DNA was quantified fluorometrically using PICOGREEN dye (Molecular Probes) and a FLUOROSKAN II fluorescence scanner (Labsystems Oy, Helsinki Finland).

cDNA sequencing reactions were processed using standard methods or high-throughput instrumentation such as the ABI CATALYST 800 thermal cycler (Applied Biosystems) or the DNA ENGINE thermal cycler (MJ Research, Watertown Mass.) in conjunction with the HYDRA microdispenser (Robbins Scientific, Sunnyvale Calif.) or the MICROLAB 2200 system (Hamilton, Reno NV). cDNA sequencing reactions were prepared using reagents provided by APB or supplied in ABI sequencing kits such as the ABI PRISM BIGDYE cycle sequencing kit (Applied Biosystems). Electrophoretic separation of cDNA sequencing reactions and detection of labeled cDNAs were carried out using the MEGABACE 1000 DNA sequencing system (APB); the ABI PRISM 373 or 377 sequencing systems (Applied Biosystems) in conjunction with standard ABI protocols and base calling software; or other sequence analysis systems known in the art. Reading frames within the cDNA sequences were identified using standard methods (reviewed in Ausubel, supra, Unit 7.7).

III Extension of cDNA Sequences

Nucleic acid sequences were extended using the cDNA clones and oligonucleotide primers. One primer was synthesized to initiate 5′ extension of the known fragment, and the other, to initiate 3′ extension of the known fragment. The initial primers were designed using OLIGO 4.06 software (National Biosciences, Plymouth Minn.), or another appropriate program, to be about 22 to 30 nucleotides in length, to have a GC content of about 50% or more, and to anneal to the target sequence at temperatures of about 68° C. to about 72° C. Any stretch of nucleotides which would result in hairpin structures and primer-primer dimerizations was avoided.

Selected human cDNA libraries were used to extend the sequence. If more than one extension was necessary or desired, additional or nested sets of primers were designed. Preferred libraries are ones that have been size-selected to include larger cDNAs. Also, random primed libraries are preferred because they will contain more sequences with the 5′ and upstream regions of genes. A randomly primed library is particularly useful if an oligo d(T) library does not yield a full-length cDNA.

High fidelity amplification was obtained by PCR using methods well known in the art. PCR was performed in 96-well plates using the DNA ENGINE thermal cycler (MJ Research). The reaction mix contained DNA template, 200 nmol of each primer, reaction buffer containing Mg²⁺, (NH₄)₂SO₄, and β-mercaptoethanol, Taq DNA polymerase (APB), ELONGASE enzyme (Life Technologies). and Pfu DNA polymerase (Stratagene), with the following parameters for primer pair PCI A and PCI B (Incyte Genomics): Step 1: 94° C., 3 min; Step 2: 94° C., 15 sec; Step 3: 60° C., 1 min; Step 4: 68° C., 2 min; Step 5: Steps 2, 3, and 4 repeated 20 times; Step 6: 68° C., 5 min; Step 7: storage at 4° C. In the alternative, the parameters for primer pair T7 and SK+ (Stratagene) were as follows: Step 1: 94° C., 3 min; Step 2: 94° C., 15 sec; Step 3: 57° C., 1 min; Step 4: 68° C., 2 min; Step 5: Steps 2,3, and 4 repeated 20 times; Step 6: 68° C., 5 min; Step 7: storage at 4° C.

The concentration of DNA in each well was determined by dispensing 100 μl PICOGREEN reagent (0.25% reagent in 1× TE, v/v; Molecular Probes) and 0.5 μl of undiluted PCR product into each well of an opaque fluorimeter plate (Corning Costar, Acton Mass.) and allowing the DNA to bind to the reagent. The plate was scanned in a FLUOROSKAN II (Labsystems Oy) to measure the fluorescence of the sample and to quantify the concentration of DNA. A 5 μl to 10 μl aliquot of the reaction mixture was analyzed by electrophoresis on a 1% agarose mini-gel to determine which reactions were successful in extending the sequence.

The extended nucleic acids were desalted and concentrated, transferred to 384-well plates, digested with CviJI cholera virus endonuclease (Molecular Biology Research, Madison Wis. ), and sonicated or sheared prior to religation into pUC18 vector (APB). For shotgun sequencing, the digested nucleic acids were separated on low concentration (0.6 to 0.8%) agarose gels, fragments were excised, and agar digested with AGARACE enzyme (Promega). Extended clones were religated using T4 DNA ligase (New England Biolabs, Beverly Mass.) into pUC18 vector (APB), treated with Pfu DNA polymerase (Stratagene) to fill-in restriction site overhangs, and transformed into competent E. coli cells. Transformed cells were selected on antibiotic-containing media, and individual colonies were picked and cultured overnight at 37° C. in 384-well plates in LB/2× carbenicillin liquid media.

The cells were lysed, and DNA was amplified by PCR using Taq DNA polymerase (APB) and Pfu DNA polymerase (Stratagene) with the following parameters: Step 1: 94° C., 3 min; Step 2: 94° C., 15 sec; Step 3: 60° C., 1 min; Step 4: 72° C., 2 min; Step 5: steps 2, 3, and 4 repeated 29 times; Step 6: 72° C., 5 min; Step 7: storage at 4° C. DNA was quantified using PICOGREEN reagent (Molecular Probes) as described above. Samples with low DNA recoveries were reamplified using the same conditions described above. Samples were diluted with 20% dimethylsulfoxide (DMSO; 1:2, v/v), and sequenced using DYENAMIC energy transfer sequencing primers and the DYENAMIC DIRECT cycle sequencing kit (APB) or the ABI PRISM BIGDYE terminator cycle sequencing kit (Applied Biosystems).

IV Assembly and Analysis of Sequences

Component nucleotide sequences from chromatograms were subjected to PHRED analysis (Phil Green, University of Washington, Seattle Wash.) and assigned a quality score. The sequences having at least a required quality score were subject to various pre-processing algorithms to eliminate low quality 3′ ends, vector and linker sequences, polyA tails, Alu repeats, mitochondrial and ribosomal sequences, bacterial contamination sequences, and sequences smaller than 50 base pairs. Sequences were screened using the BLOCK 2 program (Incyte Genomics), a motif analysis program based on sequence information contained in the SWISS-PROT and PROSITE databases (Bairoch et al. (1997) Nucleic Acids Res 25:217-221; Attwood et al. (1997) J Chem Inf Comput Sci 37:417-424).

Processed sequences were subjected to assembly procedures in which the sequences were assigned to bins, one sequence per bin. Sequences in each bin were assembled to produce consensus sequences, templates. Subsequent new sequences were added to existing bins using BLAST (Altschul (supra); Altschul et al (supra); Karlin et al (1988) Proc Natl Acad Sci 85:841-845), BLASTn (vers.1.4, WashU), and CROSSMATCH software (Phil Green, supra). Candidate pairs were identified as all BLAST hits having a quality score greater than or equal to 150. Alignments of at least 82% local identity were accepted into the bin. The component sequences from each bin were assembled using PHRAP (Phil Green, supra). Bins with several overlapping component sequences were assembled using DEEP PHRAP (Phil Green, supra).

Bins were compared against each other, and those having local similarity of at least 82% were combined and reassembled. Reassembled bins having templates of insufficient overlap (less than 95% local identity) were re-split. Assembled templates were also subjected to analysis by STITCHER/EXON MAPPER algorithms which analyzed the probabilities of the presence of splice variants, alternatively spliced exons, splice junctions, differential expression of alternative spliced genes across tissue types, disease states, and the like. These resulting bins were subjected to several rounds of the above assembly procedures to generate the template sequences found in the LIFESEQ GOLD database (Incyte Genomics).

The assembled templates were annotated using the following procedure. Template sequences were analyzed using BLASTn (vers. 2.0, NCBI) versus GBpri (GenBank version 117). “Hits” were defined as an exact match having from 95% local identity over 200 base pairs through 100% local identity over 100 base pairs, or a homolog match having an E-value equal to or greater than 1×10⁻⁸. (The “E-value” quantifies the statistical probability that a match between two sequences occurred by chance). The hits were subjected to frameshift FASTx versus GENPEPT (GenBank version 117). In this analysis, a homolog match was defined as having an E-value of 1×10⁻⁸. The assembly method used above was described in U.S. Ser. No. 09/276,534, filed March 25, 1999, and the LIFESEQ GOLD user manual (Incyte Genomics).

Following assembly, template sequences were subjected to motif, BLAST, Hidden Markov Model (HMM; Pearson and Lipman (1988) Proc Natl Acad Sci 85:2444-2448; Smith and Waterman (1981) J Mol Biol 147:195-197), and functional analyses, and categorized in protein hierarchies using methods described in U.S. Ser. No. 08/812,290, filed Mar. 6,1997; U.S. Ser. No. 08/947,845, filed Oct. 9, 1997; U.S. Pat. No. 5,953,727; and U.S. Pat. No. 09/034,807, filed Mar. 4, 1998. Template sequences may be further queried against public databases such as the GenBank rodent, mammalian, vertebrate, eukaryote, prokaryote, and human EST databases.

V Selection of Sequences, Microarray Preparation and Use

Incyte clones represent template sequences derived from the LIFESEQ GOLD assembled human sequence database (Incyte Genomics). In cases where more than one clone was available for a particular template, the 5′-most clone in the template was used on the microarray. The HUMAN GENOME GEM series 1-3 microarrays (Incyte Genomics) contain 28,626 array elements which represent 10,068 annotated clusters and 18,558 unannotated clusters. For the UNIGEM series microarrays (Incyte Genomics), Incyte clones were mapped to non-redundant Unigene clusters (Unigene database (build 46), NCBI; Shuler (1997) J Mol Med 75:694-698), and the 5′ clone with the strongest BLAST alignment (at least 90% identity and 100 bp overlap) was chosen, verified, and used in the construction of the microarray. The UNIGEM V microarray (Incyte Genomics) contains 7075 array elements which represent 4610 annotated genes and 2,184 unannotated clusters. Tables 1 and 2 show the GenBank annotations for SEQ ID NOs:1-138 of this invention as produced by BLAST analysis.

To construct microarrays, cDNAs were amplified from bacterial cells using primers complementary to vector sequences flanking the cDNA insert. Thirty cycles of PCR increased the initial quantity of cDNAs from 1-2 ng to a final quantity of greater than 5 μg. Amplified cDNAs were then purified using SEPHACRYL-400 columns (APB). Purified cDNAs were immobilized on polymer-coated glass slides. Glass microscope slides (Corning, Corning N.Y.) were cleaned by ultrasound in 0.1% SDS and acetone, with extensive distilled water washes between and after treatments. Glass slides were etched in 4% hydrofluoric acid (VWR Scientific Products, West Chester Pa.), washed thoroughly in distilled water, and coated with 0.05% aminopropyl silane (Sigma Aldrich) in 95% ethanol. Coated slides were cured in a 110° C. oven. cDNAs were applied to the coated glass substrate using a procedure described in U.S. Pat. No. 5,807,522. One microliter of the cDNA at an average concentration of 100 ng/μl was loaded into the open capillary printing element by a high-speed robotic apparatus which then deposited about 5 nl of cDNA per slide.

Microarrays were UV-crosslinked using a STRATALINKER UV-crosslinker (Stratagene), and then washed at room temperature once in 0.2% SDS and three times in distilled water. Non-specific binding sites were blocked by incubation of microarrays in 0.2% casein in phosphate buffered saline (Tropix, Bedford Mass.) for 30 minutes at 60° C. followed by washes in 0.2% SDS and distilled water as before.

VI Preparation of Samples

Tissue Samples

Matched normal colon and cancerous colon or colon polyp tissue samples were provided by the Huntsman Cancer Institute, (Salt Lake City, Utah). Donor 3754 is an individual diagnosed with a pendunculated colon polyp; age and sex of the donor is unknown. Donor 3755 is an individual diagnosed with colon polyps and having a family history of colon cancer; age and sex of the donor is unknown. Donor 3583 is a 58 year-old male diagnosed with a tubulovillous adenoma hyperplastic polyp. Donor 3311 is an 85 year-old male diagnosed with an invasive, poorly differentiated adenocarcinoma with metastases to the lymph nodes. Donor 3756 is a 78 year-old female diagnosed with an invasive, moderately differentiated adenocarcinoma. Donor 3757 is a 75 year-old female diagnosed with an invasive, moderate to poorly differentiated adenocarcinoma with metastases to the lymph nodes. Donor 3649 is an 86 year-old individual, sex unknown, diagnosed with an invasive, well-differentiated adenocarcinoma. Donor 3647 is an 83 year-old individual, sex unknown, diagnosed with an invasive, moderately well-differentiated adenocarcinoma with metastases to the lymph nodes. Donor 3839 is a 60 year-old individual, sex unknown, diagnosed with colon cancer. Donor 3581 is a male of unknown age diagnosed with a colorectal tumor. Donors 3754, 3755, 3311, 3756, and 3757 were matched against a common control sample comprising a pool of normal colon tissue from three additional donors. All other comparisons were done with matched normal and tumor or polyp tissue from the same donor.

Isolation and Labeling of Sample cDNAs

Tissues were homogenized and lysed in 1 ml of TRIZOL reagent (5×10⁶ cells/ml; Life Technologies). The lysates were vortexed thoroughly and incubated at room temperature for 2-3 minutes and extracted with 0.5 ml chloroform. The extract was mixed, incubated at room temperature for 5 minutes, and centrifuged at 15,000 rpm for 15 minutes at 4° C. The aqueous layer was collected and an equal volume of isopropanol was added. Samples were mixed, incubated at room temperature for 10 minutes, and centrifuged at 15,000 rpm for 20 minutes at 4° C. The supernatant was removed and the RNA pellet was washed with 1 ml of 70% ethanol, centrifuged at 15,000 rpm at 4° C., and resuspended in RNAse-free water. The concentration of the RNA was determined by measuring the optical density at 260 nm.

Poly(A) RNA was prepared using an OLIGOTEX mRNA kit (QIAGEN) with the following modifications: OLIGOTEX beads were washed in tubes instead of on spin columns, resuspended in elution buffer, and then loaded onto spin columns to recover mRNA. To obtain maximum yield, the mRNA was eluted twice.

Each poly(A) RNA sample was reverse transcribed using MMLV reverse-transcriptase, 0.05 pg/μl oligo-d(T) primer (21 mer), 1× first strand buffer. 0.03 units/ul RNase inhibitor, 500 uM dATP, 500 uM dGTP, 500 uM dTTP, 40 uM dCTP, and 40 uM either dCTP-Cy3 or dCTP-Cy5 (APB). The reverse transcription reaction was performed in a 25 ml volume containing 200 ng poly(A) RNA using the GEMBRIGHT kit (Incyte Genomics). Specific control poly(A) RNAs (YCFR06, YCFR45, YCFR67, YCFR85, YCFR43, YCFR22, YCFR23, YCFR25, YCFR44, YCFR26) were synthesized by in vitro transcription from non-coding yeast genomic DNA (W. Lei, unpublished). As quantitative controls, control mRNAs (YCFR06, YCFR45, YCFR67, and YCFR85) at 0.002ng, 0.02ng, 0.2 ng, and 2ng were diluted into reverse transcription reaction at ratios of 1 :100,000, 1 :10,000, 1 :1000, 1 :100 (w/w) to sample mRNA, respectively. To sample differential expression patterns, control mRNAs (YCFR43, YCFR22, YCFR23, YCFR25, YCFR44, YCFR26) were diluted into reverse transcription reaction at ratios of 1:3, 3:1, 1:10, 10:1, 1:25, 25:1 (w/w) to sample mRNA. Reactions were incubated at 37° C. for 2 hr, treated with 2.5 ml of 0.5M sodium hydroxide, and incubated for 20 minutes at 85° C. to the stop the reaction and degrade the RNA.

cDNAs were purified using two successive CHROMA SPIN 30 gel filtration spin columns (Clontech). Cy3- and Cy5-labeled reaction samples were combined as described below and ethanol precipitated using 1 ml of glycogen (1 mg/ml), 60 ml sodium acetate, and 300 ml of 100% ethanol. The cDNAs were then dried to completion using a SpeedVAC system (Savant Instruments, Holbrook N.Y.) and resuspended in 14 μl 5× SSC, 0.2% SDS.

VII Hybridization and Detection

Hybridization reactions contained 9 μl of sample mixture containing 0.2 μg each of Cy3 and Cy5 labeled cDNA synthesis products in 5× SSC, 0.2% SDS hybridization buffer. The mixture was heated to 65° C. for 5 minutes and was aliquoted onto the microarray surface and covered with an 1.8 cm² coverslip. The microarrays were transferred to a waterproof chamber having a cavity just slightly larger than a microscope slide. The chamber was kept at 100% humidity internally by the addition of 140 μl of 5× SSC in a corner of the chamber. The chamber containing the microarrays was incubated for about 6.5 hours at 60° C. The microarrays were washed for 10 min at 45° C. in low stringency wash buffer (1× SSC, 0.1% SDS), three times for 10 minutes each at 45° C. in high stringency wash buffer (0.1× SSC), and dried.

Reporter-labeled hybridization complexes were detected with a microscope equipped with an Innova 70 mixed gas 10 W laser (Coherent, Santa Clara Calif.) capable of generating spectral lines at 488 nm for excitation of Cy3 and at 632 nm for excitation of Cy5. The excitation laser light was focused on the microarray using a 20× microscope objective (Nikon, Melville N.Y.). The slide containing the microarray was placed on a computer-controlled X-Y stage on the microscope and raster-scanned past the objective. The 1.8 cm×1.8 cm microarray used in the present example was scanned with a resolution of 20 micrometers.

In two separate scans, the mixed gas multiline laser excited the two fluorophores sequentially. Emitted light was spilt, based on wavelength, into two photomultiplier tube detectors (PMT R1477; Hamamatsu Photonics Systems, Bridgewater N.J.) corresponding to the two fluorophores. Appropriate filters positioned between the microarray and the photomultiplier tubes were used to filter the signals. The emission maxima of the fluorophores used were 565 nm for Cy3 and 650 nm for Cy5. Each microarray was typically scanned twice, one scan per fluorophore using the appropriate filters at the laser source, although the apparatus was capable of recording the spectra from both fluorophores simultaneously.

The sensitivity of the scans was calibrated using the signal intensity generated by a cDNA control species. Samples of the calibrating cDNA were separately labeled with the two fluorophores and identical amounts of each were added to the hybridization mixture. A specific location on the microarray contained a complementary DNA sequence, allowing the intensity of the signal at that location to be correlated with a weight ratio of hybridizing species of 1:100,000.

The output of the photomultiplier tube was digitized using a 12-bit RTI-835H analog-to-digital (A/D) conversion board (Analog Devices, Norwood, Mass.) installed in an IBM-compatible PC computer. The digitized data were displayed as an image where the signal intensity was mapped using a linear 20-color transformation to a pseudocolor scale ranging from blue (low signal) to red (high signal). The data was also analyzed quantitatively. Where two different fluorophores were excited and measured simultaneously, the data were first corrected for optical crosstalk (due to overlapping emission spectra) between the fluorophores using each fluorophore's emission spectrum.

A grid was superimposed over the fluorescence signal image such that the signal from each spot was centered in each element of the grid. The fluorescence signal within each element was then integrated to obtain a numerical value corresponding to the average intensity of the signal. The software used for signal analysis was the GEMTOOLS gene expression analysis program (Incyte Genomics). Significance was defined as signal to background ratio exceeding 2× and area hybridization exceeding 40%.

VIII Data Analysis and Results

Array elements that exhibited at least 2-fold change in expression, a signal intensity over 250 units, a signal-to-background ratio of at least 2.5, and an element spot size of at least 40% were identified as differentially expressed using the GEMTOOLS program (Incyte Genomics). Differential expression values were converted to log base 2 scale. The cDNAs that are differentially expressed are shown in Tables 1 and 2. The cDNAs identified in Table 1 are differentially expressed at least 2-fold in at least 50% of patient samples tested. These genes are useful diagnostic markers or as potential therapeutic targets for premalignant colon polyps or colon cancer. The cDNAs identified in Table 2 showed a statistically greater differential expression pattern in colon cancer than colon polyps by t-test analysis. These genes are useful diagnostic markers for colon tumor progression from premalignant colon polyps to cancer or as potential therapeutic targets for colon cancer.

IX Other Hybridization Technologies and Analyses

Other hybridization technologies utilize a variety of substrates such as nylon membranes, capillary tubes, etc. Arranging cDNAs on polymer coated slides is described in Example V; sample cDNA preparation and hybridization and analysis using polymer coated slides is described in examples VI and VII, respectively.

The cDNAs are applied to a membrane substrate by one of the following methods. A mixture of cDNAs is fractionated by gel electrophoresis and transferred to a nylon membrane by capillary transfer. Alternatively, the cDNAs are individually ligated to a vector and inserted into bacterial host cells to form a library. The cDNAs are then arranged on a substrate by one of the following methods. In the first method, bacterial cells containing individual clones are robotically picked and arranged on a nylon membrane. The membrane is placed on LB agar containing selective agent (carbenicillin, kanamycin, ampicillin, or chloramphenicol depending on the vector used) and incubated at 37° C. for 16 hr. The membrane is removed from the agar and consecutively placed colony side up in 10% SDS, denaturing solution (1.5 M NaCl, 0.5 M NaOH), neutralizing solution (1.5 M NaCl, 1 M Tris, pH 8.0), and twice in 2× SSC for 10 min each. The membrane is then UV irradiated in a STRATALINKER UV-crosslinker (Stratagene).

In the second method, cDNAs are amplified from bacterial vectors by thirty cycles of PCR using primers complementary to vector sequences flanking the insert. PCR amplification increases a starting concentration of 1-2 ng nucleic acid to a final quantity greater than 5 μg. Amplified nucleic acids from about 400 bp to about 5000 bp in length are purified using SEPHACRYL-400 beads (APB). Purified nucleic acids are arranged on a nylon membrane manually or using a dot/slot blotting manifold and suction device and are immobilized by denaturation, neutralization, and UV irradiation as described above.

Hybridization probes derived from cDNAs of the Sequence Listing are employed for screening cDNAs, mRNAs, or genomic DNA in membrane-based hybridizations. Probes are prepared by diluting the cDNAs to a concentration of 40-50 ng in 45 μl TE buffer, denaturing by heating to 100° C. for five min and briefly centrifuging. The denatured cDNA is then added to a REDIPRIME tube (APB), gently mixed until blue color is evenly distributed, and briefly centrifuged. Five microliters of [³²P]dCTP is added to the tube, and the contents are incubated at 37° C. for 10 min. The labeling reaction is stopped by adding 5 μl of 0.2M EDTA, and probe is purified from unincorporated nucleotides using a PROBEQUANT G-50 microcolumn (APB). The purified probe is heated to 100° C. for five min and then snap cooled for two min on ice.

Membranes are pre-hybridized in hybridization solution containing 1% Sarkosyl and 1× high phosphate buffer (0.5 M NaCl, 0.1 M NA₂HPO₄, 5 mM EDTA, pH 7) at 55° C. for two hr. The probe, diluted in 15 ml fresh hybridization solution, is then added to the membrane. The membrane is hybridized with the probe at 55° C. for 16 hr. Following hybridization, the membrane is washed for 15 min at 25° C. in 1 mM Tris (pH 8.0), 1% Sarkosyl, and four times for 15 min each at 25° C. in 1 mM Tris (pH 8.0). To detect hybridization complexes, XOMAT-AR film (Eastman Kodak, Rochester N.Y.) is exposed to the membrane overnight at −70° C., developed, and examined.

X Further Characterization of Differentially Expressed cDNAs and Proteins

Clones were blasted against the LIFESEQ Gold 5.1 database (Incyte Genomics) and an Incyte template and its sequence variants were chosen for each clone. The template and variant sequences were blasted against GenBank database to acquire annotation. The nucleotide sequences were translated into amino acid sequence which was blasted against the GenPept and other protein databases to acquire annotation and characterization, i.e., structural motifs.

Percent sequence identity can be determined electronically for two or more amino acid or nucleic acid sequences using the MEGALIGN program (DNASTAR). The percent identity between two amino acid sequences is calculated by dividing the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage identity.

Sequences with conserved protein motifs may be searched using the BLOCKS search program. This program analyses sequence information contained in the Swiss-Prot and PROSITE databases and is useful for determining the classification of uncharacterized proteins translated from genomic or cDNA sequences (Bairoch et al., supra; Attwood et al., supra). PROSITE database is a useful source for identifying functional or structural domains that are not detected using motifs due to extreme sequence divergence. Using weight matrices, these domains are calibrated against the SWISS-PROT database to obtain a measure of the chance distribution of the matches.

The PRINTS database can be searched using the BLIMPS search program to obtain protein family “fingerprints”. The PRINTS database complements the PROSITE database by exploiting groups of conserved motifs within sequence alignments to build characteristic signatures of different protein families. For both BLOCKS and PRINTS analyses, the cutoff scores for local similarity were: >1300=strong, 1000-1300=suggestive; for global similarity were: p<exp-3; and for strength (degree of correlation) were: >1300=strong, 1000-1300=weak.

XI Expression of the Encoded Protein

Expression and purification of a protein encoded by a cDNA of the invention is achieved using bacterial or virus-based expression systems. For expression in bacteria, cDNA is subcloned into a vector containing an antibiotic resistance gene and an inducible promoter that directs high levels of cDNA transcription. Examples of such promoters include, but are not limited to, the trp-lac (tac) hybrid promoter and the T5 or T7 bacteriophage promoter in conjunction with the lac operator regulatory element. Recombinant vectors are transformed into bacterial hosts, such as BL21 (DE3). Antibiotic resistant bacteria express the protein upon induction with IPTG. Expression in eukaryotic cells is achieved by infecting Spodoptera frugiperda (Sf9) insect cells with recombinant baculovirus, Autographica califormica nuclear polyhedrosis virus. The polyhedrin gene of baculovirus is replaced with the cDNA by either homologous recombination or bacterial-mediated transposition involving transfer plasmid intermediates. Viral infectivity is maintained and the strong polyhedrin promoter drives high levels of transcription.

For ease of purification, the protein is synthesized as a fusion protein with glutathione-S-transferase (GST; APB) or a similar alternative such as FLAG. The fusion protein is purified on immobilized glutathione under conditions that maintain protein activity and antigenicity. After purification, the GST moiety is proteolytically cleaved from the protein with thrombin. A fusion protein with FLAG, an 8-amino acid peptide, is purified using commercially available monoclonal and polyclonal anti-FLAG antibodies (Eastman Kodak, Rochester N.Y.).

XII Production of Specific Antibodies

A denatured protein from a reverse phase HPLC separation is obtained in quantities up to 75 mg. This denatured protein is used to immunize mice or rabbits following standard protocols. About 100 μg is used to immunize a mouse, while up to 1 mg is used to immunize a rabbit. The denatured protein is radioiodinated and incubated with murine B-cell hybridomas to screen for monoclonal antibodies. About 20 mg of protein is sufficient for labeling and screening several thousand clones.

In another approach, the amino acid sequence translated from a cDNA of the invention is analyzed using PROTEAN software (DNASTAR) to determine regions of high antigenicity, essentially antigenically-effective epitopes of the protein. The optimal sequences for immunization are usually at the C-terminus, the N-terminus, and those intervening, hydrophilic regions of the protein that are likely to be exposed to the external environment when the protein is in its natural conformation. Typically, oligopeptides about 15 residues in length are synthesized using an ABI 431 peptide synthesizer (Applied Biosystems) using Fmoc-chemistry and then coupled to keyhole limpet hemocyanin (KLH; Sigma Aldrich) by reaction with M-maleimidobenzoyl-N-hydroxysuccinimide ester. If necessary, a cysteine may be introduced at the N-terminus of the peptide to permit coupling to KLH. Rabbits are immunized with the oligopeptide-KLH complex in complete Freund's adjuvant. The resulting antisera are tested for antipeptide activity by binding the peptide to plastic, blocking with 1% BSA, reacting with rabbit antisera, washing, and reacting with radioiodinated goat anti-rabbit IgG.

Hybridomas are prepared and screened using standard techniques. Hybridomas of interest are detected by screening with radioiodinated protein to identify those fusions producing a monoclonal antibody specific for the protein. In a typical protocol, wells of 96 well plates (FAST, Becton-Dickinson, Palo Alto Calif.) are coated with affinity-purified, specific rabbit-anti-mouse (or suitable anti-species Ig) antibodies at 10 mg/ml. The coated wells are blocked with 1% BSA and washed and exposed to supernatants from hybridomas. After incubation, the wells are exposed to radiolabeled protein at 1 mg/ml. Clones producing antibodies bind a quantity of labeled protein that is detectable above background.

Such clones are expanded and subjected to 2 cycles of cloning at 1 cell/3 wells. Cloned hybridomas are injected into pristane-treated mice to produce ascites, and monoclonal antibody is purified from the ascitic fluid by affinity chromatography on protein A (APB). Monoclonal antibodies with affinities of at least 10⁸ M⁻¹, preferably 10⁹ to 10¹⁰ M⁻¹ or stronger, are made by procedures well known in the art.

XIII Purification of Naturally Occurring Protein Using Specific Antibodies

Naturally occurring or recombinant protein is substantially purified by immunoaffinity chromatography using antibodies specific for the protein. An immunoaffinity column is constructed by covalently coupling the antibody to CNBr-activated SEPHAROSE resin (APB). Media containing the protein is passed over the immunoaffinity column, and the column is washed using high ionic strength buffers in the presence of detergent to allow preferential absorbance of the protein. After coupling, the protein is eluted from the column using a buffer of pH 2-3 or a high concentration of urea or thiocyanate ion to disrupt antibody/protein binding, and the protein is collected.

XIV Screening Molecules for Specific Binding with the cDNA or Protein

The cDNA or fragments thereof and the protein or portions thereof are labeled with ³²P-dCTP, Cy3-dCTP, Cy5-dCTP (APB), or BIODIPY or FITC (Molecular Probes), respectively. Candidate molecules or compounds previously arranged on a substrate are incubated in the presence of labeled nucleic or amino acid. After incubation under conditions for either a cDNA or a protein, the substrate is washed, and any position on the substrate retaining label, which indicates specific binding or complex formation, is assayed. The binding molecule is identified by its arrayed position on the substrate. Data obtained using different concentrations of the nucleic acid or protein are used to calculate affinity between the labeled nucleic acid or protein and the bound molecule. High throughput screening using very small assay volumes and very small amounts of test compound is fully described in Burbaum et al U.S. Pat. No. 5,876,946.

All patents and publications mentioned in the specification are incorporated herein by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the field of molecular biology or related fields are intended to be within the scope of the following claims.

TABLE 1

| | 3754 | 3755 | 3583 | 3311 | 3756 | 3757 | 3649 | 3647 | 3839 | 3581 |
|---|---|---|---|---|---|---|---|---|---|---|
| Clone ID | polyp | polyp | polyp | tumor | tumor | tumor | tumor | tumor | tumor | tumor |
| 1554043 | | | −0.77 | | | | −1.25 | −2.55 | −0.94 | |
| 2344730 | −2.26 | −1.55 | −1.65 | −1.72 | −1.71 | −2.14 | −1.29 | −2.61 | −2.06 | −0.25 |
| 2921991 | −1.30 | −1.13 | −1.10 | −1.61 | −0.08 | −1.40 | −1.50 | 0.12 | | −0.91 |
| 1805613 | −1.18 | −1.01 | −0.10 | −1.75 | −1.62 | −1.75 | −0.66 | −1.62 | | −0.93 |
| 1583076 | −2.84 | −3.10 | −1.31 | −2.93 | −2.50 | −0.95 | −1.39 | −2.01 | | −1.39 |
| 2771046 | −2.19 | −1.94 | −2.50 | −2.09 | −1.92 | −0.79 | −0.63 | −2.38 | | 0.00 |
| 1804503 | −3.45 | −2.83 | −1.85 | −2.69 | −2.58 | −2.92 | −2.91 | −1.64 | −3.99 | 0.17 |
| 1804503 | −3.12 | −2.56 | −2.03 | −2.70 | −2.84 | −3.09 | −1.71 | −0.98 | | −0.06 |
| 1560987 | −1.46 | −2.01 | −0.13 | −2.11 | −1.44 | −1.11 | −0.90 | −1.21 | | −0.19 |
| 1626523 | −1.58 | −2.42 | −0.87 | −1.92 | −2.13 | −2.02 | 0.07 | −1.71 | | −1.23 |
| 4540779 | −1.63 | −1.76 | −1.89 | −0.93 | −1.58 | −1.98 | −0.93 | −2.05 | −1.62 | −1.38 |
| 3699582 | −1.20 | −1.03 | −0.67 | −1.16 | −1.21 | −1.08 | −0.37 | −1.57 | −1.80 | −0.63 |
| 698665 | 1.44 | 2.54 | 1.81 | 2.04 | 2.06 | 1.24 | 0.83 | 0.35 | | 0.65 |
| 4002745 | 1.38 | 2.26 | 1.39 | 1.67 | 2.03 | 0.98 | 0.78 | 0.33 | 1.08 | 0.25 |
| 1630650 | −1.55 | −2.03 | −0.98 | −2.82 | −2.24 | −1.50 | −1.42 | −1.14 | −1.50 | −0.23 |
| 1630650 | −1.89 | −2.07 | −1.26 | −2.66 | −2.71 | −2.46 | −1.30 | −1.37 | | 0.05 |
| 2129558 | −1.70 | −1.62 | −1.32 | −1.93 | −1.51 | −1.64 | −1.14 | −1.22 | 0.00 | 0.00 |
| 1738354 | −2.24 | −3.24 | −1.82 | −4.63 | −3.77 | −3.32 | −2.94 | −4.31 | −3.65 | −1.58 |
| 2767646 | −2.17 | −2.84 | −1.92 | −3.19 | −3.51 | −2.94 | −2.94 | −4.32 | 0.00 | 0.00 |
| 1932453 | −4.31 | −4.80 | −1.38 | −4.48 | −4.29 | −3.71 | −2.03 | −1.29 | −3.46 | −2.45 |
| 2516950 | −1.16 | −1.59 | −1.40 | −1.01 | −0.95 | −1.13 | −1.80 | −2.78 | −2.38 | −2.80 |
| 1806417 | −3.06 | −3.02 | −1.06 | −2.74 | −2.98 | −2.79 | 0.00 | 0.00 | | 0.00 |
| 2512879 | −2.78 | −2.72 | −1.22 | −2.40 | −2.57 | −1.14 | −1.58 | −2.03 | | −1.35 |
| 5392053 | −3.09 | −2.11 | 0.01 | −2.67 | −2.09 | −2.38 | −1.90 | 0.00 | 0.00 | −1.26 |
| 2054053 | −1.24 | −1.30 | −1.26 | −1.08 | −0.83 | −1.67 | −0.21 | −1.17 | | −1.74 |
| 1841735 | −3.16 | −3.31 | −1.40 | −3.10 | −3.22 | −2.69 | −1.34 | −1.69 | −2.96 | −1.19 |
| 1842009 | −5.10 | −4.43 | −2.84 | −5.14 | −5.04 | −4.45 | −3.11 | −4.90 | | −3.53 |
| 2697455 | −1.62 | −2.17 | −1.68 | −1.55 | −1.30 | −1.45 | −0.83 | −2.13 | −1.37 | −1.45 |
| 1403294 | −1.23 | −2.18 | −0.65 | −2.07 | −1.10 | −1.56 | −0.54 | −1.88 | | −2.03 |
| 496003 | −1.14 | −1.90 | −1.18 | −1.85 | −0.77 | −1.83 | −0.76 | −1.87 | | −2.27 |
| 1800114 | −0.36 | −1.11 | −0.21 | −2.40 | −2.14 | −1.30 | −0.31 | −1.55 | | −1.21 |
| 27775 | −2.53 | −1.70 | −1.93 | −1.73 | −0.80 | −1.13 | −1.95 | −1.15 | | −1.12 |
| 5038171 | −2.18 | −1.50 | −0.30 | −1.78 | −1.59 | −1.85 | −1.17 | 0.00 | 0.00 | −2.98 |
| 1417020 | | | | | | | 0.51 | 1.16 | 1.40 | |
| 1800311 | −4.11 | −3.77 | −2.13 | −3.68 | −3.36 | −3.03 | −2.31 | −3.12 | −3.24 | −2.65 |
| 1846428 | −3.24 | −2.92 | −2.22 | −2.91 | −2.94 | −2.94 | −2.41 | −2.71 | | −1.78 |
| 1903267 | 2.43 | −1.23 | −0.23 | 1.22 | −0.94 | 3.09 | 1.65 | −3.50 | | 0.00 |
| 609115 | −1.52 | −1.32 | −1.38 | −1.17 | −0.77 | −1.77 | −0.49 | −2.78 | | −0.54 |
| 3054669 | | | | | | | −1.38 | −2.36 | −0.41 | |
| 2921194 | −1.44 | −1.93 | −0.97 | −1.34 | −1.37 | −1.25 | −0.65 | −2.37 | | −1.13 |
| 2150288 | −1.32 | −1.72 | −1.26 | −1.23 | −1.14 | −1.05 | −0.56 | −1.63 | −0.99 | −1.29 |
| 3977425 | −1.69 | −2.11 | −1.68 | −1.50 | −1.27 | −1.07 | −0.71 | −2.11 | | −1.18 |
| 990375 | −1.95 | −3.12 | −1.76 | −3.97 | −4.35 | −2.10 | −1.60 | −3.20 | | −1.74 |
| 2757583 | −2.74 | −2.54 | −1.33 | −2.58 | −1.56 | −1.39 | −1.43 | −2.30 | | −1.38 |
| 279898 | −1.90 | −2.36 | −0.88 | −1.78 | −1.09 | −1.38 | −1.01 | −2.07 | −0.86 | 0.00 |
| 2955163 | 1.40 | 1.30 | −0.08 | 1.96 | 2.09 | 1.69 | 1.37 | 0.81 | | 0.00 |
| 1761086 | −1.27 | −1.66 | −0.64 | −1.95 | −1.17 | −1.20 | −0.22 | −1.53 | −1.35 | −1.31 |
| 3878420 | | | −0.37 | | | | −0.79 | −1.35 | −1.11 | |
| 1807085 | −1.14 | −1.71 | −1.28 | −1.19 | −0.98 | −1.04 | −0.73 | −1.35 | −1.23 | −0.97 |
| 3888832 | −2.15 | −1.77 | −0.17 | −2.07 | −1.31 | −1.75 | −1.20 | −1.02 | | −0.92 |
| 1701847 | −0.70 | −1.73 | −1.02 | 1.56 | −1.85 | −1.53 | 1.09 | −1.03 | | 0.00 |
| 1981145 | −1.30 | −1.30 | −0.95 | −1.18 | −1.31 | −1.54 | 0.39 | −1.93 | | −0.23 |
| 1695477 | −1.50 | −1.58 | −0.18 | −1.42 | −0.46 | −1.62 | −1.24 | −1.26 | | −0.34 |
| 2060823 | 1.38 | 0.85 | 1.95 | 2.13 | 1.67 | −0.09 | 2.72 | −1.07 | 3.14 | −0.96 |
| 2060823 | 1.55 | 0.91 | 1.88 | 2.10 | 1.73 | 0.06 | 2.13 | −0.55 | | −1.01 |
| 1286257 | −1.27 | −1.42 | −1.13 | −1.32 | −1.39 | −1.01 | −0.59 | −1.35 | −1.04 | −0.42 |
| 1734393 | −2.81 | −3.60 | −2.36 | −2.72 | −4.49 | −2.75 | −1.86 | −3.65 | | −2.90 |
| 560466 | 1.77 | 1.38 | 1.79 | 1.70 | 1.85 | 1.30 | 1.33 | 0.53 | | 1.21 |
| 2215563 | −2.05 | −2.40 | −1.45 | −1.41 | −2.33 | −1.90 | −0.90 | −2.79 | −1.97 | −2.14 |
| 2215563 | −1.90 | −2.17 | −1.81 | −1.31 | −2.29 | −1.67 | −0.76 | −2.62 | | −2.01 |
| 2513883 | −2.44 | −2.23 | −0.49 | −2.02 | −1.21 | −1.51 | −0.66 | −1.51 | | −0.47 |
| 1747339 | | | | | | | −1.05 | −1.94 | −0.08 | |
| 417817 | −2.48 | −2.25 | −1.13 | −1.56 | −1.42 | −1.89 | −0.96 | −1.49 | −1.86 | −0.95 |
| 3206210 | −1.43 | −1.99 | −1.28 | −1.42 | −0.27 | −1.29 | −0.33 | −2.20 | | −1.30 |
| 957523 | −1.43 | −2.44 | −0.95 | −1.65 | −0.75 | −1.10 | −0.15 | −1.48 | −1.45 | −1.68 |
| 1988239 | −1.28 | −2.03 | −1.22 | −1.73 | −1.59 | −1.56 | −0.31 | −1.95 | −1.70 | −0.88 |
| 1988239 | −1.26 | −1.36 | −1.11 | −1.27 | −1.36 | −1.32 | −0.62 | −1.38 | | −0.56 |
| 2959255 | −2.07 | −2.27 | −0.15 | −1.63 | −1.85 | −1.50 | −1.17 | −2.02 | | −0.09 |
| 1806071 | −1.96 | −2.72 | −2.08 | −1.32 | −2.19 | −2.75 | −0.97 | −2.18 | | −1.30 |
| 1500810 | −2.63 | −2.53 | −1.72 | −3.79 | −3.65 | −2.80 | −2.31 | −2.77 | −2.74 | 0.00 |
| 1945315 | −2.09 | −2.02 | −1.50 | −2.74 | −2.42 | −2.45 | −2.08 | −2.44 | −1.95 | 0.00 |
| 3560862 | −2.20 | −2.18 | −1.72 | −3.02 | −2.87 | −2.36 | −2.06 | −2.67 | −2.08 | −0.16 |
| 4796795 | −2.85 | −2.10 | −1.56 | −2.72 | −2.01 | −1.68 | −1.88 | −1.92 | −2.32 | −0.62 |
| 4289557 | | | | | | | 0.43 | 1.77 | 1.13 | |
| 1986901 | | | −0.79 | | | | −1.37 | −1.48 | −1.65 | |
| 4151758 | −1.26 | −1.03 | −1.16 | −1.50 | −0.80 | −1.12 | −0.27 | −2.32 | −0.98 | −1.20 |
| 1431273 | −1.04 | −1.16 | −1.97 | −1.22 | −1.29 | −1.29 | −0.68 | −1.48 | | 0.00 |
| 1578941 | −1.79 | −1.78 | −1.69 | −1.42 | −1.24 | −1.27 | −1.71 | −3.34 | −2.51 | 0.00 |
| 1578941 | −1.59 | −1.61 | −1.83 | −1.38 | −1.27 | −1.73 | −1.51 | −2.77 | | −0.29 |
| 2900277 | −1.32 | −1.67 | −0.08 | −0.62 | −2.25 | −2.02 | −1.27 | −1.51 | | −0.16 |
| 1501080 | −1.71 | −1.94 | 0.04 | −2.00 | −1.79 | −2.46 | −0.53 | −1.02 | | 0.00 |
| 3119893 | | | | | | | −0.70 | −1.92 | −1.37 | |
| 3552835 | −2.19 | −1.46 | −1.25 | −1.84 | −2.00 | −2.49 | 0.01 | −3.39 | 1.35 | −1.74 |
| 699410 | −2.22 | −1.49 | −1.12 | −1.81 | −2.17 | −2.75 | −0.57 | −2.68 | | −1.42 |
| 1604425 | | | | | | | −1.05 | −1.61 | −0.05 | |
| 1632863 | | | | | | | −1.24 | −1.75 | −0.30 | |
| 2771895 | | | | | | | −1.17 | −2.15 | −0.10 | |
| 3395923 | | | | | | | −1.78 | −2.31 | −0.37 | |
| 2046165 | −1.97 | −1.85 | −1.05 | −0.73 | −1.08 | −1.55 | −1.22 | −1.29 | −0.89 | −0.43 |
| 1998428 | −1.01 | −1.44 | −0.52 | −0.90 | 0.88 | −1.33 | 1.39 | −2.30 | | −1.09 |
| 605219 | | | −0.24 | | | | −2.24 | −1.62 | −0.74 | |
| 1809178 | −1.55 | −1.62 | −1.61 | −1.43 | −1.10 | −1.90 | −0.89 | −2.49 | −0.70 | −1.92 |
| 991163 | −1.65 | −1.46 | −2.04 | −1.32 | −1.34 | −2.17 | −0.83 | −2.20 | | −2.20 |
| 444676 | −2.08 | −2.15 | −0.77 | −1.65 | −2.10 | −2.56 | −0.49 | −1.29 | | −0.83 |
| 3031144 | −2.02 | −1.46 | −1.09 | −1.49 | −1.95 | −1.10 | −1.10 | −1.29 | | −1.26 |
| 3075739 | −1.92 | −1.31 | −1.53 | −1.22 | −1.56 | −1.11 | −1.08 | −1.65 | −1.85 | −2.40 |
| 1424624 | −3.51 | −2.59 | −0.76 | −3.18 | −3.28 | −2.68 | −2.54 | −1.39 | | 0.00 |
| 1772981 | −2.30 | −1.86 | −1.20 | −2.06 | −1.74 | −1.91 | −1.73 | 0.00 | −2.28 | 0.00 |
| 2061014 | −1.20 | −1.32 | −0.30 | −1.09 | −1.22 | −1.09 | −0.09 | −1.44 | −1.05 | 0.00 |
| 2989680 | −1.35 | −2.12 | −1.72 | −2.04 | −1.80 | −2.13 | −0.80 | −3.22 | | −1.66 |
| 3096030 | | | | | | | −0.78 | −1.04 | −1.05 | |
| 1870876 | | | −1.58 | | | | −1.05 | −2.14 | −1.87 | |
| 2132203 | −1.63 | −1.47 | −0.87 | −1.28 | −1.82 | −1.63 | −0.91 | −2.55 | | −1.20 |
| 1522716 | −1.28 | −1.84 | −1.15 | −1.20 | −0.95 | −0.75 | −1.03 | −1.47 | | 0.00 |
| 2189062 | −1.21 | −1.12 | −1.31 | −1.26 | −0.38 | −1.03 | −0.63 | −1.58 | | −1.17 |
| 1962235 | −1.68 | −2.12 | −0.91 | −1.45 | −1.73 | −2.66 | −0.68 | −1.71 | | 0.00 |
| 1933073 | −2.83 | −2.96 | −1.56 | −2.56 | −2.69 | −1.69 | −2.03 | −1.77 | −3.32 | −0.73 |
| 1226538 | −2.85 | −2.96 | −1.41 | −2.89 | −2.59 | −2.32 | −1.69 | −1.95 | −3.11 | −1.68 |
| 1226538 | −3.41 | −3.50 | −1.65 | −3.28 | −3.16 | −2.70 | −1.88 | −2.57 | | −1.20 |
| 1498363 | −3.12 | −3.64 | −0.54 | −0.56 | −3.06 | −2.73 | 1.10 | −2.18 | 1.29 | −2.86 |
| 1582976 | | | −2.31 | | | | −1.30 | −1.69 | −2.79 | |
| 1820882 | | | −0.70 | | | | −1.15 | −1.96 | −0.82 | |
| 1845590 | −3.87 | −3.12 | −2.00 | −3.98 | −3.82 | −2.74 | −2.83 | −1.94 | | 0.00 |
| 1845915 | −3.29 | −2.71 | −1.66 | −2.85 | −2.57 | −2.67 | −1.78 | −2.15 | −2.07 | 0.00 |
| 1963854 | −1.49 | −1.35 | −0.31 | −1.95 | −1.58 | −1.35 | −0.94 | −2.07 | | −0.52 |
| 2055371 | | | −1.03 | | | | −0.76 | −1.41 | −1.06 | |
| 2964448 | −1.41 | −1.61 | −0.84 | −1.92 | −1.65 | −1.87 | −2.29 | −1.04 | | 0.00 |
| 3222815 | | | 0.31 | | | | −1.42 | −1.80 | −0.89 | |
| 3732960 | | | | | | | −2.02 | −2.63 | −1.63 | |
| 4175376 | | | −0.37 | | | | −0.91 | −2.49 | −1.95 | |
| 4872725 | | | | | | | −1.34 | −1.15 | −0.34 | |
| 5266376 | | | | | | | −1.31 | −2.23 | −1.85 | |
| 607958 | −1.10 | −1.88 | −0.81 | −1.40 | −1.54 | −1.17 | −1.11 | −0.98 | −1.30 | −1.43 |
| 2101663 | −4.85 | −4.72 | −2.19 | −4.48 | −4.71 | −4.71 | −3.43 | −2.75 | | −3.28 |
| 3681722 | −1.51 | −1.99 | −0.83 | −1.44 | −1.15 | −1.65 | −1.10 | −2.00 | −0.45 | −0.84 |
| 3229449 | | | 0.18 | | | | −1.59 | −1.74 | −0.98 | |
| 3090127 | | | −1.43 | | | | −0.32 | −1.26 | −0.93 | |

TABLE 2

| | 3754 | 3755 | 3583 | 3311 | 3756 | 3757 | 3649 | 3647 | t-test |
|---|---|---|---|---|---|---|---|---|---|
| Clone ID | polyp | polyp | polyp | tumor | tumor | tumor | tumor | tumor | polyp vs tumor |
| 1633719 | −1.98 | −1.98 | −2.32 | −3.47 | −3.10 | −2.99 | −2.19 | −2.89 | 0.0138 |
| 2785701 | 0.56 | 0.04 | −0.27 | 2.23 | 3.65 | 0.83 | 1.03 | 0.90 | 0.0392 |
| 3732536 | 0.34 | −0.06 | 0.03 | 3.46 | 3.42 | 0.50 | 1.24 | 0.91 | 0.0464 |
| 2767646 | −2.17 | −2.84 | −1.92 | −3.19 | −3.51 | −2.94 | −2.94 | −4.32 | 0.0349 |
| 1628788 | −0.67 | −2.16 | −0.28 | −4.78 | −3.55 | −2.37 | −2.17 | −3.48 | 0.0329 |
| 1921393 | −0.79 | −0.53 | −0.52 | −1.60 | −1.29 | −2.02 | −1.76 | −2.18 | 0.0008 |
| 1846463 | −1.78 | −1.67 | −2.05 | −1.65 | −1.01 | −1.18 | −1.10 | −0.95 | 0.0086 |
| 2532486 | 0.07 | −0.29 | 0.28 | −0.51 | −1.48 | −0.66 | −0.31 | −0.41 | 0.0422 |
| 2042056 | −0.04 | −0.21 | 0.43 | 0.12 | 0.89 | 0.87 | 1.31 | 1.34 | 0.0283 |
| 2595754 | −0.58 | −0.73 | −0.28 | 0.93 | 0.55 | 1.61 | 0.93 | −0.52 | 0.0215 |
| 2591494 | −0.51 | −0.83 | −0.04 | −1.96 | −2.42 | −2.27 | −2.38 | −1.32 | 0.0034 |
| 2845102 | 0.07 | −0.61 | −0.32 | 0.83 | 1.33 | −0.07 | 1.41 | 0.32 | 0.0235 |
| 4107476 | −2.19 | −1.87 | −2.31 | −3.11 | −3.24 | −3.33 | −2.32 | −2.93 | 0.0082 |
| 1861456 | 0.31 | −0.03 | −0.03 | 0.38 | 1.37 | 0.40 | 0.77 | 0.59 | 0.0278 |
| 3679667 | 0.53 | 0.19 | 0.22 | 0.90 | 1.39 | 0.39 | 0.72 | 0.90 | 0.0316 |
| 461001 | −0.78 | −0.58 | −0.83 | −2.31 | −1.61 | −1.94 | −2.19 | −2.58 | 0.0004 |

TABLE 3

| SEQ ID NO: | Template ID | Clone ID | Start | Stop |
|---|---|---|---|---|
| 1 | 184081.24 | 27775 | 188 | 424 |
| 2 | 995839.2 | 279898 | 41 | 352 |
| 3 | 3200830CB1 | 417817 | 260 | 581 |
| 5 | 006512.8 | 417817 | 1 | 457 |
| 6 | 3819039CB1 | 444676 | 997 | 2332 |
| 8 | 330923.5 | 496003 | 1363 | 3659 |
| 8 | 330923.5 | 1403294 | 2948 | 3595 |
| 9 | 234630.57 | 560466 | 1372 | 2397 |
| 10 | 611514CB1 | 605219 | 1131 | 1679 |
| 12 | 2072479CB1 | 607958 | 88 | 566 |
| 14 | 410911.5 | 609115 | 1616 | 3686 |
| 15 | 1285632CB1 | 698665 | 1377 | 4119 |
| 17 | 474322.36 | 699410 | 386 | 735 |
| 18 | 3040213CB1 | 957523 | 1383 | 1818 |
| 18 | 3040213CB1 | 3206210 | 6 | 1816 |
| 20 | 1282225CB1 | 990375 | 61 | 524 |
| 22 | 991163CB1 | 991163 | 861 | 1702 |
| 22 | 991163CB1 | 1809178 | 933 | 1346 |
| 24 | 3220207CB1 | 1226538 | 67 | 616 |
| 26 | 203309.2 | 1286257 | 2071 | 2124 |
| 27 | 998971.1 | 1417020 | 5 | 512 |
| 28 | 333076.1 | 1424624 | 2647 | 2951 |
| 28 | 333076.1 | 1772981 | 3371 | 3664 |
| 29 | 989613CB1 | 1431273 | 864 | 2869 |
| 31 | 2921920CB1 | 1498363 | 520 | 1103 |
| 33 | 997080.1 | 1500810 | 882 | 2910 |
| 34 | 5517972CB1 | 1501080 | 1186 | 2519 |
| 36 | 1397781.7 | 1522716 | 1328 | 1966 |
| 37 | 236655.3 | 1554043 | 1 | 351 |
| 38 | 345275.4 | 1560987 | 2023 | 2317 |
| 39 | 124600CB1 | 1578941 | 38 | 611 |
| 41 | 978410.7 | 1582976 | 297 | 718 |
| 42 | 1401116.1 | 1604425 | 1 | 830 |
| 43 | 2921009CB1 | 1626523 | 1461 | 2096 |
| 45 | 255115.4 | 1630650 | 1360 | 1852 |
| 46 | 1213592.1 | 1632863 | 318 | 686 |
| 47 | 1376382CB1 | 1695477 | 969 | 1499 |
| 49 | 2264641CB1 | 1701847 | 1088 | 1647 |
| 51 | 237547CB1 | 1734393 | 771 | 1305 |
| 53 | 2771481CB1 | 1738354 | 2208 | 3146 |
| 53 | 2771481CB1 | 2767646 | 1 | 3151 |
| 55 | 1400916.1 | 1747339 | 1 | 846 |
| 56 | 253986.11 | 1761086 | 873 | 1379 |
| 57 | 253986.17 | 1761086 | 230 | 743 |
| 58 | 2680109CB1 | 1800114 | 1993 | 2783 |
| 60 | 1800311CB1 | 1800311 | 74 | 636 |
| 60 | 1800311CB1 | 1846428 | 136 | 602 |
| 62 | 1804734CB1 | 1804503 | 607 | 1274 |
| 64 | 3231154CB1 | 1805613 | 421 | 797 |
| 66 | 210095.11 | 1806071 | 2788 | 3437 |
| 67 | 2719813CB1 | 1806417 | 220 | 1116 |
| 69 | 2886583CB1 | 1807085 | 329 | 922 |
| 71 | 025685.3 | 1820882 | 159 | 623 |
| 72 | 1808144CB1 | 1841735 | 2373 | 2829 |
| 72 | 1808144CB1 | 1842009 | 1461 | 2816 |
| 74 | 201356.1 | 1845590 | 1439 | 3456 |
| 75 | 978178.7 | 1845915 | 1022 | 1511 |
| 76 | 237563.31 | 1870876 | 1456 | 2075 |
| 77 | 1100412.5 | 1903267 | 2382 | 2833 |
| 78 | 1100412.4 | 1903267 | 2509 | 2860 |
| 79 | 2101663CB1 | 1932453 | 688 | 1205 |
| 81 | 611082CB1 | 1933073 | 177 | 1285 |
| 83 | 255002.4 | 1945315 | 1 | 355 |
| 84 | 1092257.2 | 1962235 | 2348 | 2796 |
| 84 | 1092257.2 | 3440567 | 2334 | 2777 |
| 85 | 1102315.3 | 1963854 | 1746 | 2059 |
| 86 | 1543330CB1 | 1981145 | 1138 | 1664 |
| 88 | 232992.1 | 1986901 | 2365 | 3110 |
| 89 | 1281620CB1 | 1988239 | 129 | 1192 |
| 91 | 343502.10 | 1998428 | 617 | 1062 |
| 92 | 1635966CB1 | 2046165 | 492 | 846 |
| 94 | 2054053CB1 | 2054053 | 332 | 869 |
| 96 | 096954.5 | 2055371 | 1585 | 3052 |
| 97 | 1422432CB1 | 2060823 | 397 | 842 |
| 99 | 409895.2 | 2060823 | 1224 | 1458 |
| 100 | 4874364CB1 | 2061014 | 60 | 611 |
| 102 | 239568.4 | 2101663 | 6 | 491 |
| 103 | 255041.1 | 2129558 | 1 | 494 |
| 104 | 2555628CB1 | 2132203 | 744 | 2019 |
| 106 | 255803.1 | 2150288 | 1 | 411 |
| 107 | 900341CB1 | 2189062 | 1102 | 1647 |
| 109 | 273879CB1 | 2215563 | 596 | 1817 |
| 111 | 141804.1 | 2344730 | 239 | 694 |
| 112 | 2512879CB1 | 2512879 | 130 | 1418 |
| 114 | 2685676CB1 | 2513883 | 465 | 882 |
| 116 | 2742913CB1 | 2516950 | 75 | 1693 |
| 118 | 429183.1 | 2697455 | 1 | 363 |
| 119 | 2757583CB1 | 2757583 | 61 | 431 |
| 121 | 1344279CB1 | 2771046 | 1591 | 3649 |
| 123 | 1329472.2 | 2771895 | 1 | 337 |
| 124 | 474457.35 | 2900277 | 94 | 684 |
| 125 | 474457.45 | 2900277 | 249 | 596 |
| 126 | 898779CB1 | 2921194 | 169 | 1098 |
| 128 | 1843408CB1 | 2921991 | 191 | 626 |
| 130 | 351241.1 | 2955163 | 312 | 757 |
| 131 | 413348.40 | 2959255 | 1704 | 2129 |
| 132 | 983354.2 | 2964448 | 4140 | 4432 |
| 133 | 235845.20 | 2989680 | 165 | 1424 |
| 134 | 266360.18 | 3031144 | 197 | 645 |
| 135 | 266360.15 | 3031144 | 371 | 657 |
| 136 | 1310030.1 | 3054669 | 10 | 180 |
| 137 | 2804864CB1 | 3075739 | 14 | 793 |
| 139 | 349615.7 | 3090127 | 616 | 1031 |
| 140 | 632664CB1 | 3096030 | 531 | 1076 |
| 142 | 995929.22 | 3119893 | 72 | 455 |
| 143 | 995929.27 | 3119893 | 663 | 1053 |
| 144 | 1397029.1 | 3222815 | 19 | 1212 |
| 145 | 403560.1 | 3229449 | 129 | 810 |
| 146 | 1329606.3 | 3395923 | 306 | 1480 |
| 147 | 1092257.12 | 3440567 | 1 | 532 |
| 148 | 474322.38 | 3552835 | 998 | 1797 |
| 149 | 255002.3 | 3560862 | 1 | 334 |
| 150 | 1330137.1 | 3681722 | 1 | 237 |
| 151 | 3699582CB1 | 3699582 | 1 | 494 |
| 153 | 344537.24 | 3699582 | 2049 | 2339 |
| 154 | 016124.2 | 3732960 | 31 | 391 |
| 155 | 104423.33 | 3878420 | 2339 | 2649 |
| 156 | 406977.2 | 3888832 | 573 | 1739 |
| 157 | 3355973CB1 | 3977425 | 25 | 1734 |
| 159 | 406457.3 | 4002745 | 4314 | 4773 |
| 160 | 2190217CB1 | 4151758 | 168 | 1116 |
| 162 | 029061.1 | 4175376 | 176 | 1158 |
| 163 | 1262593.2 | 4289557 | 1156 | 1510 |
| 164 | 1094812.1 | 4540779 | 29 | 679 |
| 165 | 2434655CB1 | 4796795 | 34 | 1027 |
| 167 | 206344.1 | 4872725 | 270 | 937 |
| 168 | 1075717.7 | 5038171 | 977 | 1374 |
| 169 | 1075717.1 | 5038171 | 556 | 1147 |
| 170 | 372647.1 | 5266376 | 81 | 792 |
| 171 | 148512.1 | 5392053 | 581 | 857 |
| 172 | 2023119CB1 | 1846463 | 2355 | 3242 |
| 174 | 1973832CB1 | 3732536 | 426 | 1678 |
| 176 | 241888.54 | 2785701 | 180 | 1759 |
| 177 | 1736965CB1 | 461001 | 2 | 765 |
| 179 | 412065.17 | 2591494 | 763 | 1681 |
| 180 | 988660.32 | 1921393 | 284 | 703 |
| 181 | 1434821CB1 | 2595754 | 432 | 846 |
| 183 | 464689.64 | 2845102 | 1659 | 2266 |
| 184 | 464689.59 | 2845102 | 413 | 891 |
| 185 | 1384719.3 | 1861456 | 3177 | 3584 |
| 185 | 1384719.3 | 3679667 | 3030 | 3522 |
| 186 | 407463.1 | 2532486 | 4549 | 5048 |
| 187 | 522433CB1 | 2042056 | 854 | 1234 |
| 189 | 480489.5 | 4107476 | 1 | 598 |
| 190 | 480489.2 | 4107476 | 212 | 586 |
| 191 | 1737775CB1 | 1628788 | 2293 | 2794 |
| 193 | 088078CB1 | 1633719 | 1021 | 1682 |

TABLE 4

| | Nucleotide | | Protein |
|---|---|---|---|
| SEQ ID NO: | Template ID | SEQ ID NO: | Template ID |
| 3 | 3200830CB1 | 4 | 3200830CD1 |
| 6 | 3819039CB1 | 7 | 3819039CD1 |
| 10 | 611514CB1 | 11 | 611514CD1 |
| 12 | 2072479CB1 | 13 | 2072479CD1 |
| 15 | 1285632CB1 | 16 | 1285632CD1 |
| 18 | 3040213CB1 | 19 | 3040213CD1 |
| 20 | 1282225CB1 | 21 | 1282225CD1 |
| 22 | 991163CB1 | 23 | 991163CD1 |
| 24 | 3220207CB1 | 25 | 3220207CD1 |
| 29 | 989613CB1 | 30 | 989613CD1 |
| 31 | 2921920CB1 | 32 | 2921920CD1 |
| 34 | 5517972CB1 | 35 | 5517972CD1 |
| 39 | 124600CB1 | 40 | 124600CD1 |
| 43 | 2921009CB1 | 44 | 2921009CD1 |
| 47 | 1376382CB1 | 48 | 1376382CD1 |
| 49 | 2264641CB1 | 50 | 2264641CD1 |
| 51 | 237547CB1 | 52 | 237547CD1 |
| 53 | 2771481CB1 | 54 | 2771481CD1 |
| 58 | 2680109CB1 | 59 | 2680109CD1 |
| 60 | 1800311CB1 | 61 | 1800311CD1 |
| 62 | 1804734CB1 | 63 | 1804734CD1 |
| 64 | 3231154CB1 | 65 | 3231154CD1 |
| 67 | 2719813CB1 | 68 | 2719813CD1 |
| 69 | 2886583CB1 | 70 | 2886583CD1 |
| 72 | 1808144CB1 | 73 | 1808144CD1 |
| 79 | 2101663CB1 | 80 | 2101663CD1 |
| 81 | 611082CB1 | 82 | 611082CD1 |
| 86 | 1543330CB1 | 87 | 1543330CD1 |
| 89 | 1281620CB1 | 90 | 1281620CD1 |
| 92 | 1635966CB1 | 93 | 1635966CD1 |
| 94 | 2054053CB1 | 95 | 2054053CD1 |
| 97 | 1422432CB1 | 98 | 1422432CD1 |
| 100 | 4874364CB1 | 101 | 4874364CD1 |
| 104 | 2555628CB1 | 105 | 2555628CD1 |
| 107 | 900341CB1 | 108 | 900341CD1 |
| 109 | 273879CB1 | 110 | 273879CD1 |
| 112 | 2512879CB1 | 113 | 2512879CD1 |
| 114 | 2685676CB1 | 115 | 2685676CD1 |
| 116 | 2742913CB1 | 117 | 2742913CD1 |
| 119 | 2757583CB1 | 120 | 2757583CD1 |
| 121 | 1344279CB1 | 122 | 1344279CD1 |
| 126 | 898779CB1 | 127 | 898779CD1 |
| 128 | 1843408CB1 | 129 | 1843408CD1 |
| 137 | 2804864CB1 | 138 | 2804864CD1 |
| 140 | 632664CB1 | 141 | 632664CD1 |
| 151 | 3699582CB1 | 152 | 3699582CD1 |
| 157 | 3355973CB1 | 158 | 3355973CD1 |
| 160 | 2190217CB1 | 161 | 2190217CD1 |
| 165 | 2434655CB1 | 166 | 2434655CD1 |
| 172 | 2023119CB1 | 173 | 2023119CD1 |
| 174 | 1973832CB1 | 175 | 1973832CD1 |
| 177 | 1736965CB1 | 178 | 1736965CD1 |
| 181 | 1434821CB1 | 182 | 1434821CD1 |
| 187 | 522433CB1 | 188 | 522433CD1 |
| 191 | 1737775CB1 | 192 | 1737775CD1 |
| 193 | 088078CB1 | 194 | 088078CD1 |

TABLE 5

| SEQ ID NO | Template ID | GI Number | E-Value | GenBank Annotation |
|---|---|---|---|---|
| 1 | 184081.24 | g306743 | 0 | Human ferritin heavy chain mRNA, complete cds. |
| 2 | 995839.2 | g37120 | 0 | Human mRNA for metallothionein from cadmium-treated cells. |
| 3 | 3200830CB1 | g285948 | 0 | Human mRNA fro KIAA0106 gene, complete cds. |
| 4 | 3200830CD1 | g285948 | 0 | Human mRNA for KIAA0106 gene, complete cds. |
| 5 | 6512.8 | g285948 | 0 | Human mRNA for KIAA0106 gene, complete cds. |
| 6 | 3819039CB1 | g5231142 | 0 | Human serine/threonine protein kinase sgk mRNA, complete cds. |
| 7 | 3819039CD1 | g5231142 | 0 | Human serine/threonine protein kinase sgk mRNA, complete cds. |
| 8 | 330923.5 | g30507 | 0 | Human DSC2 mRNA for desmocollins type 2a and 2b. |
| 9 | 234630.57 | g31190 | 0 | Human mRNA for epican. |
| 10 | 611514CB1 | g1374791 | 0 | Human selenium-binding protein (hSBP) mRNA, complete cds. |
| 11 | 611514CD1 | g1374791 | 0 | Human selenium-binding protein (hSBP) mRNA, complete cds. |
| 12 | 2072479CB1 | | 0 | Incyte Unique. |
| 13 | 2072479CD1 | | 0 | Incyte Unique. |
| 14 | 410911.5 | g405229 | 0 | Human I-plastin mRNA, complete cds. |
| 15 | 1285632CB1 | g903681 | 0 | Human bumetanide-sensitive Na-K-Cl cotransporter (NKCC1) mRNA, complete |
| | | | | cds. |
| 16 | 1285632CD1 | g903681 | 0 | Human bumetanide-sensitive Na-K-Cl cotransporter (NKCC1) mRNA, complete |
| | | | | cds. |
| 17 | 474322.36 | g190888 | 0 | Human RASF-A PLA2 mRNA, complete cds. |
| 18 | 3040213CB1 | g28937 | 0 | Human mRNA for mitochondrial ATP synthase (F1-ATPase) alpha subunit. |
| 19 | 3040213CD1 | g28937 | 0 | Human mRNA for mitochondrial ATP synthase (F1-ATPase) alpha subunit. |
| 20 | 1282225CB1 | g182355 | 0 | Human liver fatty acid binding protein (FABP) mRNA, complete cds. |
| 21 | 1282225CD1 | g182355 | 0 | Human liver fatty acid binding protein (FABP) mRNA, complete cds. |
| 22 | 991163CB1 | g2507612 | 0 | Human serine protease mRNA, complete cds. |
| 23 | 991163CD1 | g2507612 | 0 | Human serine protease mRNA, complete cds. |
| 24 | 3220207CB1 | g187241 | 3.00E−54 | Human lymphocyte surface protein exons 1-5, complete cds. |
| 25 | 3220207CD1 | g187241 | 3.00E−54 | Human lymphocyte surface protein exons 1-5, complete cds. |
| 26 | 203309.2 | g406853 | 0 | Human mRNA for cytokeratin 20. |
| 27 | 998971.1 | g5931520 | 0 | Human genomic DNA, chromosome 22q11.2, Cat Eye Syndrome region, |
| | | | | clone: c60D12. |
| 28 | 333076.1 | g549987 | 0 | Human sulfate transporter (DTD) mRNA, complete cds. |
| 29 | 989613CB1 | g535474 | 0 | Human N-benozyl-L-tyrosyl-p-amino-benzoic acid hydrolase alpha subunit (PPH |
| | | | | alpha) mRNA, complete cds. |
| 30 | 989613CD1 | g535474 | 0 | Human N-benzoyl-L-tyrosyl-p-amino-benzoic acid hydrolase alpha submit (PPH |
| | | | | alpha) mRNA, complete cds. |
| 31 | 2921920CB1 | g7019845 | 0 | Human cDNA FLJ20022 fis, clone ADSE01331. |
| 32 | 2921920CD1 | g7019845 | 0 | Human cDNA FLJ20022 fis, clone ADSE01331. |
| 33 | 997080.1 | g4753765 | 0 | Human mRNA for UDP-glucuronosyltransferase. |
| 34 | 5517972CB1 | g4185795 | 0 | Human placenta-specific ATP-binding cassette transporter (ABCP) mRNA, |
| | | | | complete cds. |
| 35 | 5517972CD1 | g4185795 | 0 | Human placenta-specific ATP-binding cassette transporter (ABCP) mRNA, |
| | | | | complete cds. |
| 36 | 1397781.7 | g37851 | 0 | Human vimentin gene. |
| 37 | 236655.3 | g33140 | 2.00E−09 | C-terminal part of Human Ig kappa gene coding for amino acids 109 to 214. |
| 38 | 345275.4 | g1000711 | 0 | Human BENE mRNA, partial cds. |
| 39 | 124600CB1 | g1203983 | 0 | Human NAD+-dependent 15 hydroxyprostaglandin dehydrogenase (PGDH) |
| | | | | mRNA, complete cds. |
| 40 | 124600CD1 | g1203983 | 0 | Human NAD+-dependent 15 hydroxyprostaglandin dehydrogenase (PGDH) |
| | | | | mRNA, complete cds. |
| 41 | 978410.7 | | 0 | Incyte Unique |
| 42 | 1401116.1 | g33737 | 0 | Human rearranged Ig lambda light chain mRNA. |
| 43 | 2921009CB1 | g4204683 | 0 | Human beta-1,6-N-acetylglucosaminyltransferase mRNA, complete cds. |
| 44 | 2921009CD1 | g4204683 | 0 | Human beta-1,6-N-acetylglucosaminyltransferase mRNA, complete cds. |
| 45 | 255115.4 | g3287472 | 0 | Human C19steroid specific UDP-glucuronosyltransferase mRNA, complete cds. |
| 46 | 1213592.1 | g33394 | 0 | Human mRNA for Ig lambda-chain. |
| 47 | 1376382CB1 | g7020103 | 0 | Human cDNA FLJ20177 fis, clone COL09966, highly similar to Y08136 H. |
| 48 | 1376382CD1 | g7020103 | 0 | Human cDNA FLJ20177 fis, clone COL09966, highly similar to Y08136 H. |
| 49 | 2264641CB1 | g2447035 | 0 | Human mRNA for APS, complete cds. |
| 50 | 2264641CD1 | g2447035 | 0 | Human mRNA for APS, complete cds. |
| 51 | 237547CB1 | g406853 | 0 | Human mRNA for cytokeratin 20. |
| 52 | 237547CD1 | g406853 | 0 | Human mRNA for cytokeratin 20. |
| 53 | 2771481CB1 | g7019922 | 0 | Human cDNA FLJ20065 fis, clone COL01613, highly similar to ECLC_BOVIN |
| | | | | EPITHELIAL CHLORIDE CHANNEL PROTEIN. |
| 54 | 2771481CD1 | g7019922 | 0 | Human cDNA FLJ20065 fis, clone COL01613, highly similar to ECLC_BOVIN |
| | | | | EPITHELIAL CHLORIDE CHANNEL PROTEIN. |
| 55 | 1400916.1 | g2765426 | 0 | Human mRNA for Ig lambda light chain. |
| 56 | 253986.11 | g180589 | 0 | Human mitochondrial creatine kinase (CKMT) gene, complete cds. |
| 57 | 253986.17 | g180589 | 0 | Human mitochondrial creatine kinase (CKMT) gene, complete cds. |
| 58 | 2680109CB1 | g514365 | 0 | Human poly-Ig receptor transmembrane secretory component mRNA, 3′ end |
| 59 | 2680109CD1 | g514365 | 0 | Human poly-Ig receptor transmembrane secretory component mRNA, 3′ end |
| 60 | 1800311CB1 | g183414 | 0 | Human guanylin mRNA, complete cds. |
| 61 | 1800311CD1 | g183414 | 0 | Human guanylin mRNA, complete cds. |
| 62 | 1804734CB1 | g6606075 | 0 | Human aquaporin 8 (AQP8) mRNA, complete cds. |
| 63 | 1804734CD1 | g6606075 | 0 | Human aquaporin 8 (AQP8) mRNA, complete cds. |
| 64 | 3231154CB1 | g1814276 | 0 | Human A33 antigen precursor mRNA, complete cds. |
| 65 | 3231154CD1 | g1814276 | 0 | Human A33 antigen precursor mRNA, complete cds. |
| 66 | 210095.11 | g37197 | 0 | Human mRNA for transmembrane carcinoembryonic antigen BGPa (formerly |
| | | | | TM1-GEA). |
| 67 | 2719813CB1 | g179790 | 0 | Human carbonic anhydrase IV mRNA, complete cds. |
| 68 | 2719813CD1 | g179790 | 0 | Human carbonic anhydrase IV mRNA, complete cds. |
| 69 | 2886583CB1 | g3893156 | 0 | Human mRNA expressed in thyroid gland. |
| 70 | 2886583CD1 | g3893156 | 0 | Human mRNA expressed in thyroid gland. |
| 71 | 25685.3 | | 0 | Incyte Unique |
| 72 | 1808144CB1 | g291963 | 0 | Human colon mucosa-associated (DRA) mRNA, complete cds. |
| 73 | 1808144CD1 | g291963 | 0 | Human colon mucosa-associated (DRA) mRNA, complete cds. |
| 74 | 201356.1 | | 0 | Incyte Unique |
| 75 | 978178.7 | g200497 | 6.00E−23 | protein kinase inhibitor |
| 76 | 237563.31 | g881393 | 0 | Human uridine diphosphoglucose pyrophosphorylase mRNA, complete cds. |
| 77 | 1100412.5 | g3860076 | 0 | Human GW112 protein (GW112) mRNA, complete cds. |
| 78 | 1100412.4 | g7020929 | 0 | Human cDNA FLJ20676 fis, clone KAIA4294, highly similar to AF097021 |
| | | | | Human GW112 protein. |
| 79 | 2101663CB1 | g179792 | 0 | Human carbonic anhydrase I (CAI) mRNA, complete cds. |
| 80 | 2101663CD1 | g179792 | 0 | Human carbonic anhydrase I (CAI) mRNA, complete cds. |
| 81 | 611082CB1 | g7020167 | 0 | Human cDNA FLJ20217 fis, clone COLF3334. |
| 82 | 611082CD1 | g7020167 | 0 | Human cDNA FLJ20217 fis, clone COLF3334. |
| 83 | 255002.4 | g4753765 | 0 | Human mRNA for UDP-glucuronosyltransferase. |
| 84 | 1092257.2 | g5353532 | 0 | Human zinc finger transcription factor GKLF mRNA, complete cds. |
| 85 | 1102315.3 | g7259292 | 0 | contains transmembrane (TM) region |
| 86 | 1543330CB1 | g28871 | 0 | Human mRNA for argininosuccinate synthetase. |
| 87 | 1543330CD1 | g28871 | 0 | Human mRNA for argininosuccinate synthetase. |
| 88 | 232992.1 | g8247253 | 0 | Human mRNA for TRAF and TNF receptor associated protein (ttrap gene). |
| 89 | 1281620CB1 | g1877030 | 0 | Human mRNA for rhodanese, complete cds. |
| 90 | 1281620CD1 | g1877030 | 0 | Human mRNA for rhodanese, complete cds. |
| 91 | 343502.1 | g3779225 | 0 | Human secreted cement gland protein XAG-2 homolog (hAG-2/R) mRNA, |
| | | | | complete cds. |
| 92 | 1635966CB1 | g6318543 | 0 | Human retinal short-chain dehydrogenase/reductase retSDR2 mRNA, complete |
| | | | | cds. |
| 93 | 1635966CD1 | g6318543 | 0 | Human retinal short-chain dehydrogenase/reductase retSDR2 mRNA, complete |
| | | | | cds. |
| 94 | 2054053CB1 | g181122 | 0 | Human cleavage signal 1 protein mRNA, complete cds. |
| 95 | 2054053CD1 | g181122 | 0 | Human cleavage signal 1 protein mRNA, complete cds. |
| 96 | 96954.5 | g2804592 | 0 | F21856_2 |
| 97 | 1422432CB1 | g36177 | 0 | Human mRNA for calcium-binding protein S100P. |
| 98 | 1422432CD1 | g36177 | 0 | Human mRNA for calcium-binding protein S100P. |
| 99 | 409895.2 | g36177 | 0 | Human mRNA for calcium-binding protein S100P. |
| 100 | 4874364CB1 | g2826145 | 0 | Human mRNA for ST1B2, complete cds. |
| 101 | 4874364CD1 | g2826145 | 0 | Human mRNA for ST1B2, complete cds. |
| 102 | 239568.4 | g407977 | 0 | Macaque carbonic anhydrase I mRNA, complete cds. |
| 103 | 255041.1 | g3287472 | 1.00E−36 | Human C19steroid specific UDP-glucuronosyltransferase mRNA, complete cds. |
| 104 | 2555628CB1 | g881393 | 0 | Human uridine diphosphoglucose pyrophosphorylase mRNA, complete cds. |
| 105 | 2555628CD1 | g881393 | 0 | Human uridine diphosphoglucose pyrophosphorylase mRNA, complete cds. |
| 106 | 255803.1 | g6599184 | 0 | Human mRNA; cDNA DKFZp434C107 (from clone DKFZp434C107). |
| 107 | 900341CB1 | g5114259 | 0 | Human voltage-dependent anion channel isoform 2 (VDAC2) gene, exon 10 and |
| | | | | complete cds. |
| 108 | 900341CD1 | g5114259 | 0 | Human voltage-dependent anion channel isoform 2 (VDAC2) gene, exon 10 and |
| | | | | complete cds. |
| 109 | 273879CB1 | g2385453 | 0 | Human mRNA for galectin-4, complete cds. |
| 110 | 273879CD1 | g2385453 | 0 | Human mRNA for galectin-4, complete cds. |
| 111 | 141801.1 | g2529737 | 2.00E−67 | ER1 |
| 112 | 2512879CB1 | g178089 | 0 | Human class I alcohol dehydrogenase (ADH1) alpha subunit mRNA, complete |
| | | | | cds. |
| 113 | 2512879CD1 | g178089 | 0 | Human class I alcohol dehydrogenase (ADH1) alpha subunit mRNA, complete |
| | | | | cds. |
| 114 | 2685676CB1 | g517350 | 0 | Human MT1X gene for metallothionein 1X. |
| 115 | 2685676CD1 | g517350 | 0 | Human MT1X gene for metallothionein 1X. |
| 116 | 2742913CB1 | g179771 | 0 | Human carbonic anhydrase II mRNA, complete cds. |
| 117 | 2742913CD1 | g179771 | 0 | Human carbonic anhydrase II mRNA, complete cds. |
| 118 | 429183.1 | g1673574 | 0 | Human cytokeratin 8 mRNA, complete cds. |
| 119 | 2757583CB1 | g187542 | 0 | Human metallothionein (MT)I-F gene, complete cds. |
| 120 | 2757583CD1 | g187542 | 0 | Human metallothionein (MT)I-F gene, complete cds. |
| 121 | 1344279CB1 | g178535 | 0 | Human aminopeptidase N/CD13 mRNA encoding aminopeptidase N, complete |
| | | | | cds. |
| 122 | 1344279CD1 | g178535 | 0 | Human aminopeptidase N/CD13 mRNA encoding aminopeptidase N, complete |
| | | | | cds. |
| 123 | 1329472.2 | g808003 | 0 | Human Ig light chain variable region (lambda-IIIb subgroup) from IgM |
| | | | | rheumatoid factor. |
| 124 | 474457.35 | g35183 | 0 | Human p27 mRNA. |
| 125 | 474457.45 | g35183 | 0 | Human p27 mRNA. |
| 126 | 898779CB1 | g179530 | 0 | Human IgE-binding protein (epsilon-BP) mRNA, complete cds. |
| 127 | 898779CD1 | g179530 | 0 | Human IgE-binding protein (epsilon-BP) mRNA, complete cds. |
| 128 | 1843408CB1 | g189944 | 0 | Human (clone lamda-hPEC-3) phosphoenolpyruvate carboxykinase (PCK1) |
| | | | | mRNA, complete cds. |
| 129 | 1843408CD1 | g189944 | 0 | Human (clone lamda-hPEC-3) phosphoenolpyruvate carboxykinase (PCK1) |
| | | | | mRNA, complete cds. |
| 130 | 351241.1 | g2935483 | 4.00E−56 | Human minisatellite ceb 1 repeat region. |
| 131 | 413348.4 | g36425 | 0 | Human mRNA for selenoprotein P. |
| 132 | 983354.2 | g7331874 | 2.00E−14 | contains similarity to TR: O13786 |
| 133 | 235845.2 | g3152700 | 0 | Human tetraspan NET-1 mRNA, complete cds. |
| 134 | 266360.18 | g338481 | 0 | Human sorcin CP-22 mRNA, complete cds. |
| 135 | 266360.15 | g459835 | 0 | Human sorcin (SRI) mRNA, complete cds. |
| 136 | 1310030.1 | g34204 | 0 | Human rearranged Humigla1L1 gene encoding IgG light chain. |
| 137 | 2804864CB1 | g338481 | 0 | Human sorcin CP-22 mRNA, complete cds. |
| 138 | 2804864CD1 | g338481 | 0 | Human sorcin CP-22 mRNA, complete cds. |
| 139 | 349615.7 | g7291735 | 6.00E−46 | CG3209 gene product |
| 140 | 632664CB1 | g7658294 | 0 | Human transmembrane protein BRI mRNA, complete cds. |
| 141 | 632664CD1 | g7658294 | 0 | Human transmembrane protein BRI mRNA, complete cds. |
| 142 | 995929.22 | g4406655 | 0 | Human clone 25077 mRNA sequence, complete cds. |
| 143 | 995929.27 | g5531840 | 0 | Human PTD010 mRNA, complete cds. |
| 144 | 1397029.1 | g7020022 | 0 | Human cDNA FLJ20127 fis, clone COL06176. |
| 145 | 403560.1 | g7020022 | 0 | Human cDNA FLJ20127 fis, clone COL06176. |
| 146 | 1329606.3 | g33737 | 0 | Human rearranged Ig lambda light chain mRNA. |
| 147 | 1092257.12 | g2897953 | 0 | Human Kruppel-like zinc finger protein (EZF) mRNA, complete cds. |
| 148 | 474322.38 | g190885 | 0 | Human RASF-A PLA2 gene encoding synovial phospholipase, exons 2 through 5. |
| 149 | 255002.3 | g4753765 | 0 | Human mRNA for UDP-glucuronosyltransferase. |
| 150 | 1330137.1 | g177064 | 3.00E−79 | Gorilla gorilla beta-2-microglobulin mRNA (GOGOB2M). |
| 151 | 3699582CB1 | g184472 | 0 | Human bilirubin UDP-glucuronosyltransferase isozyme 1 mRNA, complete cds. |
| 152 | 3699582CD1 | g184472 | 0 | Human bilirubin UDP-glucuronosyltransferase isozyme 1 mRNA, complete cds. |
| 153 | 344537.24 | g184474 | 0 | Human bilirubin UDP-glucuronosyltransferase isozyme 2 mRNA, complete cds. |
| 154 | 16124.2 | | 0 | Incyte Unique |
| 155 | 104423.33 | g5441359 | 0 | Human mRNA activated in tumor suppression, clone TSAP19. |
| 156 | 406977.2 | g219917 | 0 | Human mRNA for acetoacetyl-coenzyme A thiolase (EC 2.3.1.9). |
| 157 | 3355973CB1 | g400415 | 0 | Human KRT8 mRNA for keratin 8. |
| 158 | 3355973CD1 | g400415 | 0 | Human KRT8 mRNA for keratin 8. |
| 159 | 406457.3 | g903681 | 0 | Human bumetanide-sensitive Na-K-Cl cotransporter (NKCC1) mRNA, complete |
| | | | | cds. |
| 160 | 2190217CB1 | g34755 | 0 | Human mRNA for myosin regulatory light chain. |
| 161 | 2190217CD1 | g34755 | 0 | Human mRNA for myosin regulatory light chain. |
| 162 | 29061.1 | | 0 | Incyte Unique |
| 163 | 1262593.2 | g4914599 | 0 | Human mRNA; cDNA DKFZp564A126 (from clone DKFZp564A126); partial |
| | | | | cds. |
| 164 | 1094812.1 | g179478 | 0 | Human biliary glycoprotein (BGP) gene, partial cds. |
| 165 | 2434655CB1 | g4753765 | 0 | Human mRNA for UDP-glucuronosyltransferase. |
| 166 | 2434655CD1 | g4753765 | 0 | Human mRNA for UDP-glucuronosyltransferase. |
| 167 | 206344.1 | | 0 | Incyte Unique |
| 168 | 1075717.7 | g31777 | 0 | Human gene encoding preproglucagon. |
| 169 | 1075717.1 | g183269 | 0 | Human glucagon mRNA, complete cds. |
| 170 | 372647.1 | | 0 | Incyte Unique |
| 171 | 148512.1 | g7717317 | 0 | Human chromosome 21 segment HS21C052. |
| 172 | 2023119CB1 | g306769 | 0 | Human leukemia virus receptor 1 (GLVR1) mRNA, complete cds. |
| 173 | 2023119CD1 | g306769 | 0 | Human leukemia virus receptor 1 (GLVR1) mRNA, complete cds. |
| 174 | 1973832CB1 | g179579 | 0 | Human beta-thromboglobulin-like protein mRNA, complete cds. |
| 175 | 1973832CD1 | g179579 | 0 | Human beta-thromboglobulin-like protein mRNA, complete cds. |
| 176 | 241888.54 | g179579 | 0 | Human beta-thromboglobulin-like protein mRNA, complete cds. |
| 177 | 1736965CB1 | | 0 | Incyte Unique |
| 178 | 1736965CD1 | | 0 | Incyte Unique |
| 179 | 412065.17 | g1374791 | 0 | Human selenium-binding protein (hSBP) mRNA, complete cds. |
| 180 | 988660.32 | g500848 | 0 | Human CD24 signal transducer mRNA, complete cds and 3′ region |
| 181 | 1434821CB1 | g35706 | 0 | Human pS2 mRNA induced by estrogen from Human breast cancer cell line MCF-7. |
| 182 | 1434821CD1 | g35706 | 0 | Human pS2 mRNA induced by estrogen from Human breast cancer cell line MCF-7. |
| 183 | 464689.64 | g7415720 | 0 | Human Scd mRNA for stearoyl-CoA desaturase, complete cds. |
| 184 | 464689.59 | g4808600 | 0 | Human stearoyl-CoA desaturase (SCD) mRNA, complete cds. |
| 185 | 1384719.3 | g3719220 | 0 | Human vascular endothelial growth factor mRNA, complete cds. |
| 186 | 407463.1 | g3882150 | 0 | Human mRNA for KIAA0715 protein, partial cds. |
| 187 | 522433CB1 | g2674084 | 0 | Human macrophage inhibitory cytokine-1 (MIC-1) mRNA, complete cds. |
| 188 | 522433CD1 | g2674084 | 0 | Human macrophage inhibitory cytokine-1 (MIC-1) mRNA, complete cds. |
| 189 | 480489.5 | g3360272 | 0 | Human UDP-glucuronosyltransferase 2B mRNA, complete cds. |
| 190 | 480489.2 | g3360272 | 0 | Human UDP-glucuronosyltransferase 2B mRNA, complete cds. |
| 191 | 1737775CB1 | g4009457 | 0 | Human calcium-dependent chloride channel-1 (hCLCA1) mRNA, complete cds. |
| 192 | 1737775CD1 | g4009457 | 0 | Human calcium-dependent chloride channel-1 (hCLCA1) mRNA, complete cds. |
| 193 | 088078CB1 | g340079 | 0 | Human 3,4-catechol estrogen UDP-glucuronosyltransferase mRNA, complete cds. |
| 194 | 088078CD1 | g340079 | 0 | Human 3,4-catechol estrogen UDP-glucuronosyltransferase mRNA, complete cds. |

TABLE 6

| SEQ ID NO | Template ID | Start | Stop | Frame | Pfam Hit | Pfam Description | E-Value |
|---|---|---|---|---|---|---|---|
| 2 | 995839.2 | 99 | 248 | forward 3 | metalthio | Metallothionein | 6.80E−08 |
| 4 | 3200830CD1 | 7 | 162 | | AhpC-TSA | AhpC/TSA family | 6.70E−71 |
| 7 | 3819039CD1 | 98 | 355 | | pkinase | Eukaryotic protein kinase domain | 1.10E−85 |
| 7 | 3819039CD1 | 356 | 430 | | pkinase_C | Protein kinase C terminal domain | 5.60E−16 |
| 8 | 330923.5 | 38 | 349 | forward 2 | cadherin | Cadherin domain | 2.30E−27 |
| 9 | 234630.57 | 253 | 519 | forward 1 | Xlink | Extracellular link domain | 3.00E−68 |
| 14 | 410911.5 | 539 | 889 | forward 2 | CH | Calponin homology (CH) domain | 6.90E−39 |
| 14 | 410911.5 | 338 | 424 | forward 2 | efhand | EF hand | 1.50E−07 |
| 17 | 474322.36 | 382 | 576 | forward 1 | phoslip | Phospholipase A2 | 8.90E−27 |
| 17 | 474322.36 | 287 | 343 | forward 2 | phoslip | Phospholipase A2 | 3.70E−06 |
| 19 | 3040213CD1 | 417 | 551 | | ATP-synt_A-c | ATP synthase Alpha chain, C terminal | 4.30E−106 |
| 19 | 3040213CD1 | 70 | 416 | | ATP-synt_ab | ATP synthase alpha/beta family | 9.90E−200 |
| 21 | 1282225CD1 | 2 | 127 | | lipocalin | Lipocalin/cytosolic fatty-acid binding protein family | 6.90E−25 |
| 23 | 991163CD1 | 148 | 376 | | trypsin | Trypsin | 1.20E−83 |
| 25 | 3220207CD1 | 47 | 170 | | Jacalin | Jacalin-like lectin domain | 1.20E−21 |
| 26 | 203309.2 | 255 | 1190 | forward 3 | filament | Intermediate filament proteins | 2.40E−155 |
| 28 | 333076.1 | 1883 | 2299 | forward 2 | STAS | STAS domain | 2.40E−15 |
| 28 | 333076.1 | 973 | 1782 | forward 1 | Sulfate_transp | Sulfate transporter family | 2.00E−06 |
| 30 | 989613CD1 | 73 | 261 | | Astacin | Astacin (Peptidase family M12A) | 3.80E−101 |
| 30 | 989613CD1 | 674 | 709 | | EGF | EGF-like domain | 1.40E−10 |
| 30 | 989613CD1 | 269 | 433 | | MAM | MAM domain. | 3.80E−58 |
| 30 | 989613CD1 | 437 | 595 | | MATH | MATH domain | 1.90E−26 |
| 32 | 2921920CD1 | 49 | 94 | | fibrinogen_C | Fibrinogen beta and gamma chains, C-terminal globular | 2.10E−04 |
| | | | | | | domain | |
| 33 | 997080.1 | 99 | 1604 | forward 3 | UDPGT | UDP-glucoronosyl and UDP-glucosyl transferases | 1.50E−285 |
| 35 | 5517972CD1 | 77 | 262 | | ABC_tran | ABC transporter | 9.80E−30 |
| 36 | 1397781.7 | 582 | 1508 | forward 3 | filament | Intermediate filament proteins | 1.20E−174 |
| 40 | 124600CD1 | 6 | 189 | | adh_short | short chain dehydrogenase | 2.60E−72 |
| 42 | 1401116.1 | 114 | 338 | forward 3 | ig | Immunoglobulin domain | 1.90E−11 |
| 45 | 255115.4 | 121 | 1023 | forward 1 | UDPGT | UDP-glucoronosyl and UDP-glucosyl transferases | 1.70E−177 |
| 45 | 255115.4 | 977 | 1573 | forward 2 | UDPGT | UDP-glucoronosyl and UDP-glucosyl transferases | 2.60E−158 |
| 46 | 1213592.1 | 187 | 357 | forward 1 | ig | Immunoglobulin domain | 9.90E−06 |
| 50 | 2264641CD1 | 194 | 306 | | PH | PH domain | 2.10E−12 |
| 50 | 2264641CD1 | 417 | 466 | | SH2 | Src homology domain 2 | 4.20E−16 |
| 52 | 237547CD1 | 69 | 380 | | filament | Intermediate filament proteins | 2.40E−155 |
| 55 | 1400916.1 | 120 | 344 | forward 3 | ig | Immunoglobulin domain | 1.90E−09 |
| 56 | 253986.11 | 448 | 1617 | forward 1 | ATP-gua_Ptrans | ATP: guanido phosphotransferase | 2.40E−265 |
| 57 | 253986.17 | 1 | 642 | forward 1 | ATP-gua_Ptrans | ATP: guanido phosphotransferase | 5.20E−156 |
| 59 | 2680109CD1 | 33 | 112 | | ig | Immunoglobulin domain | 1.30E−08 |
| 61 | 1800311CD1 | 1 | 115 | | Guanylin | Guanylin precursor | 6.00E−73 |
| 63 | 1804734CD1 | 35 | 246 | | MIP | Major intrinsic protein | 4.40E−58 |
| 65 | 3231154CD1 | 36 | 119 | | ig | Immunoglobulin domain | 2.60E−06 |
| 66 | 210095.11 | 1114 | 1287 | forward 1 | ig | Immunoglobulin domain | 7.40E−13 |
| 68 | 2719813CD1 | 23 | 285 | | carb_anhydrase | Eukaryotic-type carbonic anhydrase | 4.20E−153 |
| 70 | 2886583CD1 | 30 | 195 | | lactamase_B | Metallo-beta-lactamase superfamily | 1.30E−40 |
| 73 | 1808144CD1 | 526 | 716 | | STAS | STAS domain | 7.90E−28 |
| 73 | 1808144CD1 | 193 | 503 | | Sulfate_transp | Sulfate transporter family | 1.00E−123 |
| 76 | 237563.31 | 391 | 1671 | forward 1 | UDPGP | UTP-glucose-1-phosphate uridylyltransferase | 1.10E−255 |
| 77 | 1100412.5 | 896 | 1486 | forward 2 | OLF | Olfactomedin-like domain | 1.10E−09 |
| 77 | 1100412.5 | 736 | 1506 | forward 1 | OLF | Olfactomedin-like domain | 1.30E−08 |
| 78 | 1100412.4 | 818 | 1594 | forward 2 | OLF | Olfactomedin-like domain | 1.80E−89 |
| 80 | 2101663CD1 | 6 | 261 | | carb_anhydrase | Eukaryotic-type carbonic anhydrase | 2.20E−190 |
| 83 | 255002.4 | 150 | 1340 | forward 3 | UDPGT | UDP-glucoronosyl and UDP-glucosyl transferases | 3.50E−210 |
| 83 | 255002.4 | 1270 | 1590 | forward 1 | UDPGT | UDP-glucoronosyl and UDP-glucosyl transferases | 6.90E−76 |
| 84 | 1092257.2 | 1659 | 1733 | forward 3 | zf-C2H2 | Zinc finger, C2H2 type | 4.60E−06 |
| 87 | 1543330CD1 | 8 | 405 | | Arginosuc_synth | Arginosuccinate synthase | 4.80E−277 |
| 90 | 1281620CD1 | 164 | 282 | | Rhodanese | Rhodanese-like domain | 1.40E−31 |
| 93 | 1635966CD1 | 37 | 224 | | adh_short | short chain dehydrogenase | 2.20E−48 |
| 98 | 1422432CD1 | 53 | 81 | | efhand | EF hand | 1.80E−04 |
| 98 | 1422432CD1 | 4 | 47 | | S_100 | S-100/ICaBP type calcium binding domain | 2.70E−21 |
| 99 | 409895.2 | 1198 | 1284 | forward 1 | efhand | EF hand | 1.80E−04 |
| 101 | 4874364CD1 | 16 | 284 | | Sulfotransfer | Sulfotransferase proteins | 1.60E−176 |
| 102 | 239568.4 | 97 | 504 | forward 1 | carb_anhydrase | Eukaryotic-type carbonic anhydrase | 3.90E−90 |
| 103 | 255041.1 | 6 | 113 | forward 3 | UDPGT | UDP-glucoronosyl and UDP-glucosyl transferases | 6.20E−07 |
| 105 | 2555628CD1 | 39 | 465 | | UDPGP | UTP-glucose-1-phosphate uridylyltransferase | 1.10E−255 |
| 106 | 255803.1 | 159 | 227 | forward 3 | filament | Intermediate filament proteins | 5.70E−06 |
| 106 | 255803.1 | 2 | 130 | forward 2 | filament | Intermediate filament proteins | 1.90E−05 |
| 108 | 900341CD1 | 13 | 294 | | Euk_porin | Eukaryotic porin | 5.00E−182 |
| 110 | 273879CD1 | 212 | 315 | | Gal-bind_lectin | Vertebrate galactoside-binding lectins | 2.30E−46 |
| 111 | 141804.1 | 520 | 831 | forward 1 | ELM2 | ELM2 domain | 8.40E−17 |
| 113 | 2512879CD1 | 21 | 375 | | adh_zinc | Zinc-binding dehydrogenases | 7.90E−141 |
| 115 | 2685676CD1 | 1 | 61 | | metalthio | Metallothionein | 8.00E−24 |
| 117 | 2742913CD1 | 5 | 259 | | carb_anhydrase | Eukaryotic-type carbonic anhydrase | 3.90E−193 |
| 120 | 2757583CD1 | 1 | 61 | | metalthio | Metallothionein | 1.20E−24 |
| 122 | 1344279CD1 | 76 | 480 | | Peptidase_M1 | Peptidase family M1 | 2.60E−249 |
| 123 | 1329472.2 | 151 | 609 | forward 1 | ig | Immunoglobulin domain | 1.50E−08 |
| 127 | 898779CD1 | 136 | 239 | | Gal-bind_lectin | Vertebrate galactoside-binding lectins | 3.80E−50 |
| 129 | 1843408CD1 | 29 | 622 | | PEPCK | Phosphoenolpyruvate carboxykinase | 0.00E+00 |
| 132 | 983354.2 | 7 | 141 | forward 1 | FYVE | FYVE zinc finger | 2.20E−15 |
| 133 | 235845.2 | 300 | 998 | forward 3 | transmembrane4 | Transmembrane 4 family | 2.70E−42 |
| 146 | 1329606.3 | 386 | 592 | forward 2 | ig | Immunoglobulin domain | 1.40E−07 |
| 148 | 474322.38 | 1405 | 1542 | forward 1 | phoslip | Phospholipase A2 | 2.80E−18 |
| 149 | 255002.3 | 2 | 253 | forward 2 | UDPGT | UDP-glucoronosyl and UDP-glucosyl transferases | 1.30E−24 |
| 149 | 255002.3 | 229 | 312 | forward 1 | UDPGT | UDP-glucoronosyl and UDP-glucosyl transferases | 1.10E−10 |
| 152 | 3699582CD1 | 28 | 525 | | UDPGT | UDP-glucoronosyl and UDP-glucosyl transferases | 0.00E+00 |
| 153 | 344537.24 | 114 | 1607 | forward 3 | UDPGT | UDP-glucoronosyl and UDP-glucosyl transferases | 0.00E+00 |
| 156 | 406977.2 | 189 | 1355 | forward 3 | thiolase | Thiolase | 1.00E−230 |
| 158 | 3355973CD1 | 90 | 401 | | filament | Intermediate filament proteins | 5.40E−173 |
| 161 | 2190217CD1 | 32 | 60 | | efhand | EF hand | 6.50E−09 |
| 163 | 1262593.2 | 467 | 568 | forward 2 | TPR | TPR Domain | 3.30E−11 |
| 166 | 2434655CD1 | 24 | 525 | | UDPGT | UDP-glucoronosyl and UDP-glucosyl transferases | 1.50E−285 |
| 168 | 1075717.7 | 1131 | 1214 | forward 3 | hormone2 | Peptide hormone | 7.60E−10 |
| 169 | 1075717.1 | 292 | 375 | forward 1 | hormone2 | Peptide hormone | 1.90E−15 |
| 171 | 148512.1 | 122 | 658 | forward 2 | PMP22_Claudin | PMP-22/EMP/MP20/Claudin family | 8.70E−37 |
| 173 | 2023119CD1 | 39 | 665 | | PHO4 | Phosphate transporter family | 7.60E−268 |
| 175 | 1973832CD1 | 25 | 93 | | IL8 | Small cytokines (intecrine/chemokine), interleukin-8 like | 2.50E−34 |
| 176 | 241888.54 | 299 | 505 | forward 2 | IL8 | Small cytokines (intecrine/chemokine), interleukin-8 like | 2.50E−34 |
| 182 | 1434821CD1 | 30 | 71 | | trefoil | Trefoil (P-type) domain | 1.00E−24 |
| 183 | 464689.64 | 608 | 1342 | forward 2 | Desaturase | Fatty acid desaturase | 1.20E−163 |
| 185 | 1384719.3 | 1221 | 1457 | forward 3 | PDGF | Platelet-derived growth factor (PDGF) | 2.50E−53 |
| 188 | 522433CD1 | 211 | 308 | | TGF-beta | Transforming growth factor beta like domain | 6.80E−19 |
| 189 | 480489.5 | 813 | 1547 | forward 3 | UDPGT | UDP-glucoronosyl and UDP-glucosyl transferases | 6.50E−189 |
| 189 | 480489.5 | 86 | 859 | forward 2 | UDPGT | UDP-glucoronosyl and UDP-glucosyl transferases | 9.00E−138 |
| 190 | 480489.2 | 235 | 504 | forward 1 | UDPGT | UDP-glucoronosyl and UDP-glucosyl transferases | 1.60E−65 |
| 194 | 088078CD1 | 24 | 527 | | UDPGT | UDP-glucoronosyl and UDP-glucosyl transferases | 0.00E+00 |

TABLE 7

| SEQ ID | | | | | |
|---|---|---|---|---|---|
| NO | TEMPLATE ID | START | STOP | FRAME | DOMAIN |
| 2 | 995839.2 | 140 | 202 | forward 2 | SP |
| 5 | 6512.8 | 65 | 127 | forward 2 | SP |
| 5 | 6512.8 | 65 | 139 | forward 2 | SP |
| 5 | 6512.8 | 65 | 142 | forward 2 | SP |
| 5 | 6512.8 | 65 | 130 | forward 2 | SP |
| 8 | 330923.5 | 1073 | 1120 | forward 2 | SP |
| 8 | 330923.5 | 1750 | 1809 | forward 1 | TM |
| 8 | 330923.5 | 2203 | 2259 | forward 1 | TM |
| 8 | 330923.5 | 1049 | 1111 | forward 2 | TM |
| 8 | 330923.5 | 1049 | 1126 | forward 2 | TM |
| 8 | 330923.5 | 2519 | 2581 | forward 2 | SP |
| 8 | 330923.5 | 1055 | 1111 | forward 2 | SP |
| 8 | 330923.5 | 2519 | 2590 | forward 2 | TM |
| 8 | 330923.5 | 2377 | 2427 | forward 1 | TM |
| 8 | 330923.5 | 3512 | 3565 | forward 2 | SP |
| 8 | 330923.5 | 3512 | 3571 | forward 2 | SP |
| 8 | 330923.5 | 2379 | 2432 | forward 3 | TM |
| 8 | 330923.5 | 2373 | 2432 | forward 3 | TM |
| 8 | 330923.5 | 3512 | 3580 | forward 2 | SP |
| 8 | 330923.5 | 2519 | 2590 | forward 2 | SP |
| 8 | 330923.5 | 2883 | 2960 | forward 3 | TM |
| 8 | 330923.5 | 2531 | 2578 | forward 2 | TM |
| 8 | 330923.5 | 2381 | 2431 | forward 2 | TM |
| 8 | 330923.5 | 1058 | 1111 | forward 2 | TM |
| 8 | 330923.5 | 2375 | 2431 | forward 2 | TM |
| 8 | 330923.5 | 2510 | 2569 | forward 2 | TM |
| 8 | 330923.5 | 3517 | 3579 | forward 1 | TM |
| 8 | 330923.5 | 2510 | 2584 | forward 2 | TM |
| 8 | 330923.5 | 2501 | 2584 | forward 2 | TM |
| 8 | 330923.5 | 1049 | 1108 | forward 2 | TM |
| 9 | 234630.57 | 1978 | 2040 | forward 1 | SP |
| 9 | 234630.57 | 163 | 228 | forward 1 | SP |
| 9 | 234630.57 | 1981 | 2040 | forward 1 | TM |
| 9 | 234630.57 | 3108 | 3173 | forward 3 | SP |
| 9 | 234630.57 | 1987 | 2049 | forward 1 | TM |
| 9 | 234630.57 | 1975 | 2040 | forward 1 | SP |
| 9 | 234630.57 | 1966 | 2037 | forward 1 | TM |
| 9 | 234630.57 | 1975 | 2028 | forward 1 | SP |
| 9 | 234630.57 | 1978 | 2022 | forward 1 | SP |
| 9 | 234630.57 | 1984 | 2046 | forward 1 | TM |
| 9 | 234630.57 | 1972 | 2025 | forward 1 | TM |
| 9 | 234630.57 | 3126 | 3182 | forward 3 | TM |
| 9 | 234630.57 | 163 | 222 | forward 1 | SP |
| 13 | 2072479CD1 | 8 | 31 | | SP |
| 13 | 2072479CD1 | 8 | 25 | | SP |
| 13 | 2072479CD1 | 8 | 27 | | SP |
| 13 | 2072479CD1 | 8 | 29 | | SP |
| 14 | 410911.5 | 2421 | 2483 | forward 3 | TM |
| 14 | 410911.5 | 2421 | 2471 | forward 3 | SP |
| 14 | 410911.5 | 2421 | 2504 | forward 3 | TM |
| 14 | 410911.5 | 2421 | 2480 | forward 3 | TM |
| 16 | 1285632CD1 | 428 | 447 | | SP |
| 16 | 1285632CD1 | 651 | 674 | | TM |
| 16 | 1285632CD1 | 595 | 615 | | SP |
| 16 | 1285632CD1 | 595 | 617 | | SP |
| 16 | 1285632CD1 | 438 | 458 | | TM |
| 16 | 1285632CD1 | 296 | 318 | | SP |
| 16 | 1285632CD1 | 406 | 426 | | TM |
| 16 | 1285632CD1 | 1 | 28 | | SP |
| 16 | 1285632CD1 | 364 | 383 | | TM |
| 16 | 1285632CD1 | 303 | 320 | | SP |
| 16 | 1285632CD1 | 308 | 326 | | TM |
| 16 | 1285632CD1 | 654 | 672 | | TM |
| 16 | 1285632CD1 | 436 | 452 | | TM |
| 16 | 1285632CD1 | 437 | 455 | | TM |
| 16 | 1285632CD1 | 428 | 452 | | SP |
| 16 | 1285632CD1 | 713 | 733 | | TM |
| 16 | 1285632CD1 | 303 | 326 | | TM |
| 16 | 1285632CD1 | 711 | 731 | | TM |
| 16 | 1285632CD1 | 595 | 617 | | SP |
| 16 | 1285632CD1 | 713 | 734 | | SP |
| 16 | 1285632CD1 | 317 | 334 | | TM |
| 16 | 1285632CD1 | 303 | 325 | | TM |
| 16 | 1285632CD1 | 713 | 728 | | TM |
| 16 | 1285632CD1 | 655 | 675 | | TM |
| 16 | 1285632CD1 | 725 | 748 | | TM |
| 16 | 1285632CD1 | 522 | 541 | | TM |
| 16 | 1285632CD1 | 433 | 455 | | TM |
| 16 | 1285632CD1 | 658 | 678 | | TM |
| 16 | 1285632CD1 | 713 | 731 | | TM |
| 16 | 1285632CD1 | 595 | 622 | | SP |
| 16 | 1285632CD1 | 714 | 739 | | TM |
| 17 | 474322.36 | 169 | 249 | forward 1 | SP |
| 17 | 474322.36 | 184 | 237 | forward 1 | TM |
| 17 | 474322.36 | 190 | 255 | forward 1 | SP |
| 17 | 474322.36 | 190 | 237 | forward 1 | SP |
| 17 | 474322.36 | 190 | 243 | forward 1 | SP |
| 17 | 474322.36 | 190 | 249 | forward 1 | SP |
| 25 | 3220207CD1 | 10 | 32 | | SP |
| 25 | 3220207CD1 | 12 | 32 | | SP |
| 25 | 3220207CD1 | 1 | 26 | | SP |
| 25 | 3220207CD1 | 12 | 29 | | SP |
| 25 | 3220207CD1 | 1 | 27 | | SP |
| 25 | 3220207CD1 | 12 | 27 | | SP |
| 26 | 203309.2 | 724 | 768 | forward 1 | SP |
| 26 | 203309.2 | 691 | 762 | forward 1 | SP |
| 26 | 203309.2 | 691 | 756 | forward 1 | SP |
| 26 | 203309.2 | 2023 | 2082 | forward 1 | TM |
| 27 | 998971.1 | 758 | 814 | forward 2 | TM |
| 27 | 998971.1 | 281 | 337 | forward 2 | TM |
| 28 | 333076.1 | 1777 | 1836 | forward 1 | SP |
| 28 | 333076.1 | 1372 | 1428 | forward 1 | SP |
| 28 | 333076.1 | 1085 | 1162 | forward 2 | SP |
| 28 | 333076.1 | 1730 | 1795 | forward 2 | SP |
| 28 | 333076.1 | 1777 | 1845 | forward 1 | SP |
| 28 | 333076.1 | 625 | 690 | forward 1 | SP |
| 28 | 333076.1 | 1730 | 1801 | forward 2 | SP |
| 28 | 333076.1 | 3801 | 3857 | forward 3 | TM |
| 28 | 333076.1 | 1807 | 1863 | forward 1 | TM |
| 28 | 333076.1 | 1275 | 1325 | forward 3 | SP |
| 28 | 333076.1 | 1275 | 1331 | forward 3 | SP |
| 28 | 333076.1 | 3227 | 3277 | forward 2 | TM |
| 30 | 989613CD1 | 718 | 737 | | TM |
| 30 | 989613CD1 | 1 | 19 | | SP |
| 30 | 989613CD1 | 1 | 24 | | SP |
| 30 | 989613CD1 | 1 | 21 | | SP |
| 30 | 989613CD1 | 1 | 23 | | SP |
| 32 | 2921920CD1 | 4 | 20 | | SP |
| 32 | 2921920CD1 | 1 | 17 | | TM |
| 32 | 2921920CD1 | 4 | 23 | | SP |
| 32 | 2921920CD1 | 1 | 28 | | SP |
| 32 | 2921920CD1 | 8 | 26 | | SP |
| 32 | 2921920CD1 | 4 | 26 | | SP |
| 32 | 2921920CD1 | 1 | 26 | | SP |
| 33 | 997080.1 | 1473 | 1550 | forward 3 | TM |
| 33 | 997080.1 | 2741 | 2791 | forward 2 | TM |
| 33 | 997080.1 | 2726 | 2791 | forward 2 | SP |
| 33 | 997080.1 | 1668 | 1724 | forward 3 | TM |
| 33 | 997080.1 | 1503 | 1565 | forward 3 | TM |
| 33 | 997080.1 | 501 | 554 | forward 3 | SP |
| 33 | 997080.1 | 2747 | 2809 | forward 2 | TM |
| 33 | 997080.1 | 300 | 362 | forward 3 | TM |
| 33 | 997080.1 | 2704 | 2766 | forward 1 | SP |
| 33 | 997080.1 | 1677 | 1730 | forward 3 | TM |
| 33 | 997080.1 | 30 | 92 | forward 3 | SP |
| 33 | 997080.1 | 30 | 104 | forward 3 | SP |
| 33 | 997080.1 | 30 | 86 | forward 3 | SP |
| 33 | 997080.1 | 2729 | 2785 | forward 2 | TM |
| 33 | 997080.1 | 30 | 98 | forward 3 | SP |
| 35 | 5517972CD1 | 494 | 522 | | TM |
| 35 | 5517972CD1 | 548 | 568 | | SP |
| 35 | 5517972CD1 | 548 | 566 | | SP |
| 35 | 5517972CD1 | 481 | 505 | | SP |
| 35 | 5517972CD1 | 514 | 535 | | SP |
| 35 | 5517972CD1 | 548 | 569 | | SP |
| 35 | 5517972CD1 | 541 | 557 | | TM |
| 35 | 5517972CD1 | 396 | 417 | | TM |
| 35 | 5517972CD1 | 544 | 564 | | TM |
| 35 | 5517972CD1 | 543 | 570 | | TM |
| 35 | 5517972CD1 | 538 | 556 | | TM |
| 36 | 1397781.7 | 2189 | 2254 | forward 2 | SP |
| 36 | 1397781.7 | 2207 | 2290 | forward 2 | TM |
| 36 | 1397781.7 | 1444 | 1554 | forward 1 | SP |
| 36 | 1397781.7 | 2204 | 2257 | forward 2 | TM |
| 36 | 1397781.7 | 2207 | 2263 | forward 2 | TM |
| 37 | 236655.3 | 102 | 149 | forward 3 | SP |
| 37 | 236655.3 | 84 | 140 | forward 3 | SP |
| 37 | 236655.3 | 84 | 149 | forward 3 | SP |
| 37 | 236655.3 | 84 | 149 | forward 3 | SP |
| 37 | 236655.3 | 84 | 155 | forward 3 | SP |
| 38 | 345275.4 | 2128 | 2211 | forward 1 | SP |
| 38 | 345275.4 | 415 | 483 | forward 1 | TM |
| 38 | 345275.4 | 1276 | 1335 | forward 1 | SP |
| 38 | 345275.4 | 115 | 171 | forward 1 | TM |
| 38 | 345275.4 | 1276 | 1344 | forward 1 | SP |
| 38 | 345275.4 | 226 | 282 | forward 1 | SP |
| 38 | 345275.4 | 226 | 285 | forward 1 | TM |
| 38 | 345275.4 | 2152 | 2205 | forward 1 | TM |
| 38 | 345275.4 | 2146 | 2208 | forward 1 | TM |
| 38 | 345275.4 | 442 | 501 | forward 1 | TM |
| 38 | 345275.4 | 2237 | 2299 | forward 2 | TM |
| 38 | 345275.4 | 97 | 168 | forward 1 | TM |
| 38 | 345275.4 | 226 | 288 | forward 1 | SP |
| 42 | 1401116.1 | 313 | 369 | forward 1 | SP |
| 42 | 1401116.1 | 313 | 381 | forward 1 | SP |
| 42 | 1401116.1 | 15 | 71 | forward 3 | SP |
| 42 | 1401116.1 | 313 | 375 | forward 1 | SP |
| 42 | 1401116.1 | 313 | 384 | forward 1 | SP |
| 42 | 1401116.1 | 15 | 74 | forward 3 | SP |
| 44 | 2921009CD1 | 1 | 26 | | SP |
| 45 | 255115.4 | 360 | 419 | forward 3 | TM |
| 45 | 255115.4 | 52 | 123 | forward 1 | SP |
| 45 | 255115.4 | 52 | 120 | forward 1 | SP |
| 45 | 255115.4 | 52 | 108 | forward 1 | SP |
| 45 | 255115.4 | 52 | 114 | forward 1 | SP |
| 45 | 255115.4 | 1511 | 1570 | forward 2 | TM |
| 46 | 1213592.1 | 39 | 95 | forward 3 | SP |
| 46 | 1213592.1 | 39 | 98 | forward 3 | SP |
| 48 | 1376382CD1 | 1 | 20 | | SP |
| 48 | 1376382CD1 | 1 | 19 | | SP |
| 48 | 1376382CD1 | 1 | 22 | | SP |
| 54 | 2771481CD1 | 893 | 913 | | TM |
| 54 | 2771481CD1 | 2 | 26 | | TM |
| 54 | 2771481CD1 | 895 | 913 | | TM |
| 54 | 2771481CD1 | 1 | 24 | | SP |
| 54 | 2771481CD1 | 898 | 915 | | TM |
| 54 | 2771481CD1 | 1 | 18 | | SP |
| 54 | 2771481CD1 | 1 | 22 | | SP |
| 54 | 2771481CD1 | 1 | 21 | | SP |
| 55 | 1400916.1 | 21 | 83 | forward 3 | SP |
| 55 | 1400916.1 | 21 | 77 | forward 3 | SP |
| 56 | 253986.11 | 379 | 459 | forward 1 | SP |
| 56 | 253986.11 | 379 | 462 | forward 1 | SP |
| 59 | 2680109CD1 | 1 | 20 | | SP |
| 59 | 2680109CD1 | 642 | 663 | | TM |
| 59 | 2680109CD1 | 1 | 17 | | SP |
| 59 | 2680109CD1 | 1 | 18 | | SP |
| 61 | 1800311CD1 | 1 | 24 | | SP |
| 61 | 1800311CD1 | 1 | 23 | | SP |
| 61 | 1800311CD1 | 1 | 19 | | SP |
| 61 | 1800311CD1 | 1 | 21 | | SP |
| 61 | 1800311CD1 | 1 | 16 | | SP |
| 63 | 1804734CD1 | 231 | 251 | | TM |
| 63 | 1804734CD1 | 41 | 60 | | TM |
| 65 | 3231154CD1 | 5 | 21 | | SP |
| 65 | 3231154CD1 | 241 | 257 | | TM |
| 65 | 3231154CD1 | 238 | 258 | | TM |
| 65 | 3231154CD1 | 234 | 256 | | TM |
| 65 | 3231154CD1 | 237 | 256 | | TM |
| 65 | 3231154CD1 | 236 | 259 | | TM |
| 66 | 210095.11 | 1405 | 1455 | forward 1 | TM |
| 66 | 210095.11 | 1366 | 1425 | forward 1 | TM |
| 66 | 210095.11 | 1393 | 1440 | forward 1 | SP |
| 66 | 210095.11 | 2633 | 2692 | forward 2 | SP |
| 66 | 210095.11 | 1378 | 1455 | forward 1 | TM |
| 66 | 210095.11 | 1384 | 1455 | forward 1 | TM |
| 66 | 210095.11 | 1384 | 1446 | forward 1 | TM |
| 66 | 210095.11 | 94 | 195 | forward 1 | SP |
| 68 | 2719813CD1 | 1 | 20 | | SP |
| 68 | 2719813CD1 | 1 | 17 | | SP |
| 68 | 2719813CD1 | 1 | 18 | | SP |
| 71 | 25685.3 | 177 | 245 | forward 3 | SP |
| 71 | 25685.3 | 177 | 233 | forward 3 | SP |
| 71 | 25685.3 | 177 | 239 | forward 3 | SP |
| 73 | 1808144CD1 | 420 | 443 | | TM |
| 73 | 1808144CD1 | 474 | 495 | | SP |
| 73 | 1808144CD1 | 342 | 361 | | TM |
| 73 | 1808144CD1 | 413 | 433 | | TM |
| 73 | 1808144CD1 | 419 | 441 | | SP |
| 73 | 1808144CD1 | 474 | 493 | | SP |
| 73 | 1808144CD1 | 257 | 272 | | TM |
| 73 | 1808144CD1 | 188 | 208 | | TM |
| 73 | 1808144CD1 | 412 | 438 | | TM |
| 73 | 1808144CD1 | 419 | 433 | | TM |
| 73 | 1808144CD1 | 471 | 497 | | TM |
| 73 | 1808144CD1 | 412 | 431 | | TM |
| 73 | 1808144CD1 | 477 | 500 | | TM |
| 73 | 1808144CD1 | 469 | 487 | | TM |
| 73 | 1808144CD1 | 409 | 429 | | TM |
| 73 | 1808144CD1 | 471 | 495 | | TM |
| 74 | 201356.1 | 3004 | 3063 | forward 1 | SP |
| 74 | 201356.1 | 3057 | 3122 | forward 3 | SP |
| 74 | 201356.1 | 3189 | 3263 | forward 3 | TM |
| 74 | 201356.1 | 330 | 416 | forward 3 | TM |
| 74 | 201356.1 | 1772 | 1828 | forward 2 | TM |
| 74 | 201356.1 | 2364 | 2426 | forward 3 | TM |
| 74 | 201356.1 | 1772 | 1855 | forward 2 | TM |
| 74 | 201356.1 | 2122 | 2184 | forward 1 | TM |
| 75 | 978178.7 | 1174 | 1221 | forward 1 | TM |
| 75 | 978178.7 | 979 | 1032 | forward 1 | TM |
| 75 | 978178.7 | 1414 | 1476 | forward 1 | TM |
| 75 | 978178.7 | 970 | 1026 | forward 1 | TM |
| 75 | 978178.7 | 1420 | 1494 | forward 1 | TM |
| 75 | 978178.7 | 1144 | 1200 | forward 1 | TM |
| 77 | 1100412.5 | 27 | 83 | forward 3 | TM |
| 77 | 1100412.5 | 2217 | 2270 | forward 3 | TM |
| 77 | 1100412.5 | 2715 | 2774 | forward 3 | SP |
| 77 | 1100412.5 | 15 | 68 | forward 3 | SP |
| 77 | 1100412.5 | 2640 | 2723 | forward 3 | TM |
| 77 | 1100412.5 | 788 | 850 | forward 2 | TM |
| 77 | 1100412.5 | 15 | 83 | forward 3 | SP |
| 77 | 1100412.5 | 15 | 83 | forward 3 | SP |
| 77 | 1100412.5 | 15 | 68 | forward 3 | SP |
| 77 | 1100412.5 | 15 | 74 | forward 3 | SP |
| 78 | 1100412.4 | 1239 | 1313 | forward 3 | SP |
| 78 | 1100412.4 | 89 | 145 | forward 2 | TM |
| 78 | 1100412.4 | 2273 | 2326 | forward 2 | TM |
| 78 | 1100412.4 | 2771 | 2830 | forward 2 | SP |
| 78 | 1100412.4 | 77 | 130 | forward 2 | SP |
| 78 | 1100412.4 | 2696 | 2779 | forward 2 | TM |
| 78 | 1100412.4 | 849 | 911 | forward 3 | TM |
| 78 | 1100412.4 | 77 | 145 | forward 2 | SP |
| 78 | 1100412.4 | 77 | 145 | forward 2 | SP |
| 78 | 1100412.4 | 77 | 130 | forward 2 | SP |
| 78 | 1100412.4 | 77 | 136 | forward 2 | SP |
| 82 | 611082CD1 | 97 | 121 | | SP |
| 82 | 611082CD1 | 207 | 229 | | SP |
| 82 | 611082CD1 | 178 | 198 | | TM |
| 82 | 611082CD1 | 164 | 190 | | SP |
| 82 | 611082CD1 | 162 | 180 | | TM |
| 82 | 611082CD1 | 160 | 177 | | TM |
| 82 | 611082CD1 | 207 | 221 | | SP |
| 82 | 611082CD1 | 164 | 187 | | SP |
| 83 | 255002.4 | 93 | 182 | forward 3 | SP |
| 83 | 255002.4 | 1564 | 1629 | forward 1 | TM |
| 83 | 255002.4 | 1564 | 1620 | forward 1 | TM |
| 83 | 255002.4 | 93 | 152 | forward 3 | SP |
| 84 | 1092257.2 | 2525 | 2581 | forward 2 | TM |
| 84 | 1092257.2 | 374 | 430 | forward 2 | TM |
| 85 | 1102315.3 | 279 | 344 | forward 3 | TM |
| 85 | 1102315.3 | 282 | 332 | forward 3 | TM |
| 85 | 1102315.3 | 910 | 999 | forward 1 | SP |
| 85 | 1102315.3 | 300 | 356 | forward 3 | TM |
| 85 | 1102315.3 | 285 | 347 | forward 3 | TM |
| 88 | 232992.1 | 433 | 492 | forward 1 | SP |
| 88 | 232992.1 | 997 | 1074 | forward 1 | SP |
| 88 | 232992.1 | 418 | 492 | forward 1 | SP |
| 88 | 232992.1 | 381 | 455 | forward 3 | TM |
| 88 | 232992.1 | 424 | 480 | forward 1 | TM |
| 88 | 232992.1 | 3009 | 3062 | forward 3 | SP |
| 88 | 232992.1 | 131 | 187 | forward 2 | TM |
| 88 | 232992.1 | 378 | 431 | forward 3 | TM |
| 88 | 232992.1 | 541 | 597 | forward 1 | TM |
| 88 | 232992.1 | 409 | 477 | forward 1 | TM |
| 88 | 232992.1 | 664 | 744 | forward 1 | SP |
| 88 | 232992.1 | 381 | 458 | forward 3 | TM |
| 88 | 232992.1 | 427 | 492 | forward 1 | SP |
| 88 | 232992.1 | 436 | 492 | forward 1 | SP |
| 88 | 232992.1 | 421 | 474 | forward 1 | TM |
| 88 | 232992.1 | 375 | 434 | forward 3 | TM |
| 91 | 343502.1 | 1777 | 1833 | forward 1 | SP |
| 91 | 343502.1 | 231 | 284 | forward 3 | SP |
| 91 | 343502.1 | 231 | 278 | forward 3 | SP |
| 91 | 343502.1 | 231 | 290 | forward 3 | SP |
| 93 | 1635966CD1 | 1 | 19 | | TM |
| 93 | 1635966CD1 | 8 | 27 | | TM |
| 93 | 1635966CD1 | 1 | 24 | | SP |
| 93 | 1635966CD1 | 1 | 17 | | SP |
| 93 | 1635966CD1 | 1 | 20 | | SP |
| 93 | 1635966CD1 | 1 | 23 | | SP |
| 93 | 1635966CD1 | 1 | 18 | | SP |
| 99 | 409895.2 | 384 | 437 | forward 3 | SP |
| 102 | 239568.4 | 416 | 478 | forward 2 | SP |
| 106 | 255803.1 | 436 | 519 | forward 1 | SP |
| 106 | 255803.1 | 283 | 360 | forward 1 | SP |
| 106 | 255803.1 | 283 | 348 | forward 1 | SP |
| 106 | 255803.1 | 337 | 396 | forward 1 | SP |
| 106 | 255803.1 | 307 | 360 | forward 1 | SP |
| 106 | 255803.1 | 307 | 396 | forward 1 | SP |
| 111 | 141804.1 | 37 | 87 | forward 1 | TM |
| 122 | 1344279CD1 | 16 | 34 | | TM |
| 122 | 1344279CD1 | 13 | 33 | | TM |
| 122 | 1344279CD1 | 9 | 31 | | TM |
| 123 | 1329472.2 | 52 | 114 | forward 1 | SP |
| 123 | 1329472.2 | 52 | 108 | forward 1 | SP |
| 124 | 474457.35 | 387 | 452 | forward 3 | SP |
| 124 | 474457.35 | 449 | 508 | forward 2 | TM |
| 125 | 474457.45 | 330 | 395 | forward 3 | SP |
| 125 | 474457.45 | 392 | 451 | forward 2 | TM |
| 130 | 351241.1 | 430 | 480 | forward 1 | TM |
| 131 | 413348.4 | 99 | 173 | forward 3 | SP |
| 131 | 413348.4 | 2300 | 2374 | forward 2 | SP |
| 131 | 413348.4 | 811 | 864 | forward 1 | SP |
| 131 | 413348.4 | 526 | 585 | forward 1 | SP |
| 131 | 413348.4 | 511 | 570 | forward 1 | TM |
| 131 | 413348.4 | 1742 | 1825 | forward 2 | TM |
| 131 | 413348.4 | 1625 | 1678 | forward 2 | TM |
| 131 | 413348.4 | 1475 | 1534 | forward 2 | TM |
| 131 | 413348.4 | 111 | 179 | forward 3 | SP |
| 131 | 413348.4 | 117 | 164 | forward 3 | SP |
| 131 | 413348.4 | 1615 | 1668 | forward 1 | TM |
| 131 | 413348.4 | 814 | 879 | forward 1 | SP |
| 131 | 413348.4 | 511 | 585 | forward 1 | SP |
| 131 | 413348.4 | 814 | 861 | forward 1 | SP |
| 131 | 413348.4 | 117 | 179 | forward 3 | SP |
| 131 | 413348.4 | 117 | 173 | forward 3 | SP |
| 132 | 983354.2 | 3750 | 3806 | forward 3 | TM |
| 132 | 983354.2 | 1905 | 1961 | forward 3 | TM |
| 132 | 983354.2 | 4122 | 4190 | forward 3 | SP |
| 132 | 983354.2 | 1197 | 1280 | forward 3 | SP |
| 132 | 983354.2 | 3188 | 3277 | forward 2 | SP |
| 132 | 983354.2 | 4122 | 4175 | forward 3 | SP |
| 132 | 983354.2 | 2994 | 3065 | forward 3 | SP |
| 132 | 983354.2 | 3221 | 3274 | forward 2 | TM |
| 132 | 983354.2 | 4236 | 4298 | forward 3 | SP |
| 132 | 983354.2 | 4236 | 4283 | forward 3 | SP |
| 132 | 983354.2 | 1197 | 1274 | forward 3 | SP |
| 133 | 235845.2 | 448 | 534 | forward 1 | SP |
| 133 | 235845.2 | 531 | 596 | forward 3 | SP |
| 133 | 235845.2 | 438 | 500 | forward 3 | SP |
| 133 | 235845.2 | 537 | 596 | forward 3 | SP |
| 133 | 235845.2 | 561 | 614 | forward 3 | TM |
| 133 | 235845.2 | 303 | 365 | forward 3 | TM |
| 133 | 235845.2 | 318 | 368 | forward 3 | SP |
| 133 | 235845.2 | 312 | 383 | forward 3 | TM |
| 133 | 235845.2 | 315 | 374 | forward 3 | SP |
| 133 | 235845.2 | 318 | 374 | forward 3 | SP |
| 133 | 235845.2 | 315 | 383 | forward 3 | SP |
| 133 | 235845.2 | 462 | 518 | forward 3 | TM |
| 133 | 235845.2 | 537 | 593 | forward 3 | TM |
| 133 | 235845.2 | 312 | 377 | forward 3 | TM |
| 133 | 235845.2 | 318 | 380 | forward 3 | TM |
| 133 | 235845.2 | 543 | 614 | forward 3 | TM |
| 133 | 235845.2 | 312 | 398 | forward 3 | TM |
| 133 | 235845.2 | 549 | 617 | forward 3 | TM |
| 134 | 266360.18 | 788 | 841 | forward 2 | TM |
| 134 | 266360.18 | 669 | 734 | forward 3 | SP |
| 134 | 266360.18 | 753 | 815 | forward 3 | TM |
| 134 | 266360.18 | 678 | 740 | forward 3 | TM |
| 135 | 266360.15 | 524 | 589 | forward 2 | SP |
| 135 | 266360.15 | 533 | 595 | forward 2 | TM |
| 136 | 1310030.1 | 31 | 102 | forward 1 | SP |
| 136 | 1310030.1 | 31 | 87 | forward 1 | SP |
| 136 | 1310030.1 | 31 | 93 | forward 1 | SP |
| 139 | 349615.7 | 596 | 655 | forward 2 | TM |
| 139 | 349615.7 | 589 | 657 | forward 1 | SP |
| 139 | 349615.7 | 45 | 119 | forward 3 | TM |
| 139 | 349615.7 | 591 | 671 | forward 3 | TM |
| 139 | 349615.7 | 585 | 644 | forward 3 | SP |
| 139 | 349615.7 | 372 | 431 | forward 3 | SP |
| 139 | 349615.7 | 550 | 663 | forward 1 | SP |
| 139 | 349615.7 | 601 | 675 | forward 1 | TM |
| 139 | 349615.7 | 585 | 638 | forward 3 | TM |
| 139 | 349615.7 | 613 | 675 | forward 1 | TM |
| 139 | 349615.7 | 372 | 425 | forward 3 | SP |
| 139 | 349615.7 | 591 | 650 | forward 3 | TM |
| 139 | 349615.7 | 590 | 643 | forward 2 | SP |
| 139 | 349615.7 | 601 | 660 | forward 1 | TM |
| 139 | 349615.7 | 586 | 636 | forward 1 | TM |
| 141 | 632664CD1 | 57 | 75 | | TM |
| 142 | 995929.22 | 590 | 655 | forward 2 | SP |
| 142 | 995929.22 | 866 | 949 | forward 2 | SP |
| 142 | 995929.22 | 602 | 655 | forward 2 | SP |
| 143 | 995929.27 | 1490 | 1549 | forward 2 | SP |
| 143 | 995929.27 | 1469 | 1543 | forward 2 | SP |
| 143 | 995929.27 | 1481 | 1549 | forward 2 | SP |
| 143 | 995929.27 | 1848 | 1907 | forward 3 | SP |
| 143 | 995929.27 | 1848 | 1892 | forward 3 | SP |
| 143 | 995929.27 | 586 | 669 | forward 1 | SP |
| 143 | 995929.27 | 1172 | 1222 | forward 2 | SP |
| 144 | 1397029.1 | 192 | 251 | forward 3 | TM |
| 146 | 1329606.3 | 778 | 849 | forward 1 | SP |
| 146 | 1329606.3 | 824 | 892 | forward 2 | SP |
| 146 | 1329606.3 | 279 | 332 | forward 3 | SP |
| 147 | 1092257.12 | 60 | 116 | forward 3 | TM |
| 148 | 474322.38 | 836 | 898 | forward 2 | SP |
| 152 | 3699582CD1 | 8 | 25 | | SP |
| 152 | 3699582CD1 | 5 | 27 | | SP |
| 152 | 3699582CD1 | 488 | 505 | | TM |
| 152 | 3699582CD1 | 492 | 512 | | TM |
| 152 | 3699582CD1 | 487 | 507 | | TM |
| 152 | 3699582CD1 | 1 | 27 | | SP |
| 152 | 3699582CD1 | 1 | 27 | | SP |
| 153 | 344537.24 | 1494 | 1547 | forward 3 | TM |
| 153 | 344537.24 | 1506 | 1568 | forward 3 | TM |
| 153 | 344537.24 | 430 | 501 | forward 1 | SP |
| 153 | 344537.24 | 1491 | 1553 | forward 3 | TM |
| 153 | 344537.24 | 30 | 107 | forward 3 | SP |
| 153 | 344537.24 | 30 | 113 | forward 3 | SP |
| 155 | 104423.33 | 168 | 224 | forward 3 | SP |
| 155 | 104423.33 | 1348 | 1395 | forward 1 | SP |
| 155 | 104423.33 | 2727 | 2780 | forward 3 | TM |
| 155 | 104423.33 | 2778 | 2867 | forward 3 | SP |
| 155 | 104423.33 | 2739 | 2792 | forward 3 | SP |
| 155 | 104423.33 | 2739 | 2813 | forward 3 | SP |
| 155 | 104423.33 | 2739 | 2798 | forward 3 | SP |
| 155 | 104423.33 | 2739 | 2807 | forward 3 | SP |
| 156 | 406977.2 | 1030 | 1089 | forward 1 | TM |
| 156 | 406977.2 | 1444 | 1518 | forward 1 | TM |
| 159 | 406457.3 | 1410 | 1469 | forward 3 | SP |
| 159 | 406457.3 | 2079 | 2150 | forward 3 | TM |
| 159 | 406457.3 | 1911 | 1973 | forward 3 | SP |
| 159 | 406457.3 | 1911 | 1979 | forward 3 | SP |
| 159 | 406457.3 | 1440 | 1502 | forward 3 | TM |
| 159 | 406457.3 | 1014 | 1082 | forward 3 | SP |
| 159 | 406457.3 | 1344 | 1406 | forward 3 | TM |
| 159 | 406457.3 | 129 | 212 | forward 3 | SP |
| 159 | 406457.3 | 1035 | 1088 | forward 3 | SP |
| 159 | 406457.3 | 4222 | 4275 | forward 1 | TM |
| 159 | 406457.3 | 1050 | 1106 | forward 3 | TM |
| 159 | 406457.3 | 2088 | 2144 | forward 3 | TM |
| 159 | 406457.3 | 1434 | 1484 | forward 3 | TM |
| 159 | 406457.3 | 1437 | 1493 | forward 3 | TM |
| 159 | 406457.3 | 1410 | 1484 | forward 3 | SP |
| 159 | 406457.3 | 2265 | 2327 | forward 3 | TM |
| 159 | 406457.3 | 1035 | 1106 | forward 3 | TM |
| 159 | 406457.3 | 2259 | 2321 | forward 3 | TM |
| 159 | 406457.3 | 1911 | 1979 | forward 3 | SP |
| 159 | 406457.3 | 2265 | 2330 | forward 3 | SP |
| 159 | 406457.3 | 1077 | 1130 | forward 3 | TM |
| 159 | 406457.3 | 1035 | 1103 | forward 3 | TM |
| 159 | 406457.3 | 4447 | 4503 | forward 1 | TM |
| 159 | 406457.3 | 2265 | 2312 | forward 3 | TM |
| 159 | 406457.3 | 2091 | 2153 | forward 3 | TM |
| 159 | 406457.3 | 2301 | 2372 | forward 3 | TM |
| 159 | 406457.3 | 1692 | 1751 | forward 3 | TM |
| 159 | 406457.3 | 1425 | 1493 | forward 3 | TM |
| 159 | 406457.3 | 2100 | 2162 | forward 3 | TM |
| 159 | 406457.3 | 2265 | 2321 | forward 3 | TM |
| 159 | 406457.3 | 1911 | 1994 | forward 3 | SP |
| 159 | 406457.3 | 5115 | 5192 | forward 3 | TM |
| 159 | 406457.3 | 2268 | 2345 | forward 3 | TM |
| 162 | 29061.1 | 179 | 238 | forward 2 | TM |
| 162 | 29061.1 | 230 | 289 | forward 2 | SP |
| 162 | 29061.1 | 984 | 1040 | forward 3 | TM |
| 162 | 29061.1 | 655 | 708 | forward 1 | TM |
| 162 | 29061.1 | 227 | 295 | forward 2 | SP |
| 162 | 29061.1 | 217 | 291 | forward 1 | TM |
| 162 | 29061.1 | 236 | 289 | forward 2 | SP |
| 162 | 29061.1 | 987 | 1067 | forward 3 | TM |
| 163 | 1262593.2 | 2388 | 2435 | forward 3 | SP |
| 163 | 1262593.2 | 2390 | 2446 | forward 2 | TM |
| 163 | 1262593.2 | 1320 | 1370 | forward 3 | TM |
| 163 | 1262593.2 | 2373 | 2435 | forward 3 | TM |
| 163 | 1262593.2 | 2388 | 2441 | forward 3 | SP |
| 163 | 1262593.2 | 2379 | 2426 | forward 3 | TM |
| 163 | 1262593.2 | 946 | 1008 | forward 1 | TM |
| 163 | 1262593.2 | 2373 | 2426 | forward 3 | TM |
| 164 | 1094812.1 | 249 | 335 | forward 3 | SP |
| 164 | 1094812.1 | 214 | 264 | forward 1 | TM |
| 164 | 1094812.1 | 175 | 234 | forward 1 | TM |
| 164 | 1094812.1 | 202 | 249 | forward 1 | SP |
| 164 | 1094812.1 | 187 | 264 | forward 1 | TM |
| 164 | 1094812.1 | 193 | 264 | forward 1 | TM |
| 164 | 1094812.1 | 193 | 255 | forward 1 | TM |
| 166 | 2434655CD1 | 482 | 507 | | TM |
| 166 | 2434655CD1 | 492 | 512 | | TM |
| 166 | 2434655CD1 | 158 | 175 | | SP |
| 166 | 2434655CD1 | 91 | 111 | | TM |
| 166 | 2434655CD1 | 1 | 21 | | SP |
| 166 | 2434655CD1 | 1 | 25 | | SP |
| 166 | 2434655CD1 | 1 | 19 | | SP |
| 166 | 2434655CD1 | 1 | 23 | | SP |
| 167 | 206344.1 | 694 | 744 | forward 1 | TM |
| 168 | 1075717.7 | 981 | 1037 | forward 3 | SP |
| 168 | 1075717.7 | 981 | 1031 | forward 3 | SP |
| 168 | 1075717.7 | 981 | 1049 | forward 3 | SP |
| 168 | 1075717.7 | 973 | 1038 | forward 1 | SP |
| 168 | 1075717.7 | 981 | 1040 | forward 3 | SP |
| 169 | 1075717.1 | 136 | 195 | forward 1 | SP |
| 170 | 372647.1 | 63 | 134 | forward 3 | TM |
| 170 | 372647.1 | 81 | 143 | forward 3 | SP |
| 170 | 372647.1 | 66 | 113 | forward 3 | TM |
| 170 | 372647.1 | 66 | 128 | forward 3 | TM |
| 171 | 148512.1 | 368 | 439 | forward 2 | SP |
| 171 | 148512.1 | 368 | 424 | forward 2 | TM |
| 171 | 148512.1 | 467 | 523 | forward 2 | TM |
| 171 | 148512.1 | 494 | 556 | forward 2 | TM |
| 171 | 148512.1 | 470 | 523 | forward 2 | TM |
| 171 | 148512.1 | 131 | 199 | forward 2 | TM |
| 173 | 2023119CD1 | 653 | 672 | | TM |
| 173 | 2023119CD1 | 23 | 50 | | TM |
| 173 | 2023119CD1 | 167 | 185 | | TM |
| 173 | 2023119CD1 | 653 | 676 | | TM |
| 173 | 2023119CD1 | 562 | 587 | | TM |
| 173 | 2023119CD1 | 160 | 184 | | TM |
| 173 | 2023119CD1 | 24 | 38 | | SP |
| 173 | 2023119CD1 | 232 | 250 | | TM |
| 173 | 2023119CD1 | 24 | 41 | | SP |
| 173 | 2023119CD1 | 19 | 37 | | TM |
| 173 | 2023119CD1 | 22 | 40 | | TM |
| 173 | 2023119CD1 | 227 | 250 | | TM |
| 173 | 2023119CD1 | 160 | 182 | | SP |
| 175 | 1973832CD1 | 1 | 26 | | SP |
| 175 | 1973832CD1 | 1 | 18 | | SP |
| 175 | 1973832CD1 | 1 | 24 | | SP |
| 175 | 1973832CD1 | 1 | 20 | | SP |
| 175 | 1973832CD1 | 1 | 22 | | SP |
| 176 | 241888.54 | 251 | 328 | forward 2 | SP |
| 176 | 241888.54 | 1149 | 1223 | forward 3 | TM |
| 176 | 241888.54 | 1697 | 1759 | forward 2 | TM |
| 176 | 241888.54 | 251 | 304 | forward 2 | SP |
| 176 | 241888.54 | 251 | 322 | forward 2 | SP |
| 176 | 241888.54 | 251 | 310 | forward 2 | SP |
| 176 | 241888.54 | 251 | 316 | forward 2 | SP |
| 178 | 1736965CD1 | 1 | 16 | | TM |
| 178 | 1736965CD1 | 1 | 20 | | TM |
| 178 | 1736965CD1 | 1 | 16 | | SP |
| 178 | 1736965CD1 | 1 | 21 | | SP |
| 178 | 1736965CD1 | 1 | 23 | | SP |
| 178 | 1736965CD1 | 1 | 24 | | SP |
| 178 | 1736965CD1 | 1 | 18 | | SP |
| 179 | 412065.17 | 1614 | 1688 | forward 3 | SP |
| 180 | 988660.32 | 115 | 159 | forward 1 | SP |
| 180 | 988660.32 | 103 | 177 | forward 1 | SP |
| 180 | 988660.32 | 795 | 857 | forward 3 | SP |
| 180 | 988660.32 | 103 | 159 | forward 1 | SP |
| 180 | 988660.32 | 103 | 165 | forward 1 | SP |
| 180 | 988660.32 | 103 | 177 | forward 1 | SP |
| 180 | 988660.32 | 103 | 171 | forward 1 | SP |
| 182 | 1434821CD1 | 4 | 21 | | TM |
| 182 | 1434821CD1 | 4 | 21 | | SP |
| 182 | 1434821CD1 | 1 | 26 | | SP |
| 182 | 1434821CD1 | 4 | 26 | | SP |
| 182 | 1434821CD1 | 4 | 24 | | SP |
| 182 | 1434821CD1 | 1 | 24 | | SP |
| 183 | 464689.64 | 638 | 712 | forward 2 | TM |
| 183 | 464689.64 | 4701 | 4760 | forward 3 | SP |
| 185 | 1384719.3 | 2975 | 3046 | forward 2 | TM |
| 185 | 1384719.3 | 3241 | 3312 | forward 1 | TM |
| 185 | 1384719.3 | 1086 | 1154 | forward 3 | SP |
| 185 | 1384719.3 | 2613 | 2702 | forward 3 | SP |
| 185 | 1384719.3 | 3351 | 3422 | forward 3 | TM |
| 185 | 1384719.3 | 3214 | 3273 | forward 1 | TM |
| 185 | 1384719.3 | 1086 | 1148 | forward 3 | SP |
| 185 | 1384719.3 | 2999 | 3052 | forward 2 | SP |
| 185 | 1384719.3 | 2637 | 2696 | forward 3 | TM |
| 185 | 1384719.3 | 3336 | 3410 | forward 3 | TM |
| 185 | 1384719.3 | 2014 | 2088 | forward 1 | TM |
| 185 | 1384719.3 | 2999 | 3046 | forward 2 | SP |
| 185 | 1384719.3 | 180 | 233 | forward 3 | TM |
| 185 | 1384719.3 | 3204 | 3263 | forward 3 | TM |
| 185 | 1384719.3 | 2978 | 3055 | forward 2 | TM |
| 185 | 1384719.3 | 3348 | 3410 | forward 3 | TM |
| 185 | 1384719.3 | 1086 | 1157 | forward 3 | SP |
| 185 | 1384719.3 | 3366 | 3419 | forward 3 | TM |
| 185 | 1384719.3 | 3360 | 3419 | forward 3 | TM |
| 186 | 407463.1 | 3238 | 3321 | forward 1 | SP |
| 186 | 407463.1 | 4943 | 4999 | forward 2 | TM |
| 186 | 407463.1 | 2108 | 2179 | forward 2 | SP |
| 186 | 407463.1 | 2114 | 2185 | forward 2 | TM |
| 186 | 407463.1 | 1807 | 1860 | forward 1 | SP |
| 186 | 407463.1 | 2605 | 2652 | forward 1 | SP |
| 186 | 407463.1 | 2144 | 2206 | forward 2 | TM |
| 186 | 407463.1 | 3238 | 3309 | forward 1 | SP |
| 186 | 407463.1 | 4925 | 4975 | forward 2 | TM |
| 188 | 522433CD1 | 14 | 31 | | SP |
| 188 | 522433CD1 | 14 | 33 | | SP |
| 188 | 522433CD1 | 6 | 31 | | SP |
| 188 | 522433CD1 | 1 | 29 | | SP |
| 189 | 480489.5 | 1494 | 1559 | forward 3 | TM |
| 189 | 480489.5 | 17 | 73 | forward 2 | SP |
| 189 | 480489.5 | 17 | 85 | forward 2 | SP |
| 189 | 480489.5 | 1174 | 1236 | forward 1 | SP |
| 189 | 480489.5 | 1482 | 1553 | forward 3 | TM |
| 189 | 480489.5 | 17 | 79 | forward 2 | SP |
| 189 | 480489.5 | 17 | 88 | forward 2 | SP |
| 189 | 480489.5 | 1497 | 1559 | forward 3 | TM |
| 189 | 480489.5 | 1150 | 1239 | forward 1 | SP |
| 190 | 480489.2 | 184 | 246 | forward 1 | SP |
| 190 | 480489.2 | 406 | 471 | forward 1 | TM |
| 190 | 480489.2 | 184 | 234 | forward 1 | SP |
| 190 | 480489.2 | 394 | 465 | forward 1 | TM |
| 190 | 480489.2 | 409 | 471 | forward 1 | TM |
| 192 | 1737775CD1 | 1 | 25 | | SP |
| 192 | 1737775CD1 | 1 | 23 | | SP |
| 192 | 1737775CD1 | 1 | 21 | | SP |
| 194 | 088078CD1 | 8 | 28 | | TM |
| 194 | 088078CD1 | 1 | 21 | | SP |
| 194 | 088078CD1 | 493 | 514 | | TM |
| 194 | 088078CD1 | 1 | 23 | | SP |
| 194 | 088078CD1 | 1 | 19 | | SP |
| 194 | 088078CD1 | 494 | 514 | | TM |

What is claimed is:

1. A combination comprising a plurality of cDNAs that are differentially expressed in a colon disorder and selected from SEQ ID NOs:1-3, 5, 6, 8-10,12, 14, 15, 17, 18, 20, 22, 24, 26-29, 31, 33, 34, 36-39, 41-43, 45-47, 49, 51, 53. 55-58, 60, 62, 64, 66, 67, 69, 71, 72, 74-79, 81, 83-86, 88, 89, 91, 92, 94, 96, 97, 99, 100, 102-104, 106, 107, 109, 111, 112, 114, 116, 118, 119, 121, 123-126, 128, 130, 131-137, 139, 140, 142-151, 153-157, 159, 160, 162-165, 167-172, 174, 176, 177, 179-181, 183-187, 189-191, and 193 or their complements.

2. The combination of claim 1 selected from SEQ ID NOs:172, 174, 176, 177, 179-181, 183-187, 189-191, and 193, wherein the disorder is a colon cancer.

3. The combination of claim 1, wherein the cDNAs are immobilized on a substrate.

4. A high throughput method for detecting differential expression of one or more cDNAs in a sample containing nucleic acids, the method comprising:
(a) hybridizing the substrate of claim 3 with nucleic acids of the sample, thereby forming one or more hybridization complexes;
(b) detecting the hybridization complexes; and
(c) comparing the hybridization complexes with those of a standard, wherein differences between the standard and sample hybridization complexes indicate differential expression of cDNAs in the sample.

5. The method of claim 4, wherein the nucleic acids of the sample are amplified prior to hybridization.

6. The method of claim 4, wherein the sample is from a subject with a colon cancer and comparison with a standard defines an early, mid, or late stage of that disease.

7. A high throughput method of screening a plurality of molecules or compounds to identify a ligand which specifically binds a cDNA, the method comprising:
(a) combining the combination of claim 1 with the plurality of molecules or compounds under conditions to allow specific binding; and
(b) detecting specific binding between each cDNA and at least one molecule or compound, thereby identifying a ligand that specifically binds to each cDNA.

8. The method of claim 10 wherein the plurality of molecules or compounds are selected from DNA molecules, RNA molecules, peptide nucleic acid molecules, mimetics, peptides, transcription factors, repressors, and regulatory proteins.

9. An isolated cDNA selected from SEQ ID NOs:12, 41, 71, 74, 154,162, 167, 170, and 177.

10. A vector containing the cDNA of claim 9.

11. A host cell containing the vector of claim 10.

12. A method for producing a protein, the method comprising the steps of:
(a) culturing the host cell of claim 11 under conditions for expression of protein; and
(b) recovering the protein from the host cell culture.

13. A protein or a portion thereof produced by the method of claim 12.

14. The protein of claim 13 selected from SEQ ID NOs:13 and 178.

15. A high-throughput method for using a protein to screen a plurality of molecules or compounds to identify at least one ligand which specifically binds the protein, the method comprising:
(a) combining the protein of claim 13 with the plurality of molecules or compounds under conditions to allow specific binding; and
(b) detecting specific binding between the protein and a molecule or compound, thereby identifying a ligand which specifically binds the protein.

16. The method of claim 15 wherein the plurality of molecules or compounds is selected from DNA molecules, RNA molecules, peptide nucleic acid molecules, mimetics, peptides, proteins, agonists, antagonists, antibodies or their fragments, immunoglobulins, inhibitors, drug compounds, and pharmaceutical agents.

17. An antibody which specifically binds the protein produced by the method of claim 19.

18. A method of using a protein to produce a polyclonal antibody, the method comprising:
a) immunizing an animal with the protein of claim 13 under conditions to elicit an antibody response;
b) isolating animal antibodies; and
c) combining the isolated antibodies with the protein under conditions to form an antibody:protein complex; and
d) dissociating the protein from the complex, thereby obtaining purified antibody.

19. A method of using a protein to prepare a monoclonal antibody comprising:
a) immunizing a animal with a protein of claim 13 under conditions to elicit an antibody response;
b) isolating antibody producing cells from the animal;
c) fusing the antibody producing cells with immortalized cells in culture to form monoclonal antibody producing hybridoma cells;
d) culturing the hybridoma cells; and
e) isolating from culture monoclonal antibodies which specifically bind the protein.

20. A method for using an antibody to detect expression of a protein in a sample, the method comprising:
a) combining the antibody of claim 17 with a sample under conditions which allow the formation of antibody:protein complexes; and
b) detecting complex formation, wherein complex formation indicates expression of the protein in the sample.
